(19) United States
(12) Patent Application Publication
(10) Pub. No.: US 2022/0352474 A1
(43) Pub. Date: Nov. 3, 2022

(54) ORGANIC ELECTROLUMINESCENT DEVICE

(71) Applicant: ROHM AND HAAS ELECTRONIC MATERIALS KOREA LTD., CHUNGCHEONGNAM-DO (KR)

(72) Inventors: Dong-Hyung Lee, Gyeonggi-do (KR); Chi-Sik KIM, Gyeonggi-do (KR); Yoo-Jin DOH, Gyeonggi-do (KR); Seung-Hoon YOO, Gyeonggi-do (KR); Soo-Yong LEE, Gyeonggi-do (KR); Hyun-Woo KANG, Gyeonggi-do (KR)

(21) Appl. No.: 17/231,835

(22) Filed: Apr. 15, 2021

(51) Int. Cl. H01L 51/00; C07D 307/91; C09K 11/06; C07D 491/048; C07D 251/24; C07D 413/10; C07D 491/06

(52) U.S. Cl. CPC H01L 51/0073; C07D 307/91; C09K 11/06; H01L 51/0052; H01L 51/0058; C07D 491/048; H01L 51/0071; H01L 51/0067; C07D 251/24; H01L 51/0054; C07D 413/10; C07D 491/06; H01L 51/5012

(57) ABSTRACT

The present disclosure relates to an organic electroluminescent device. The organic electroluminescent device of the present disclosure may exhibit low voltage, high efficiency, and/or long lifespan by including a light-emitting layer comprising a specific combination of the compounds, and an electron transport zone.

### TECHNICAL FIELD

The present disclosure relates to an organic electroluminescent device comprising a light-emitting layer and an electron transport zone.

### BACKGROUND ART

An electroluminescent device (EL device) is a self-light-emitting display device which has advantages in that it provides a wider viewing angle, a greater contrast ratio, and a faster response time. An organic electroluminescent device (OLED) was first developed by Eastman Kodak in 1987, by using small aromatic diamine molecules and aluminum complexes as materials for forming a light-emitting layer [Appl. Phys. Lett. 51, 913, 1987].

An OLED changes electric energy into light by applying electricity to an organic electroluminescent material, and commonly comprises an anode, a cathode, and an organic layer formed between the two electrodes. The organic layer of the OLED may comprise a hole injection layer, a hole transport layer, an electron blocking layer, a light-emitting layer (containing host and dopant materials), an electron buffer layer, a hole blocking layer, an electron transport layer, an electron injection layer, etc. The materials used in the organic layer can be classified into a hole injection material, a hole transport material, an electron blocking material, a light-emitting material, an electron buffer material, a hole blocking material, an electron transport material, an electron injection material, etc., depending on their functions. In such an OLED, holes from the anode and electrons from the cathode are injected into a light-emitting layer by the application of electric voltage, and excitons having high energy are produced by the recombination of the holes and electrons. The organic light-emitting compound moves into an excited state by the energy and emits light from energy when the organic light-emitting compound returns to the ground state from the excited state.

The most important factor determining luminous efficiency in an OLED is light-emitting materials. The light-emitting materials are required to have the following features: high quantum efficiency, high movement degree of an electron and a hole, and uniformity and stability of the formed light-emitting material layer. The light-emitting material is classified into blue, green, and red light-emitting materials according to the light-emitting color, and further includes yellow or orange light-emitting materials. Furthermore, the light-emitting material is classified into a host material and a dopant material in a functional aspect. Recently, an urgent task is the development of an OLED having high efficiency and long lifespan. In particular, the development of highly excellent light-emitting material over conventional light-emitting materials is urgently required, considering the EL properties necessary for medium and large-sized OLED panels.

An electron transport material in the OLED smoothly moves electrons from the cathode to the light-emitting layer and inhibits the movement of holes that could not be combined in the light-emitting layer, thereby increasing the chance of recombination between holes and electrons in the light-emitting layer. Materials having an excellent electron affinity are used as the electron transport material. When used in an OLED due to their high electron affinities, a new electron transport material is required that exhibits fast electron movement properties so that the light emitting device can exhibit high luminous efficiency.

In addition, the electron buffer layer is a layer for improving a problem in which a decrease in light-emitting luminance may occur due to a change in current properties in a device when exposed to high temperatures in a panel manufacturing process. The properties of a compound included in the electron buffer layer are important. Further, the compound used in the electron buffer layer preferably plays a role of controlling electron injection according to the electron withdrawing property and the electron affinity LUMO (lowest unoccupied molecular orbital) energy value. Through this, it can play a role of improving the efficiency and lifespan of the OLED.

Korean Patent Application Laid-Open No. 2019-0042791 A and Korean Patent Application Laid-Open No. 2017-0130434 A disclose an OLED that combines a host based on anthracene with a dopant having a molecular structure connecting a heteroatom and an aromatic ring. However, said references do not specifically disclose an OLED including a combination of organic electroluminescent compounds specified herein.

### DISCLOSURE OF THE INVENTION

### Technical Problem

The object of the present disclosure is to provide an organic electroluminescent device having low voltage, high efficiency, and/or long lifespan by including a light-emitting layer comprising a specific combination of the compounds, and an electron transport zone.

### Solution to Problem

As a result of intensive studies to solve the technical problem above, the present inventors found that the aforementioned objective can be achieved by an organic electroluminescent device comprising a first electrode; a second electrode facing the first electrode; a light-emitting layer between the first electrode and the second electrode; and an electron transport zone between the light-emitting layer and the second electrode, wherein the electron transport zone includes a hole blocking layer, an electron buffer layer, or both of them, and an electron transport layer and an electron injection layer, wherein the electron transport layer is formed between the hole blocking layer or the electron buffer layer and the second electrode, wherein the electron injection layer is formed between the electron transport layer and the second electrode, wherein the light-emitting layer comprises a compound represented by the following formula 1 and a compound represented by the following formula 2, and wherein the hole blocking layer, the electron buffer layer, or both of them comprise(s) a compound represented by the following formula 11, so that the present invention was completed.

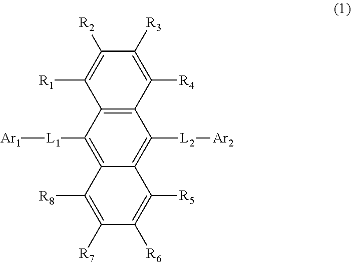

In formula 1,

L₁ and L₂ each independently represent a single bond, a substituted or unsubstituted (C6-C30)arylene or a substituted or unsubstituted (5- to 30-membered)heteroarylene:

Ar₁ and Ar₂ each independently represent a substituted or unsubstituted (C6-C30)aryl, or a substituted or unsubstituted (5- to 30-membered)heteroaryl; and

R₁ to R₈ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N-(Ar₄)(Ar₅);

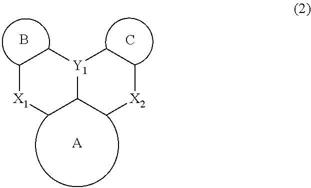

in formula 2,

Ring A, Ring B and Ring C each independently represent a substituted or unsubstituted (C6-C30)aryl or a substituted or unsubstituted (3- to 50-membered)heteroaryl;

Y₁ represents B;

X₁ and X₂ each independently represent NR; and

R represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N-(Ar₄)(Ar₅); or may be linked to at least one of the Ring A, the Ring B, and the Ring C to form a ring(s):

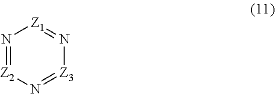

in formula 11,

Z₁ to Z₃ each independently represent CR₁₈;

R₁₈ each independently represents hydrogen, deuterium, halogen, cyano, a substituted or unsubstituted (C1-C30)alkyl, a substituted or unsubstituted (C6-C50)aryl, a substituted or unsubstituted (3- to 50-membered)heteroaryl, a substituted or unsubstituted (C3-C30)cycloalkyl, a substituted or unsubstituted (3- to 7-membered)heterocycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N-(Ar₄)(Ar₅), provided that R₁₈ does not include 2-dimethylfluorenyl, quinolinyl, and pyridyl;

L₄ each independently represents a single bond, a substituted or unsubstituted (C6-C30)arylene, or a substituted or unsubstituted (3- to 30-membered)heteroarylene:

Ar₄ and Ar₅ each independently represent hydrogen, a substituted or unsubstituted (C1-C30)alkyl, a substituted or unsubstituted (C2-C30)alkenyl, a substituted or unsubstituted fused ring of an (C3-C30) aliphatic ring and a (C6-C30) aromatic ring, a substituted or unsubstituted (C6-C30)aryl, or a substituted or unsubstituted (3- to 30-membered)heteroaryl; and when a plurality of substituents represented by the same symbol are present, each of substituents represented by the same symbol may be the same or different.

### Advantageous Effects of Invention

According to the present disclosure, an organic electroluminescent device having low voltage, high efficiency and/or long lifespan is provided, and a display device or a lighting device using the same can be manufactured.

### MODE FOR THE INVENTION

Hereinafter, the present disclosure will be described in detail. However, the following description is intended to explain the invention, and is not meant in any way to restrict the scope of the invention.

Herein, “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, an electron injection material, etc.

The organic electroluminescent device of the present disclosure includes a first electrode; a second electrode facing the first electrode; and a light-emitting layer between the first and second electrodes; and may include a hole transport zone between the first electrode and the light-emitting layer and may include an electron transport zone between the light-emitting layer and the second electrode. One of the first electrode and the second electrode may be an anode and the other may be a cathode.

The hole transport zone refers to a zone in which holes move between the first electrode and the light-emitting layer. The hole transport zone may include, for example, at least one of a hole injection layer, a hole transport layer, a hole auxiliary layer, a light-emitting auxiliary layer, and an electron blocking layer. Each of the hole injection layer, the hole transport layer, the hole auxiliary layer, the light-emitting auxiliary layer, and the electron blocking layer can be a single layer or a mufti-layer of which two or more layers are stacked.

In addition, the hole transport zone may include a p-doped hole injection layer, a hole transport layer, and a light-emitting auxiliary layer. Herein, the p-doped hole injection layer means a hole injection layer doped with a p-dopant. The p-dopant is a material that has p-semiconductor properties. The p-semiconductor property refers to the property of injecting or transporting holes at the highest occupied molecular orbital (HOMO) energy level, i.e., refers to the property of a material with high hole conductivity.

The light-emitting layer is a layer from which light is emitted, and can be a single layer or a multi-layer of which two or more layers are stacked. In the light-emitting layer, the doping concentration of the dopant compound based on the host compound may be preferably less than 20 wt %.

The electron transport zone means a zone where electrons move between the light-emitting layer and the second electrode. For example, the electron transport zone may include at least one of a hole blocking layer, an electron transport layer, an electron buffer layer, and an electron injection layer. Each of the hole blocking layer, the electron transport layer, the electron buffer layer, and the electron injection layer can be a single layer or a multi-layer of which two or more layers are stacked.

According to one embodiment of the present disclosure, the light-emitting layer comprises a compound represented by formula 1 above and a compound represented by formula 2 above. The electron transport zone may be include a hole blocking layer, an electron buffer layer, or both of them, and an electron transport layer and an electron injection layer. The compound represented by formula 11 above is included in the hole blocking layer, the electron buffer layer, or both of them. The hole blocking layer, the electron buffer layer, or both of them are formed between the light-emitting layer and the second electrode. The electron transport layer is formed between the hole blocking layer or the electron buffer layer and the second electrode, and the electron injection layer is formed between the electron transport layer and the second electrode.

The compounds represented by formulas 1, 2, and 11 above will be described below in more detail.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, and tert-butyl, etc. Herein, the term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. Herein, the term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. Herein, the term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. Herein, the term “(3- to 7-membered)heterocycloalkyl” is a cycloalkyl having 3 to 7 ring backbone atoms, preferably 5 to 7 ring backbone atoms and at least one heteroatom selected from the group consisting of B, N, O, S, Si, and P, preferably O, S, and N, and includes tetrahydrofuran, pyrrolidine, thiolan, tetrahydropyran, etc. Herein, the term “(C6-C30)aryl(ene) or (C6-C50)aryl(ene)” is a monocyclic or fused ring radical derived from an aromatic hydrocarbon having 6 to 30 or 6 to 50 ring backbone carbon atoms, in which the number of the ring backbone carbon atoms is preferably 6 to 25, more preferably 6 to 18, and may be partially saturated. The aryl may comprise a spiro structure. Examples of the aryl specifically include phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, etc. More specifically, the aryl may be phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl etc. Herein, the term “(3- to 30-membered)heteroaryl(ene) or (3- to 50-membered)heteroaryl(ene)” is an aryl having 3 to 30 or 3 to 50 ring backbone atoms, in which the number of ring backbone atoms is preferably 3 to 30, more preferably 5 to 20, including at least one, preferably 1 to 4 heteroatoms selected from the group consisting of B, N, O, S, Si, P, Se, and Ge. The above heteroaryl or heteroarylene may be a monocyclic ring, or a fused ring condensed with at least one benzene ring; and may be partially saturated. Also, the above heteroaryl or heteroarylene herein may be one formed by linking at least one heteroaryl or aryl group to a heteroaryl group via a single bond(s) and may include a spiro structure. Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, naphthobenzofuranyl, naphthobenzothiophenyl, benzoimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically, the heteroaryl may be 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazoly-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acrydinyl, 2-acrydinyl, 3-acrydinyl, 4-acrydinyl, 9-acrydinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc. Herein, the term “a fused ring of an (C3-C30) aliphatic ring and an (C6-C30) aromatic ring” means a ring formed by fusing at least one aliphatic ring having 3 to 30 ring backbone carbon atoms in which the carbon atoms number is preferably 3 to 25, more preferably 3 to 18, and at least one aromatic ring having 6 to 30 ring backbone carbon atoms in which the carbon atoms number is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of an (C3-C30) aliphatic ring and an (C6-C30) aromatic ring may be replaced with at least one heteroatoms selected from B, N, O, S, Si and P, preferably at least one heteroatoms selected from N, O and S. Herein, the term “halogen” includes F, Cl, Br, and I.

In addition, “ortho (o),” “meta (m),” and “para (p)” are meant to signify the substitution position of all substituents. Ortho position is a compound with substituents, which are adjacent to each other, e.g., at the 1 and 2 positions on benzene. Meta position is the next substitution position of the immediately adjacent substitution position, e.g., a compound with substituents at the 1 and 3 positions on benzene. Para position is the next substitution position of the meta position, e.g., a compound with substituents at the 1 and 4 positions on benzene.

In addition, “substituted” in the expression “substituted or unsubstituted” means that a hydrogen atom in a certain functional group is replaced with another atom or functional group, i.e., a substituent. The substituents of the substituted alkyl, the substituted alkenyl, the substituted aryl, the substituted arylene, the substituted heteroaryl, the substituted heteroarylene, the substituted cycloalkyl, the substituted alkoxy, the substituted heterocycloalkyl, the substituted trialkylsilyl, the substituted dialkylarylsilyl, the substituted alkyldiarylsilyl, the substituted triarylsilyl and substituted fused ring of aliphatic ring and aromatic ring in formulas of the present disclosure, each independently represent at least one selected from the group consisting of deuterium; halogen; cyano; carboxyl; nitro; hydroxy; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cyclo alkenyl; (3- to 7-membered)heterocycloalkyl; (C6-C30)aryloxy; (C6-C30)arylthio; (5- to 50-membered)heteroaryl unsubstituted or substituted with at least one of (C1-C30)alkyl, (C6-C30)aryl and di(C6-C30)arylamino; (C6-C30)aryl unsubstituted or substituted with at least one of (C1-C30)alkyl, (3- to 50-membered)heteroaryl, and mono- or di-(C6-C30)arylamino; tri(C1-C30)alkylsilyl; tri(C6-C30)arylsilyl; di(C1-C30)alkyl(C6-C30)arylsilyl; (C1-C30)alkyldi(C6-C30)arylsilyl; amino; mono- or di-(C1-C30)alkylamino; mono- or di-(C2-C30)alkenylamino; mono- or di-(C6-C30)arylamino unsubstituted or substituted with at least one of (C1-C30)alkyl, (5- to 30-membered)heteroaryl and di(C6-C30)arylamino; mono- or di-(3- to 30-membered)heteroarylamino; (C1-C30)alkyl(C2-C30)alkenylamino; (C1-C30)alkyl(C6-C30)arylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl. According to one embodiment of the present disclosure, the substituents each independently represent at least one selected from the group consisting of deuterium; (C1-C20)alkyl; (5- to 40-membered)heteroaryl unsubstituted or substituted with at least one of deuterium, (C1-C20)alkyl and (C6-C25)aryl; (C6-C25)aryl unsubstituted or substituted with at least one of deuterium, (C1-C20)alkyl, (3- to 30-membered)heteroaryl and di(C6-C25)arylamino; and mono- or di-(C6-C25)arylamino unsubstituted or substituted with at least one of deuterium, (C1-C20)alkyl, (5- to 25-membered)heteroaryl and di(C6-C25)arylamino. For example, the substituents may be at least one of deuterium; methyl; tert-butyl; phenyl unsubstituted or substituted with at least one of methyl, pyridinyl, carbazolyl, dibenzofuranyl, diphenylamino, phenoxazinyl, phenothiazinyl, and acridinyl substituted with methyl; naphthyl; biphenyl; terphenyl; triphenylenyl; dimethylfluorenyl; phenylfluorenyl; diphenylfluorenyl; phenanthrenyl; pyridinyl; triazinyl substituted with at least one of phenyl and naphthyl; indolyl substituted with diphenyl; benzoimidazolyl substituted with phenyl; quinolyl; isoquinolyl; quinazolinyl substituted with phenyl; carbazolyl unsubstituted or substituted with phenyl; dibenzofuranyl; dibenzothiophenyl; benzocarbazolyl unsubstituted or substituted with phenyl; dibenzocarbazolyl; benzophenanthrothiophenyl; phenoxazinyl; phenothiazinyl; acridinyl substituted with at least one methyl; xanthenyl substituted with at least one methyl; diphenylamino unsubstituted or substituted with at least one of methyl and diphenylamino; dimethylfluorenylphenylamino; phenylnaphthylamino; and phenylamino substituted with phenylcarbazolyl or dibenzofuranyl; triphenylsilyl; or a substituted or unsubstituted (16- to 33-membered)heteroaryl containing at least one of N, O and S.

Herein, the term “a ring formed in linking to an adjacent substituent” in formulas of the present disclosure means a substituted or unsubstituted (3- to 30-membered) mono- or polycyclic, alicyclic, aromatic ring, or a combination thereof, formed by linking or fusing two or more adjacent substituents, Further, the formed ring may be included at least one heteroatom selected from the group consisting of B, N. O, S. Si and P, preferably at least one heteroatom selected from the group consisting of N, O and S. According to one embodiment of the present disclosure, the number of atoms in the ring skeleton is 5 to 20; according to another embodiment of the present disclosure, the number of atoms in the ring skeleton is 5 to 15.

In formulas of the present disclosure, heteroaryl, heteroarylene, and heterocycloalkyl each independently may contain at least one heteroatom selected from the group consisting of B, N, O, S, Si, and P. Further, the above heteroatom may be linked with at least one substituent selected from the group consisting of hydrogen, deuterium, halogen, cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

In formulas of the present disclosure, when a plurality of substituents represented by the same symbol are present, each of substituents represented by the same symbol may be the same or different.

The light-emitting material according to one embodiment comprises at least one anthracene derivative(s) represented by formula 1 above. In one embodiment, the compound represented by formula 1 above may be a fluorescent host, for example, a fluorescent host for blue light-emitting.

In formula 1 above, L₁ and L₂ each independently represent a single bond, a substituted or unsubstituted (C6-C30)arylene, or a substituted or unsubstituted (5- to 30-membered)heteroarylene. Preferably, L₁ and L₂ each independently may be a single bond, a substituted or unsubstituted (C6-C25)arylene, or a substituted or unsubstituted (5- to 25-membered)heteroarylene, more preferably, a single bond, a substituted or unsubstituted (C6-C18)arylene, or a substituted or unsubstituted (5- to 18-membered)heteroarylene.

In one embodiment, L₁ and L₂ each independently may be a single bond, or may be selected from the substituents listed in the following Group 1.

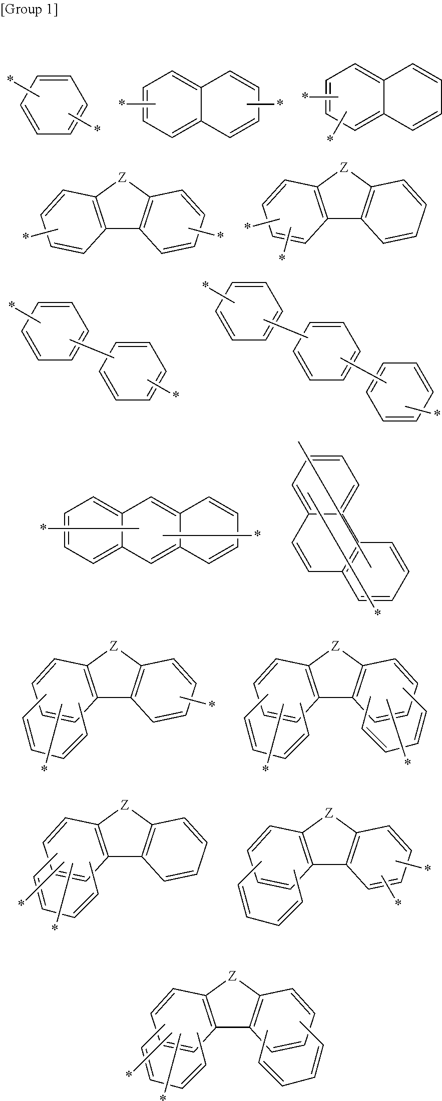

In the Group 1, Z may be O, S, NR₁₀₁, CR₁₀₂R₁₀₃, or SiR₁₀₄R₁₀₅, preferably O, S or NR₁₀₁.

In the Group 1, R₁₀₁ to R₁₀₅ each independently represent a substituted or unsubstituted (C1-C30)alkyl, a substituted or unsubstituted (C6-C30)aryl, or a substituted or unsubstituted (3- to 30-membered)heteroaryl; or may be linked to the adjacent substituent to form a ring(s); preferably, each independently may be a substituted or unsubstituted (C1-C10)alkyl, a substituted or unsubstituted (C6-C25)aryl, or a substituted or unsubstituted (5- to 25-membered)heteroaryl, more preferably, each independently may be a substituted or unsubstituted (C1-C4)alkyl, a substituted or unsubstituted (C6-C18)aryl, or a substituted or unsubstituted (5- to 18-membered)heteroaryl. For example, R₁₀₁ may be phenyl.

In any one of the Group 1, each of the two * represents a bonding site to the anthracene skeleton and a bonding site to Ar₁ or Ar₂.

In one embodiment, L₁ and L₂ each independently may be a single bond, phenylene, naphthylene, biphenylene, phenanthrenylene, benzofluorenylene substituted with at least one methyl, dibenzofuranylene, etc.

In formula 1 above, Ar₁ and Ar₂ each independently represent a substituted or unsubstituted (C6-C30)aryl or a substituted or unsubstituted (5- to 30-membered)heteroaryl, preferably may be a substituted or unsubstituted (C6-C25)aryl, or a substituted or unsubstituted (5- to 25-membered)heteroaryl, more preferably a substituted or unsubstituted (C6-C25)aryl, or a substituted or unsubstituted (5- to 20-membered)heteroaryl.

In one embodiment, Arₙ and Ar₂ each independently may be selected from the substituents listed in the following Group 2.

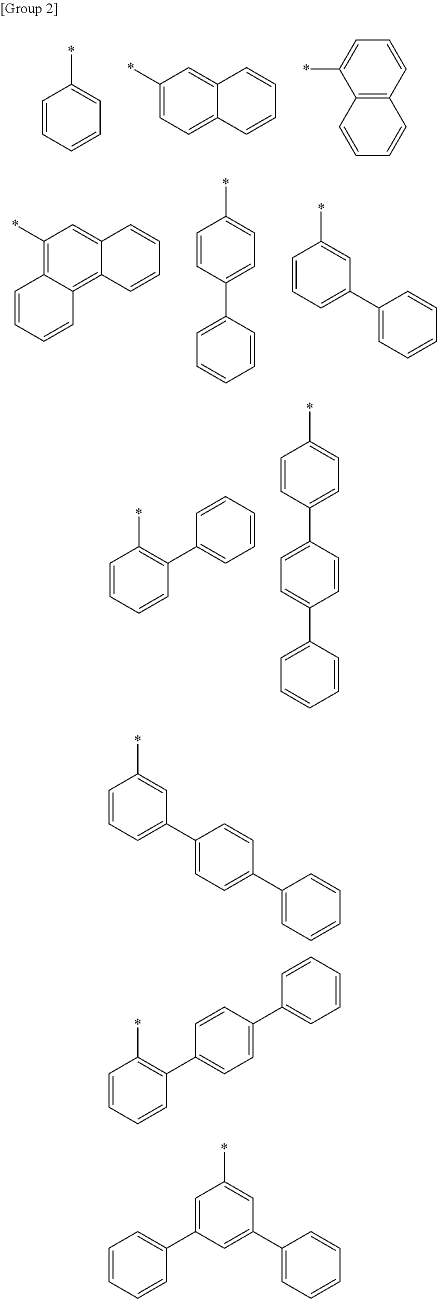

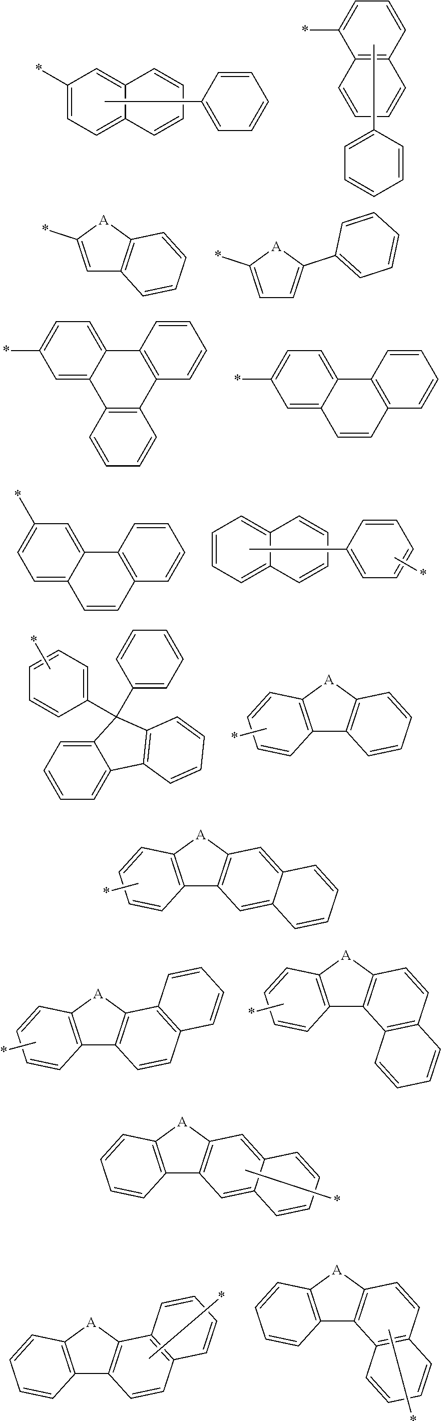

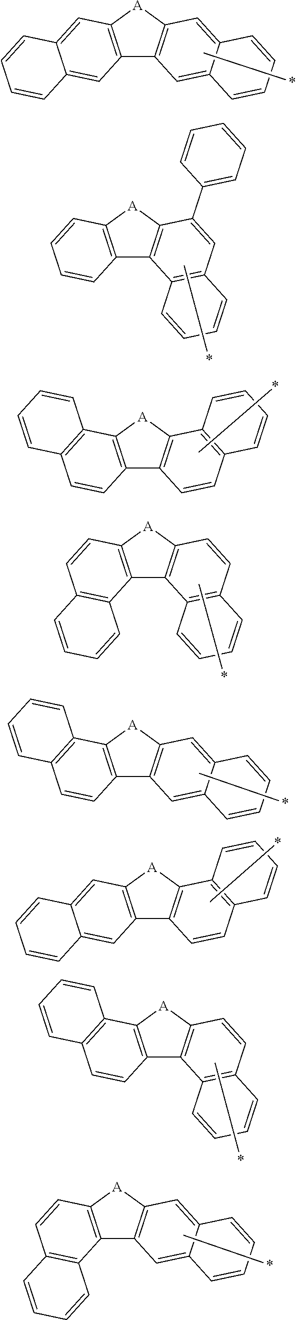

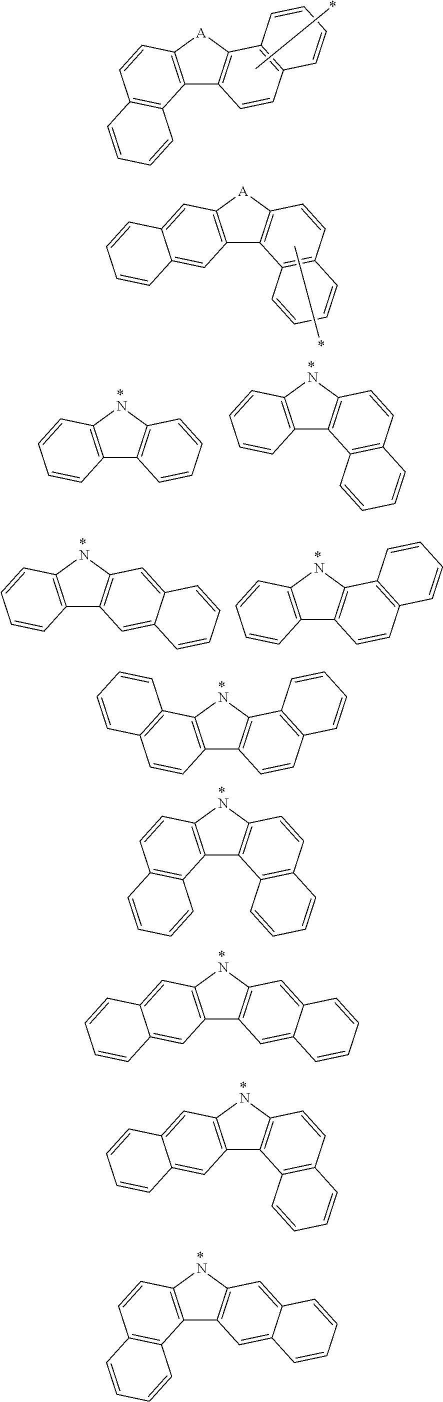

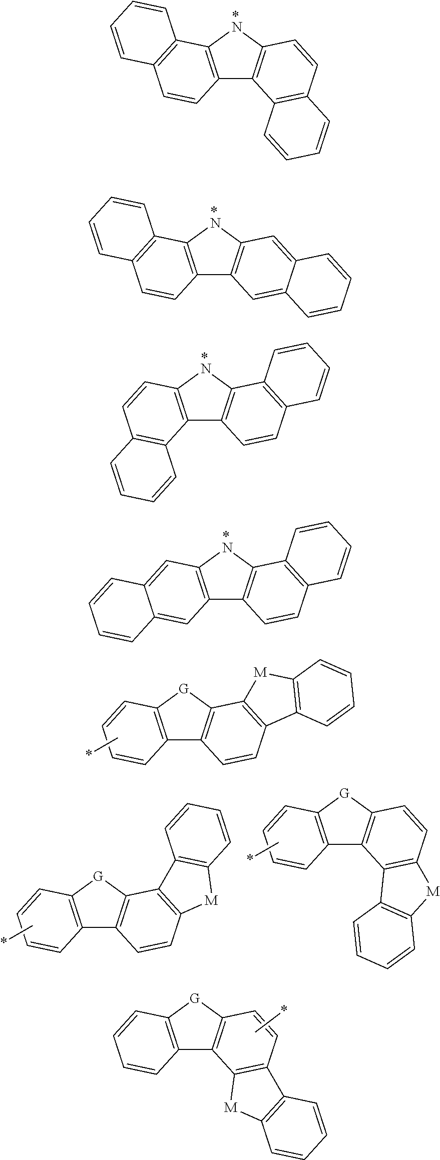

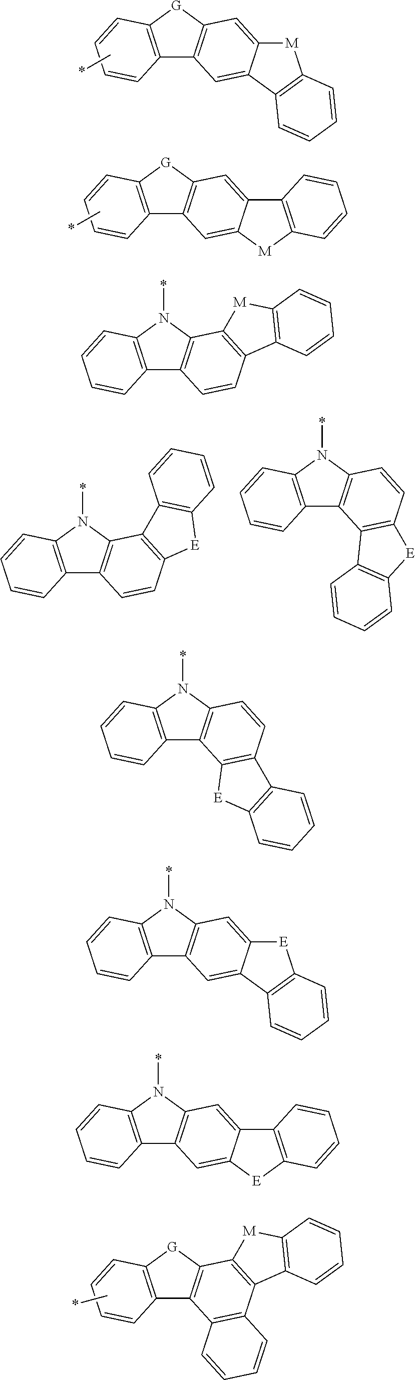

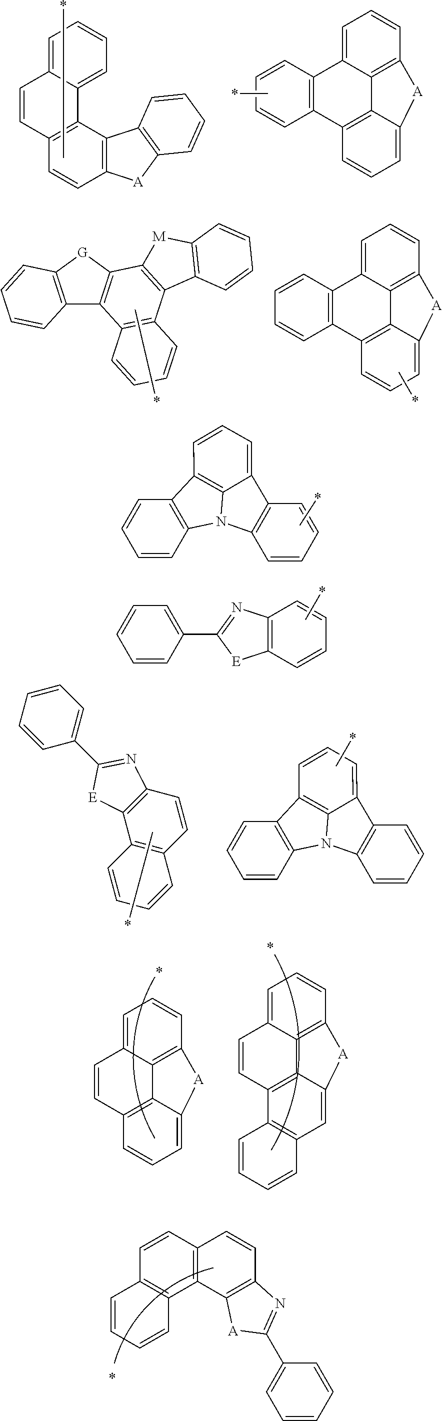

In the Group 2, A, G, E, and M each independently represent O, S, NR₁₀₆, CR₁₀₇R₁₀₈, or SiR₁₀₉R₁₁₀. For example, A may be O or CR₁₀₇R₁₀₈, E may be O or S, and each of G and M may be O, S or NR₁₀₈.

R₁₀₆ to R₁₁₀ each independently represent a substituted or unsubstituted (C1-C30)alkyl, a substituted or unsubstituted (C6-C30)aryl, or a substituted or unsubstituted (3- to 30-membered)heteroaryl; or may be linked to the adjacent substituent to form a ring(s), preferably each independently may be (C1-C10)alkyl, a substituted or unsubstituted (C6-C25)aryl, or a substituted or unsubstituted (5- to 25-membered)heteroaryl, more preferably, each independently may be unsubstituted (C1-C4)alkyl, unsubstituted (C6-C18)aryl, or unsubstituted (5- to 18-membered)heteroaryl. For example, R₁₀₆ to R₁₁₀ each independently may be unsubstituted methyl, unsubstituted naphthyl, or unsubstituted phenyl.

In the Group 2, * represents a bonding site to the anthracene skeleton, L₁, or L₂.

In one embodiment, Ar₁ and Ar₂ each independently may be a substituted or unsubstituted phenyl, a substituted or unsubstituted naphthyl, a substituted or unsubstituted m-biphenyl, a substituted or unsubstituted p-biphenyl, a substituted or unsubstituted terphenyl, a substituted or unsubstituted triphenylenyl, a substituted or unsubstituted phenanthrenyl, a substituted or unsubstituted epoxyphenanthrenyl, a substituted or unsubstituted fluorenyl, a substituted or unsubstituted benzofluorenyl, a substituted or unsubstituted spirobifluorenyl, a substituted or unsubstituted indenofluorenyl, a substituted or unsubstituted furanyl, a substituted or unsubstituted benzofuranyl, a substituted or unsubstituted dibenzofuranyl, a substituted or unsubstituted benzonaphthofuranyl, a substituted or unsubstituted dinaphthofuranyl, a substituted or unsubstituted anthrabenzofuranyl, a substituted or unsubstituted chrycenofuranyl, a substituted or unsubstituted dibenzothiophenyl, a substituted or unsubstituted benzonaphthothiophenyl, a substituted or unsubstituted benzocarbazolyl, a substituted or unsubstituted benzofurocarbazolyl, a substituted or unsubstituted benzothienocarbazolyl, a substituted or unsubstituted benzobisbenzofuranyl, a substituted or unsubstituted oxathiaindenofluorenyl, a substituted or unsubstituted dibenzocarbazolyl, a substituted or unsubstituted benzobisbenzothiophenyl, a substituted or unsubstituted carbazolyl, a substituted or unsubstituted indolocarbazolyl, a substituted or unsubstituted benzoxazolyl, a substituted or unsubstituted phenanthrooxazolyl, a substituted or unsubstituted benzothiazolyl, or a substituted or unsubstituted naphthothiazolyl.

In formula 1 above, R₁ to R₈ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N-(Ar₄)(Ar₅). Preferably, R₁ to R₈ each independently may be hydrogen, deuterium, halogen, cyano, a substituted or unsubstituted (C1-C10)alkyl, more preferably, hydrogen, deuterium, halogen, cyano, a substituted or unsubstituted (C1-C4)alkyl.

In one embodiment of the present disclosure, the compound represented by formula 1 above may be represented by the following formula 1-1.

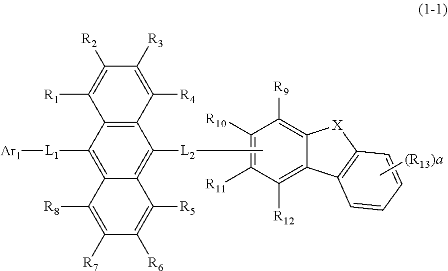

In formula 1-1,

X represents O or S;

R₉ to R₁₂ each independently represent hydrogen or deuterium, or a bonding site to L₂;

R₁₃ each independently represent hydrogen, deuterium, a substituted or unsubstituted (C6-C50)aryl, or a substituted or unsubstituted (3- to 50-membered)heteroaryl;

a represents an integer of 1 to 4, and when a is an integer of 2 or more, each of R₁₃ may be the same or different; and

L₁, L₂, Ar₁, and R₁ to R₈ are as defined in formula 1 above.

According to one embodiment, the compound represented by formula 1 above may be more specifically illustrated by the following compounds, but is not limited thereto.

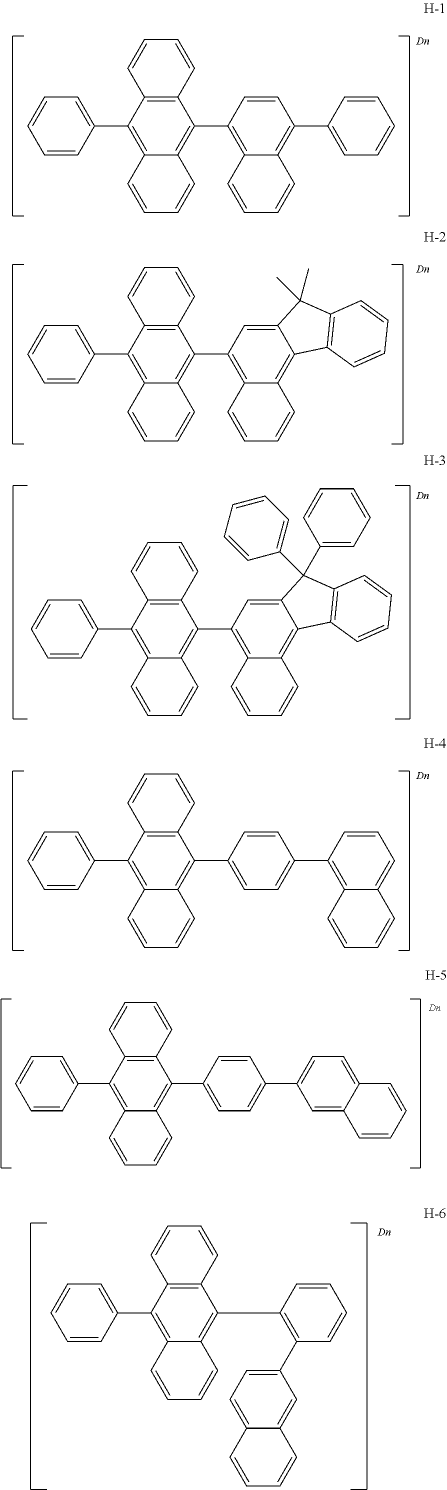

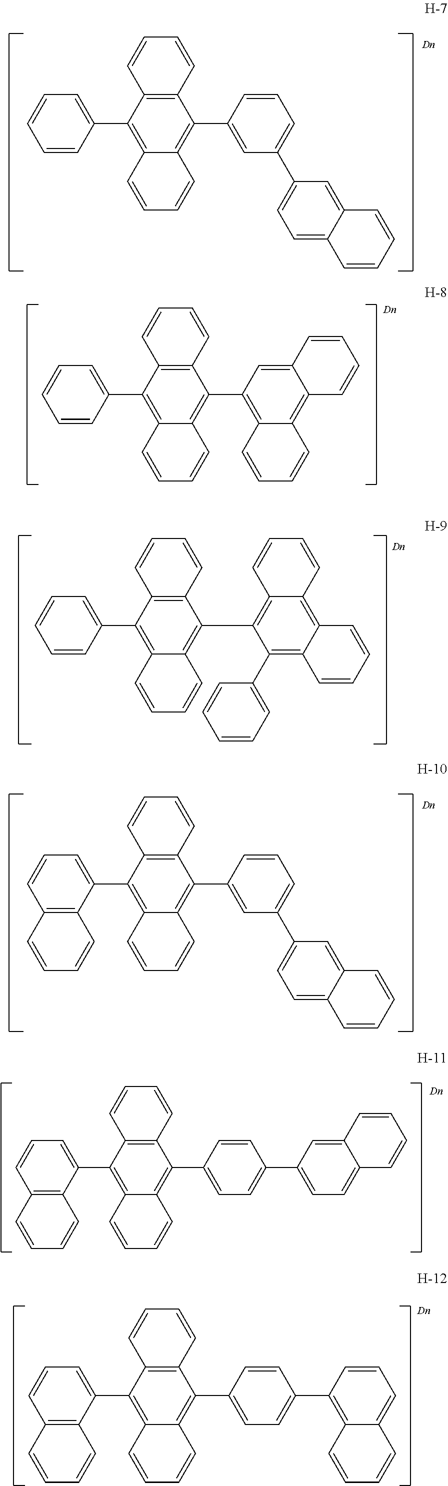

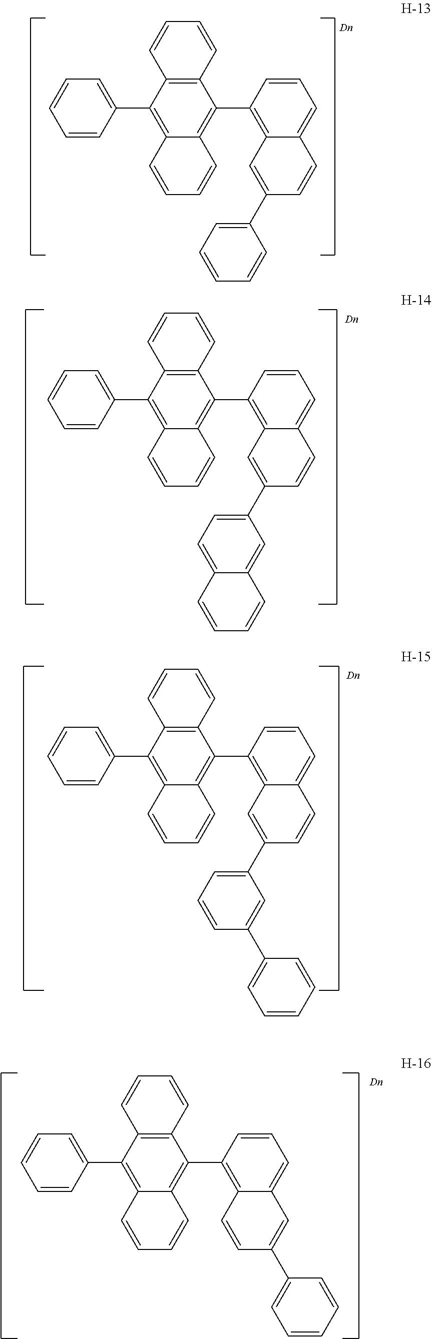

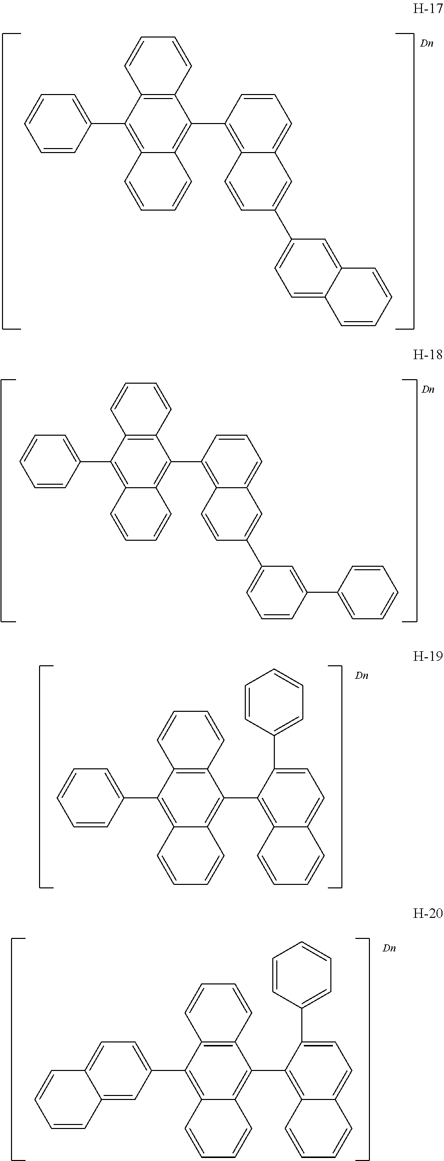

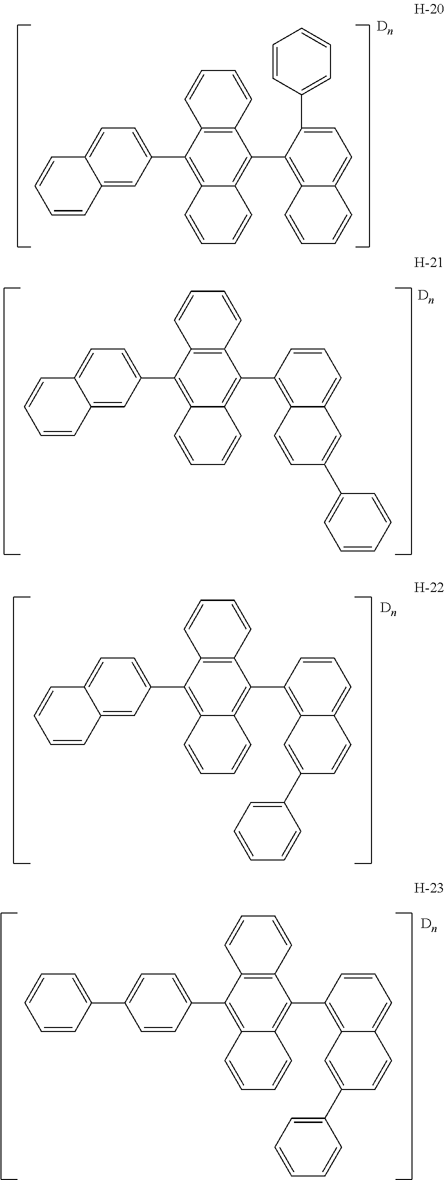

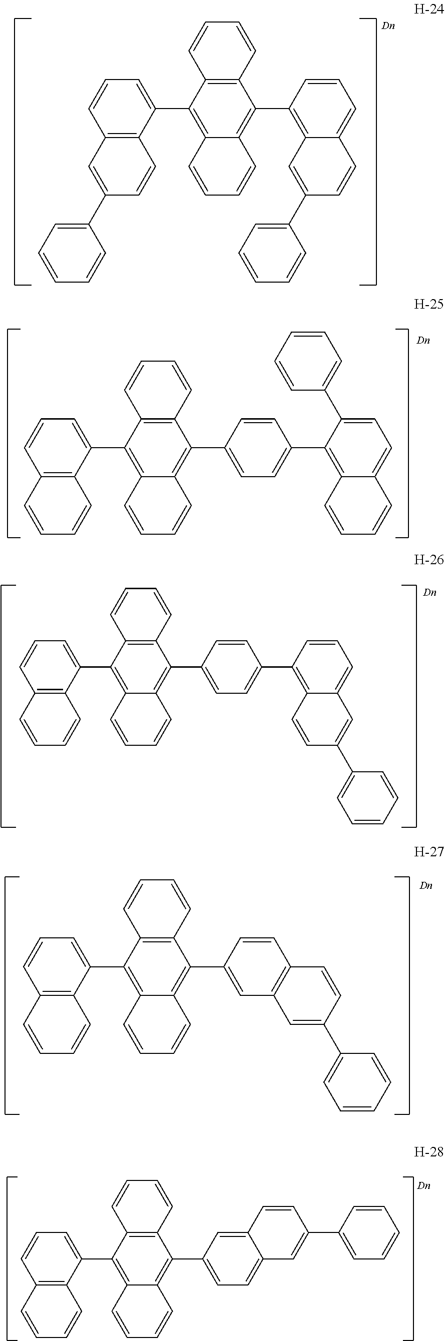

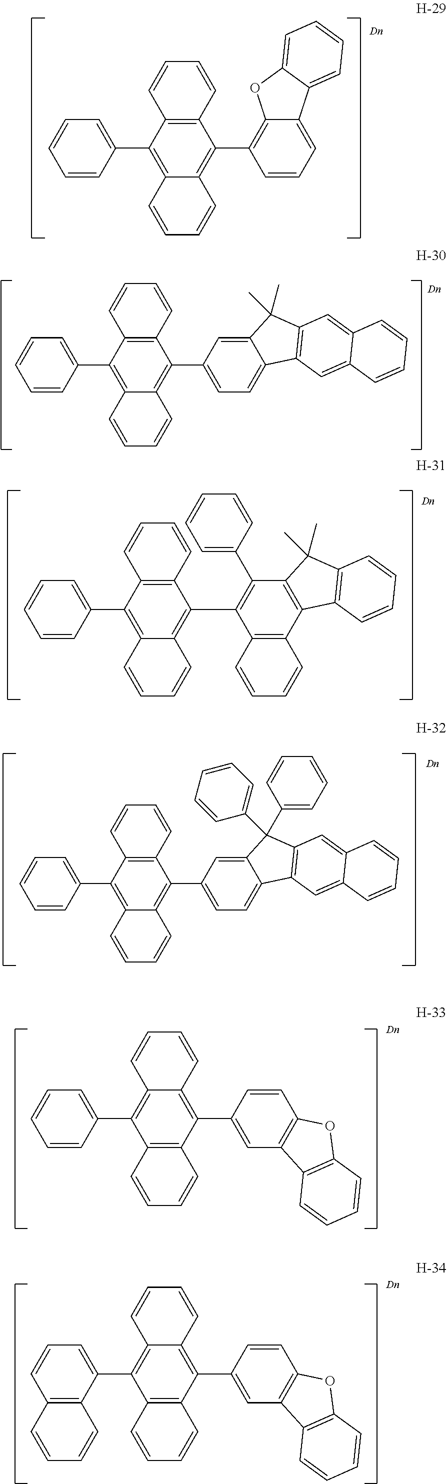

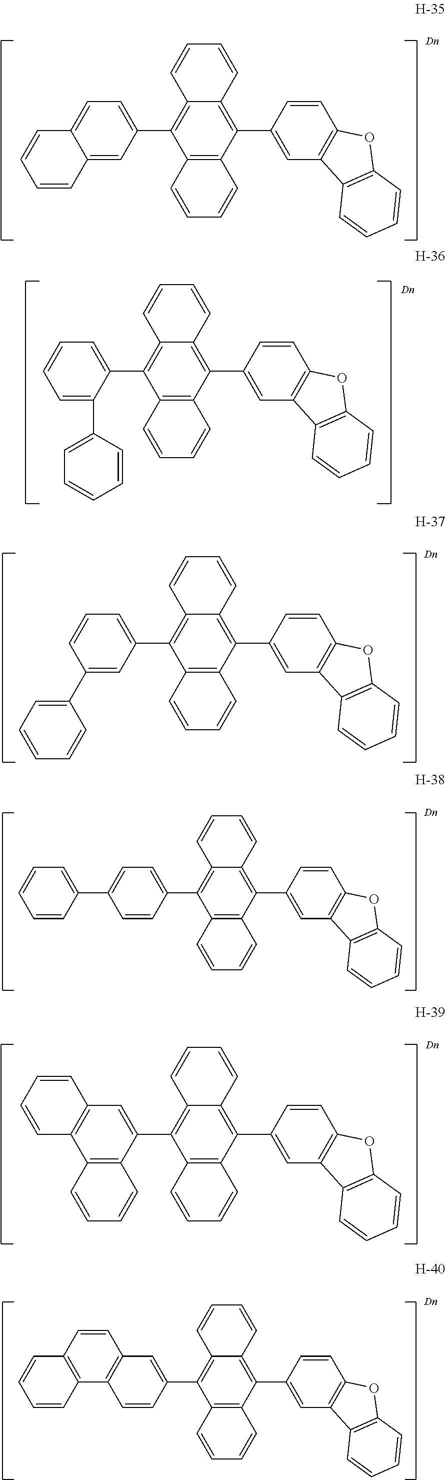

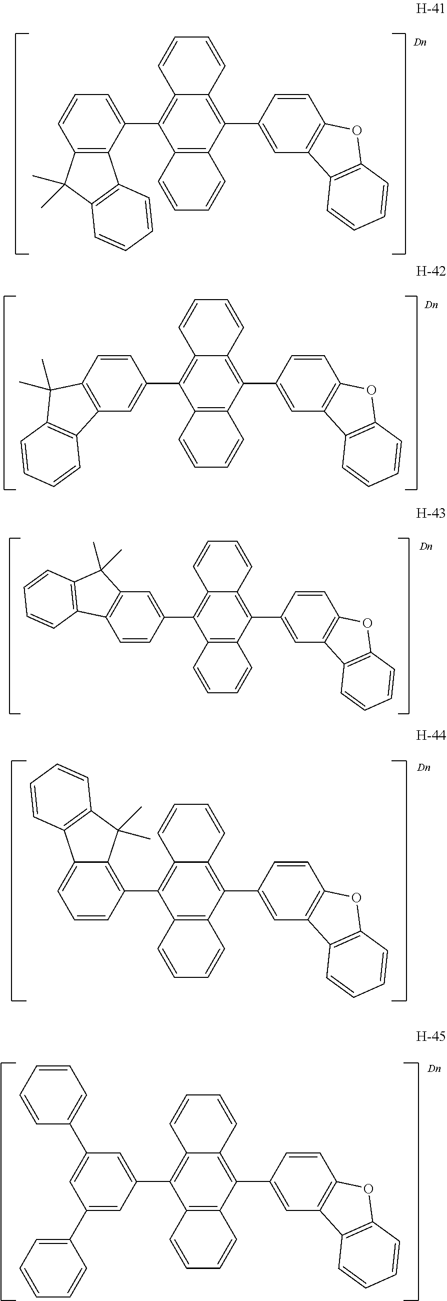

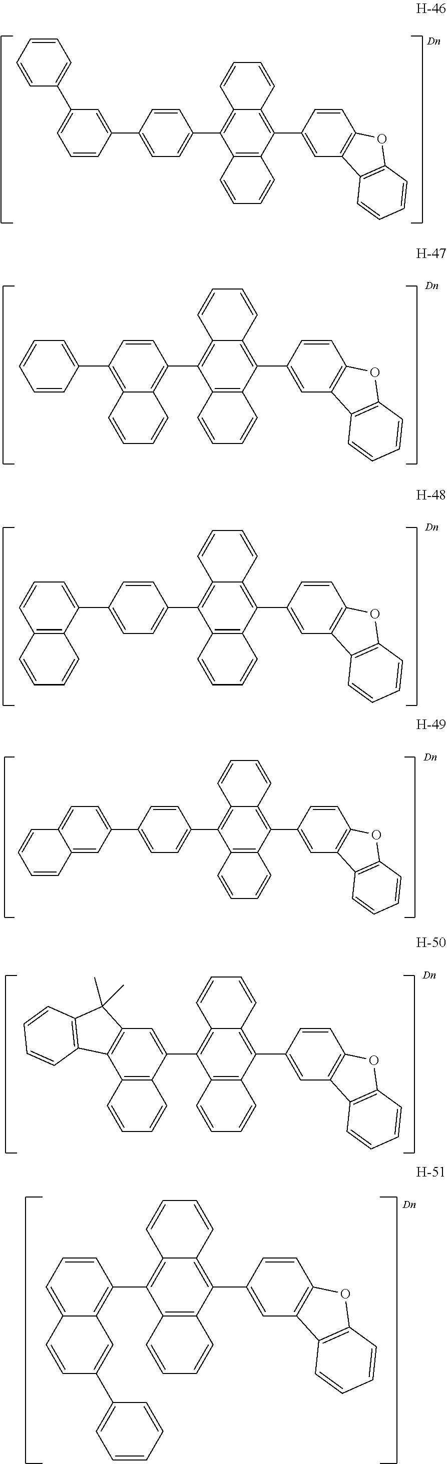

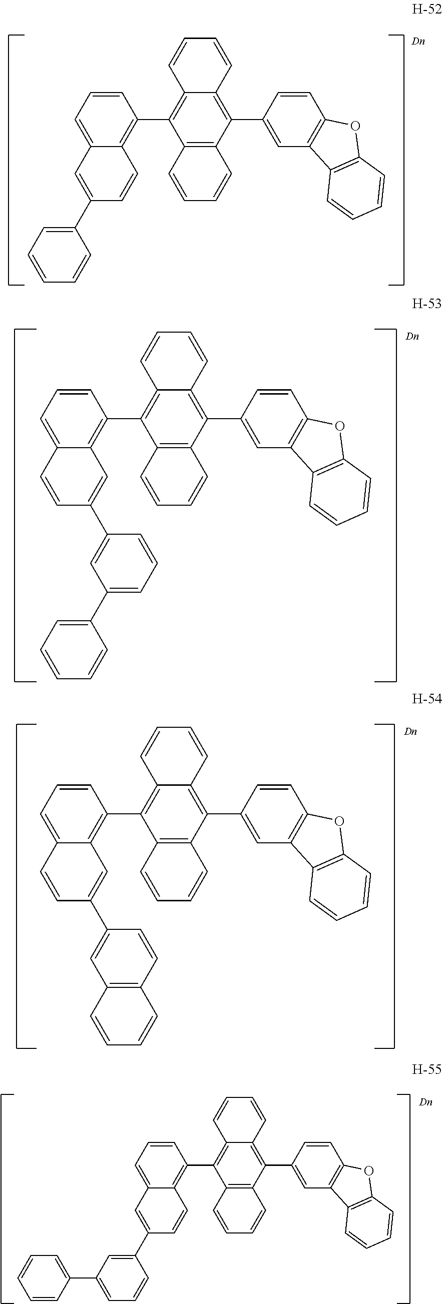

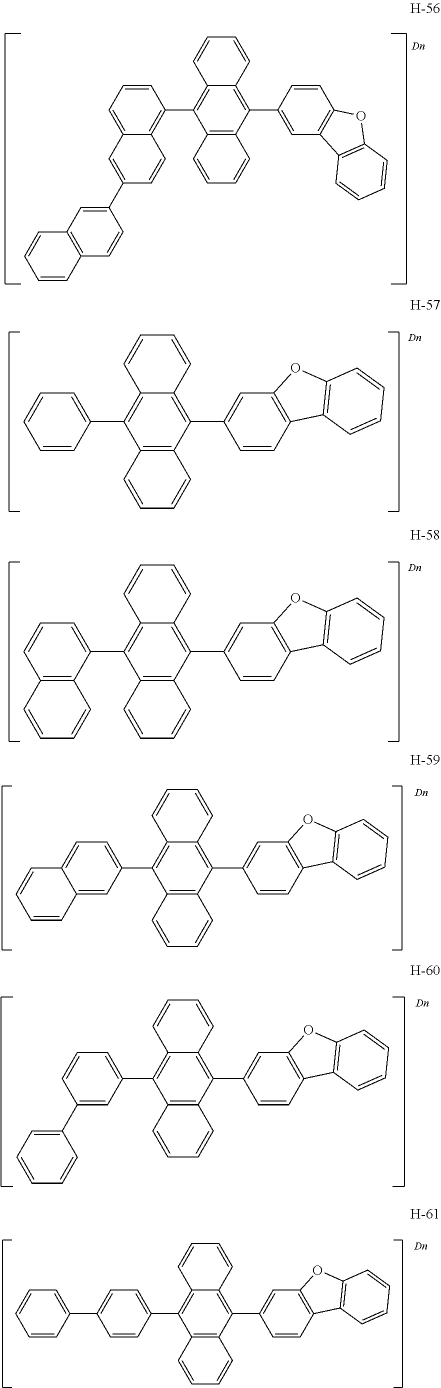

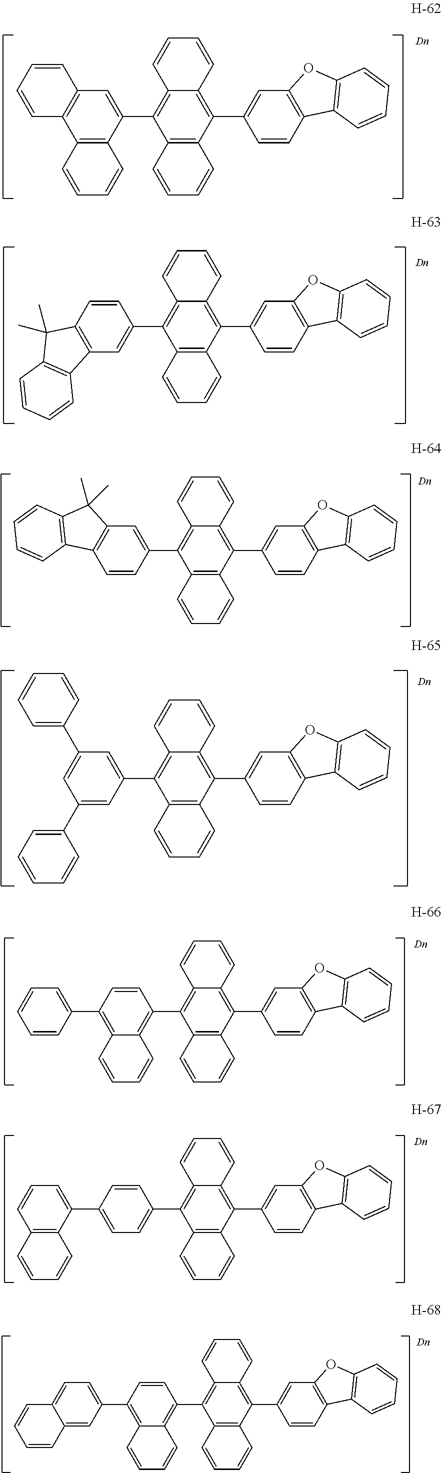

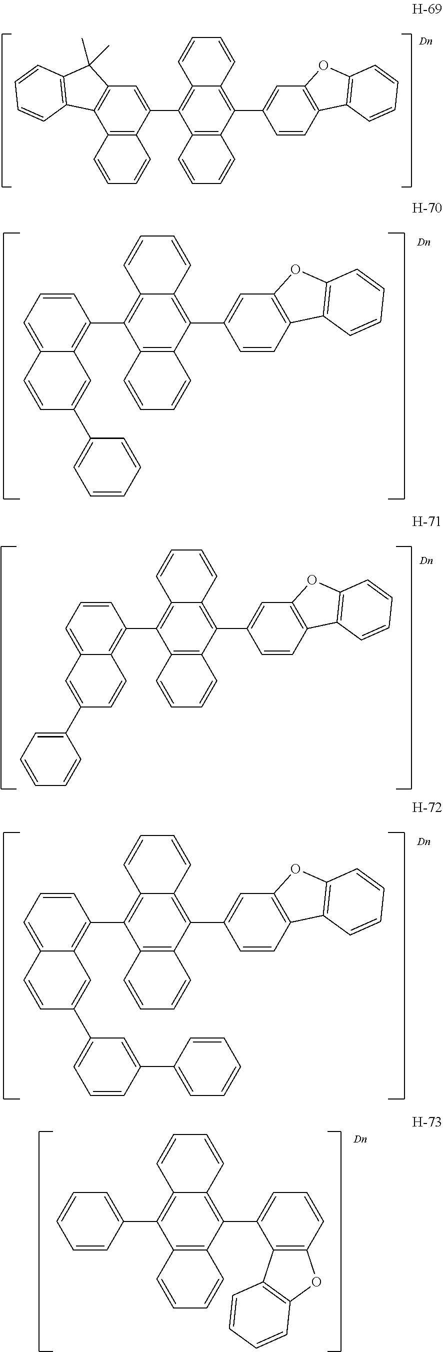

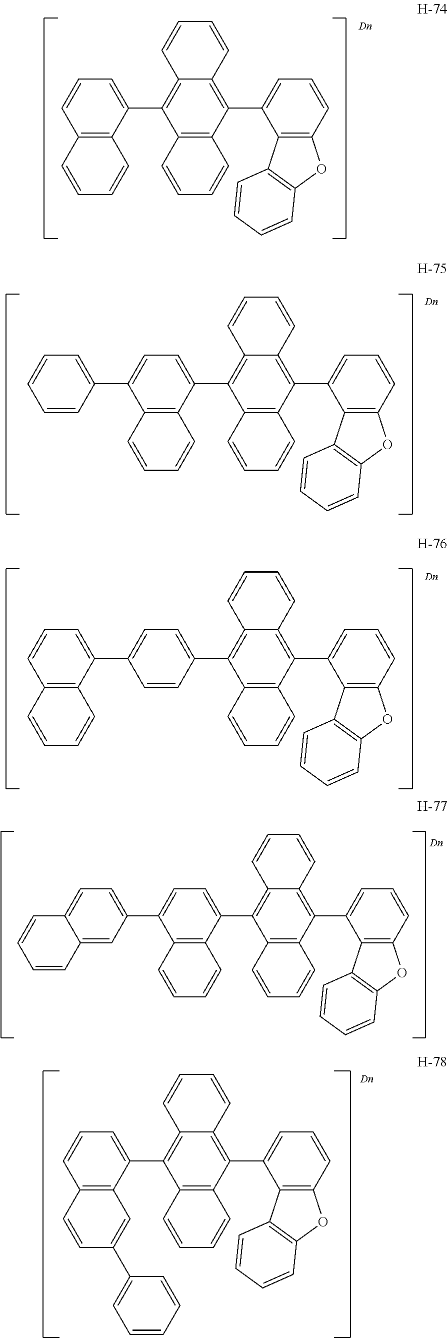

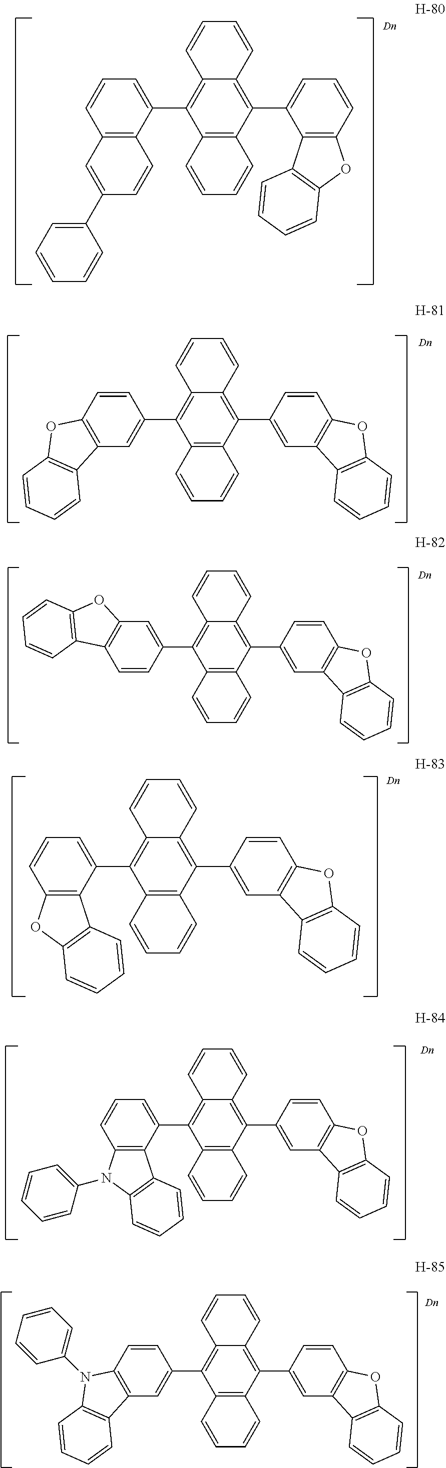

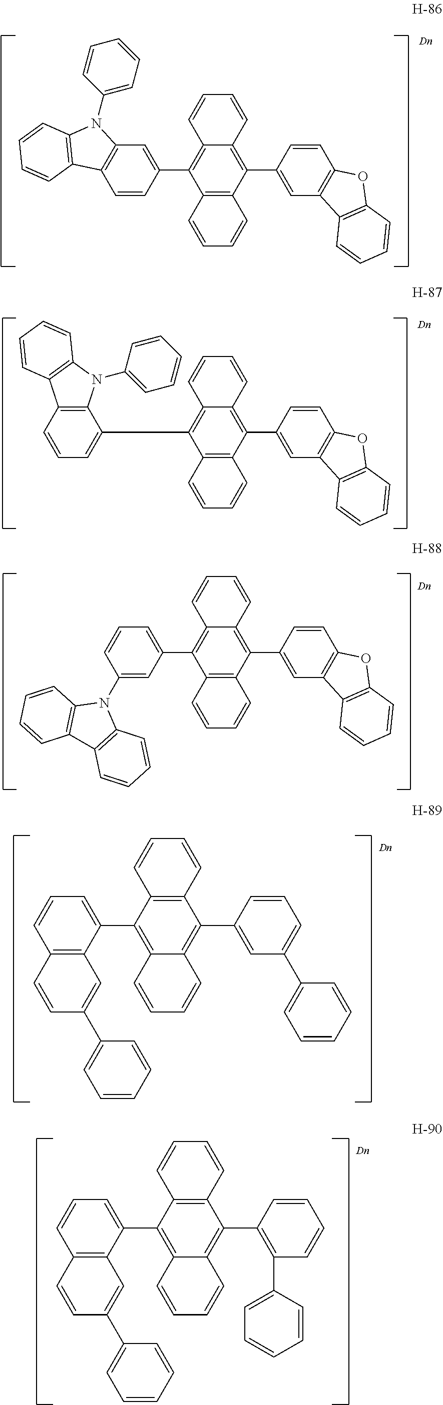

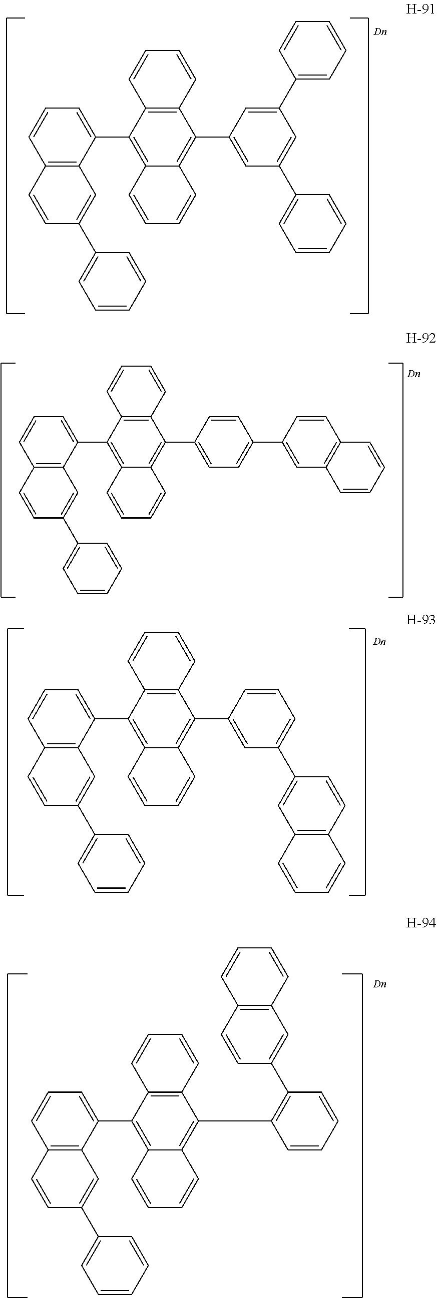

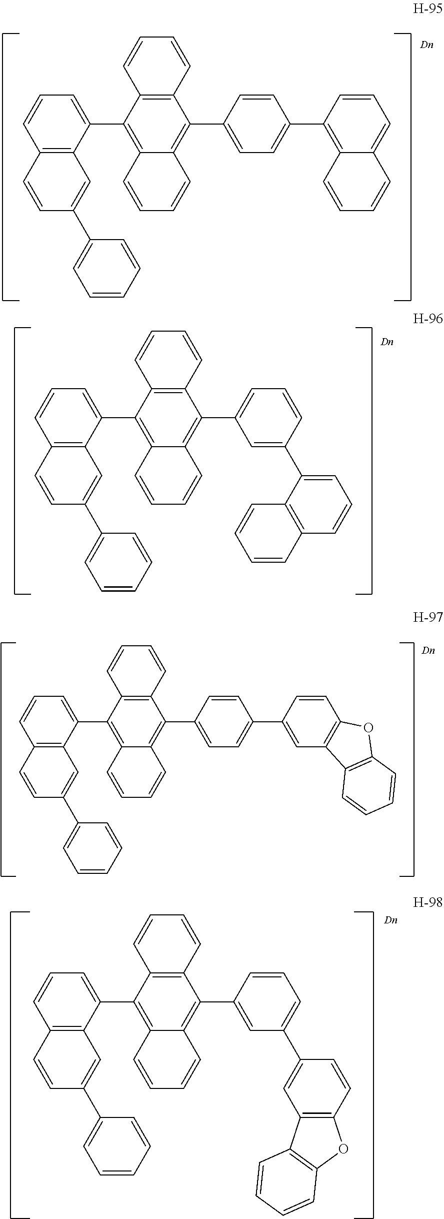

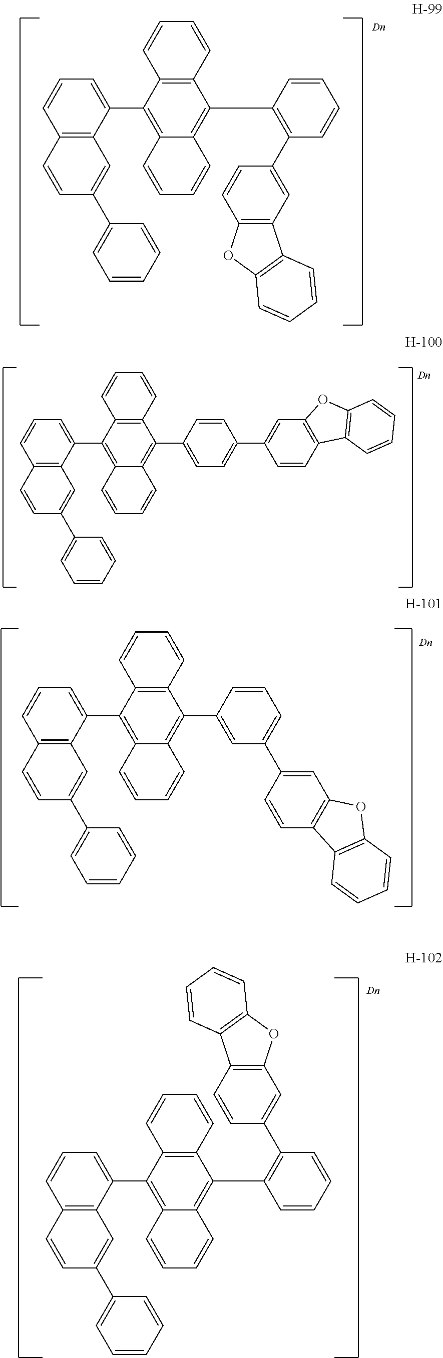

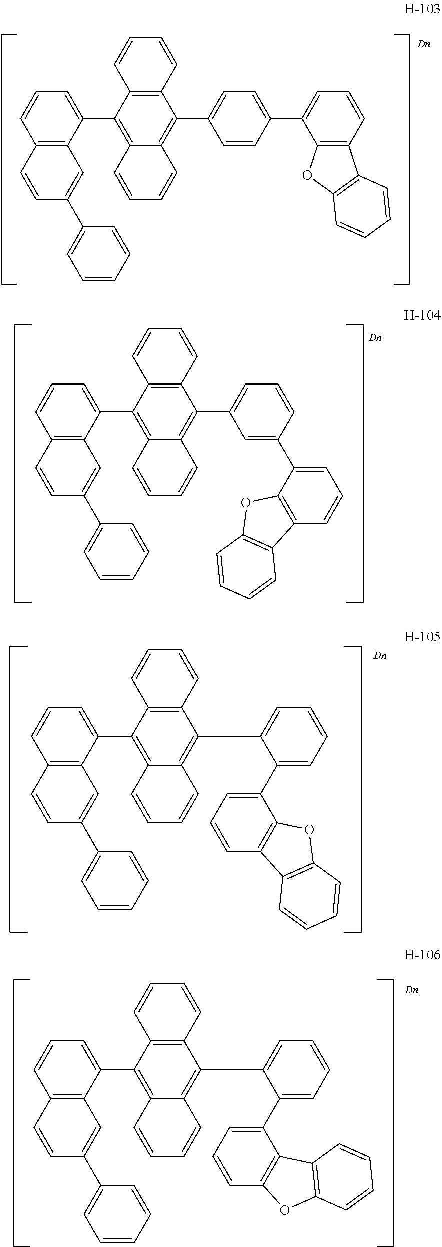

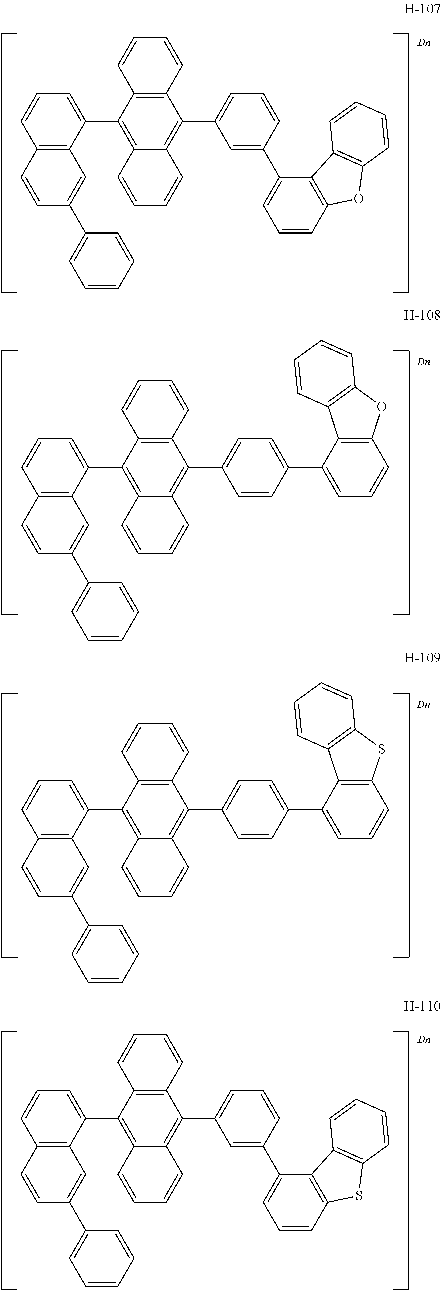

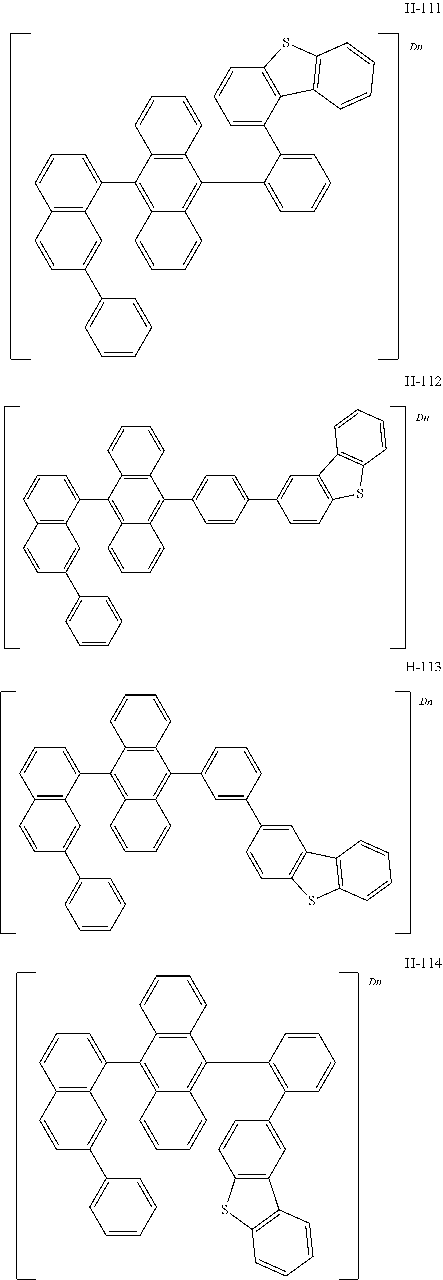

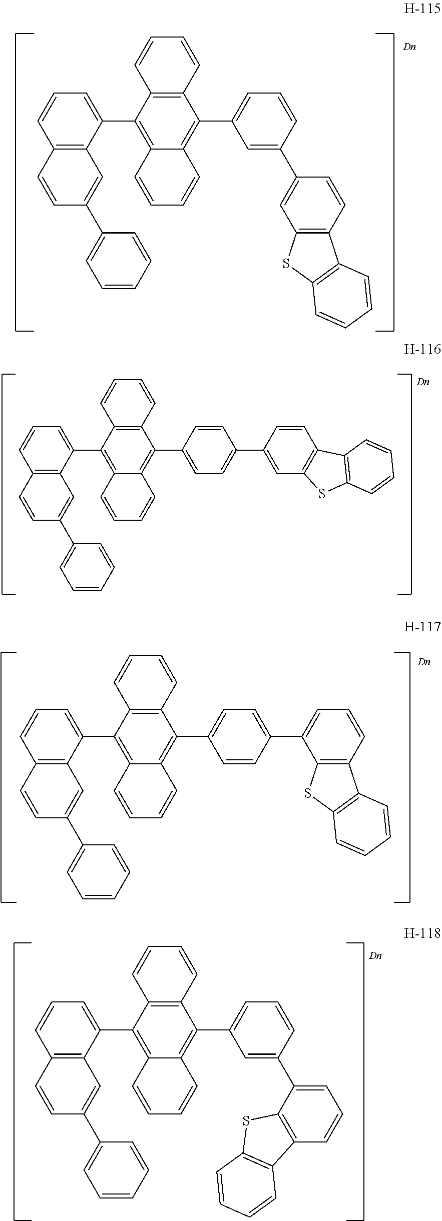

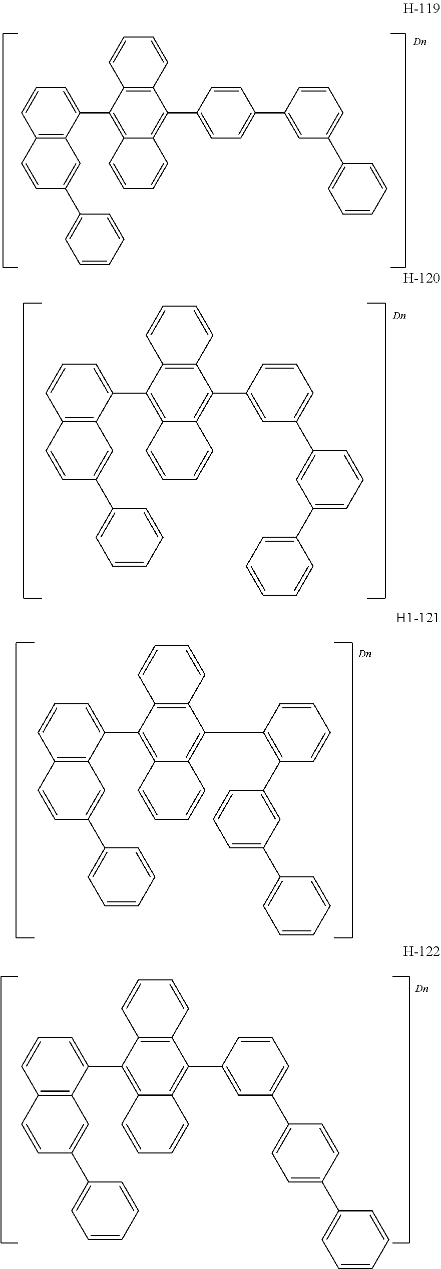

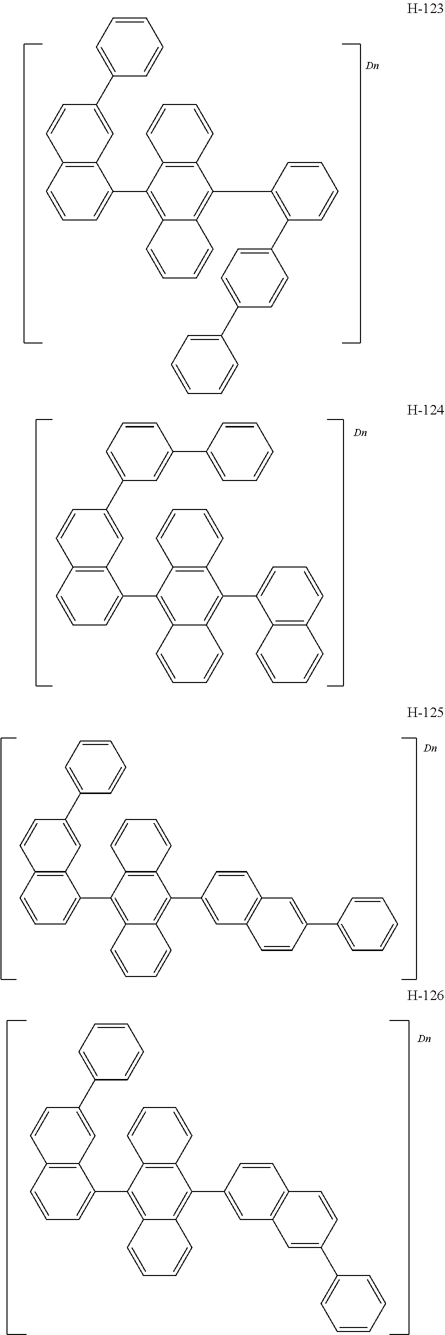

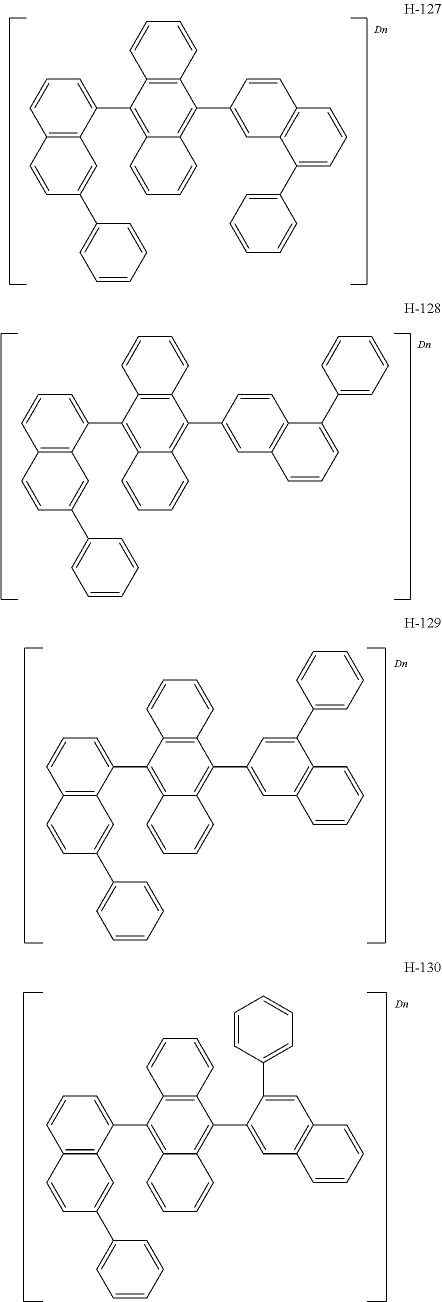

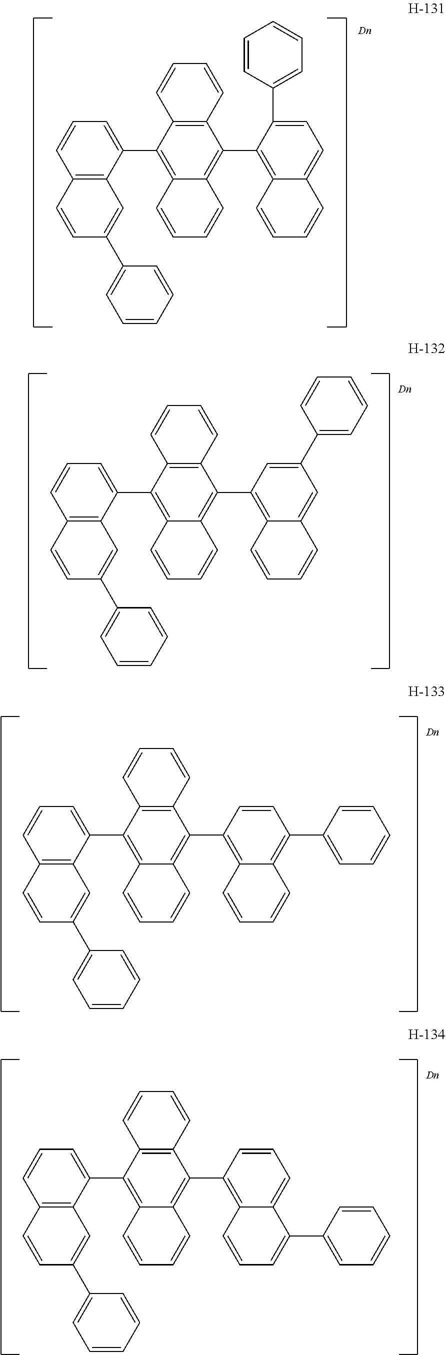

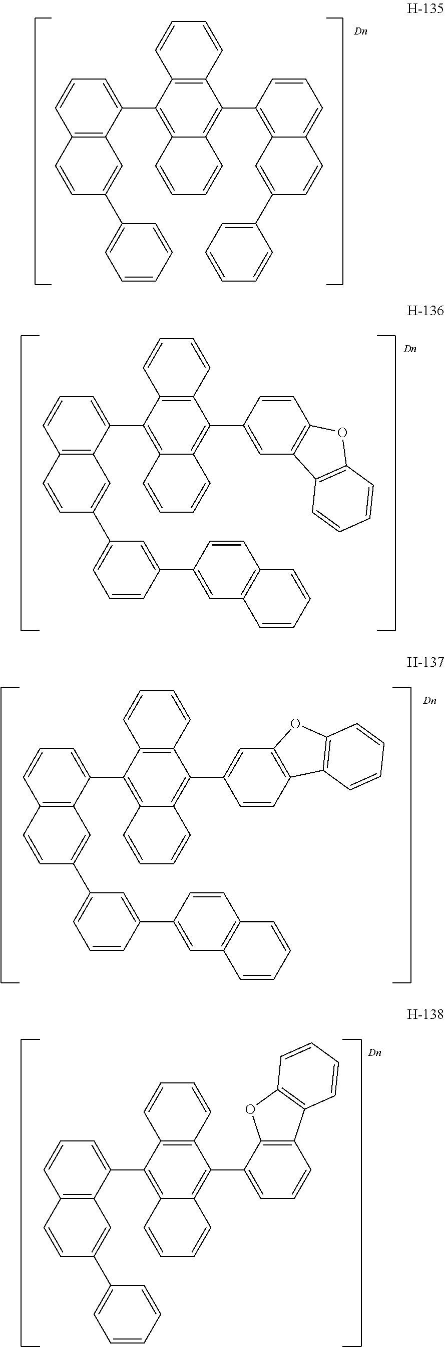

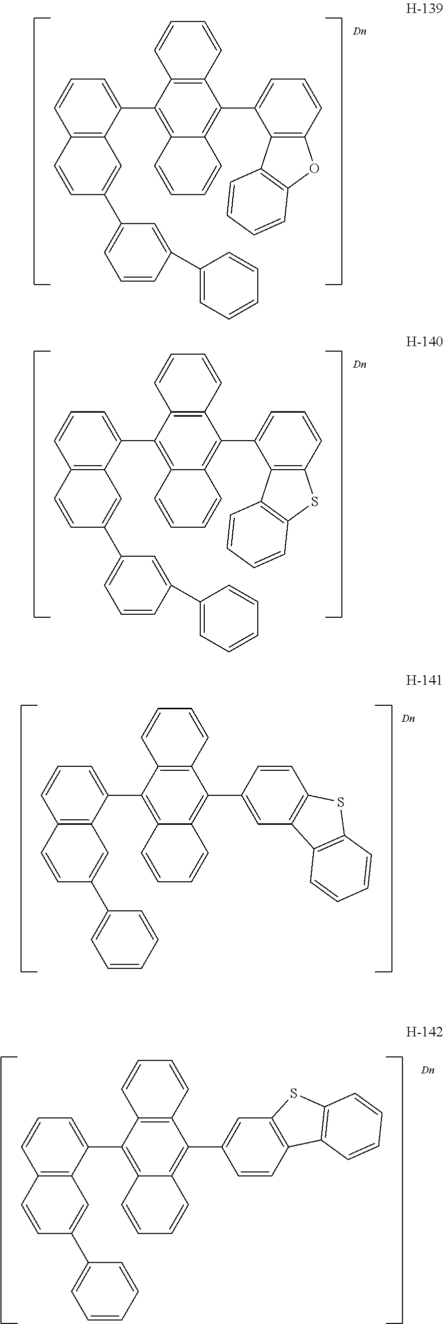

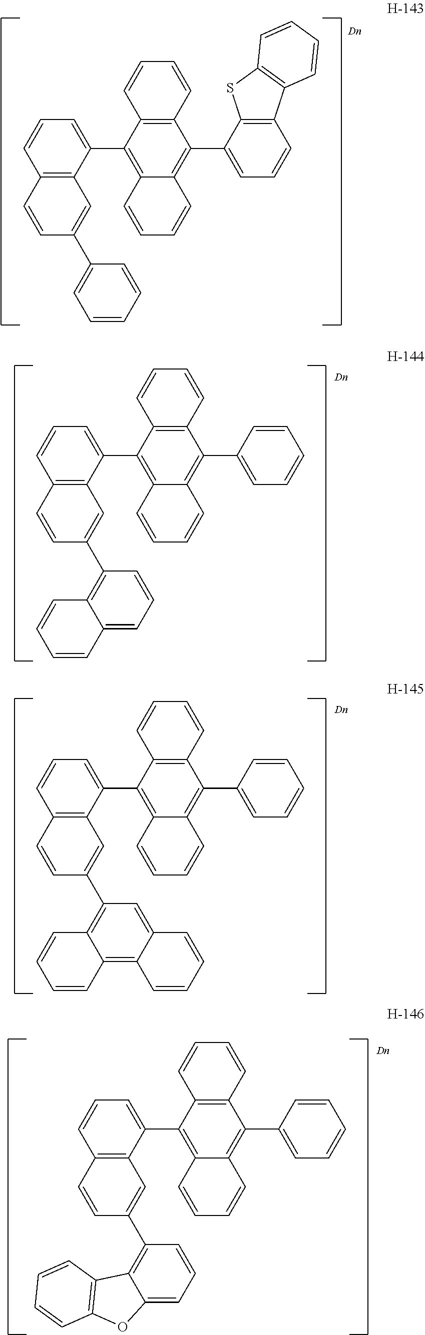

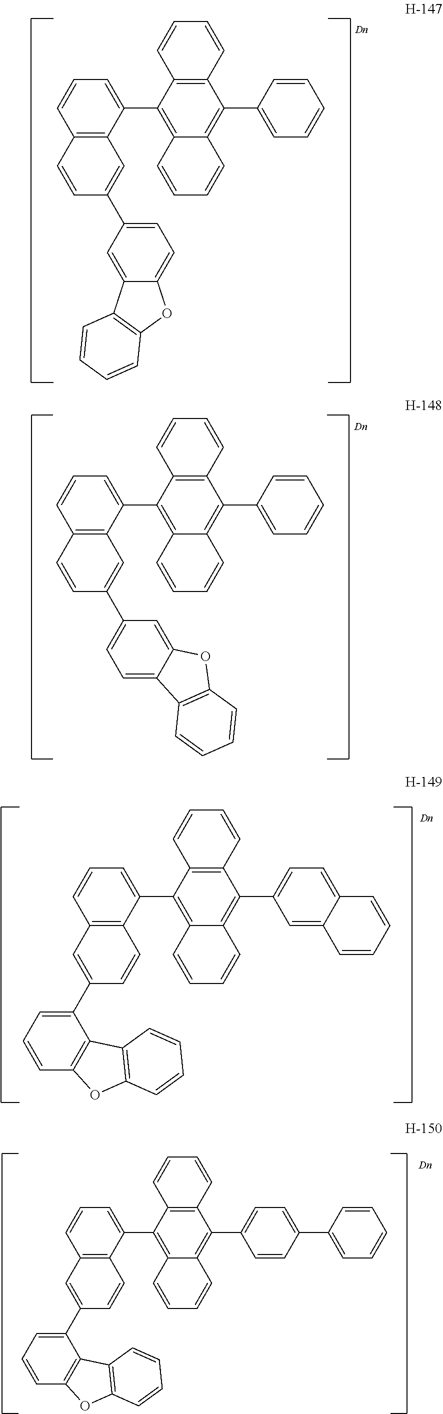

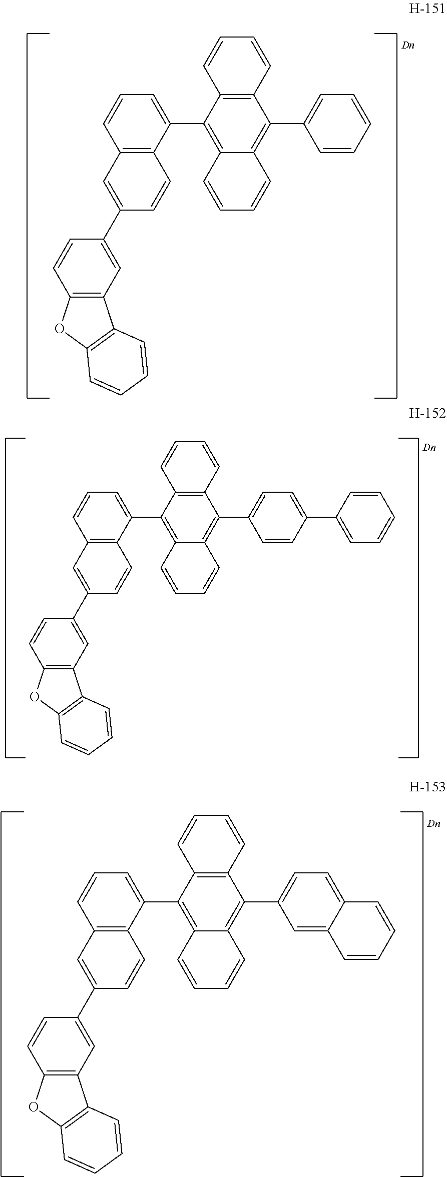

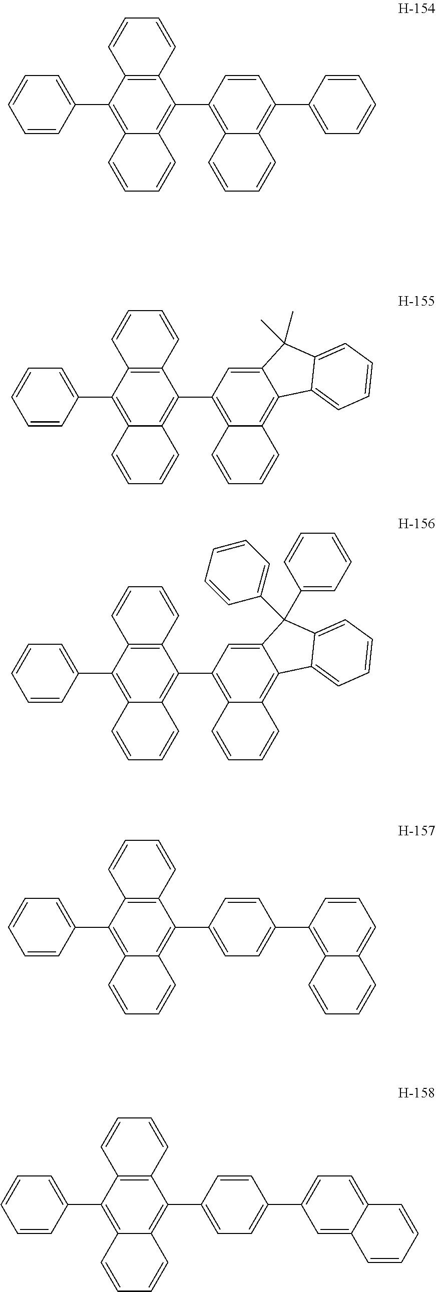

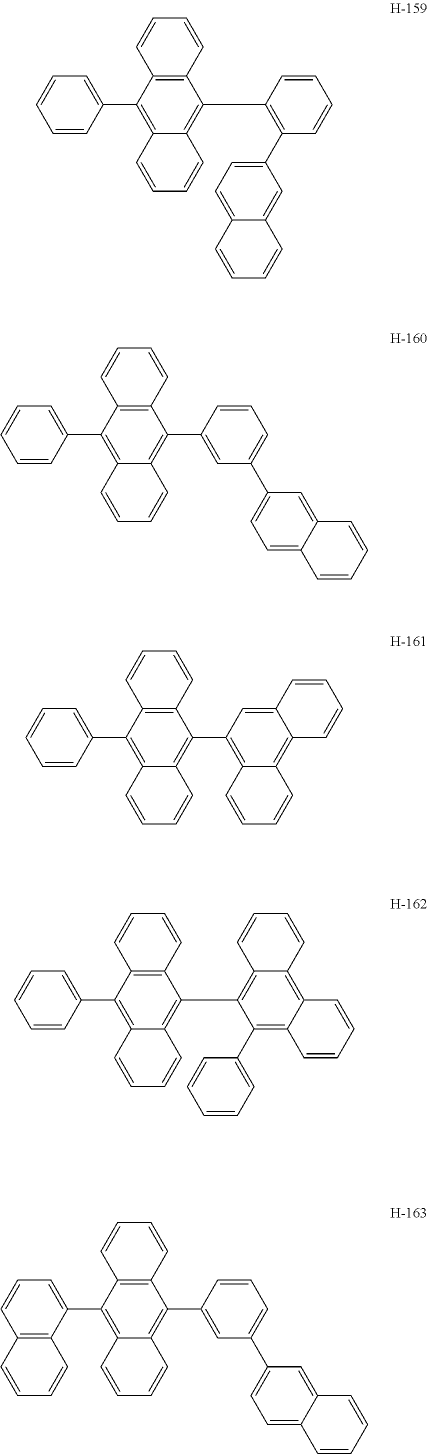

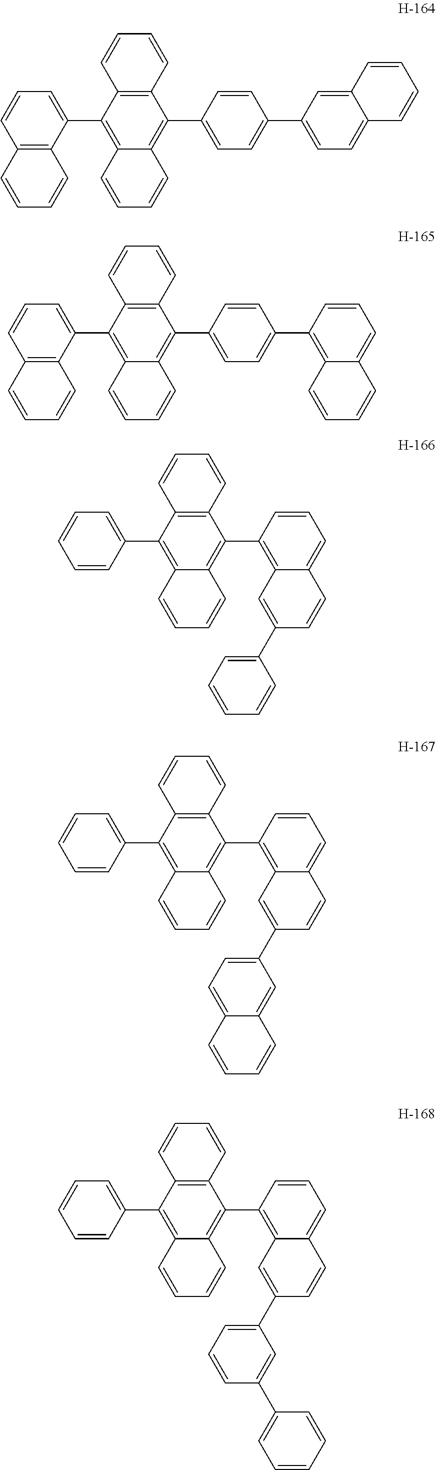

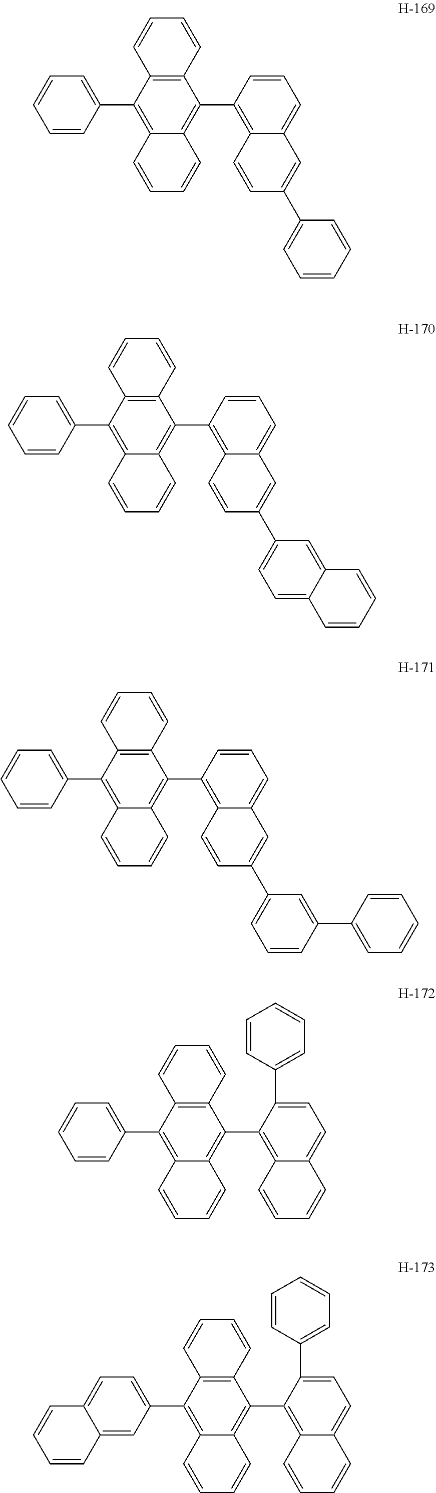

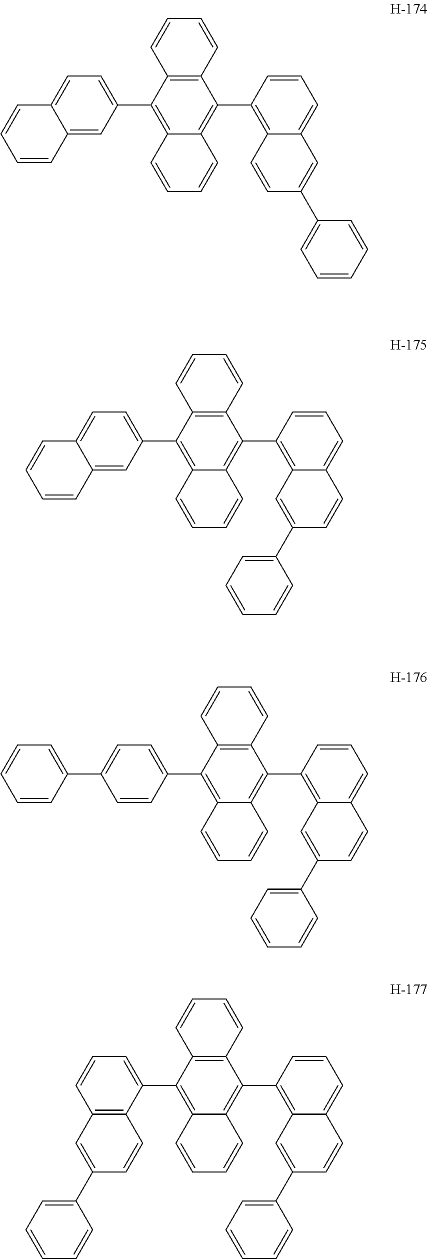

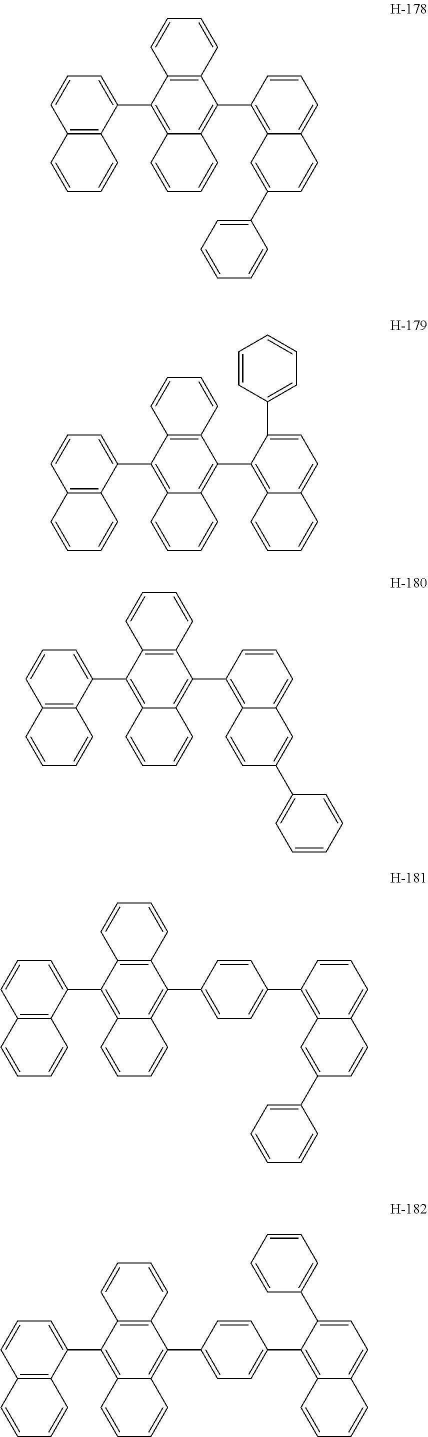

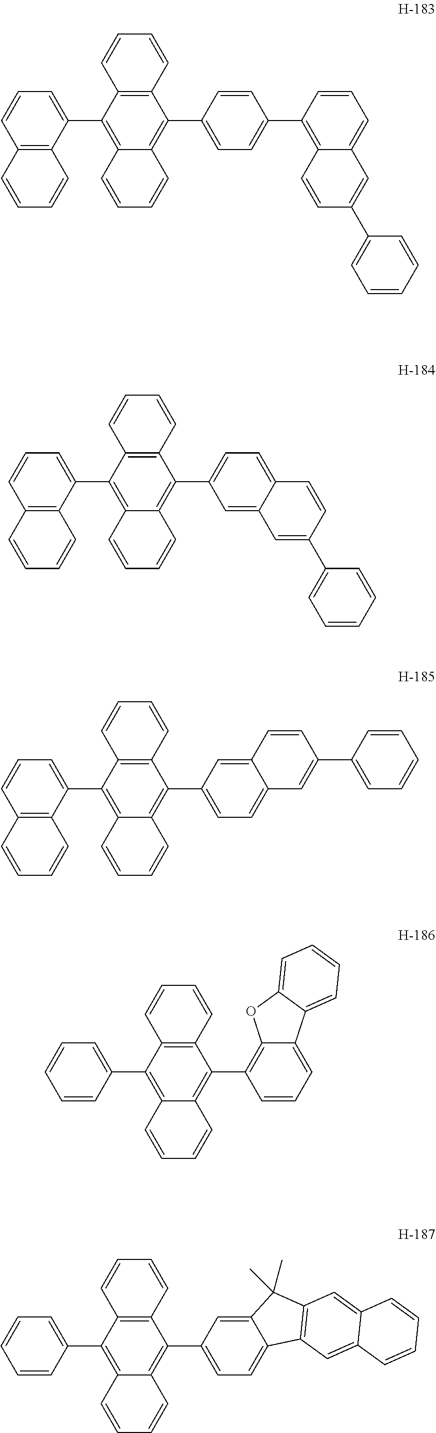

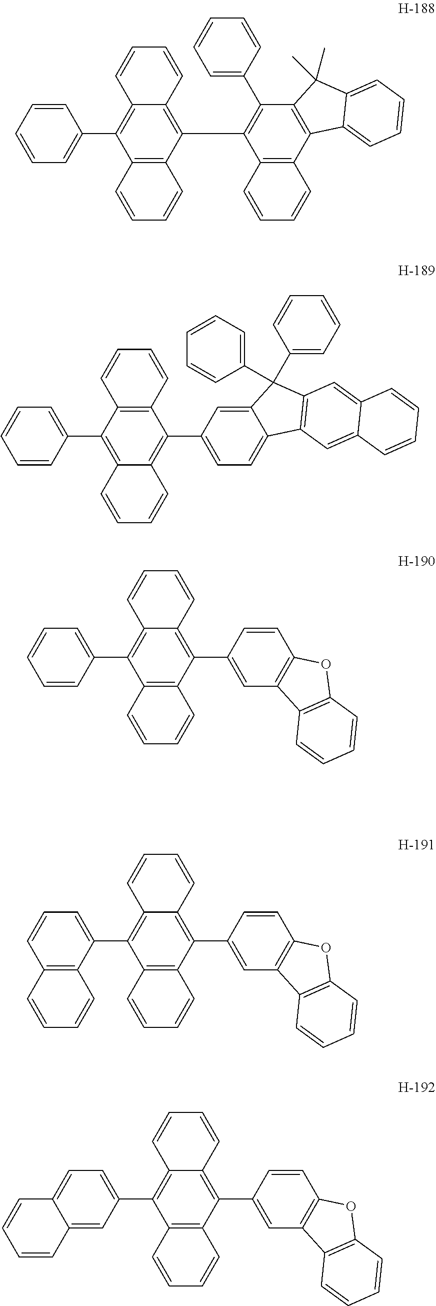

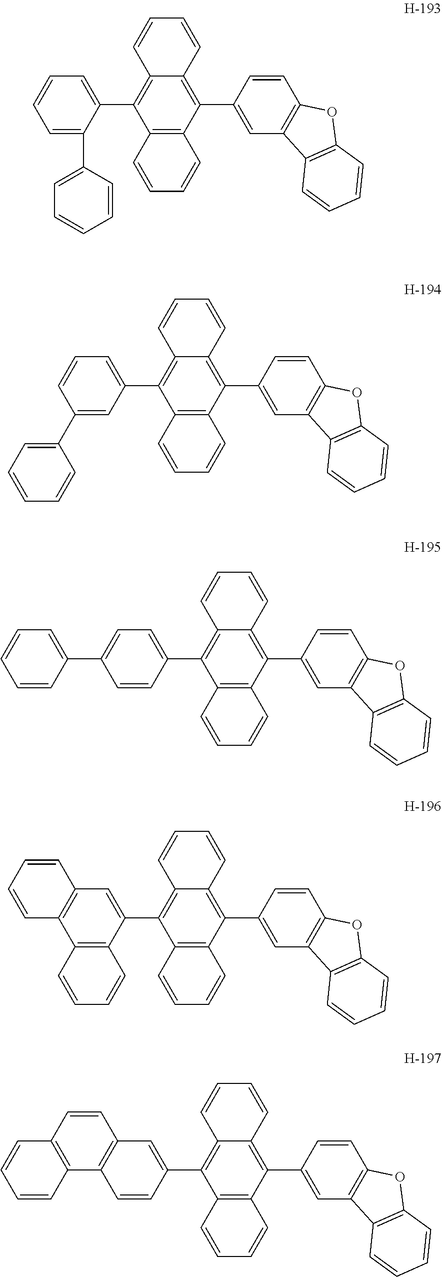 [SMILES: c1ccc(-c2ccccc2-c2c3ccccc3c(-c3ccc4oc5ccccc5c4c3)c3ccccc23)cc1 ; c1ccc(-c2cccc(-c3c4ccccc4c(-c4ccc5oc6ccccc6c5c4)c4ccccc34)c2)cc1 ; c1ccc(-c2ccc(-c3c4ccccc4c(-c4ccc5oc6ccccc6c5c4)c4ccccc34)cc2)cc1 ; c1ccc2c(c1)cc(-c1c3ccccc3c(-c3ccc4oc5ccccc5c4c3)c3ccccc13)c1ccccc12 ; c1ccc2c(c1)ccc1cc(-c3c4ccccc4c(-c4ccc5oc6ccccc6c5c4)c4ccccc34)ccc12]

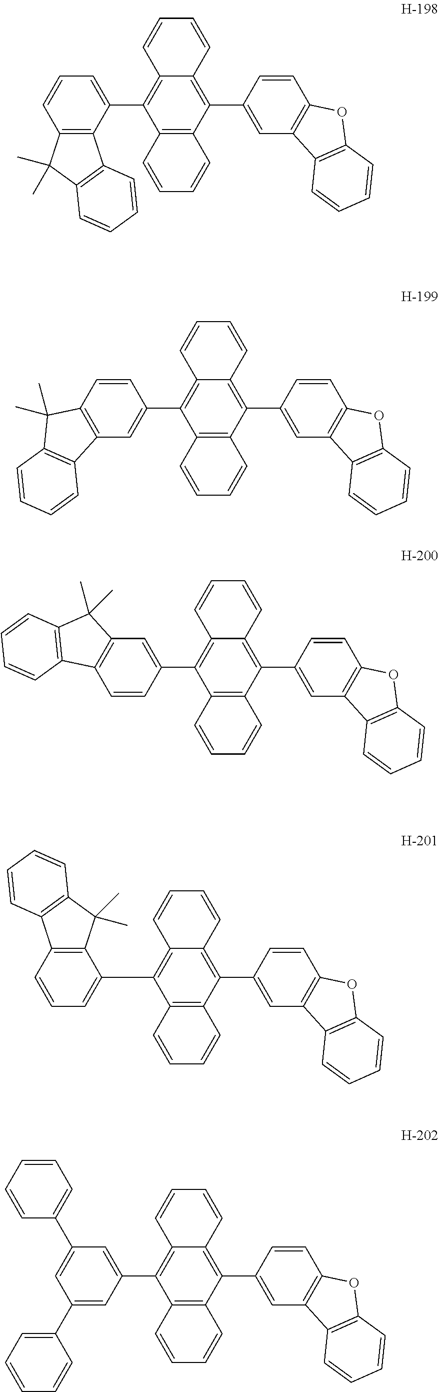 [SMILES: CC1(C)c2ccccc2-c2c(-c3c4ccccc4c(-c4ccc5oc6ccccc6c5c4)c4ccccc34)cccc21 ; CC1(C)c2ccccc2-c2cc(-c3c4ccccc4c(-c4ccc5oc6ccccc6c5c4)c4ccccc34)ccc21 ; CC1(C)c2ccccc2-c2ccc(-c3c4ccccc4c(-c4ccc5oc6ccccc6c5c4)c4ccccc34)cc21 ; CC1(C)c2ccccc2-c2cccc(-c3c4ccccc4c(-c4ccc5oc6ccccc6c5c4)c4ccccc34)c21 ; c1ccc(-c2cc(-c3ccccc3)cc(-c3c4ccccc4c(-c4ccc5oc6ccccc6c5c4)c4ccccc34)c2)cc1]

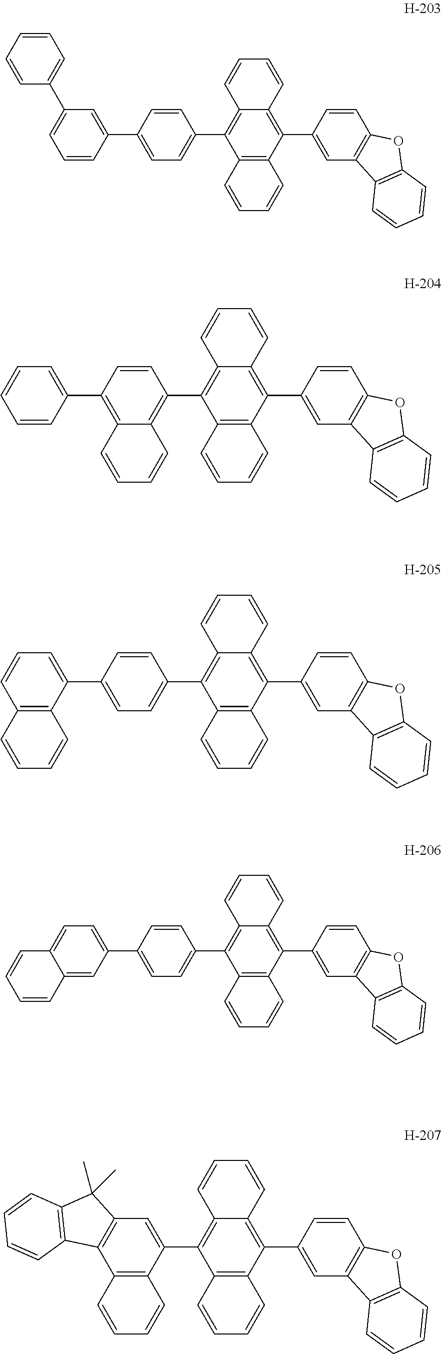 [SMILES: c1ccc(-c2cccc(-c3ccc(-c4c5ccccc5c(-c5ccc6oc7ccccc7c6c5)c5ccccc45)cc3)c2)cc1 ; c1ccc(-c2ccc(-c3c4ccccc4c(-c4ccc5oc6ccccc6c5c4)c4ccccc34)c3ccccc23)cc1 ; c1ccc2c(-c3ccc(-c4c5ccccc5c(-c5ccc6oc7ccccc7c6c5)c5ccccc45)cc3)cccc2c1 ; c1ccc2cc(-c3ccc(-c4c5ccccc5c(-c5ccc6oc7ccccc7c6c5)c5ccccc45)cc3)ccc2c1 ; CC1(C)c2ccccc2-c2c1cc(-c1c3ccccc3c(-c3ccc4oc5ccccc5c4c3)c3ccccc13)c1ccccc21]

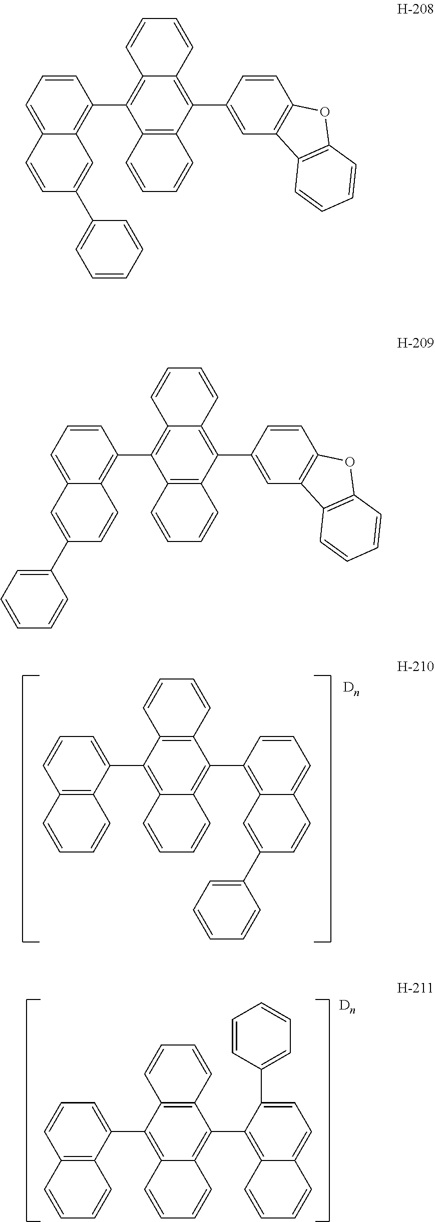

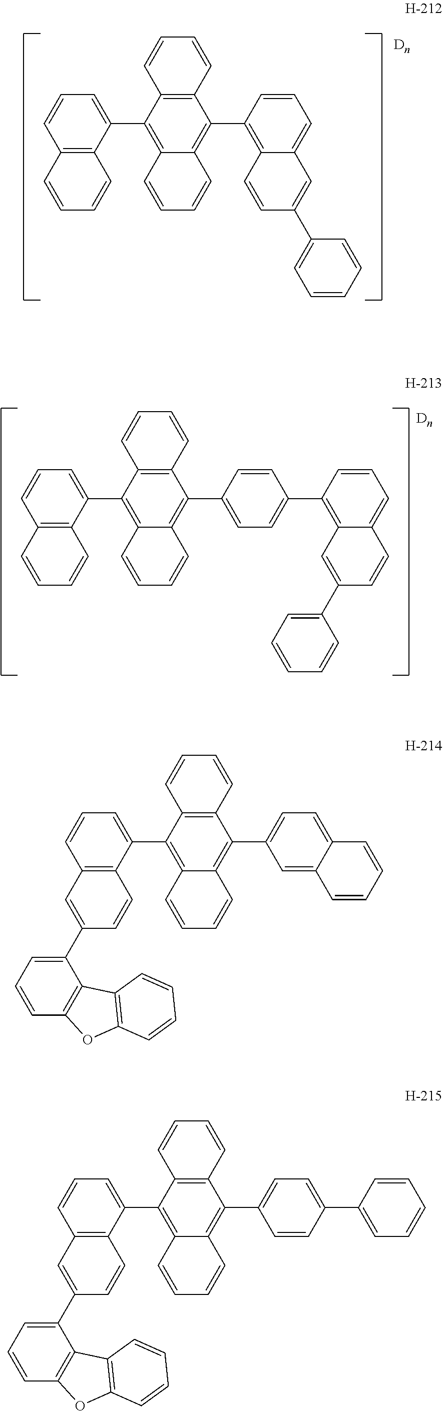

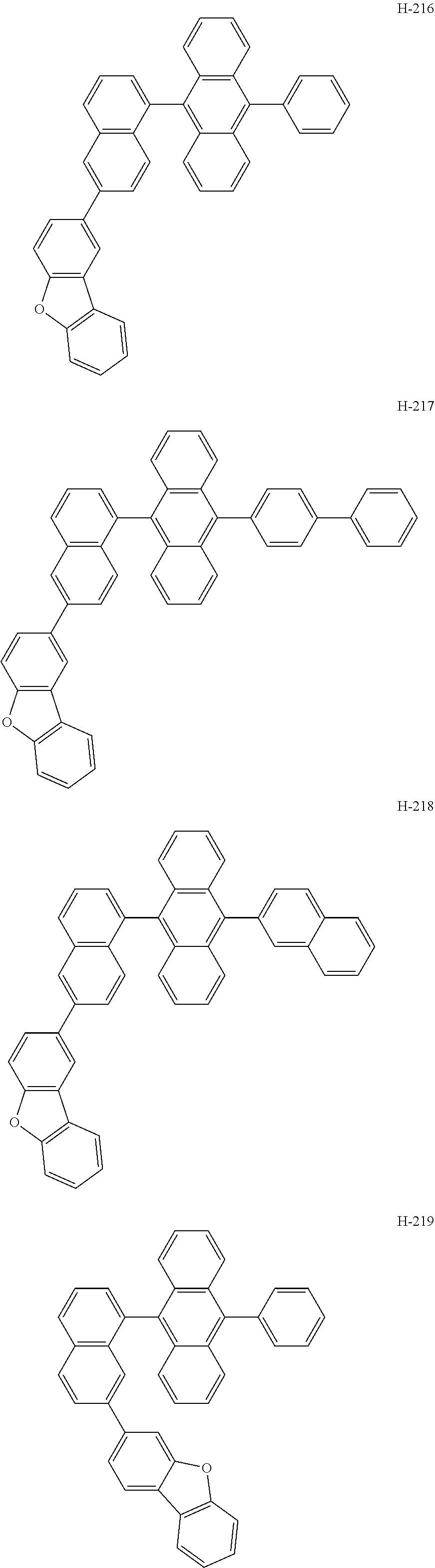 [SMILES: c1ccc(-c2c3ccccc3c(-c3cccc4cc(-c5ccc6oc7ccccc7c6c5)ccc34)c3ccccc23)cc1 ; c1ccc(-c2ccc(-c3c4ccccc4c(-c4cccc5cc(-c6ccc7oc8ccccc8c7c6)ccc45)c4ccccc34)cc2)cc1 ; c1ccc2cc(-c3c4ccccc4c(-c4cccc5cc(-c6ccc7oc8ccccc8c7c6)ccc45)c4ccccc34)ccc2c1 ; c1ccc(-c2c3ccccc3c(-c3cccc4ccc(-c5ccc6c(c5)oc5ccccc56)cc34)c3ccccc23)cc1]

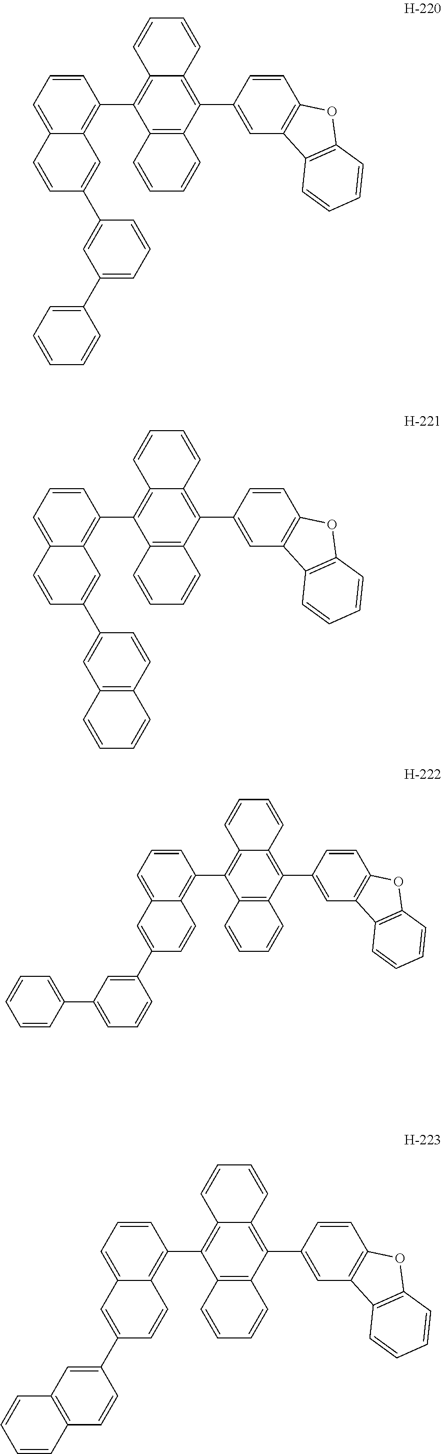 [SMILES: c1ccc(-c2cccc(-c3ccc4cccc(-c5c6ccccc6c(-c6ccc7oc8ccccc8c7c6)c6ccccc56)c4c3)c2)cc1 ; c1ccc2cc(-c3ccc4cccc(-c5c6ccccc6c(-c6ccc7oc8ccccc8c7c6)c6ccccc56)c4c3)ccc2c1 ; c1ccc(-c2cccc(-c3ccc4c(-c5c6ccccc6c(-c6ccc7oc8ccccc8c7c6)c6ccccc56)cccc4c3)c2)cc1 ; c1ccc2cc(-c3ccc4c(-c5c6ccccc6c(-c6ccc7oc8ccccc8c7c6)c6ccccc56)cccc4c3)ccc2c1]

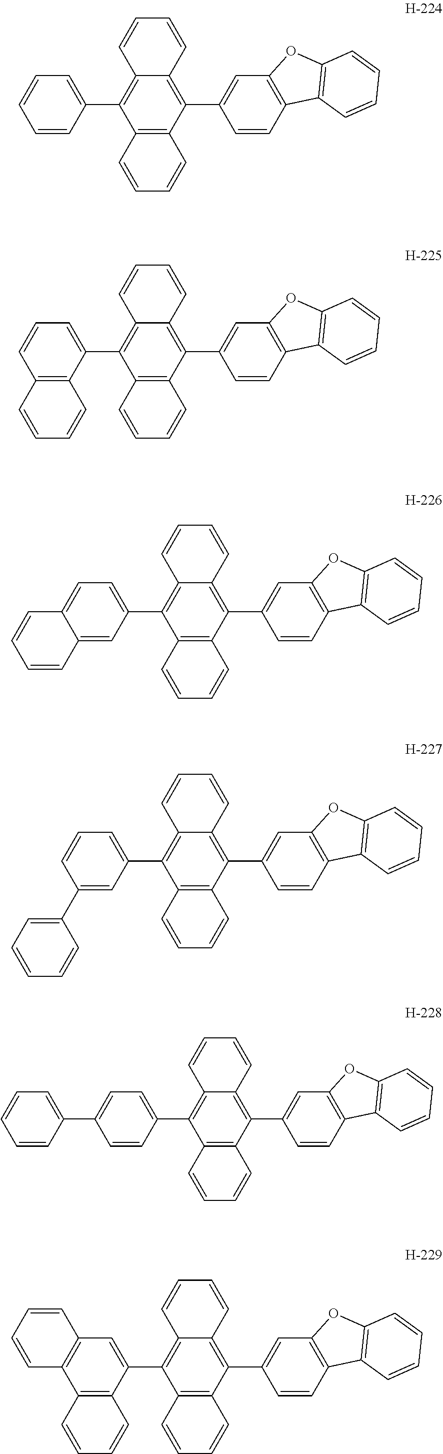 [SMILES: c1ccc(-c2c3ccccc3c(-c3ccc4c(c3)oc3ccccc34)c3ccccc23)cc1 ; c1ccc2c(-c3c4ccccc4c(-c4ccc5c(c4)oc4ccccc45)c4ccccc34)cccc2c1 ; c1ccc2cc(-c3c4ccccc4c(-c4ccc5c(c4)oc4ccccc45)c4ccccc34)ccc2c1 ; c1ccc(-c2cccc(-c3c4ccccc4c(-c4ccc5c(c4)oc4ccccc45)c4ccccc34)c2)cc1 ; c1ccc(-c2ccc(-c3c4ccccc4c(-c4ccc5c(c4)oc4ccccc45)c4ccccc34)cc2)cc1 ; c1ccc2c(c1)cc(-c1c3ccccc3c(-c3ccc4c(c3)oc3ccccc34)c3ccccc13)c1ccccc12]

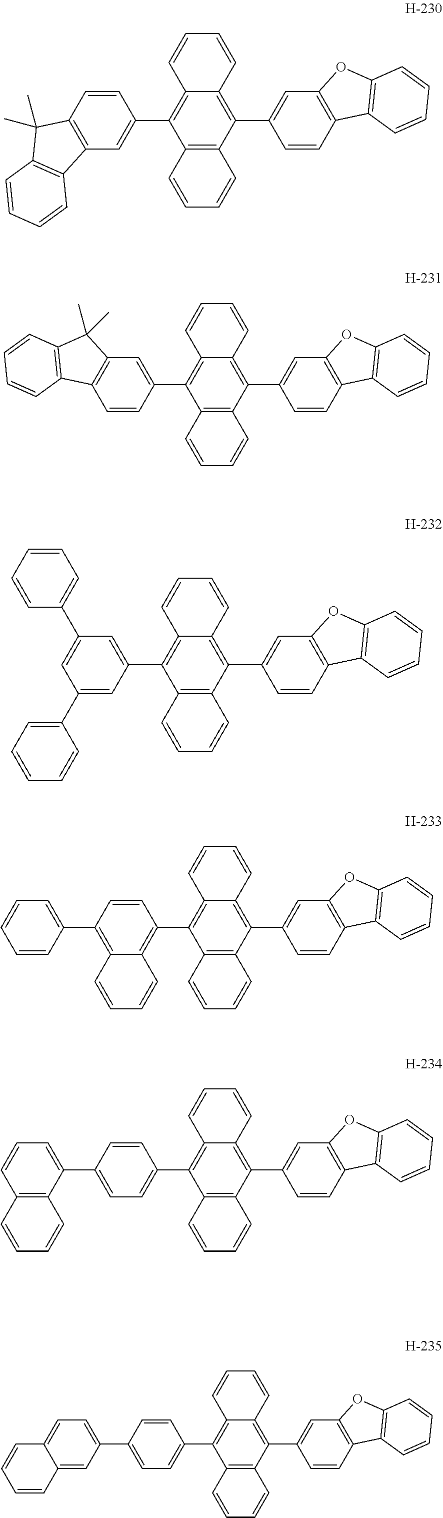

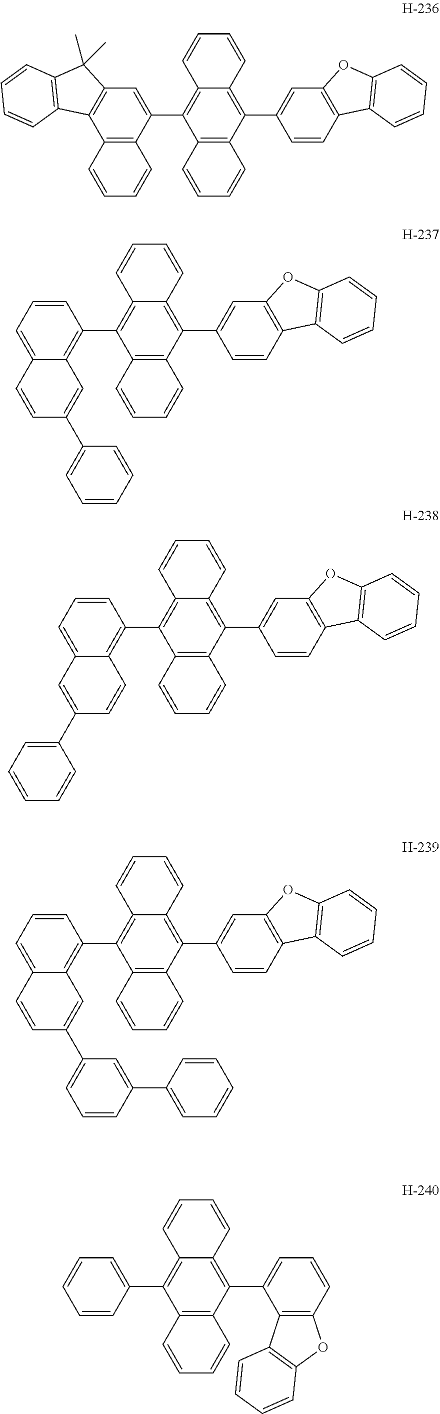 [SMILES: CC1(C)c2ccccc2-c2c1cc(-c1c3ccccc3c(-c3ccc4c(c3)oc3ccccc34)c3ccccc13)c1ccccc21 ; c1ccc(-c2ccc3cccc(-c4c5ccccc5c(-c5ccc6c(c5)oc5ccccc56)c5ccccc45)c3c2)cc1 ; c1ccc(-c2ccc3c(-c4c5ccccc5c(-c5ccc6c(c5)oc5ccccc56)c5ccccc45)cccc3c2)cc1 ; c1ccc(-c2cccc(-c3ccc4cccc(-c5c6ccccc6c(-c6ccc7c(c6)oc6ccccc67)c6ccccc56)c4c3)c2)cc1 ; c1ccc(-c2c3ccccc3c(-c3cccc4oc5ccccc5c34)c3ccccc23)cc1]

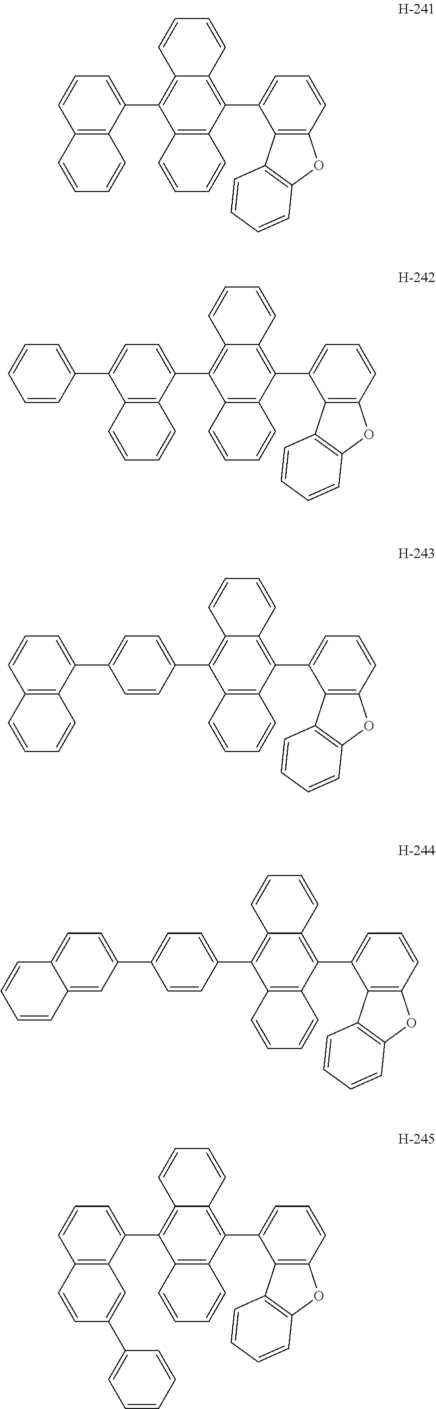 [SMILES: c1ccc2c(-c3c4ccccc4c(-c4cccc5oc6ccccc6c45)c4ccccc34)cccc2c1 ; c1ccc(-c2ccc(-c3c4ccccc4c(-c4cccc5oc6ccccc6c45)c4ccccc34)c3ccccc23)cc1 ; c1ccc2c(-c3ccc(-c4c5ccccc5c(-c5cccc6oc7ccccc7c56)c5ccccc45)cc3)cccc2c1 ; c1ccc2cc(-c3ccc(-c4c5ccccc5c(-c5cccc6oc7ccccc7c56)c5ccccc45)cc3)ccc2c1 ; c1ccc(-c2ccc3cccc(-c4c5ccccc5c(-c5cccc6oc7ccccc7c56)c5ccccc45)c3c2)cc1]

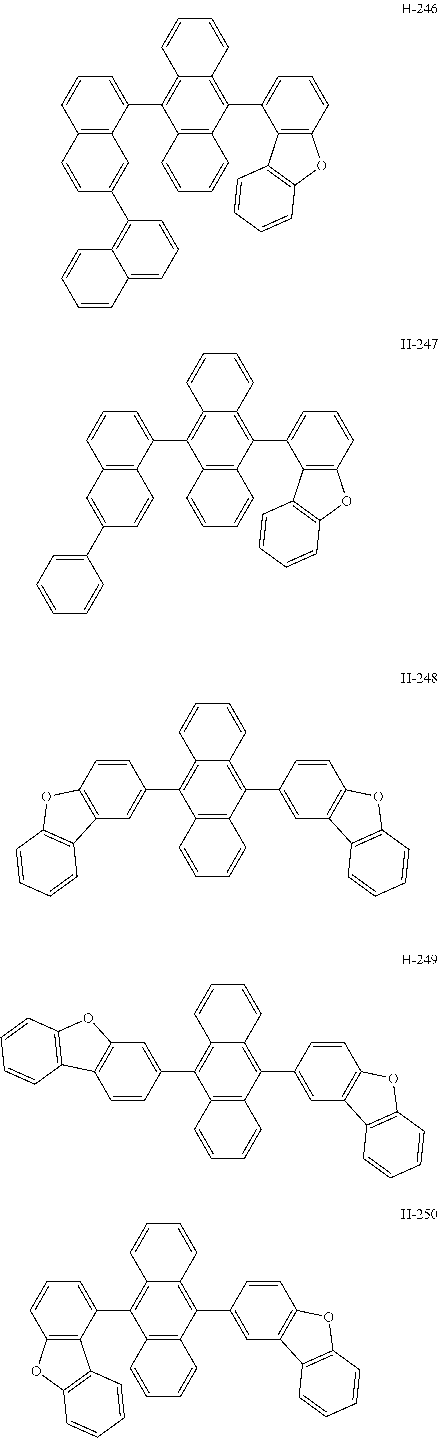 [SMILES: c1ccc2c(-c3ccc4cccc(-c5c6ccccc6c(-c6cccc7oc8ccccc8c67)c6ccccc56)c4c3)cccc2c1 ; c1ccc(-c2ccc3c(-c4c5ccccc5c(-c5cccc6oc7ccccc7c56)c5ccccc45)cccc3c2)cc1 ; c1ccc2c(c1)oc1ccc(-c3c4ccccc4c(-c4ccc5oc6ccccc6c5c4)c4ccccc34)cc12 ; c1ccc2c(c1)oc1cc(-c3c4ccccc4c(-c4ccc5oc6ccccc6c5c4)c4ccccc34)ccc12 ; c1ccc2c(c1)oc1ccc(-c3c4ccccc4c(-c4cccc5oc6ccccc6c45)c4ccccc34)cc12]

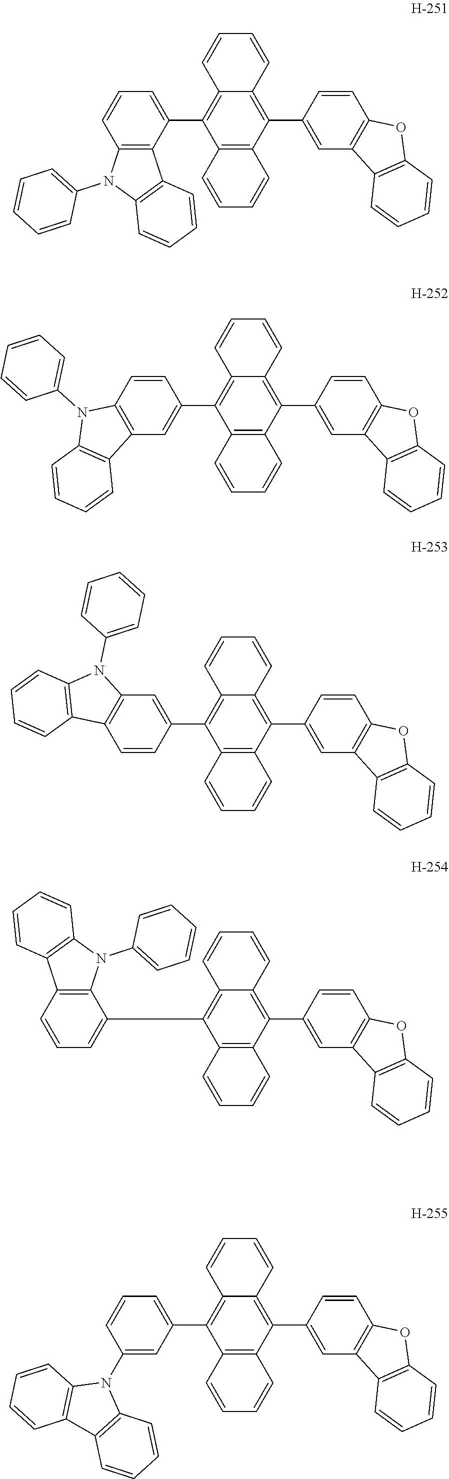 [SMILES: c1ccc(-n2c3ccccc3c3c(-c4c5ccccc5c(-c5ccc6oc7ccccc7c6c5)c5ccccc45)cccc32)cc1 ; c1ccc(-n2c3ccccc3c3cc(-c4c5ccccc5c(-c5ccc6oc7ccccc7c6c5)c5ccccc45)ccc32)cc1 ; c1ccc(-n2c3ccccc3c3ccc(-c4c5ccccc5c(-c5ccc6oc7ccccc7c6c5)c5ccccc45)cc32)cc1 ; c1ccc(-n2c3ccccc3c3cccc(-c4c5ccccc5c(-c5ccc6oc7ccccc7c6c5)c5ccccc45)c32)cc1 ; c1cc(-c2c3ccccc3c(-c3ccc4oc5ccccc5c4c3)c3ccccc23)cc(-n2c3ccccc3c3ccccc32)c1]

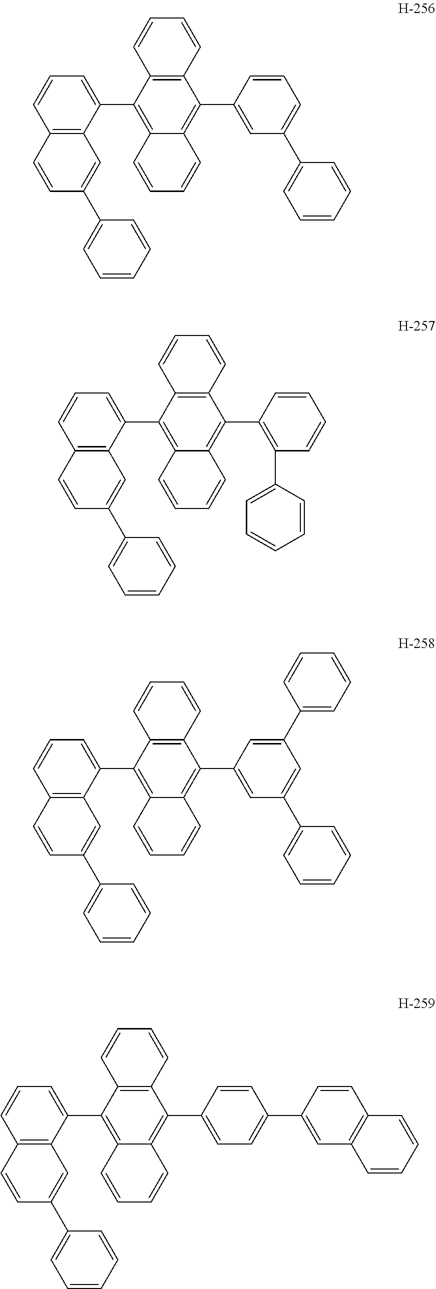

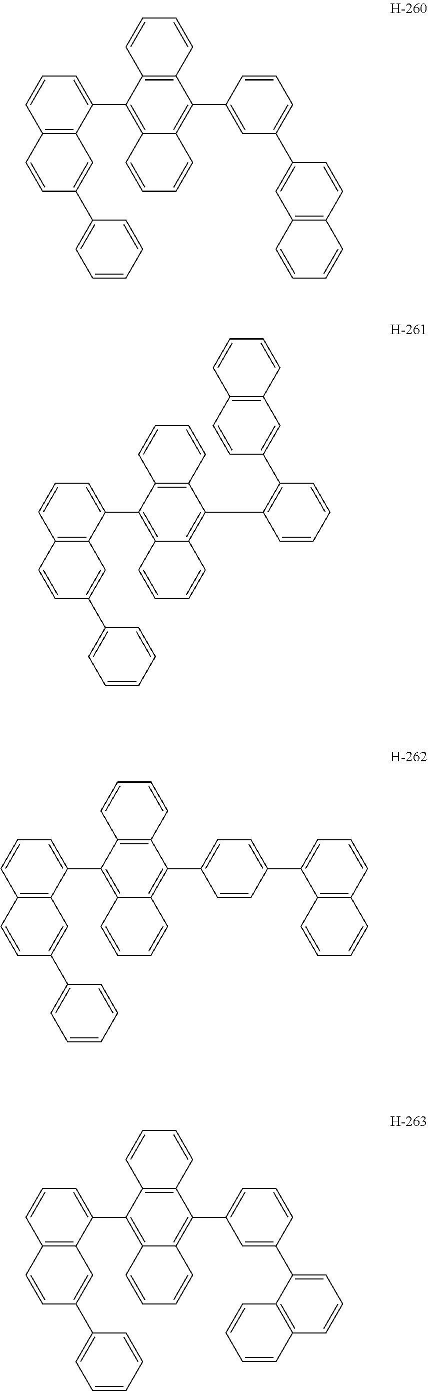

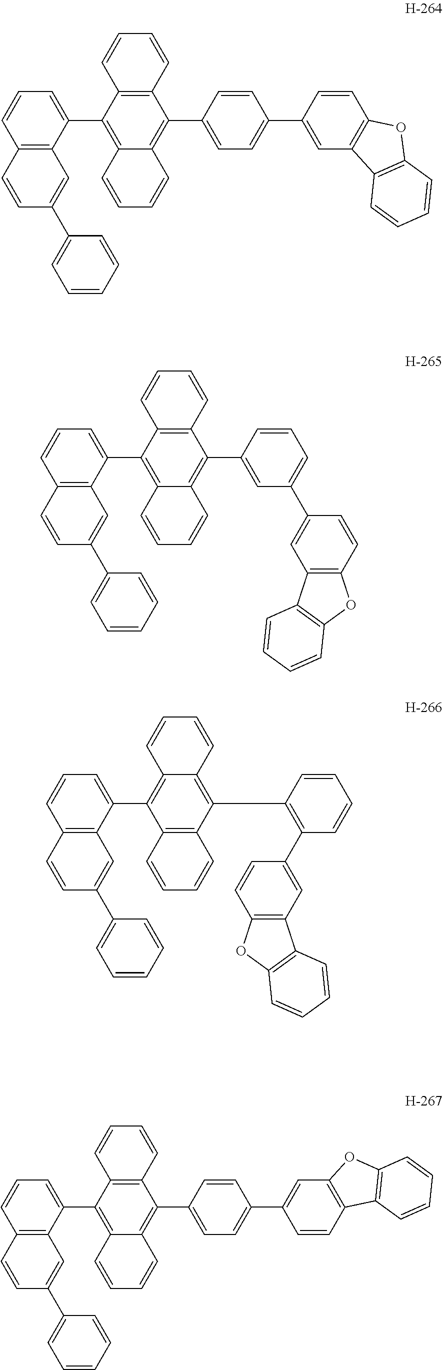 [SMILES: c1ccc(-c2ccc3cccc(-c4c5ccccc5c(-c5ccc(-c6ccc7oc8ccccc8c7c6)cc5)c5ccccc45)c3c2)cc1 ; c1ccc(-c2ccc3cccc(-c4c5ccccc5c(-c5cccc(-c6ccc7oc8ccccc8c7c6)c5)c5ccccc45)c3c2)cc1 ; c1ccc(-c2ccc3cccc(-c4c5ccccc5c(-c5ccccc5-c5ccc6oc7ccccc7c6c5)c5ccccc45)c3c2)cc1 ; c1ccc(-c2ccc3cccc(-c4c5ccccc5c(-c5ccc(-c6ccc7c(c6)oc6ccccc67)cc5)c5ccccc45)c3c2)cc1]

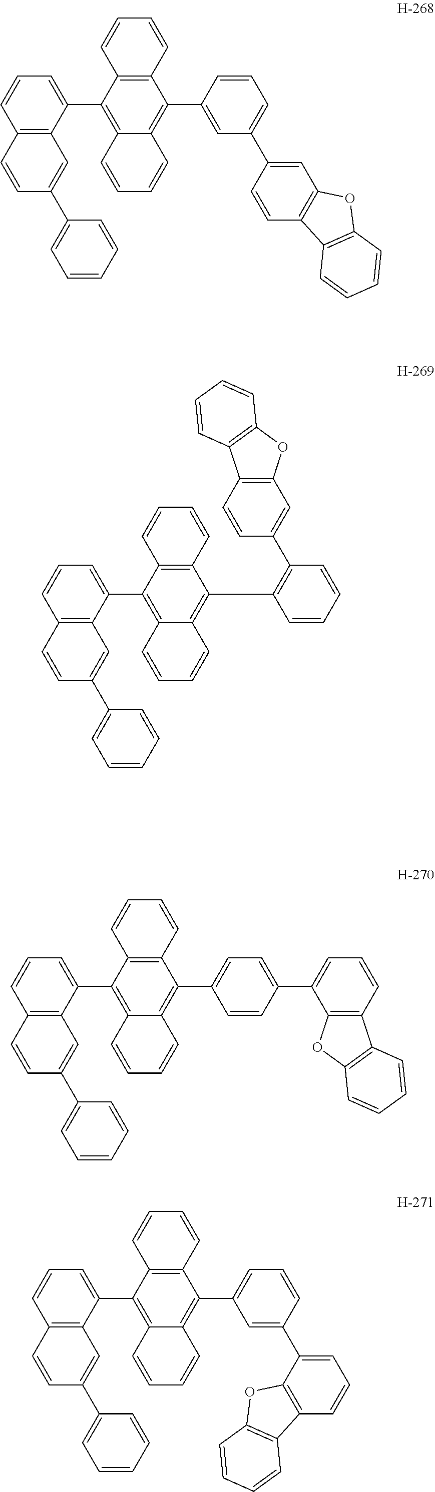 [SMILES: c1ccc(-c2ccc3cccc(-c4c5ccccc5c(-c5cccc(-c6ccc7c(c6)oc6ccccc67)c5)c5ccccc45)c3c2)cc1 ; c1ccc(-c2ccc3cccc(-c4c5ccccc5c(-c5ccccc5-c5ccc6c(c5)oc5ccccc56)c5ccccc45)c3c2)cc1 ; c1ccc(-c2ccc3cccc(-c4c5ccccc5c(-c5ccc(-c6cccc7c6oc6ccccc67)cc5)c5ccccc45)c3c2)cc1 ; c1ccc(-c2ccc3cccc(-c4c5ccccc5c(-c5cccc(-c6cccc7c6oc6ccccc67)c5)c5ccccc45)c3c2)cc1]

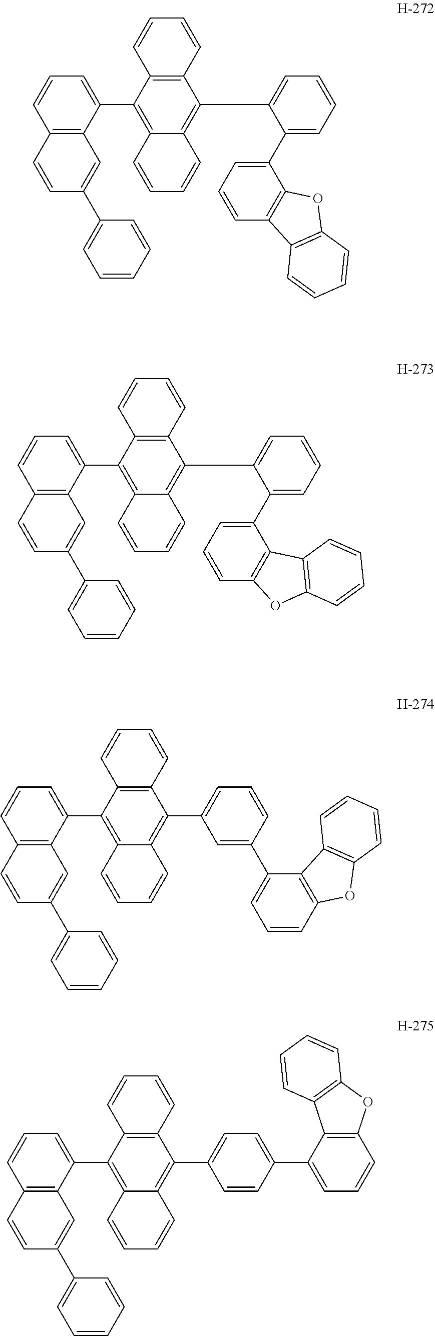 [SMILES: c1ccc(-c2ccc3cccc(-c4c5ccccc5c(-c5ccccc5-c5cccc6c5oc5ccccc56)c5ccccc45)c3c2)cc1 ; c1ccc(-c2ccc3cccc(-c4c5ccccc5c(-c5ccccc5-c5cccc6oc7ccccc7c56)c5ccccc45)c3c2)cc1 ; c1ccc(-c2ccc3cccc(-c4c5ccccc5c(-c5cccc(-c6cccc7oc8ccccc8c67)c5)c5ccccc45)c3c2)cc1 ; c1ccc(-c2ccc3cccc(-c4c5ccccc5c(-c5ccc(-c6cccc7oc8ccccc8c67)cc5)c5ccccc45)c3c2)cc1]

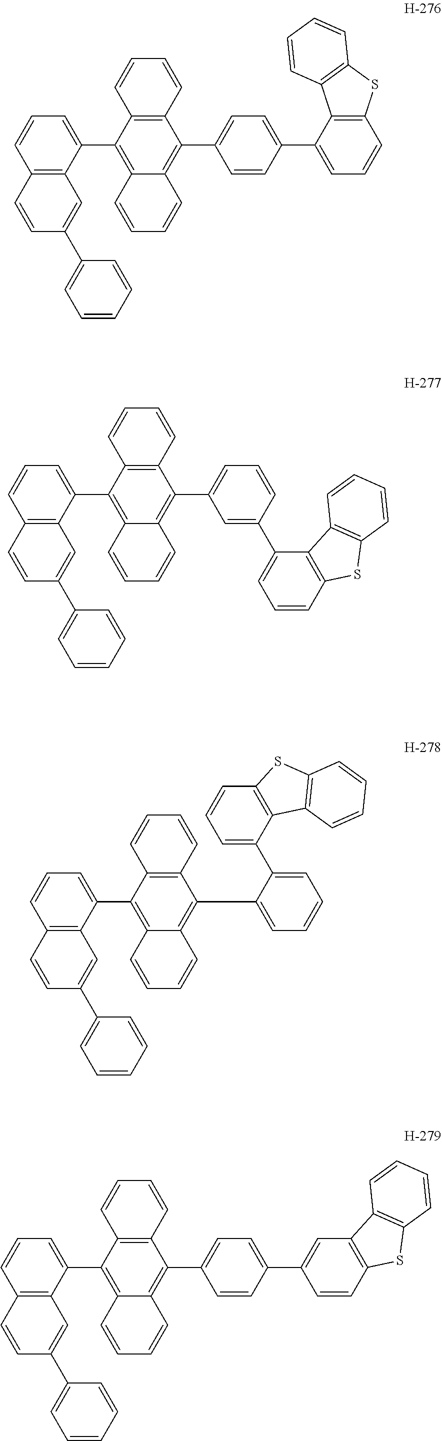 [SMILES: c1ccc(-c2ccc3cccc(-c4c5ccccc5c(-c5ccc(-c6cccc7sc8ccccc8c67)cc5)c5ccccc45)c3c2)cc1 ; c1ccc(-c2ccc3cccc(-c4c5ccccc5c(-c5cccc(-c6cccc7sc8ccccc8c67)c5)c5ccccc45)c3c2)cc1 ; c1ccc(-c2ccc3cccc(-c4c5ccccc5c(-c5ccccc5-c5cccc6sc7ccccc7c56)c5ccccc45)c3c2)cc1 ; c1ccc(-c2ccc3cccc(-c4c5ccccc5c(-c5ccc(-c6ccc7sc8ccccc8c7c6)cc5)c5ccccc45)c3c2)cc1]

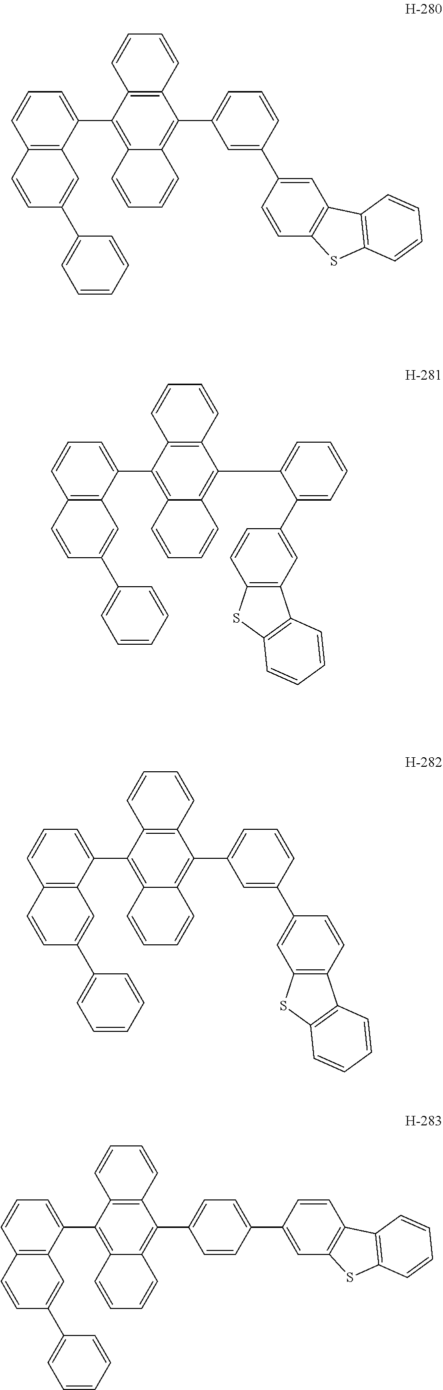 [SMILES: c1ccc(-c2ccc3cccc(-c4c5ccccc5c(-c5cccc(-c6ccc7sc8ccccc8c7c6)c5)c5ccccc45)c3c2)cc1 ; c1ccc(-c2ccc3cccc(-c4c5ccccc5c(-c5ccccc5-c5ccc6sc7ccccc7c6c5)c5ccccc45)c3c2)cc1 ; c1ccc(-c2ccc3cccc(-c4c5ccccc5c(-c5cccc(-c6ccc7c(c6)sc6ccccc67)c5)c5ccccc45)c3c2)cc1 ; c1ccc(-c2ccc3cccc(-c4c5ccccc5c(-c5ccc(-c6ccc7c(c6)sc6ccccc67)cc5)c5ccccc45)c3c2)cc1]

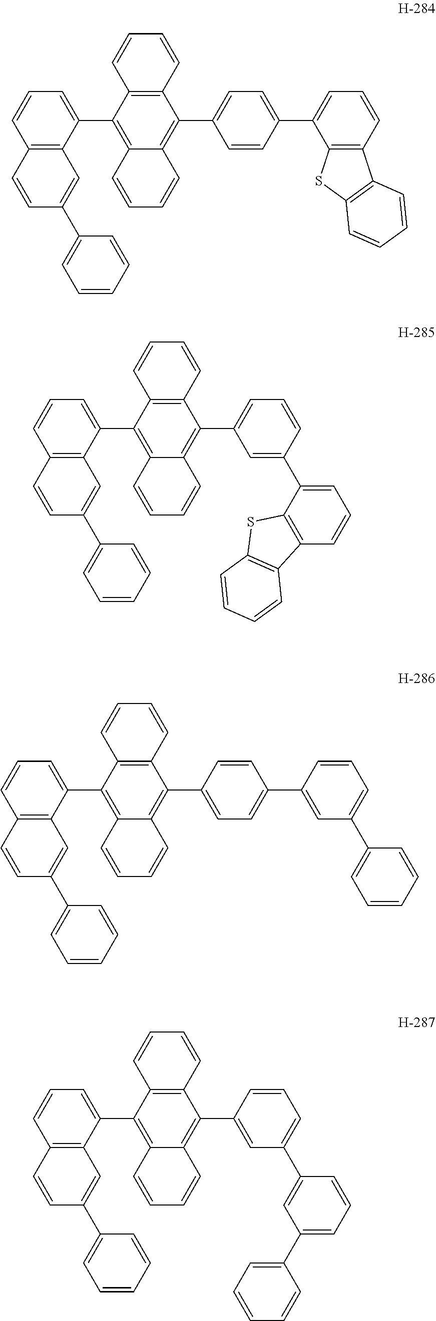

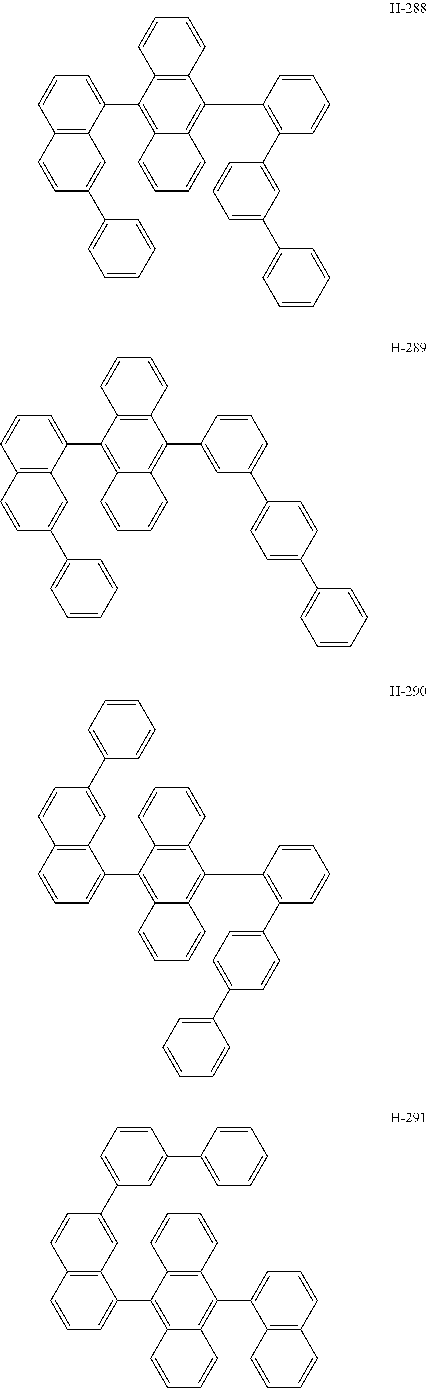

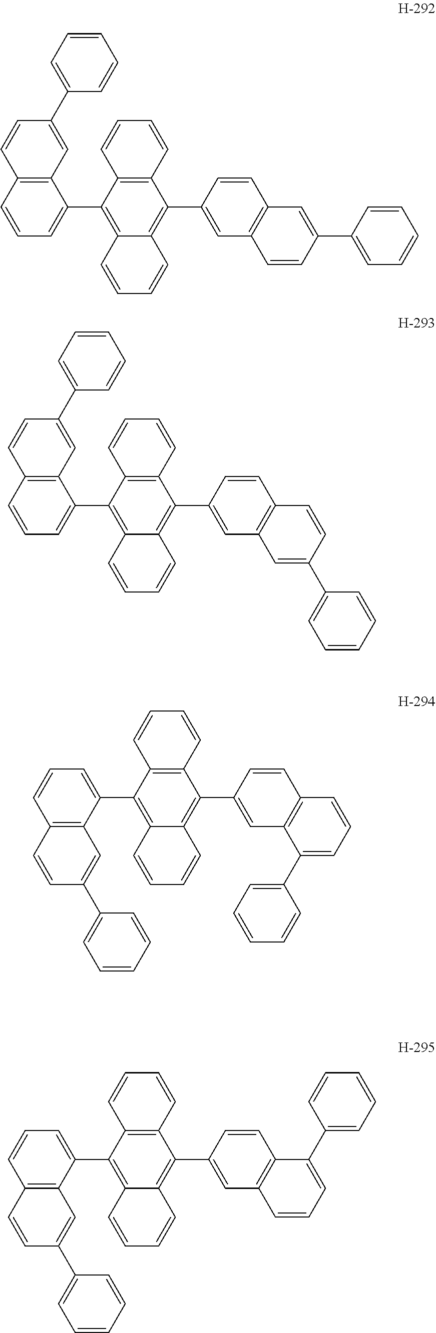

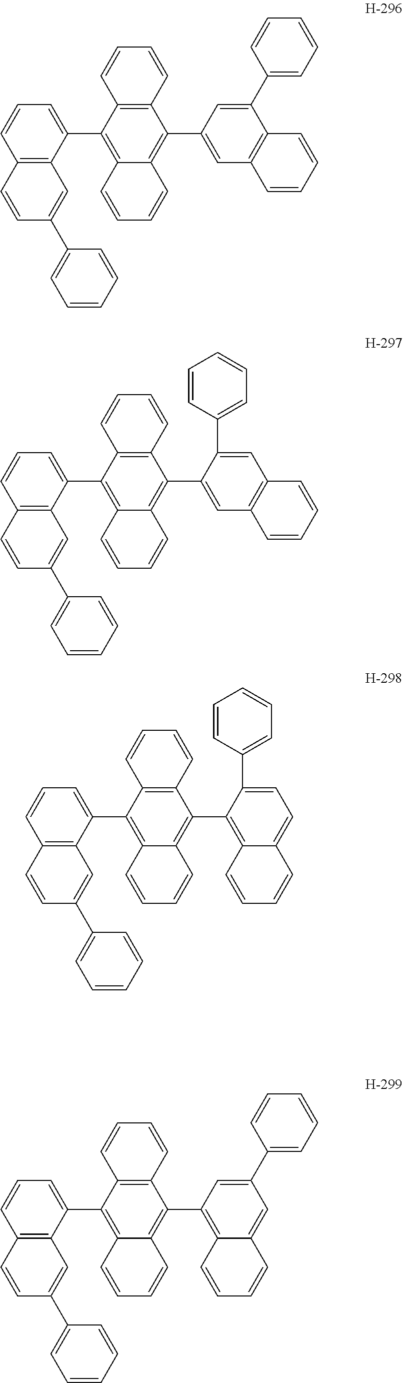

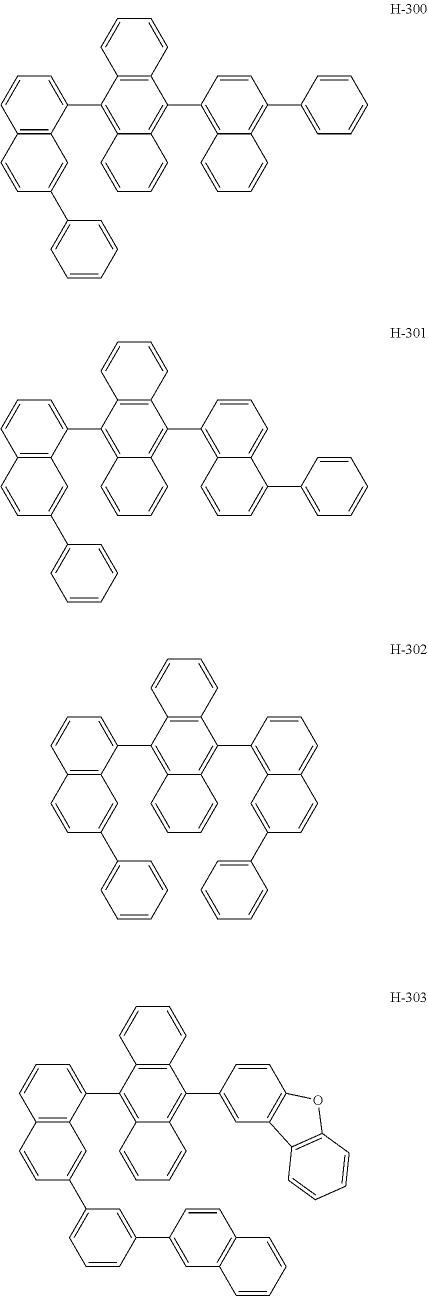

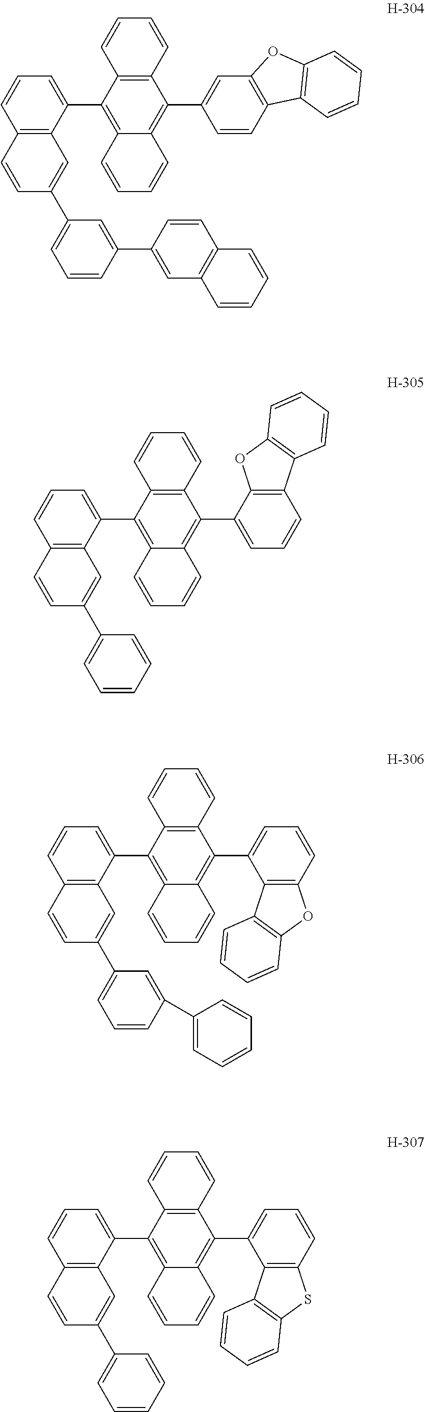 [SMILES: c1cc(-c2ccc3ccccc3c2)cc(-c2ccc3cccc(-c4c5ccccc5c(-c5ccc6c(c5)oc5ccccc56)c5ccccc45)c3c2)c1 ; c1ccc(-c2ccc3cccc(-c4c5ccccc5c(-c5cccc6c5oc5ccccc56)c5ccccc45)c3c2)cc1 ; c1ccc(-c2cccc(-c3ccc4cccc(-c5c6ccccc6c(-c6cccc7oc8ccccc8c67)c6ccccc56)c4c3)c2)cc1 ; c1ccc(-c2ccc3cccc(-c4c5ccccc5c(-c5cccc6sc7ccccc7c56)c5ccccc45)c3c2)cc1]

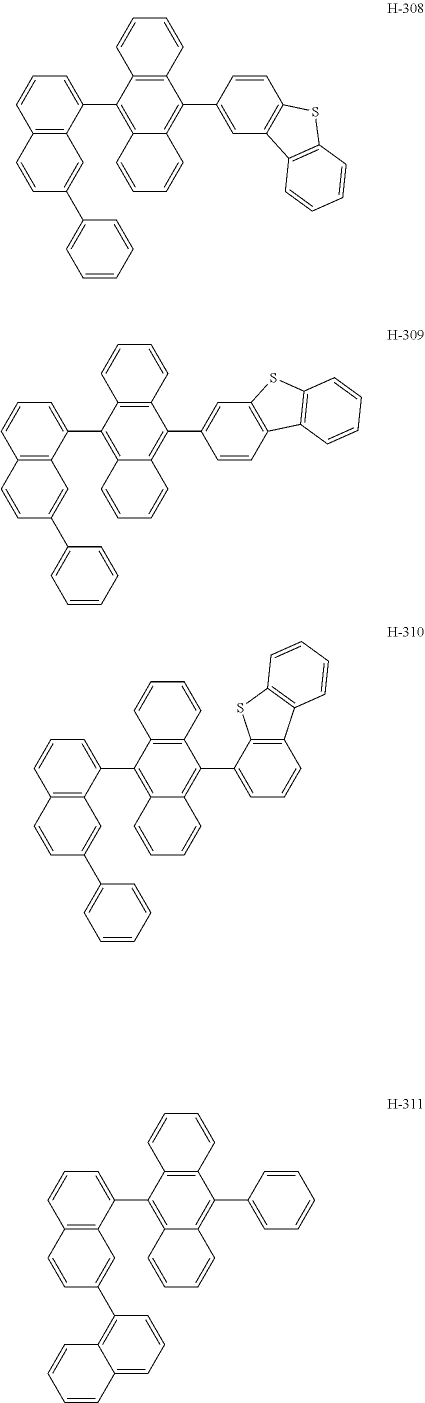

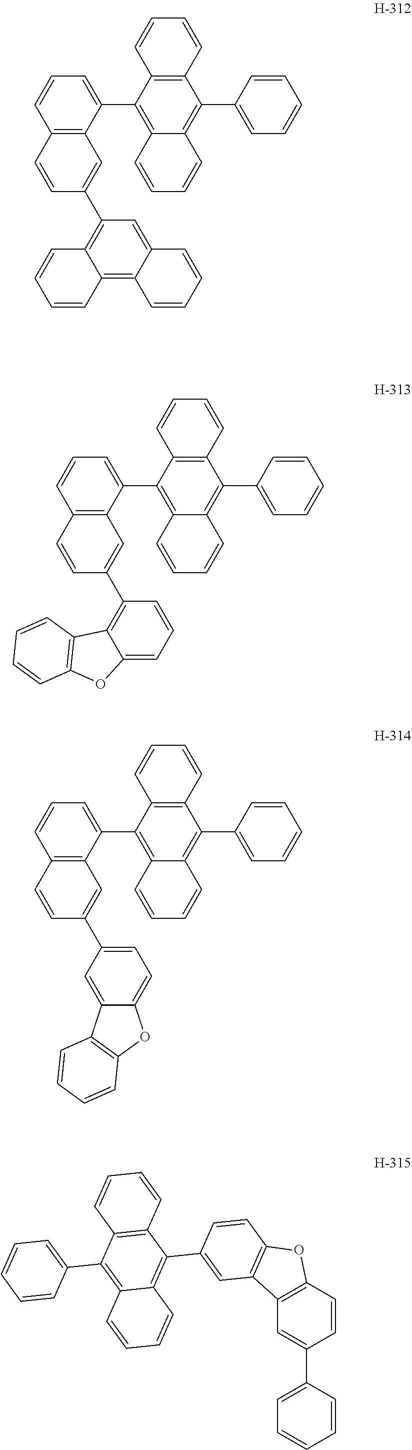

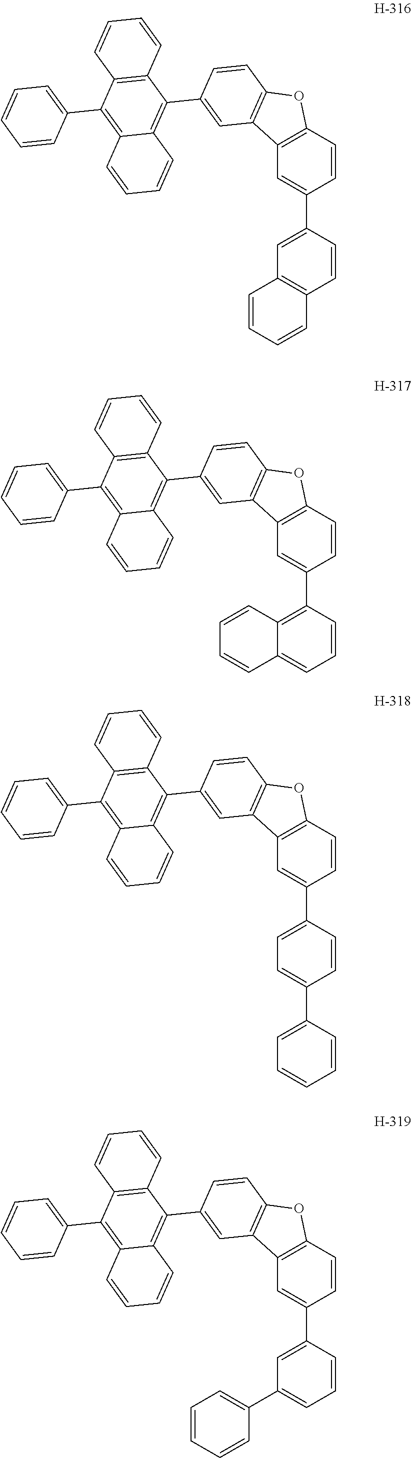 [SMILES: c1ccc(-c2c3ccccc3c(-c3ccc4oc5ccc(-c6ccc7ccccc7c6)cc5c4c3)c3ccccc23)cc1 ; c1ccc(-c2c3ccccc3c(-c3ccc4oc5ccc(-c6cccc7ccccc67)cc5c4c3)c3ccccc23)cc1 ; c1ccc(-c2ccc(-c3ccc4oc5ccc(-c6c7ccccc7c(-c7ccccc7)c7ccccc67)cc5c4c3)cc2)cc1 ; c1ccc(-c2cccc(-c3ccc4oc5ccc(-c6c7ccccc7c(-c7ccccc7)c7ccccc67)cc5c4c3)c2)cc1]

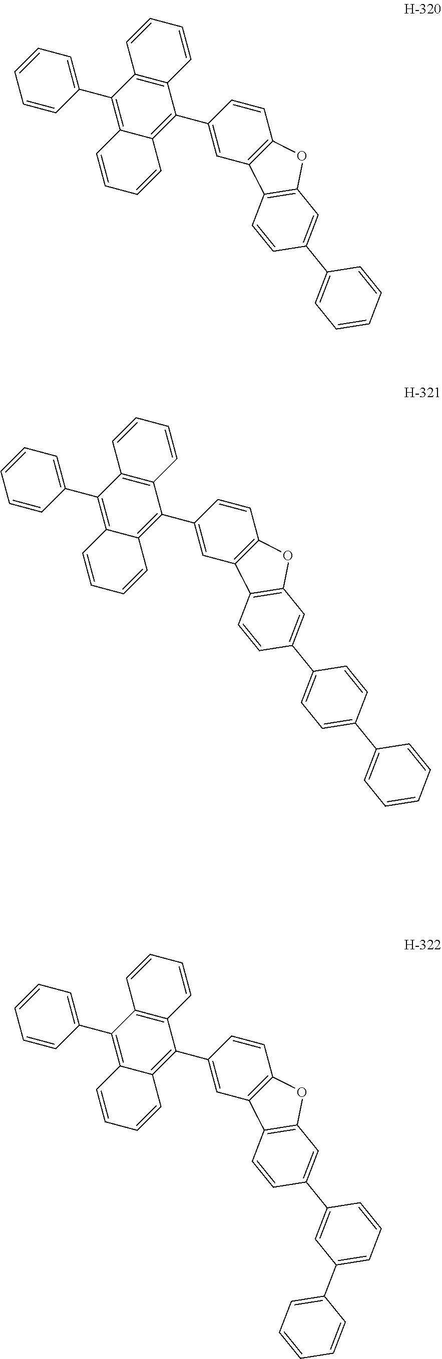 [SMILES: c1ccc(-c2ccc3c(c2)oc2ccc(-c4c5ccccc5c(-c5ccccc5)c5ccccc45)cc23)cc1 ; c1ccc(-c2ccc(-c3ccc4c(c3)oc3ccc(-c5c6ccccc6c(-c6ccccc6)c6ccccc56)cc34)cc2)cc1 ; c1ccc(-c2cccc(-c3ccc4c(c3)oc3ccc(-c5c6ccccc6c(-c6ccccc6)c6ccccc56)cc34)c2)cc1]

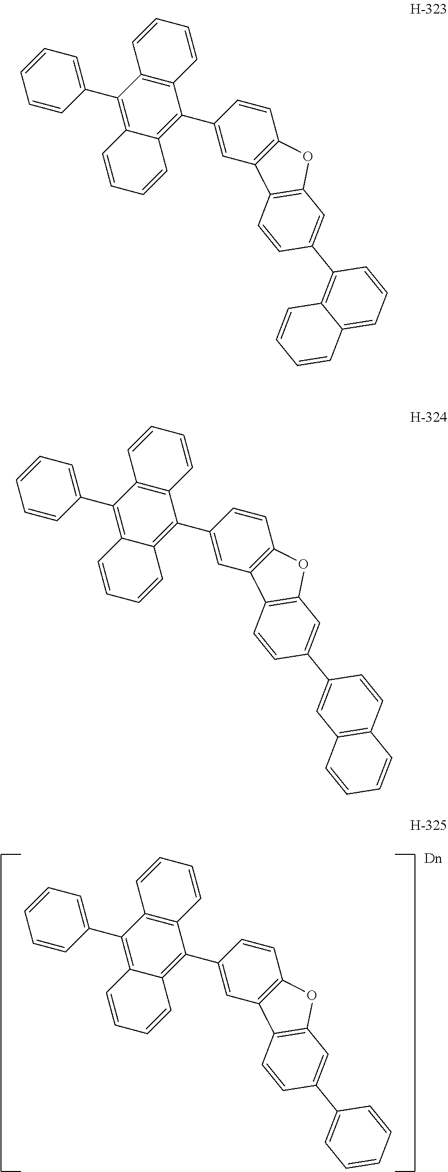

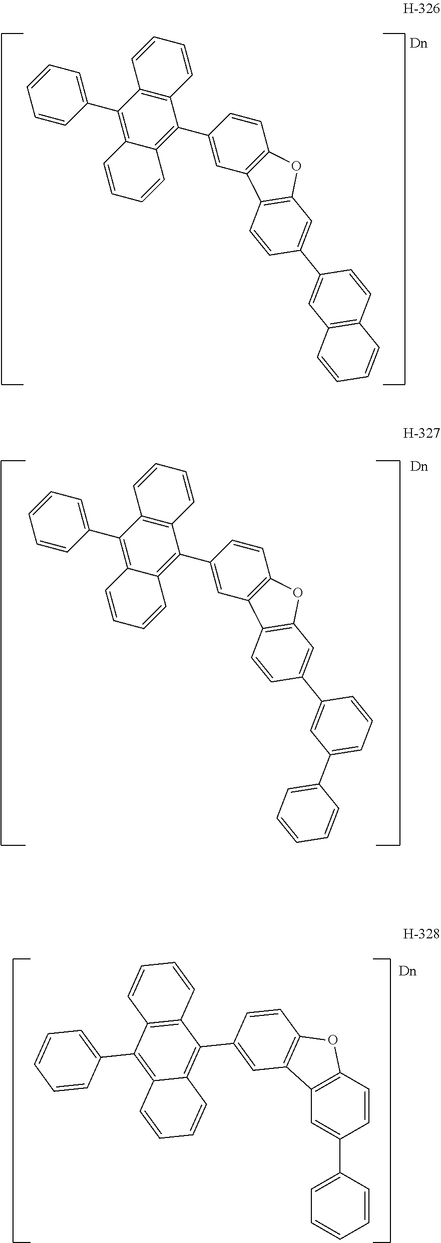

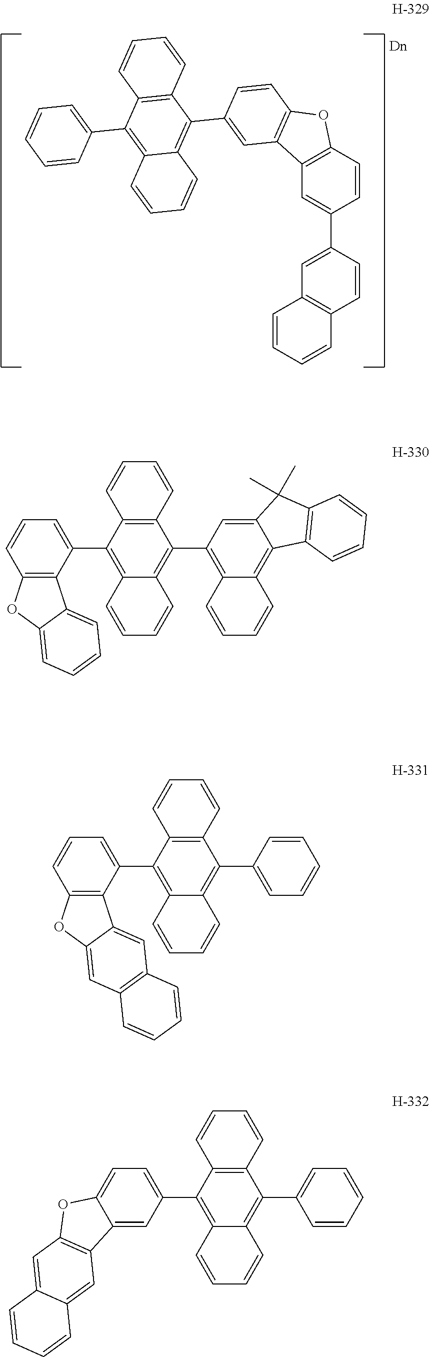

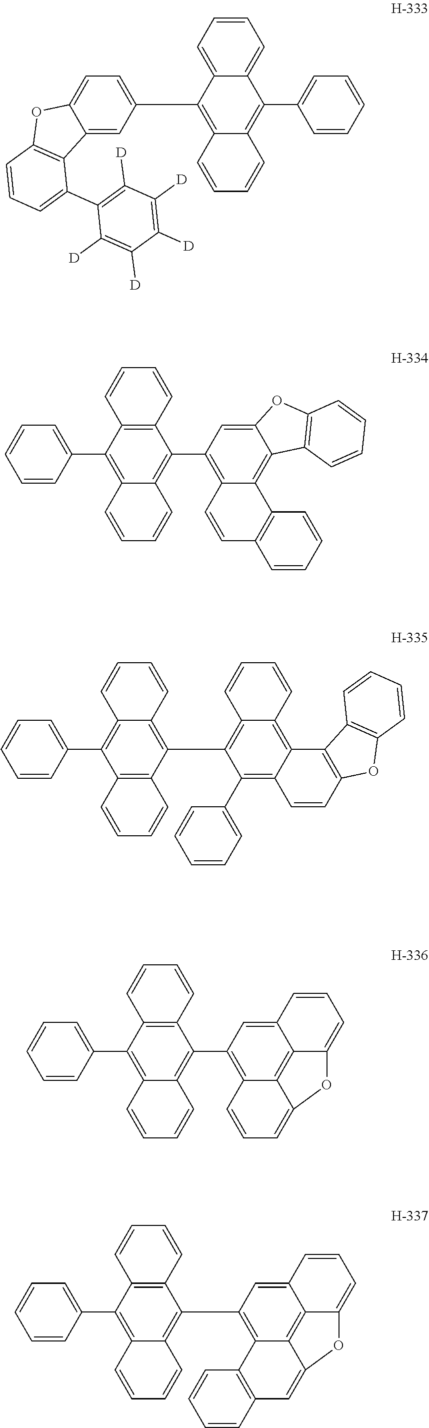 [SMILES: [2H]c1c([2H])c([2H])c(-c2cccc3oc4ccc(-c5c6ccccc6c(-c6ccccc6)c6ccccc56)cc4c23)c([2H])c1[2H] ; c1ccc(-c2c3ccccc3c(-c3cc4oc5ccccc5c4c4c3ccc3ccccc34)c3ccccc23)cc1 ; c1ccc(-c2c3ccccc3c(-c3c(-c4ccccc4)c4ccc5oc6ccccc6c5c4c4ccccc34)c3ccccc23)cc1 ; c1ccc(-c2c3ccccc3c(-c3cc4cccc5oc6cccc3c6c45)c3ccccc23)cc1 ; c1ccc(-c2c3ccccc3c(-c3cc4cccc5oc6cc7ccccc7c3c6c45)c3ccccc23)cc1]

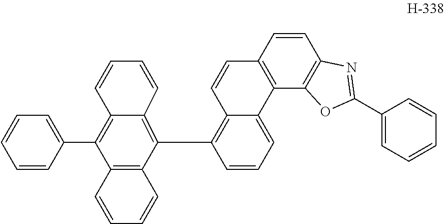 [SMILES: c1ccc(-c2nc3ccc4ccc5c(-c6c7ccccc7c(-c7ccccc7)c7ccccc67)cccc5c4c3o2)cc1]

In the compounds above, Dn means that n of the hydrogens are replaced with deuterium, wherein n represents an integer from 1 to 50. In one embodiment of the present disclosure, n is preferably an integer of 8 or more, more preferably an integer of 10 or more, and even more preferably, n is an integer of 15 or more. When deuterated with a number higher than the lower limit, the bond dissociation energy according to deuteration increases, thereby increasing the stability of the compound. When such a compound is used in an organic electroluminescent device, improved lifespan property may be exhibited.

The light-emitting material according to one embodiment includes at least one amine derivative(s) represented by formula 2 above. In one embodiment, the compound represented by formula 2 above may be a fluorescent dopant, for example, a fluorescent dopant for blue light-emitting.

In formula 2 above, Ring A, Ring B, and Ring C each independently represent a substituted or unsubstituted (C6-C30)aryl, or a substituted or unsubstituted (3- to 50-membered)heteroaryl. In one embodiment of the present disclosure, Ring A, Ring B and Ring C each independently represent a substituted or unsubstituted (C6-C25)aryl, or a substituted or unsubstituted (5- to 40-membered)heteroaryl. In another embodiment of the present disclosure, Ring A represents a substituted or unsubstituted (C6-C18)aryl, and Ring B and Ring C each independently represent a substituted or unsubstituted (C6-C18)aryl or a substituted or unsubstituted (5- to 36-membered)heteroaryl. For example, Ring A may be a substituted or unsubstituted benzene ring, unsubstituted naphthalene ring, or unsubstituted terphenyl ring, wherein the substituent of the substituted benzene ring may be at least one of deuterium; methyl unsubstituted or substituted with at least one deuterium; tert-butyl; diphenylamino unsubstituted or substituted with at least one of deuterium, methyl, and tert-phenyl; phenylnaphthylamino; dinaphthylamino; and a substituted or unsubstituted phenyl; naphthyl; biphenyl; terphenyl; triphenylenyl; carbazolyl; phenoxazinyl; phenothiazinyl; dimethylacridinyl; and dimethylxantenyl, and wherein the substituent of the substituted phenyl may be at least one of deuterium; methyl; carbazolyl; dibenzofuranyl; diphenylamino; phenoxazinyl; phenothiazinyl; and dimethylacridinyl. For example, Ring B and Ring C each independently may be a substituted or unsubstituted benzene ring, unsubstituted naphthalene ring, unsubstituted biphenyl ring, unsubstituted dibenzothiophene ring, unsubstituted dibenzofuran ring, carbazole ring substituted with at least one of phenyl and diphenylamino, 21-membered hetero ring containing boron and nitrogen substituted with at least one of methyl and phenyl 25-membered hetero ring containing boron and nitrogen substituted with at least one phenyl, or 36-membered hetero ring containing boron and nitrogen substituted with at least one methyl, wherein the substituent of the substituted benzene ring may be deuterium; methyl; tert-butyl; phenyl; a substituted or unsubstituted diphenylamino; phenylnaphthylamino; or phenylamino substituted with phenylcarbazolyl or dibenzofuranyl, and wherein the substituent of the substituted diphenylamino may be at least one of methyl and diphenylamino.

In formula 2 above, Y₁ represents B, and X₁ and X₂ each independently represent NR, wherein R represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N-(Ar₄)(Ar₅); or R may be linked to at least one of Ring A, Ring B and Ring C, to form a ring(s). In one embodiment of the present disclosure, R may be hydrogen, deuterium, a substituted or unsubstituted (C1-C20)alkyl, a substituted or unsubstituted (C6-C25)aryl, or a substituted or unsubstituted (3- to 25-membered)heteroaryl; or may be linked to at least one of Ring A, Ring B and Ring C, to form a ring(s). In another embodiment of the present disclosure, R may be hydrogen, deuterium, unsubstituted (C1-C10)alkyl, (C6-C18)aryl unsubstituted or substituted with at least one of (C1-C10)alkyl and di(C6-C18)arylamino, or (5- to 20-membered)heteroaryl substituted with (C6-C18)aryl; or may be linked to at least one of Ring A, Ring B and Ring C to form a ring(s). For example, R may be hydrogen; deuterium; methyl; phenyl unsubstituted or substituted with at least one of methyl and tert-butyl; naphthyl; biphenyl unsubstituted or substituted with diphenylamino; triphenylenyl; or carbazolyl substituted with phenyl; or may be linked to at least one of Ring A, Ring B and Ring C, to form a ring(s).

According to one embodiment of the present disclosure, the compound represented by formula 2 above may be represented by the following formula 2-1.

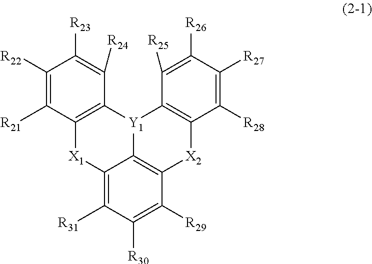

In formula 2-1,

R₂₁ to R₃₁ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N-(Ar₄)(Ar₅); or may be linked to the adjacent substituent to form a ring(s), and

Y₁, X₁, X₂, L₄, Ar₄, and Ar₅ are as defined in formula 2 above.

In one embodiment of the present disclosure, R₂₁ to R₃₁ each independently may be hydrogen, deuterium, a substituted or unsubstituted (C1-C20)alkyl, a substituted or unsubstituted (C6-C25)aryl, a substituted or unsubstituted (5- to 20-membered)heteroaryl, or -L₄-N-(Ar₄)(Ar₅); or may be linked to the adjacent substituent to form a ring(s). In another embodiment of the present disclosure, R₂₁ to R₃₁ each independently may be hydrogen, deuterium, unsubstituted (C1-C10)alkyl; (C6-C18)aryl unsubstituted or substituted with at least one of (C1-C10)alkyl, (13- to 18-membered)heteroaryl, and di(C6-C18)arylamino; (5- to 18-membered)heteroaryl unsubstituted or substituted with at least one (C1-C10)alkyl, or -L₄-N-(Ar₄)(Ar₅); or may be linked to the adjacent substituent to form a ring(s). For example, R₂₁ to R₃₁ each independently may be hydrogen, methyl, tert-butyl, a substituted or unsubstituted phenyl, biphenyl, terphenyl, triphenylenyl, carbazolyl, phenoxazinyl, phenothiazinyl, dimethylacridinyl, dimethylxanthenyl, diphenylamino unsubstituted or substituted with at least one of methyl and diphenylamino, phenylamino substituted with phenylnaphthylamino, dibiphenylamino, phenylcarbazolyl or dibenzofuranyl, or (17- to 21-membered)heteroaryl substituted with at least one of methyl and phenyl; or may be linked to the adjacent substituent to form benzene ring, indole ring substituted with at least one of phenyl and diphenylamino, benzofuran ring, benzothiophene ring, or 19-membered hetero ring substituted with at least one methyl. The substitute of the substituted phenyl may be at least one of methyl, carbazolyl, dibenzofuranyl, diphenylamino, phenoxazinyl, phenothiazinyl, and dimethylacridinyl.

According to one embodiment, the compound represented by formula 2 above may be more specifically illustrated by the following compounds, but is not limited thereto.

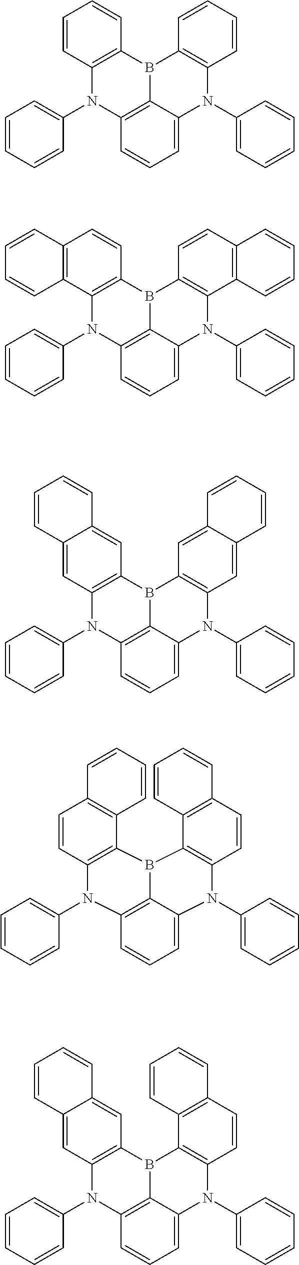 [SMILES: c1ccc(N2c3ccccc3B3c4ccccc4N(c4ccccc4)c4cccc2c43)cc1 ; c1ccc(N2c3cccc4c3B(c3ccc5ccccc5c32)c2ccc3ccccc3c2N4c2ccccc2)cc1 ; c1ccc(N2c3cc4ccccc4cc3B3c4cc5ccccc5cc4N(c4ccccc4)c4cccc2c43)cc1 ; c1ccc(N2c3cccc4c3B(c3c2ccc2ccccc32)c2c(ccc3ccccc23)N4c2ccccc2)cc1 ; c1ccc(N2c3cc4ccccc4cc3B3c4c2cccc4N(c2ccccc2)c2ccc4ccccc4c23)cc1]

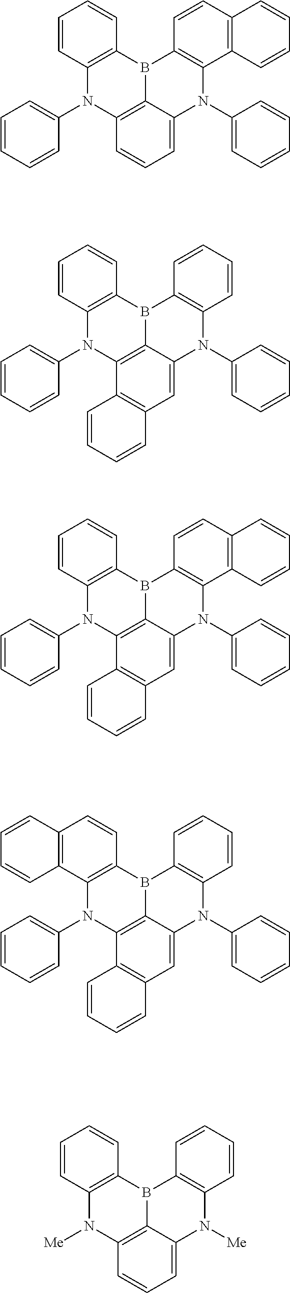 [SMILES: c1ccc(N2c3ccccc3B3c4ccc5ccccc5c4N(c4ccccc4)c4cccc2c43)cc1 ; c1ccc(N2c3ccccc3B3c4ccccc4N(c4ccccc4)c4c3c2cc2ccccc42)cc1 ; c1ccc(N2c3cc4ccccc4c4c3B(c3ccccc3N4c3ccccc3)c3ccc4ccccc4c32)cc1 ; c1ccc(N2c3ccccc3B3c4ccc5ccccc5c4N(c4ccccc4)c4c3c2cc2ccccc42)cc1 ; CN1c2ccccc2B2c3ccccc3N(C)c3cccc1c32]

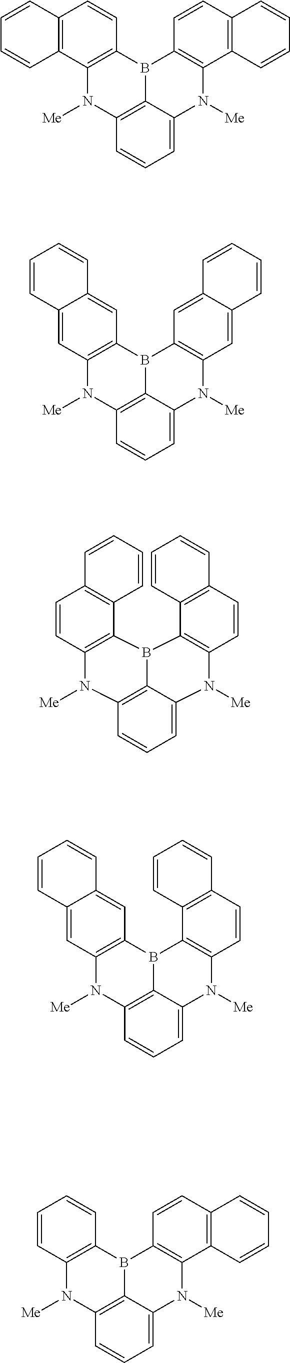 [SMILES: CN1c2cccc3c2B(c2ccc4ccccc4c21)c1ccc2ccccc2c1N3C ; CN1c2cc3ccccc3cc2B2c3cc4ccccc4cc3N(C)c3cccc1c32 ; CN1c2cccc3c2B(c2c1ccc1ccccc21)c1c(ccc2ccccc12)N3C ; CN1c2cc3ccccc3cc2B2c3c1cccc3N(C)c1ccc3ccccc3c12 ; CN1c2ccccc2B2c3ccc4ccccc4c3N(C)c3cccc1c32]

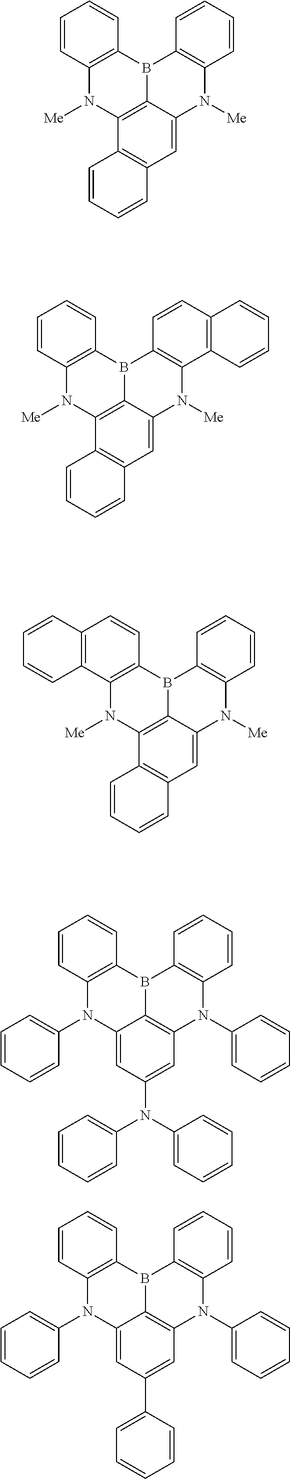

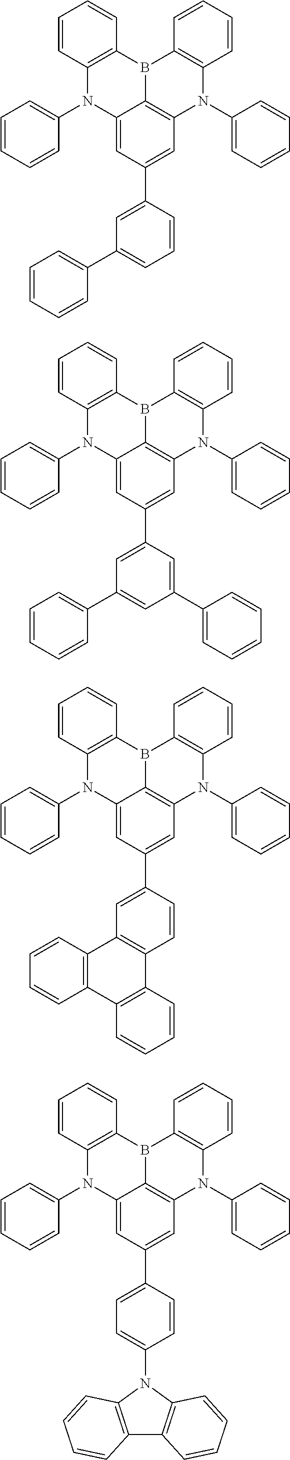

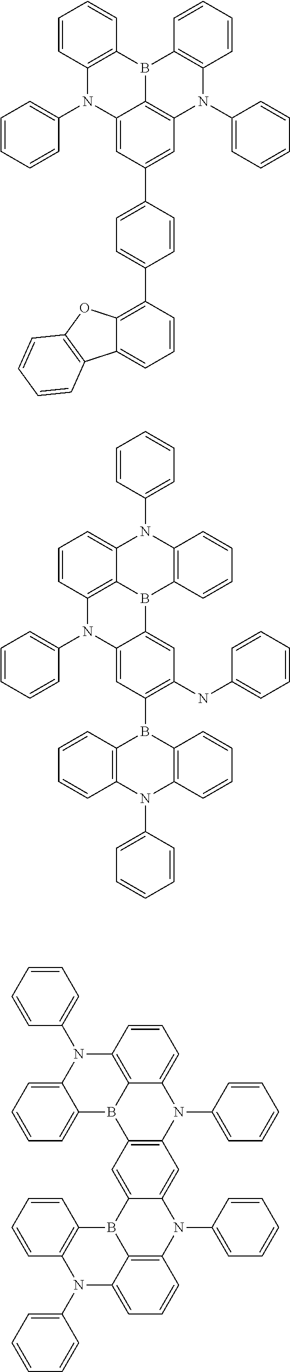 [SMILES: c1ccc(N2c3ccccc3B3c4ccccc4N(c4ccccc4)c4cc(-c5ccc(-c6cccc7c6oc6ccccc67)cc5)cc2c43)cc1 ; c1ccc(Nc2cc3c(cc2B2c4ccccc4N(c4ccccc4)c4ccccc42)N(c2ccccc2)c2cccc4c2B3c2ccccc2N4c2ccccc2)cc1 ; c1ccc(N2c3ccccc3B3c4cc5c(cc4N(c4ccccc4)c4cccc2c43)N(c2ccccc2)c2cccc3c2B5c2ccccc2N3c2ccccc2)cc1]

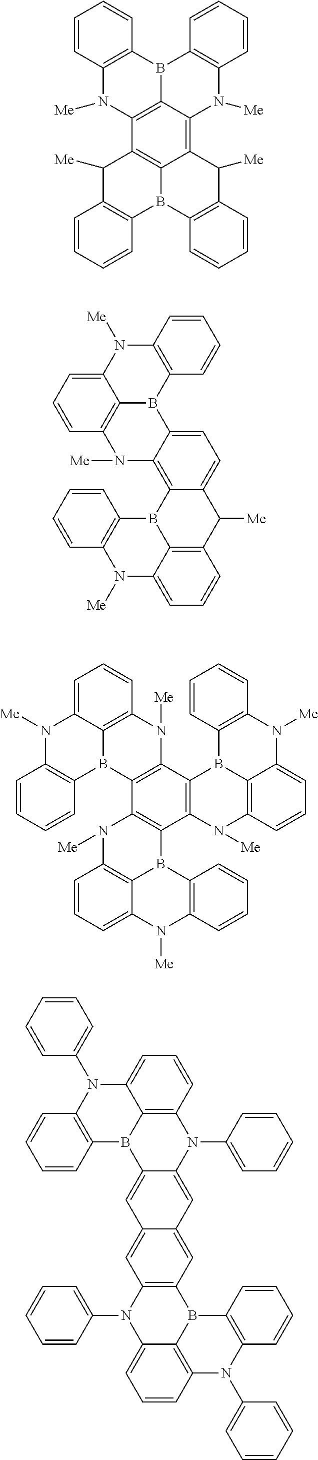 [SMILES: CC1c2ccccc2B2c3ccccc3C(C)c3c2c1c1c2c3N(C)c3ccccc3B2c2ccccc2N1C ; CC1c2cccc3c2B(c2ccccc2N3C)c2c1ccc1c2N(C)c2cccc3c2B1c1ccccc1N3C ; CN1c2ccccc2B2c3c1cccc3N(C)c1c3c(c4c(c12)N(C)c1cccc2c1B4c1ccccc1N2C)N(C)c1cccc2c1B3c1ccccc1N2C ; c1ccc(N2c3ccccc3B3c4cc5cc6c(cc5cc4N(c4ccccc4)c4cccc2c43)B2c3ccccc3N(c3ccccc3)c3cccc(c32)N6c2ccccc2)cc1]

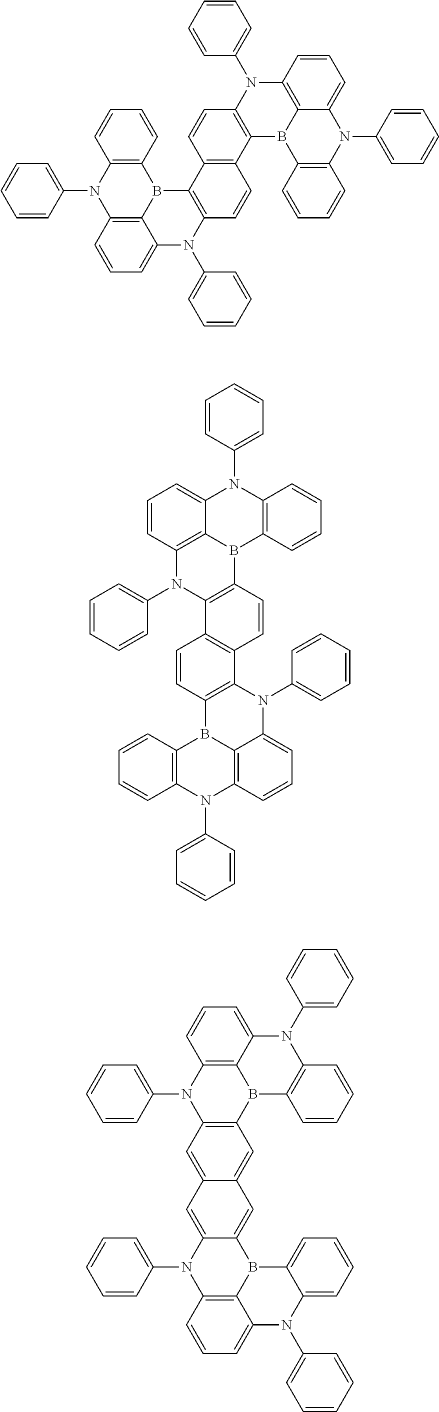 [SMILES: c1ccc(N2c3ccccc3B3c4c2cccc4N(c2ccccc2)c2ccc4c5c(ccc4c23)N(c2ccccc2)c2cccc3c2B5c2ccccc2N3c2ccccc2)cc1 ; c1ccc(N2c3ccccc3B3c4ccc5c6c(ccc5c4N(c4ccccc4)c4cccc2c43)B2c3ccccc3N(c3ccccc3)c3cccc(c32)N6c2ccccc2)cc1 ; c1ccc(N2c3ccccc3B3c4cc5cc6c(cc5cc4N(c4ccccc4)c4cccc2c43)N(c2ccccc2)c2cccc3c2B6c2ccccc2N3c2ccccc2)cc1]

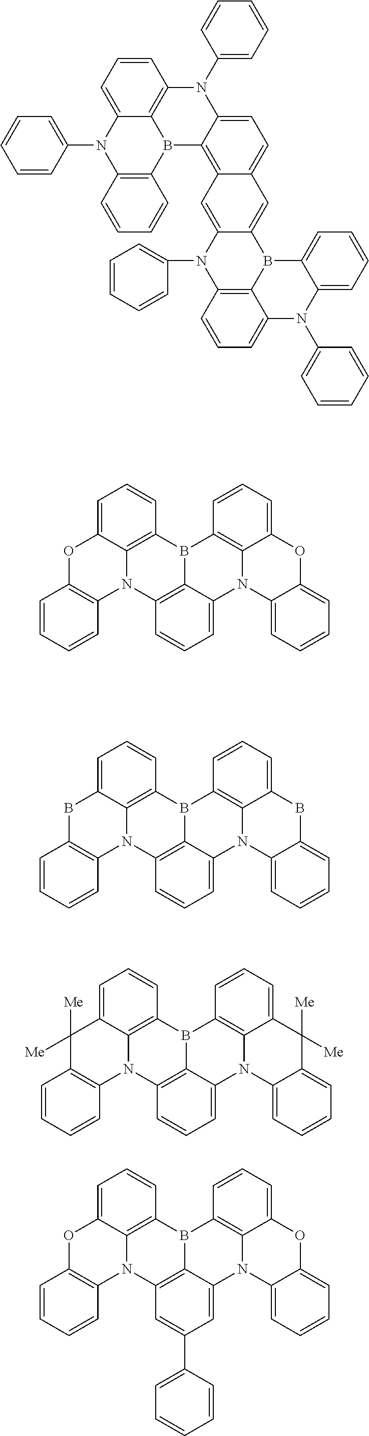

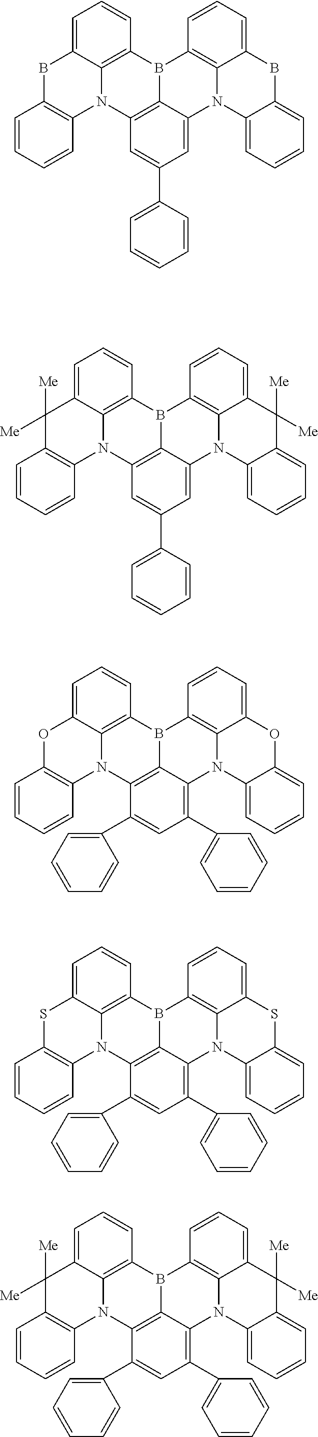

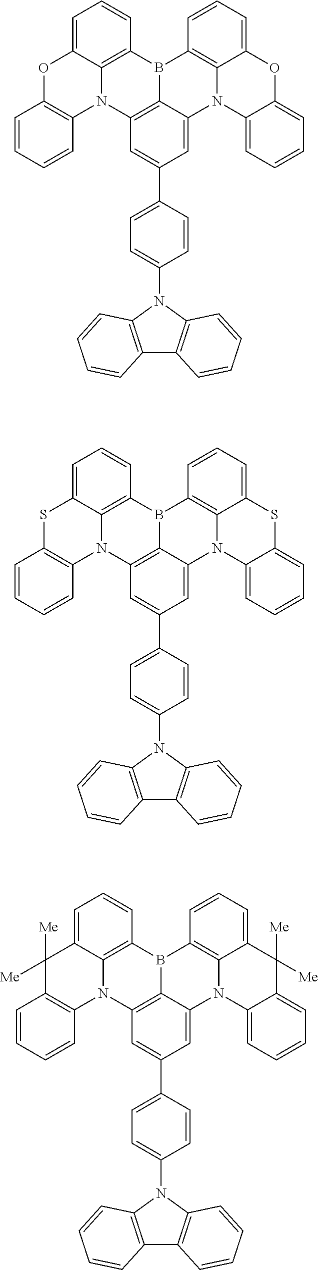 [SMILES: c1ccc2c(c1)Oc1cccc3c1N2c1cc(-c2ccc(-n4c5ccccc5c5ccccc54)cc2)cc2c1B3c1cccc3c1N2c1ccccc1O3 ; c1ccc2c(c1)Sc1cccc3c1N2c1cc(-c2ccc(-n4c5ccccc5c5ccccc54)cc2)cc2c1B3c1cccc3c1N2c1ccccc1S3 ; CC1(C)c2ccccc2N2c3cc(-c4ccc(-n5c6ccccc6c6ccccc65)cc4)cc4c3B(c3cccc1c32)c1cccc2c1N4c1ccccc1C2(C)C]

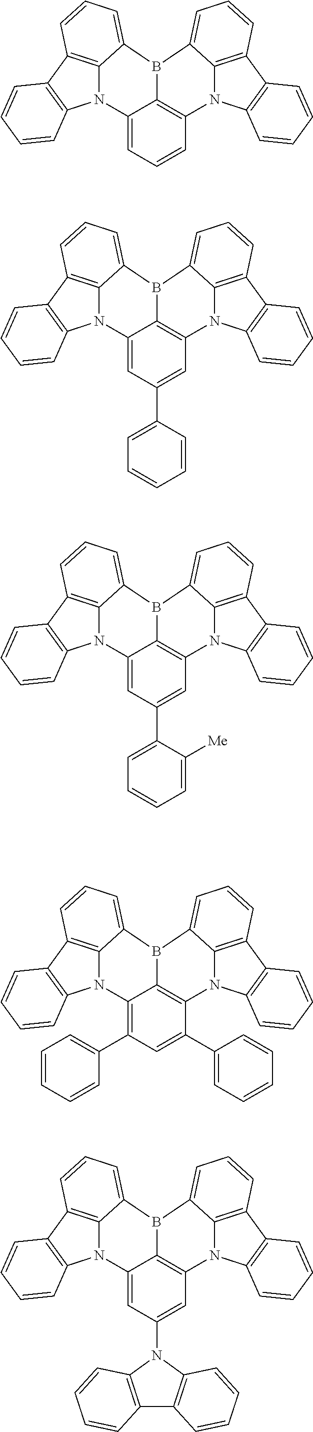 [SMILES: c1cc2c3c(c1)-n1c4ccccc4c4cccc(c41)B3c1cccc3c4ccccc4n-2c13 ; c1ccc(-c2cc3c4c(c2)-n2c5ccccc5c5cccc(c52)B4c2cccc4c5ccccc5n-3c24)cc1 ; Cc1ccccc1-c1cc2c3c(c1)-n1c4ccccc4c4cccc(c41)B3c1cccc3c4ccccc4n-2c13 ; c1ccc(-c2cc(-c3ccccc3)c3c4c2-n2c5ccccc5c5cccc(c52)B4c2cccc4c5ccccc5n-3c24)cc1 ; c1ccc2c(c1)c1ccccc1n2-c1cc2c3c(c1)-n1c4ccccc4c4cccc(c41)B3c1cccc3c4ccccc4n-2c13]

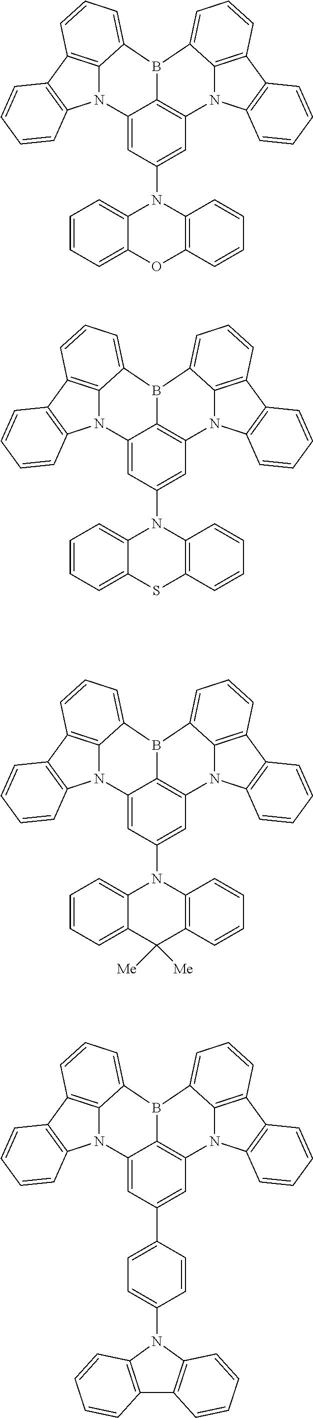 [SMILES: c1ccc2c(c1)Oc1ccccc1N2c1cc2c3c(c1)-n1c4ccccc4c4cccc(c41)B3c1cccc3c4ccccc4n-2c13 ; c1ccc2c(c1)Sc1ccccc1N2c1cc2c3c(c1)-n1c4ccccc4c4cccc(c41)B3c1cccc3c4ccccc4n-2c13 ; CC1(C)c2ccccc2N(c2cc3c4c(c2)-n2c5ccccc5c5cccc(c52)B4c2cccc4c5ccccc5n-3c24)c2ccccc21 ; c1ccc2c(c1)c1ccccc1n2-c1ccc(-c2cc3c4c(c2)-n2c5ccccc5c5cccc(c52)B4c2cccc4c5ccccc5n-3c24)cc1]

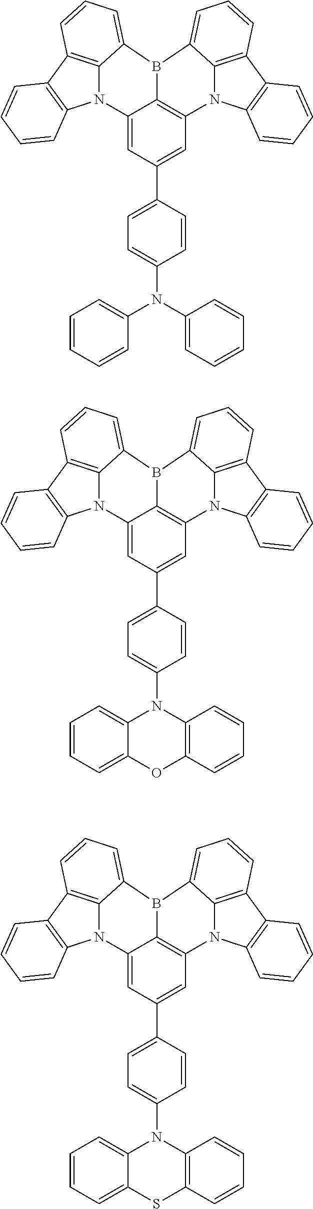

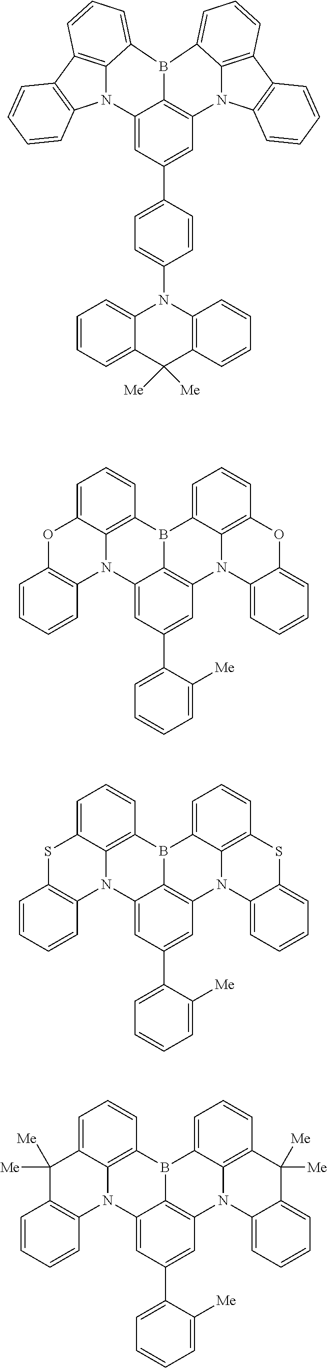 [SMILES: CC1(C)c2ccccc2N(c2ccc(-c3cc4c5c(c3)-n3c6ccccc6c6cccc(c63)B5c3cccc5c6ccccc6n-4c35)cc2)c2ccccc21 ; Cc1ccccc1-c1cc2c3c(c1)N1c4ccccc4Oc4cccc(c41)B3c1cccc3c1N2c1ccccc1O3 ; Cc1ccccc1-c1cc2c3c(c1)N1c4ccccc4Sc4cccc(c41)B3c1cccc3c1N2c1ccccc1S3 ; Cc1ccccc1-c1cc2c3c(c1)N1c4ccccc4C(C)(C)c4cccc(c41)B3c1cccc3c1N2c1ccccc1C3(C)C]

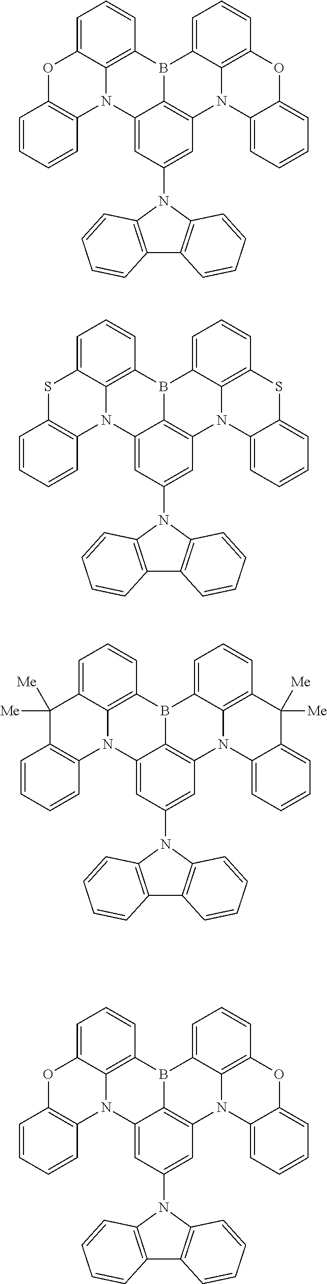

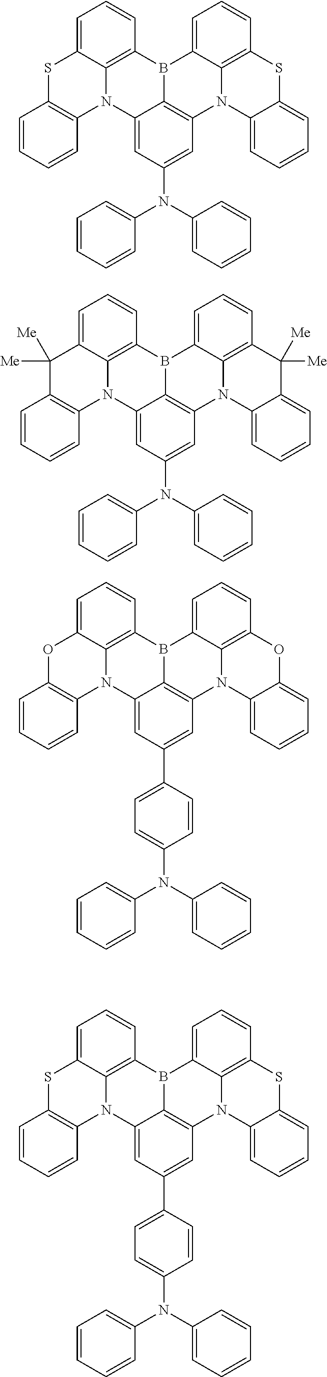

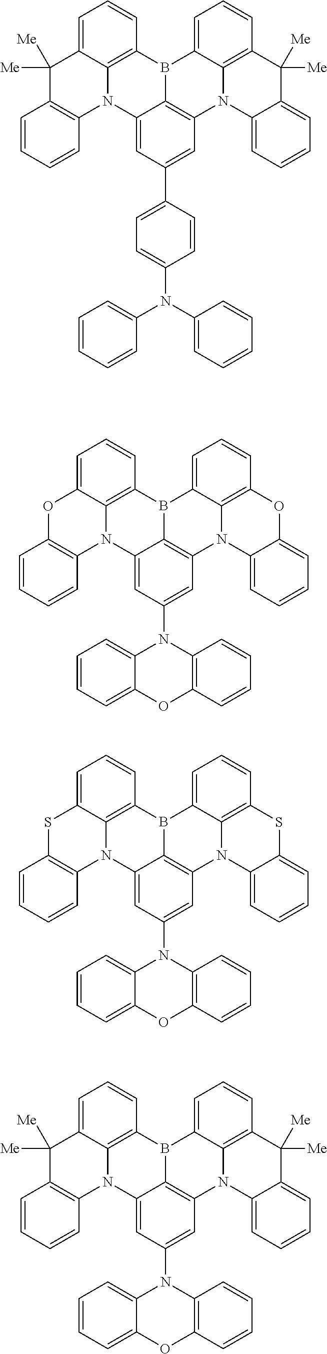 [SMILES: CC1(C)c2ccccc2N2c3cc(-c4ccc(N(c5ccccc5)c5ccccc5)cc4)cc4c3B(c3cccc1c32)c1cccc2c1N4c1ccccc1C2(C)C ; c1ccc2c(c1)Oc1ccccc1N2c1cc2c3c(c1)N1c4ccccc4Oc4cccc(c41)B3c1cccc3c1N2c1ccccc1O3 ; c1ccc2c(c1)Oc1ccccc1N2c1cc2c3c(c1)N1c4ccccc4Sc4cccc(c41)B3c1cccc3c1N2c1ccccc1S3 ; CC1(C)c2ccccc2N2c3cc(N4c5ccccc5Oc5ccccc54)cc4c3B(c3cccc1c32)c1cccc2c1N4c1ccccc1C2(C)C]

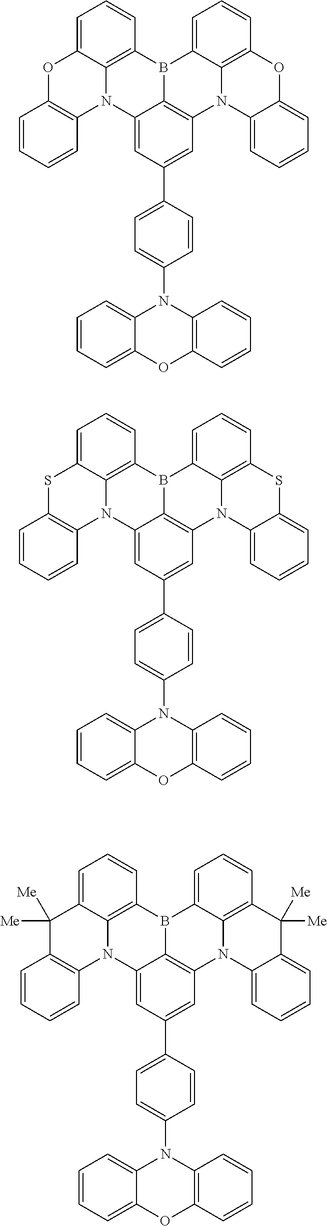

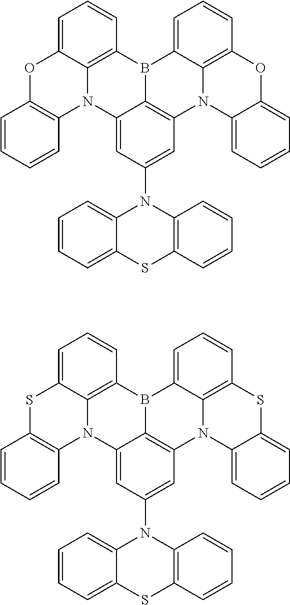 [SMILES: c1ccc2c(c1)Oc1cccc3c1N2c1cc(N2c4ccccc4Sc4ccccc42)cc2c1B3c1cccc3c1N2c1ccccc1O3 ; c1ccc2c(c1)Sc1ccccc1N2c1cc2c3c(c1)N1c4ccccc4Sc4cccc(c41)B3c1cccc3c1N2c1ccccc1S3]

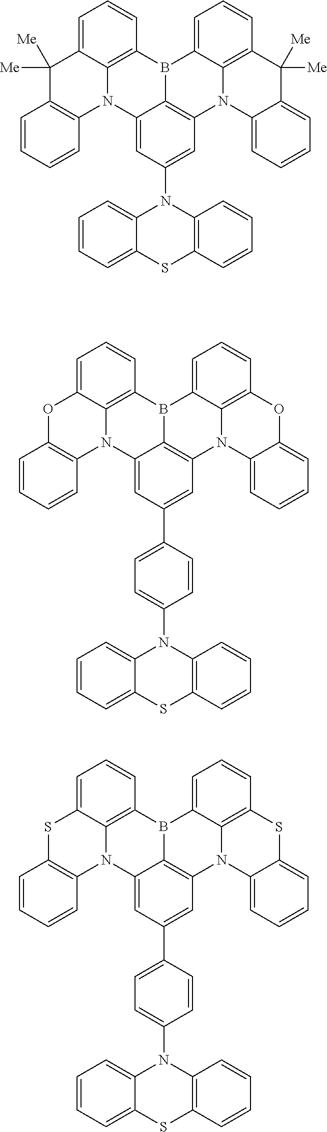 [SMILES: CC1(C)c2ccccc2N2c3cc(N4c5ccccc5Sc5ccccc54)cc4c3B(c3cccc1c32)c1cccc2c1N4c1ccccc1C2(C)C ; c1ccc2c(c1)Oc1cccc3c1N2c1cc(-c2ccc(N4c5ccccc5Sc5ccccc54)cc2)cc2c1B3c1cccc3c1N2c1ccccc1O3 ; c1ccc2c(c1)Sc1ccccc1N2c1ccc(-c2cc3c4c(c2)N2c5ccccc5Sc5cccc(c52)B4c2cccc4c2N3c2ccccc2S4)cc1]

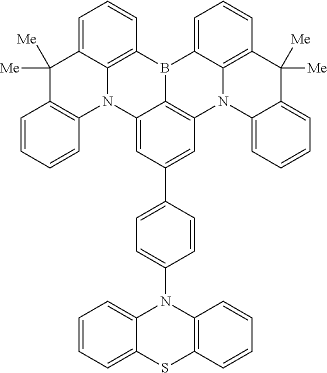 [SMILES: CC1(C)c2ccccc2N2c3cc(-c4ccc(N5c6ccccc6Sc6ccccc65)cc4)cc4c3B(c3cccc1c32)c1cccc2c1N4c1ccccc1C2(C)C]

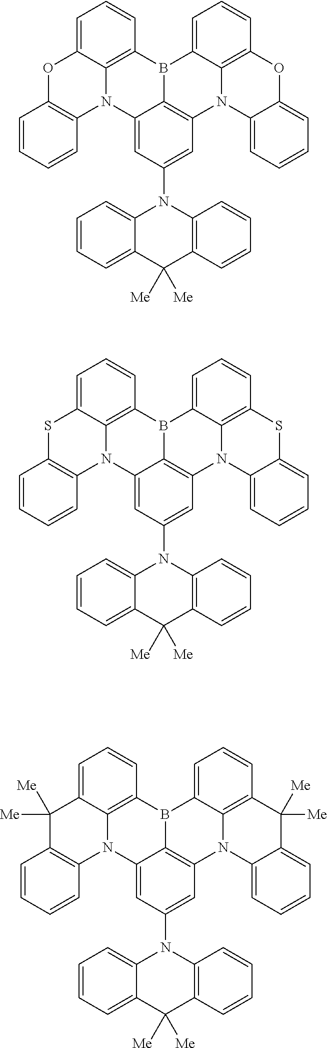 [SMILES: CC1(C)c2ccccc2N(c2cc3c4c(c2)N2c5ccccc5Oc5cccc(c52)B4c2cccc4c2N3c2ccccc2O4)c2ccccc21 ; CC1(C)c2ccccc2N(c2cc3c4c(c2)N2c5ccccc5Sc5cccc(c52)B4c2cccc4c2N3c2ccccc2S4)c2ccccc21 ; CC1(C)c2ccccc2N(c2cc3c4c(c2)N2c5ccccc5C(C)(C)c5cccc(c52)B4c2cccc4c2N3c2ccccc2C4(C)C)c2ccccc21]

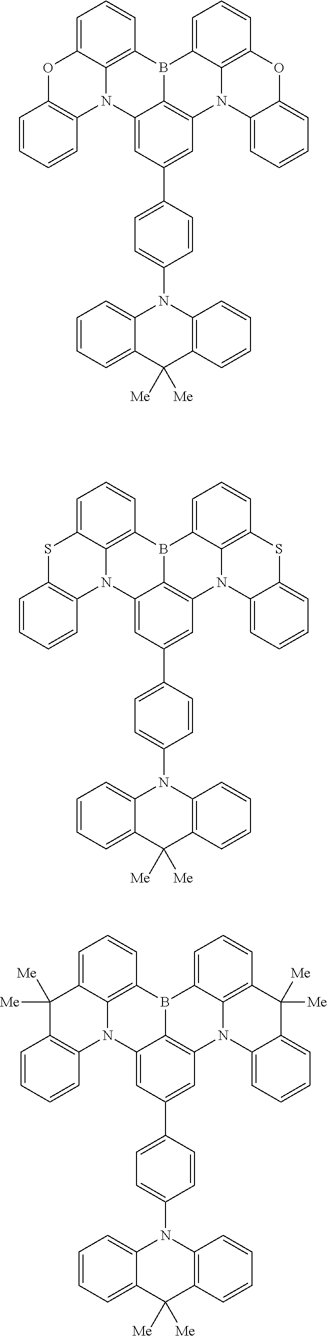 [SMILES: CC1(C)c2ccccc2N(c2ccc(-c3cc4c5c(c3)N3c6ccccc6Oc6cccc(c63)B5c3cccc5c3N4c3ccccc3O5)cc2)c2ccccc21 ; CC1(C)c2ccccc2N(c2ccc(-c3cc4c5c(c3)N3c6ccccc6Sc6cccc(c63)B5c3cccc5c3N4c3ccccc3S5)cc2)c2ccccc21 ; CC1(C)c2ccccc2N(c2ccc(-c3cc4c5c(c3)N3c6ccccc6C(C)(C)c6cccc(c63)B5c3cccc5c3N4c3ccccc3C5(C)C)cc2)c2ccccc21]

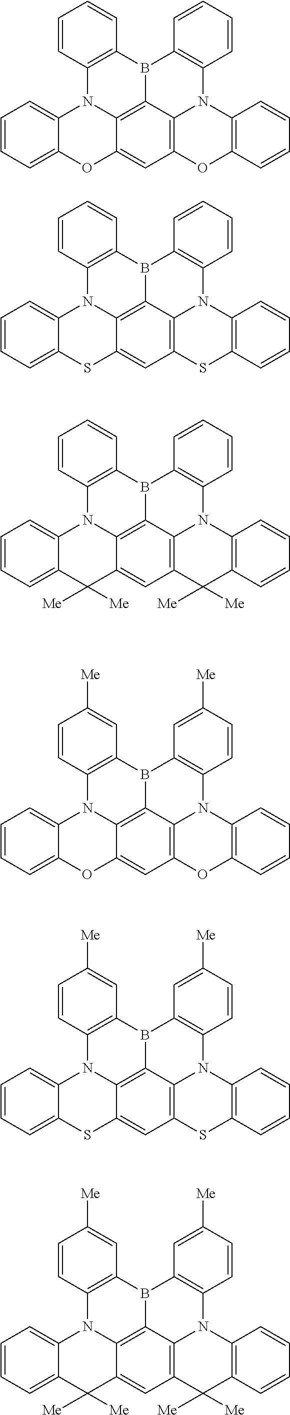

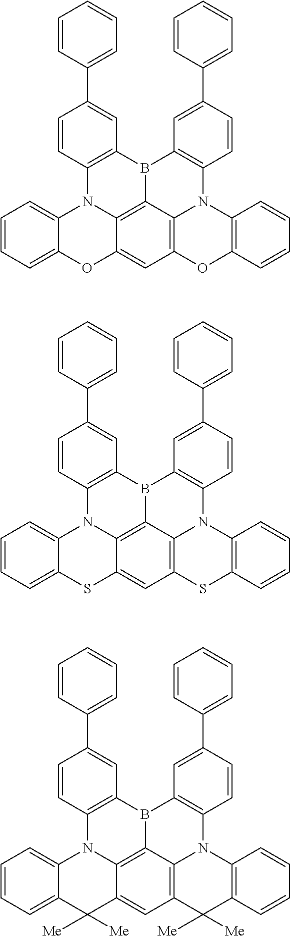 [SMILES: c1ccc(-c2ccc3c(c2)B2c4cc(-c5ccccc5)ccc4-n4c5ccccc5oc5cc6oc7ccccc7n-3c6c2c54)cc1 ; c1ccc(-c2ccc3c(c2)B2c4cc(-c5ccccc5)ccc4-n4c5ccccc5sc5cc6sc7ccccc7n-3c6c2c54)cc1 ; CC1(C)c2ccccc2N2c3ccc(-c4ccccc4)cc3B3c4cc(-c5ccccc5)ccc4N4c5ccccc5C(C)(C)c5cc1c2c3c54]

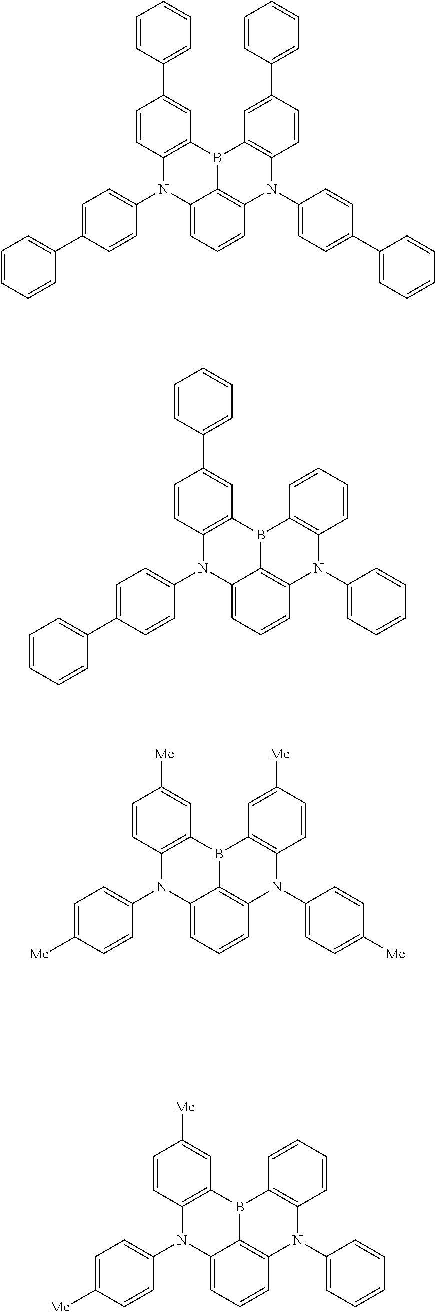 [SMILES: c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5ccccc5N(c5ccccc5)c5cccc3c54)cc2)cc1 ; Cc1ccc(N2c3ccc(C)cc3B3c4cc(C)ccc4N(c4ccc(C)cc4)c4cccc2c43)cc1 ; Cc1ccc(N2c3ccc(C)cc3B3c4ccccc4N(c4ccccc4)c4cccc2c43)cc1]

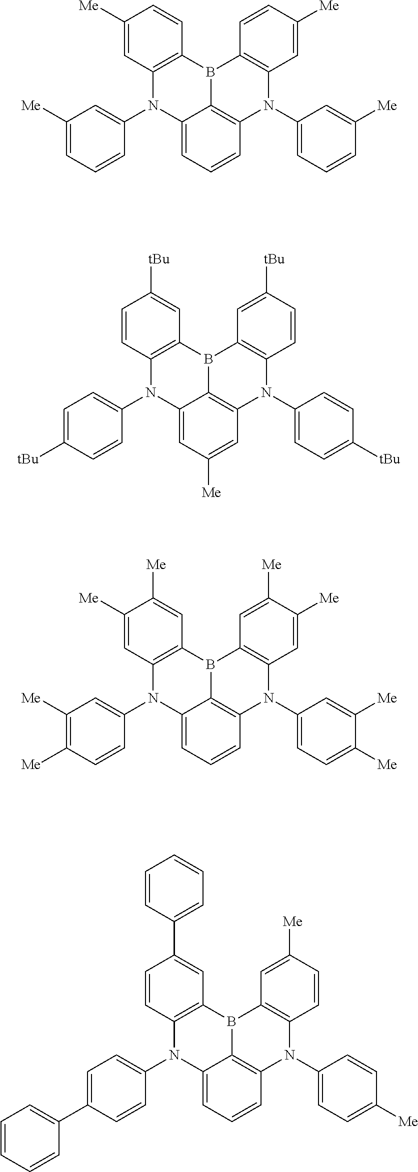 [SMILES: Cc1cccc(N2c3cc(C)ccc3B3c4ccc(C)cc4N(c4cccc(C)c4)c4cccc2c43)c1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; Cc1ccc(N2c3cc(C)c(C)cc3B3c4cc(C)c(C)cc4N(c4ccc(C)c(C)c4)c4cccc2c43)cc1C ; Cc1ccc(N2c3ccc(C)cc3B3c4cc(-c5ccccc5)ccc4N(c4ccc(-c5ccccc5)cc4)c4cccc2c43)cc1]

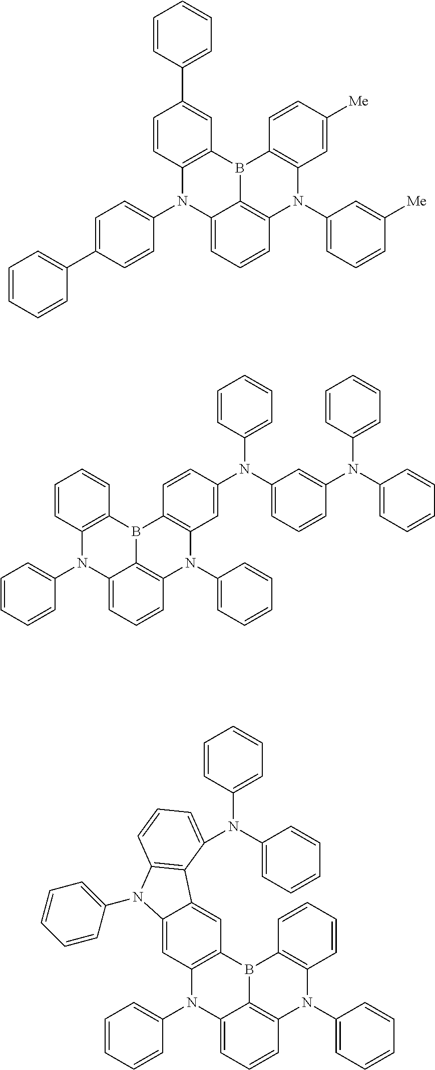 [SMILES: Cc1cccc(N2c3cc(C)ccc3B3c4cc(-c5ccccc5)ccc4N(c4ccc(-c5ccccc5)cc4)c4cccc2c43)c1 ; c1ccc(N(c2ccccc2)c2cccc(N(c3ccccc3)c3ccc4c(c3)N(c3ccccc3)c3cccc5c3B4c3ccccc3N5c3ccccc3)c2)cc1 ; c1ccc(N2c3ccccc3B3c4cc5c6c(N(c7ccccc7)c7ccccc7)cccc6n(-c6ccccc6)c5cc4N(c4ccccc4)c4cccc2c43)cc1]

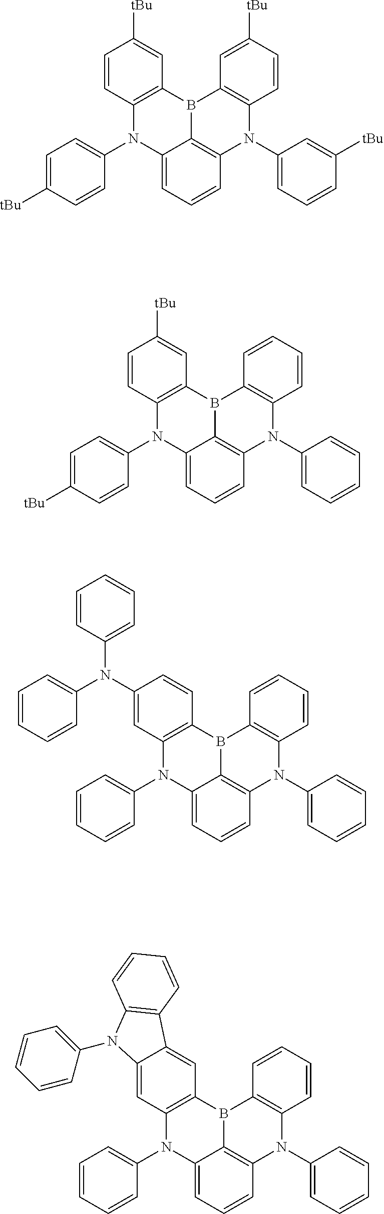 [SMILES: CC(C)(C)c1ccc(N2c3ccc(C(C)(C)C)cc3B3c4cc(C(C)(C)C)ccc4N(c4cccc(C(C)(C)C)c4)c4cccc2c43)cc1 ; CC(C)(C)c1ccc(N2c3ccc(C(C)(C)C)cc3B3c4ccccc4N(c4ccccc4)c4cccc2c43)cc1 ; c1ccc(N(c2ccccc2)c2ccc3c(c2)N(c2ccccc2)c2cccc4c2B3c2ccccc2N4c2ccccc2)cc1 ; c1ccc(N2c3ccccc3B3c4cc5c6ccccc6n(-c6ccccc6)c5cc4N(c4ccccc4)c4cccc2c43)cc1]

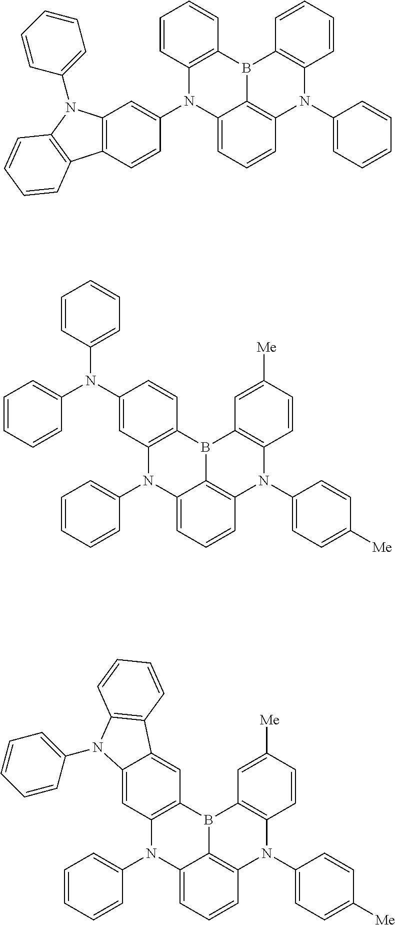 [SMILES: c1ccc(N2c3ccccc3B3c4ccccc4N(c4ccc5c6ccccc6n(-c6ccccc6)c5c4)c4cccc2c43)cc1 ; Cc1ccc(N2c3ccc(C)cc3B3c4ccc(N(c5ccccc5)c5ccccc5)cc4N(c4ccccc4)c4cccc2c43)cc1 ; Cc1ccc(N2c3ccc(C)cc3B3c4cc5c6ccccc6n(-c6ccccc6)c5cc4N(c4ccccc4)c4cccc2c43)cc1]

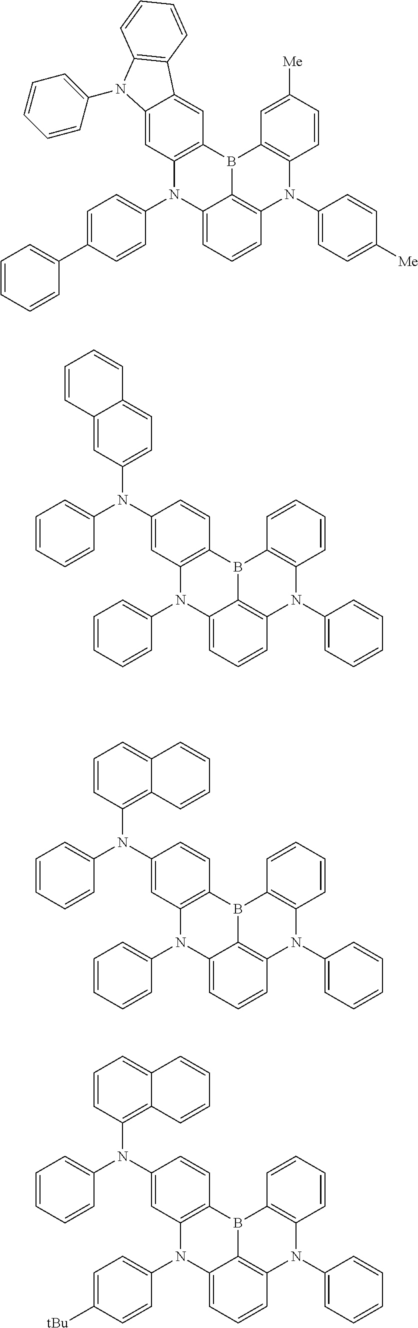 [SMILES: Cc1ccc(N2c3ccc(C)cc3B3c4cc5c6ccccc6n(-c6ccccc6)c5cc4N(c4ccc(-c5ccccc5)cc4)c4cccc2c43)cc1 ; c1ccc(N(c2ccc3c(c2)N(c2ccccc2)c2cccc4c2B3c2ccccc2N4c2ccccc2)c2ccc3ccccc3c2)cc1 ; c1ccc(N2c3ccccc3B3c4ccc(N(c5ccccc5)c5cccc6ccccc56)cc4N(c4ccccc4)c4cccc2c43)cc1 ; CC(C)(C)c1ccc(N2c3cc(N(c4ccccc4)c4cccc5ccccc45)ccc3B3c4ccccc4N(c4ccccc4)c4cccc2c43)cc1]

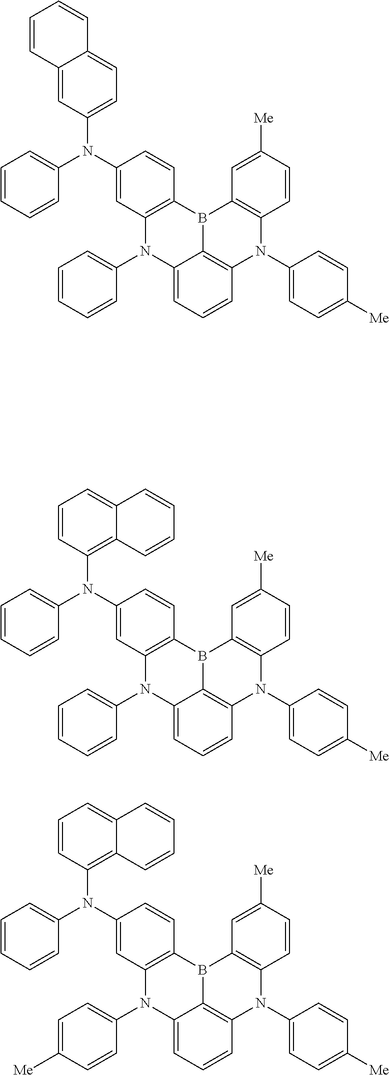 [SMILES: Cc1ccc(N2c3ccc(C)cc3B3c4ccc(N(c5ccccc5)c5ccc6ccccc6c5)cc4N(c4ccccc4)c4cccc2c43)cc1 ; Cc1ccc(N2c3ccc(C)cc3B3c4ccc(N(c5ccccc5)c5cccc6ccccc56)cc4N(c4ccccc4)c4cccc2c43)cc1 ; Cc1ccc(N2c3ccc(C)cc3B3c4ccc(N(c5ccccc5)c5cccc6ccccc56)cc4N(c4ccc(C)cc4)c4cccc2c43)cc1]

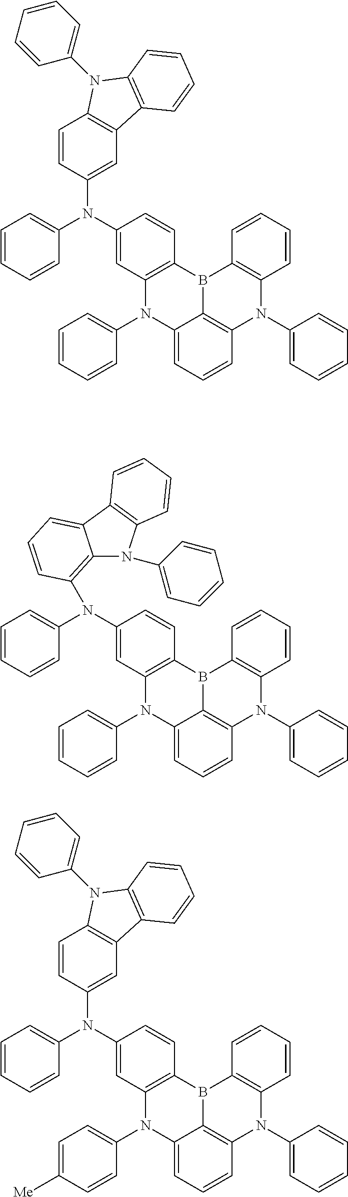 [SMILES: c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1 ; Cc1ccc(N2c3cc(N(c4ccccc4)c4ccc5c(c4)c4ccccc4n5-c4ccccc4)ccc3B3c4ccccc4N(c4ccccc4)c4cccc2c43)cc1]

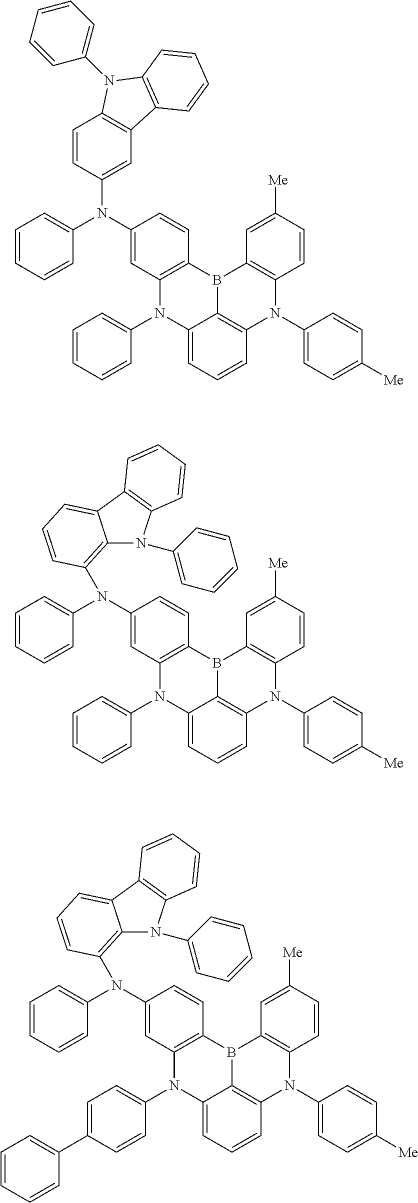 [SMILES: Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1]

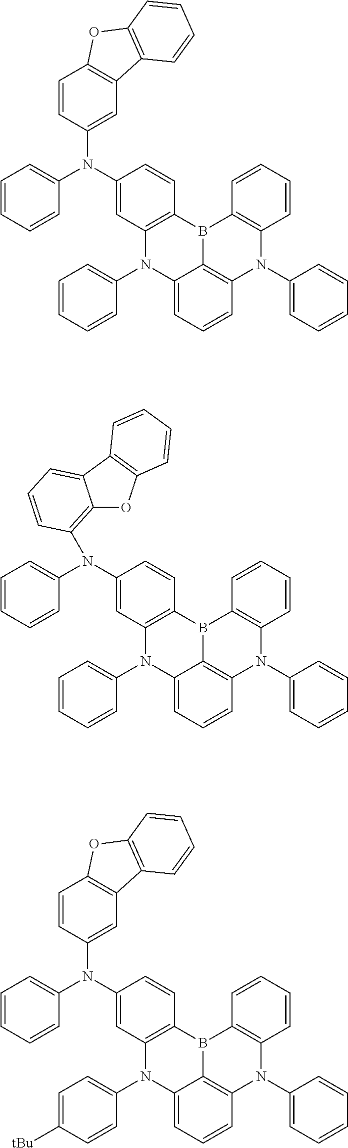 [SMILES: c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1 ; CC(C)(C)c1ccc(N2c3cc(N(c4ccccc4)c4ccc5oc6ccccc6c5c4)ccc3B3c4ccccc4N(c4ccccc4)c4cccc2c43)cc1]

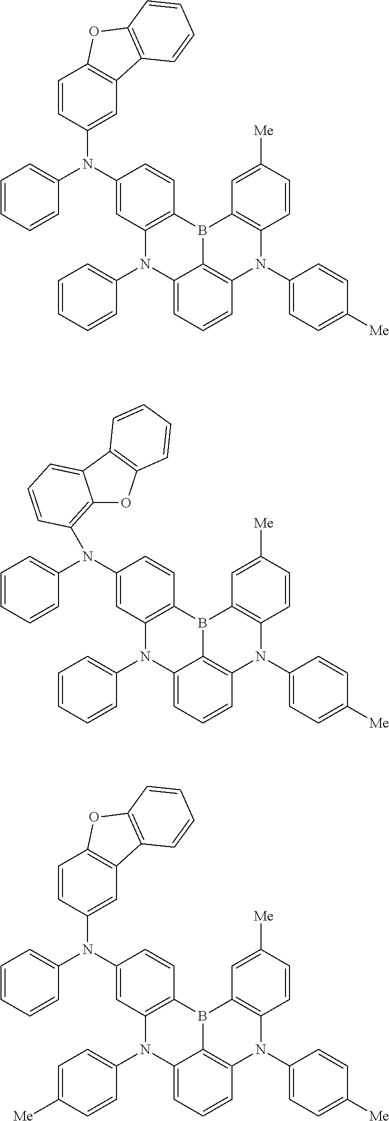 [SMILES: Cc1ccc(N2c3ccc(C)cc3B3c4ccc(N(c5ccccc5)c5ccc6oc7ccccc7c6c5)cc4N(c4ccccc4)c4cccc2c43)cc1 ; Cc1ccc(N2c3ccc(C)cc3B3c4ccc(N(c5ccccc5)c5cccc6c5oc5ccccc56)cc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccc(C)cc4)c4cccc2c43)cc1]

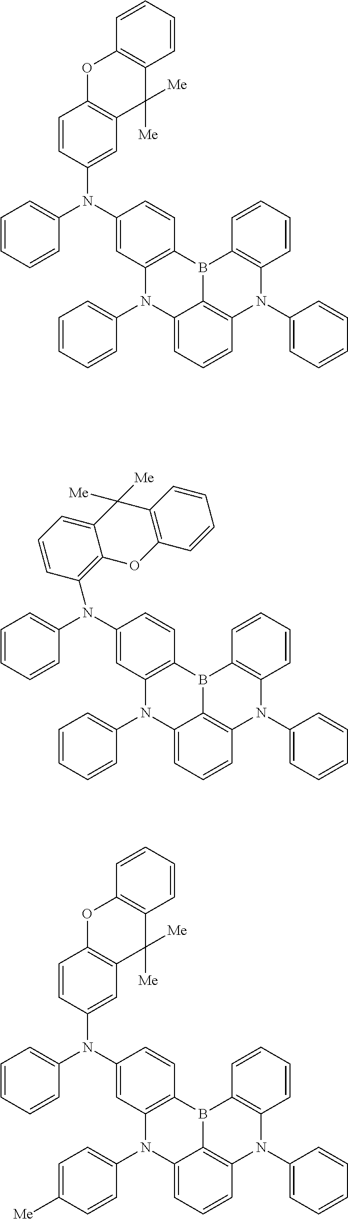 [SMILES: CC1(C)c2ccccc2Oc2ccc(N(c3ccccc3)c3ccc4c(c3)N(c3ccccc3)c3cccc5c3B4c3ccccc3N5c3ccccc3)cc21 ; CC1(C)c2ccccc2Oc2c(N(c3ccccc3)c3ccc4c(c3)N(c3ccccc3)c3cccc5c3B4c3ccccc3N5c3ccccc3)cccc21 ; Cc1ccc(N2c3cc(N(c4ccccc4)c4ccc5c(c4)C(C)(C)c4ccccc4O5)ccc3B3c4ccccc4N(c4ccccc4)c4cccc2c43)cc1]

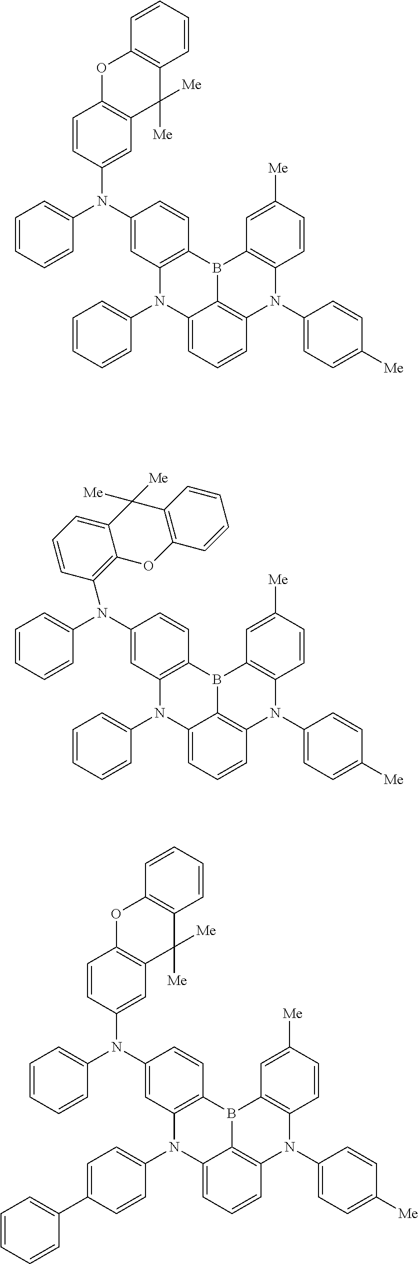 [SMILES: Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1 ; Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1]

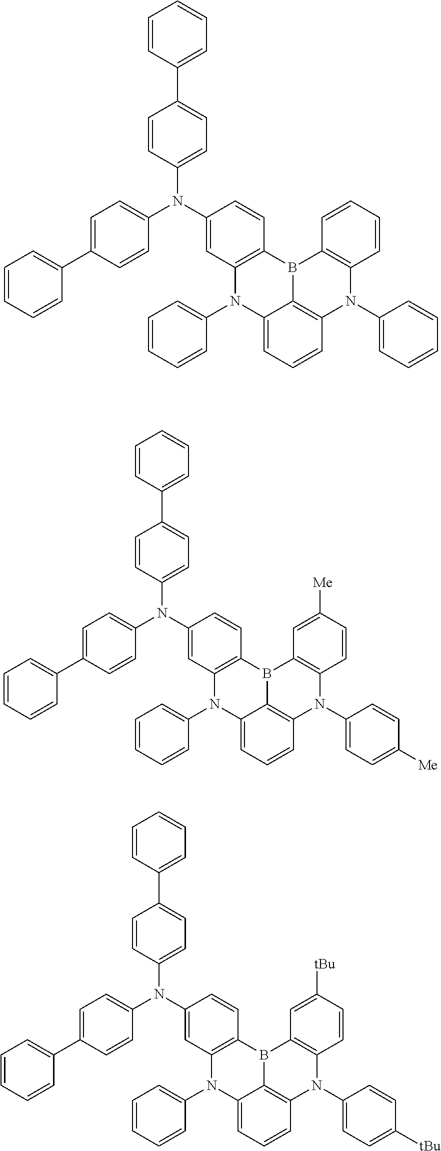 [SMILES: c1ccc(-c2ccc(N(c3ccc(-c4ccccc4)cc3)c3ccc4c(c3)N(c3ccccc3)c3cccc5c3B4c3ccccc3N5c3ccccc3)cc2)cc1 ; Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1 ; CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1]

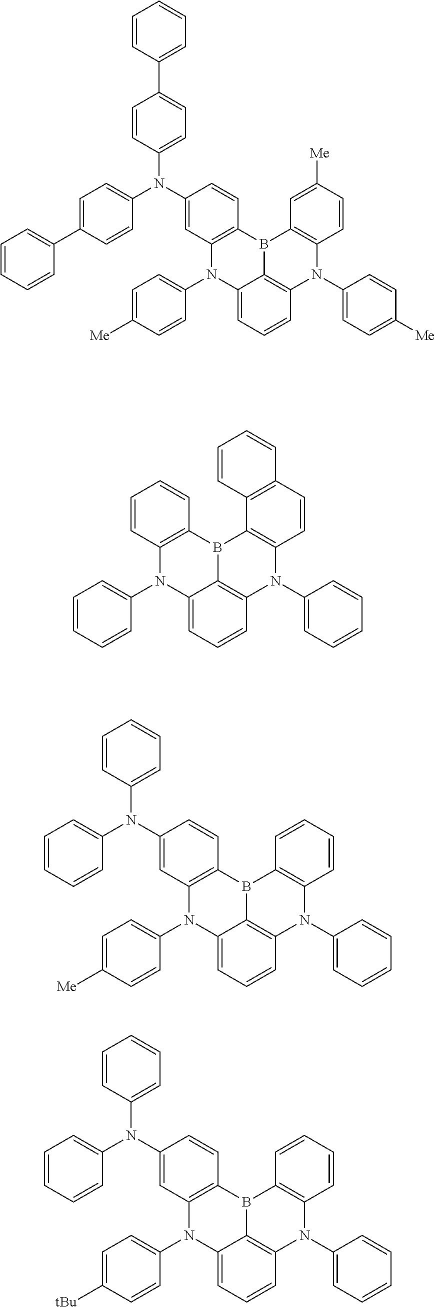 [SMILES: Cc1ccc(N2c3ccc(C)cc3B3c4ccc(N(c5ccc(-c6ccccc6)cc5)c5ccc(-c6ccccc6)cc5)cc4N(c4ccc(C)cc4)c4cccc2c43)cc1 ; c1ccc(N2c3ccccc3B3c4c2cccc4N(c2ccccc2)c2ccc4ccccc4c23)cc1 ; Cc1ccc(N2c3cc(N(c4ccccc4)c4ccccc4)ccc3B3c4ccccc4N(c4ccccc4)c4cccc2c43)cc1 ; CC(C)(C)c1ccc(N2c3cc(N(c4ccccc4)c4ccccc4)ccc3B3c4ccccc4N(c4ccccc4)c4cccc2c43)cc1]

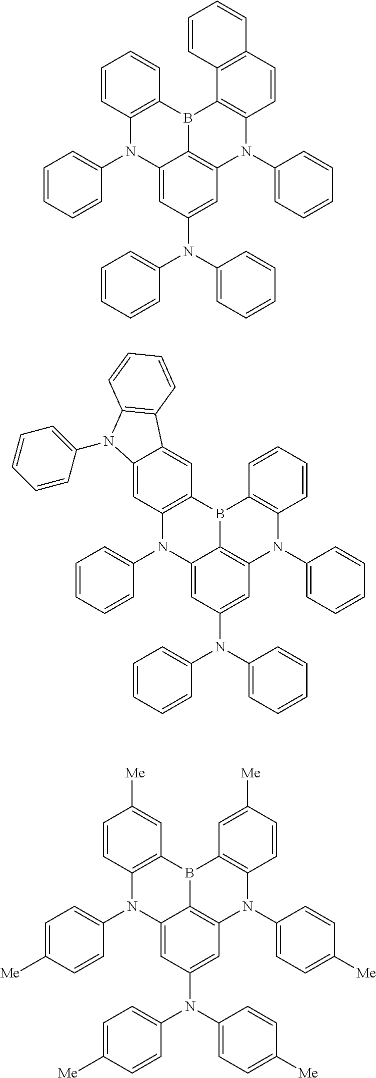

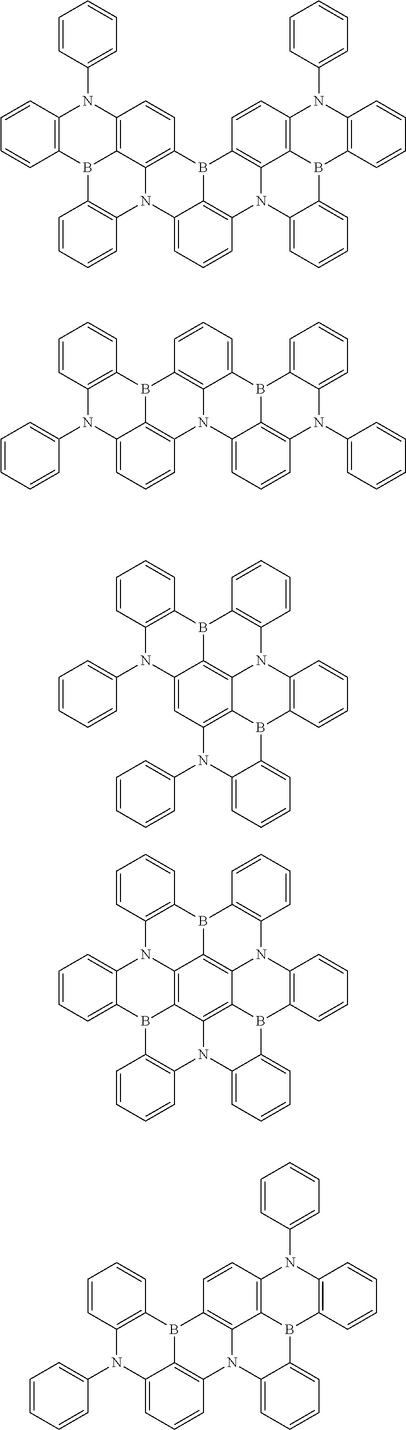

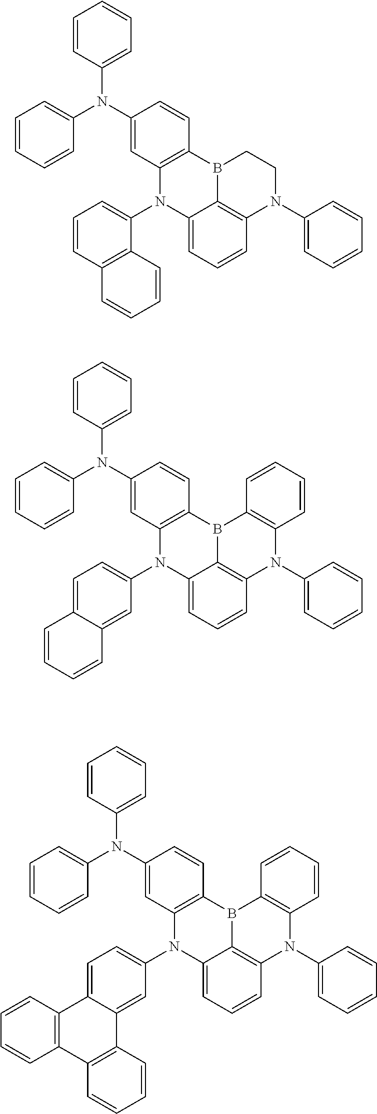 [SMILES: c1ccc(N2CCB3c4ccc(N(c5ccccc5)c5ccccc5)cc4N(c4cccc5ccccc45)c4cccc2c43)cc1 ; c1ccc(N(c2ccccc2)c2ccc3c(c2)N(c2ccc4ccccc4c2)c2cccc4c2B3c2ccccc2N4c2ccccc2)cc1 ; c1ccc(N(c2ccccc2)c2ccc3c(c2)N(c2ccc4c5ccccc5c5ccccc5c4c2)c2cccc4c2B3c2ccccc2N4c2ccccc2)cc1]

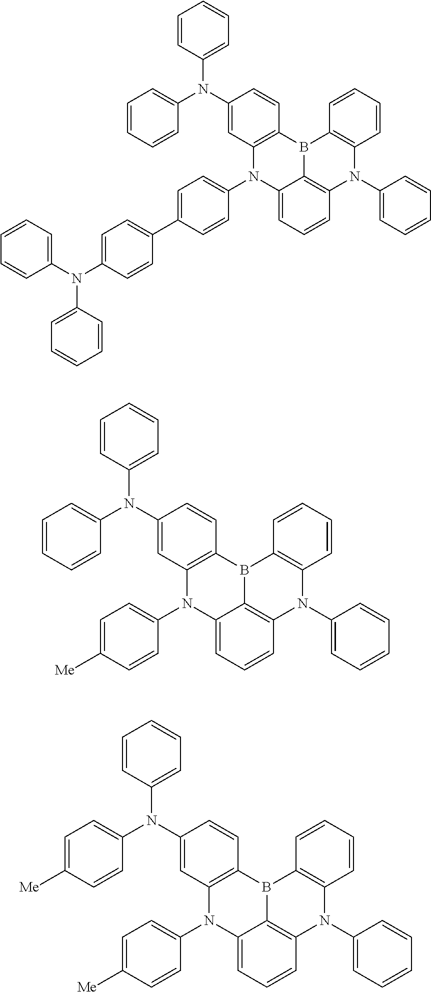 [SMILES: c1ccc(N(c2ccccc2)c2ccc(-c3ccc(N4c5cc(N(c6ccccc6)c6ccccc6)ccc5B5c6ccccc6N(c6ccccc6)c6cccc4c65)cc3)cc2)cc1 ; Cc1ccc(N2c3cc(N(c4ccccc4)c4ccccc4)ccc3B3c4ccccc4N(c4ccccc4)c4cccc2c43)cc1 ; Cc1ccc(N(c2ccccc2)c2ccc3c(c2)N(c2ccc(C)cc2)c2cccc4c2B3c2ccccc2N4c2ccccc2)cc1]

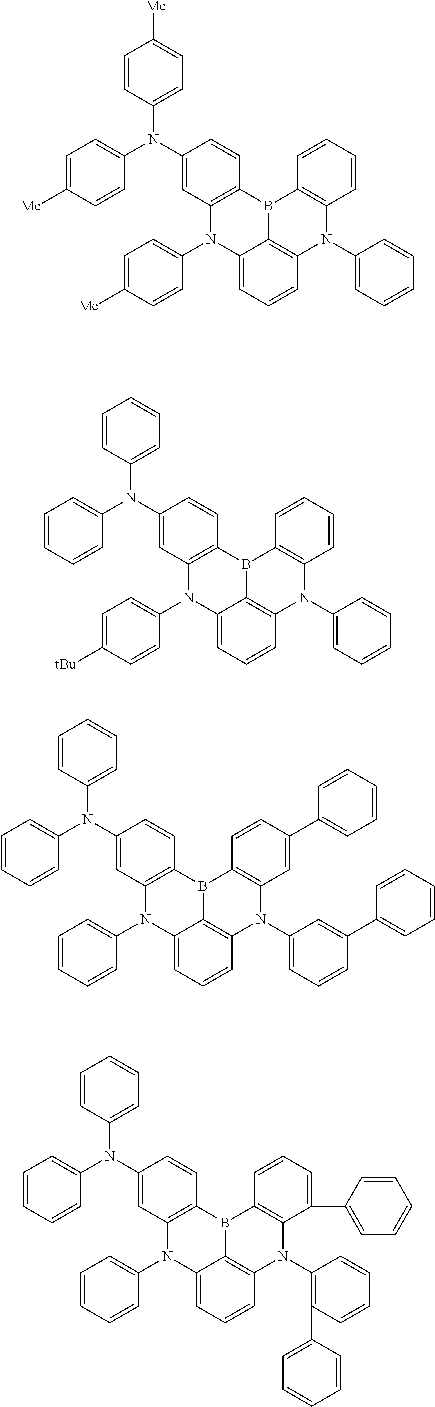 [SMILES: Cc1ccc(N(c2ccc(C)cc2)c2ccc3c(c2)N(c2ccc(C)cc2)c2cccc4c2B3c2ccccc2N4c2ccccc2)cc1 ; CC(C)(C)c1ccc(N2c3cc(N(c4ccccc4)c4ccccc4)ccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1]

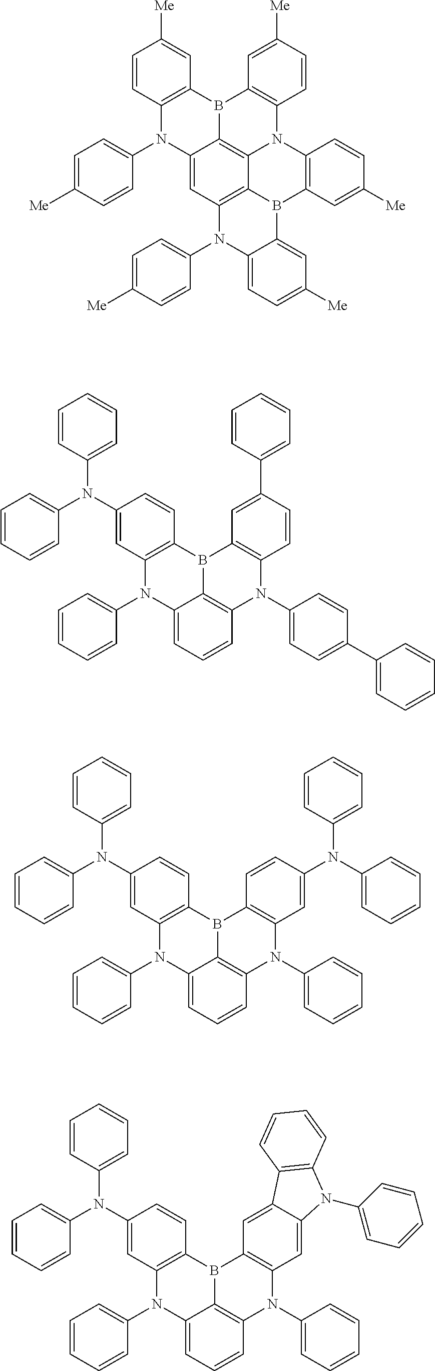 [SMILES: Cc1ccc(N2c3ccc(C)cc3B3c4cc(C)ccc4N4c5ccc(C)cc5B5c6cc(C)ccc6N(c6ccc(C)cc6)c6cc2c3c4c65)cc1 ; c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cccc4c2B3c2cc3c5ccccc5n(-c5ccccc5)c3cc2N4c2ccccc2)cc1]

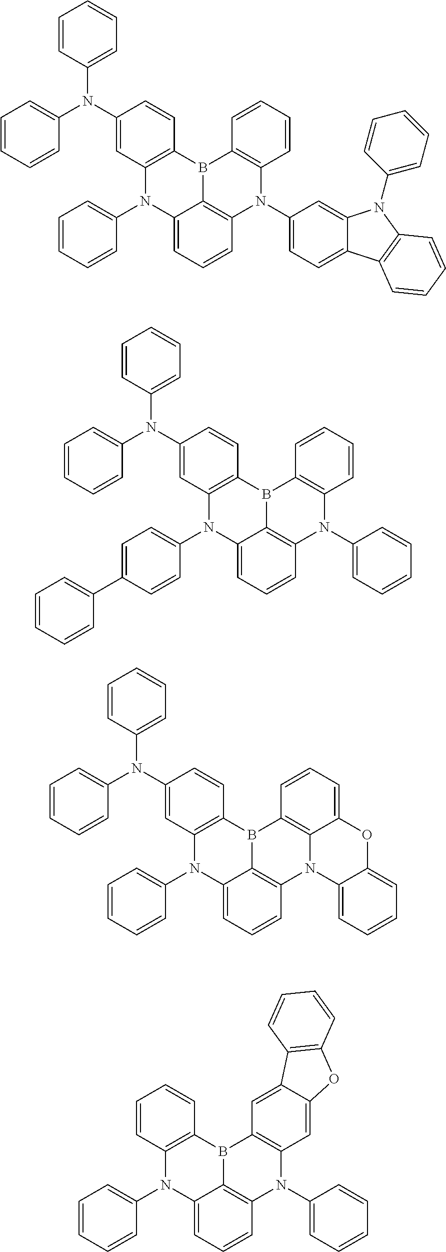 [SMILES: c1ccc(N(c2ccccc2)c2ccc3c(c2)N(c2ccccc2)c2cccc4c2B3c2ccccc2N4c2ccc3c4ccccc4n(-c4ccccc4)c3c2)cc1 ; c1ccc(-c2ccc(N3c4cc(N(c5ccccc5)c5ccccc5)ccc4B4c5ccccc5N(c5ccccc5)c5cccc3c54)cc2)cc1 ; c1ccc(N(c2ccccc2)c2ccc3c(c2)N(c2ccccc2)c2cccc4c2B3c2cccc3c2N4c2ccccc2O3)cc1 ; c1ccc(N2c3ccccc3B3c4cc5c(cc4N(c4ccccc4)c4cccc2c43)oc2ccccc25)cc1]

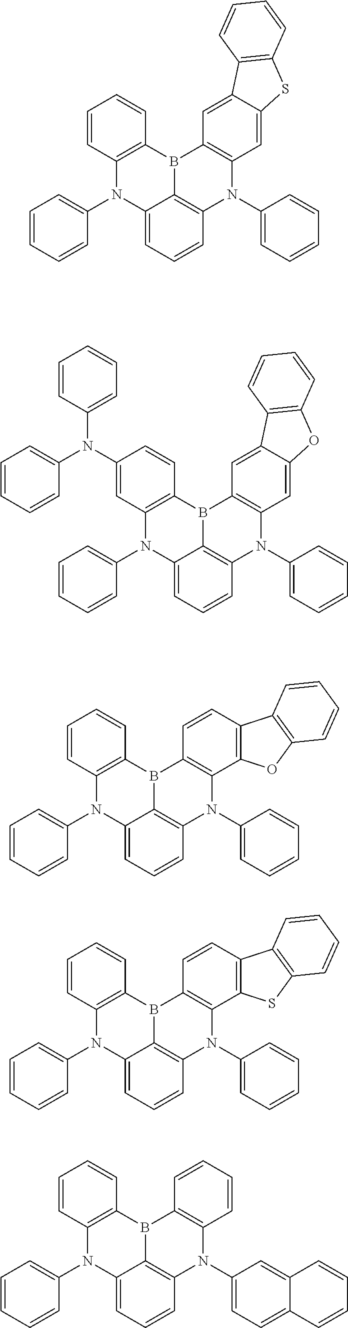

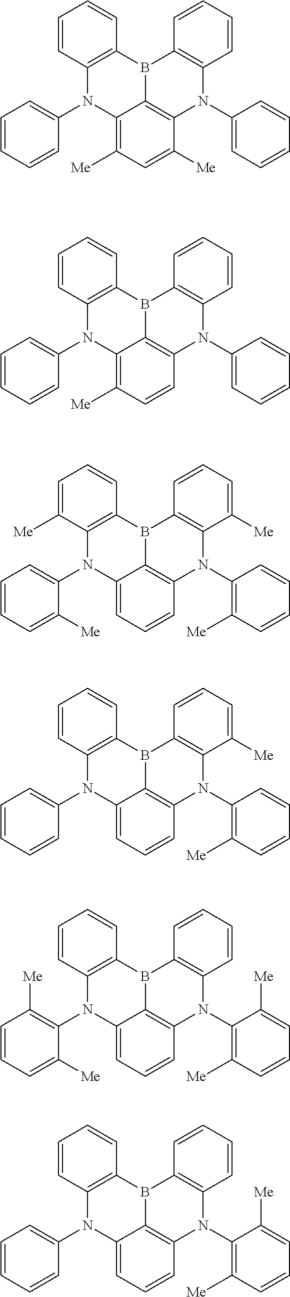 [SMILES: Cc1cc(C)c2c3c1N(c1ccccc1)c1ccccc1B3c1ccccc1N2c1ccccc1 ; Cc1ccc2c3c1N(c1ccccc1)c1ccccc1B3c1ccccc1N2c1ccccc1 ; Cc1ccccc1N1c2cccc3c2B(c2cccc(C)c21)c1cccc(C)c1N3c1ccccc1C ; Cc1ccccc1N1c2cccc3c2B(c2ccccc2N3c2ccccc2)c2cccc(C)c21 ; Cc1cccc(C)c1N1c2ccccc2B2c3ccccc3N(c3c(C)cccc3C)c3cccc1c32 ; Cc1cccc(C)c1N1c2ccccc2B2c3ccccc3N(c3ccccc3)c3cccc1c32]

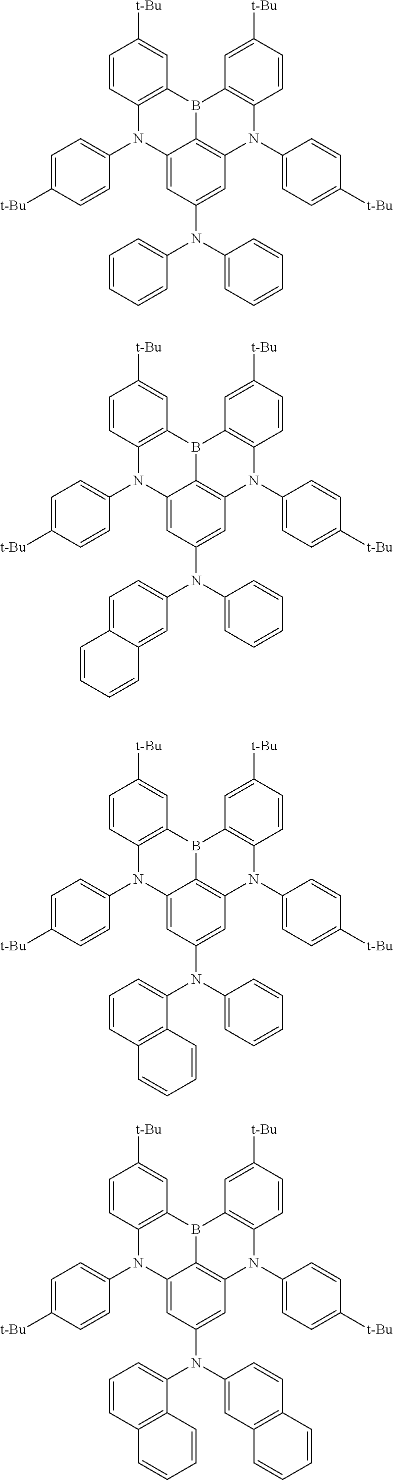

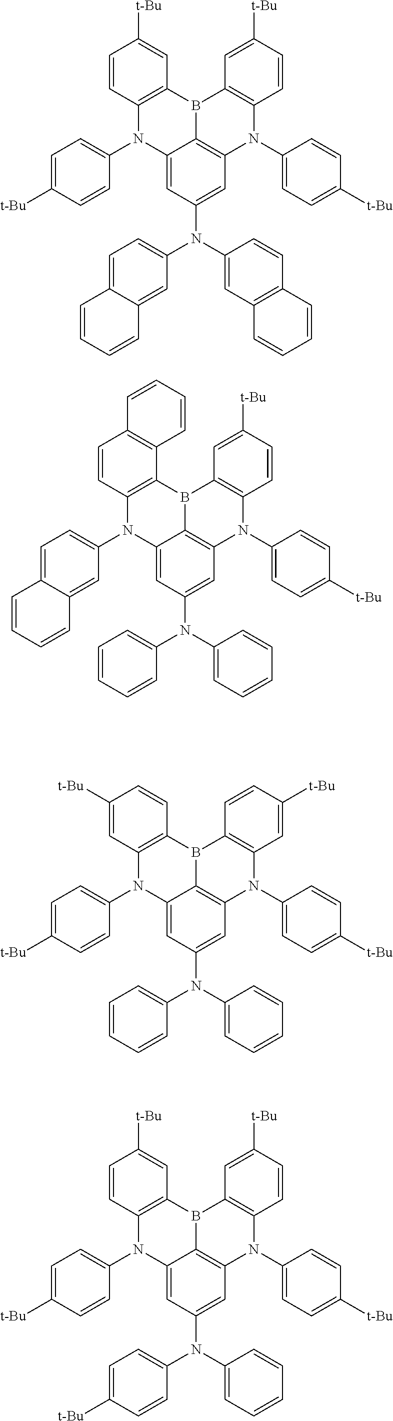

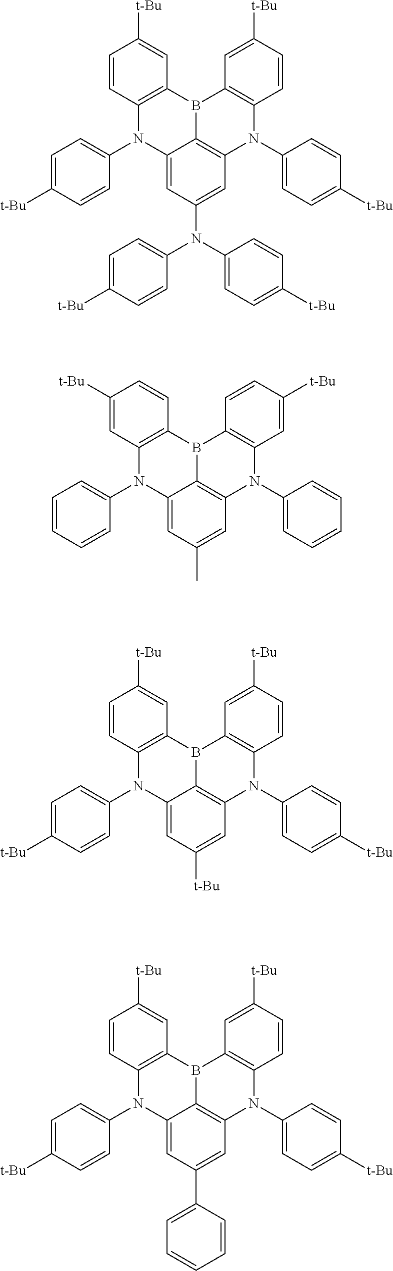 [SMILES: CC(C)(C)c1ccc(N(c2ccc(C(C)(C)C)cc2)c2cc3c4c(c2)N(c2ccc(C(C)(C)C)cc2)c2ccc(C(C)(C)C)cc2B4c2cc(C(C)(C)C)ccc2N3c2ccc(C(C)(C)C)cc2)cc1 ; Cc1cc2c3c(c1)N(c1ccccc1)c1cc(C(C)(C)C)ccc1B3c1ccc(C(C)(C)C)cc1N2c1ccccc1 ; CC(C)(C)c1ccc(N2c3ccc(C(C)(C)C)cc3B3c4cc(C(C)(C)C)ccc4N(c4ccc(C(C)(C)C)cc4)c4cc(C(C)(C)C)cc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(-c5ccccc5)cc2c43)cc1]

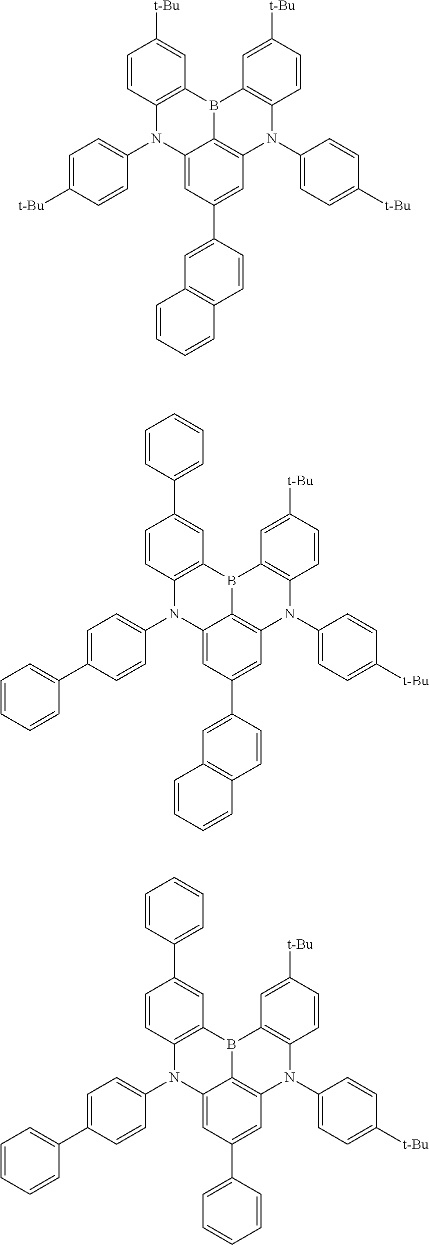 [SMILES: CC(C)(C)c1ccc(N2c3ccc(C(C)(C)C)cc3B3c4cc(C(C)(C)C)ccc4N(c4ccc(C(C)(C)C)cc4)c4cc(-c5ccc6ccccc6c5)cc2c43)cc1 ; CC(C)(C)c1ccc(N2c3ccc(C(C)(C)C)cc3B3c4cc(-c5ccccc5)ccc4N(c4ccc(-c5ccccc5)cc4)c4cc(-c5ccc6ccccc6c5)cc2c43)cc1 ; CC(C)(C)c1ccc(N2c3ccc(C(C)(C)C)cc3B3c4cc(-c5ccccc5)ccc4N(c4ccc(-c5ccccc5)cc4)c4cc(-c5ccccc5)cc2c43)cc1]

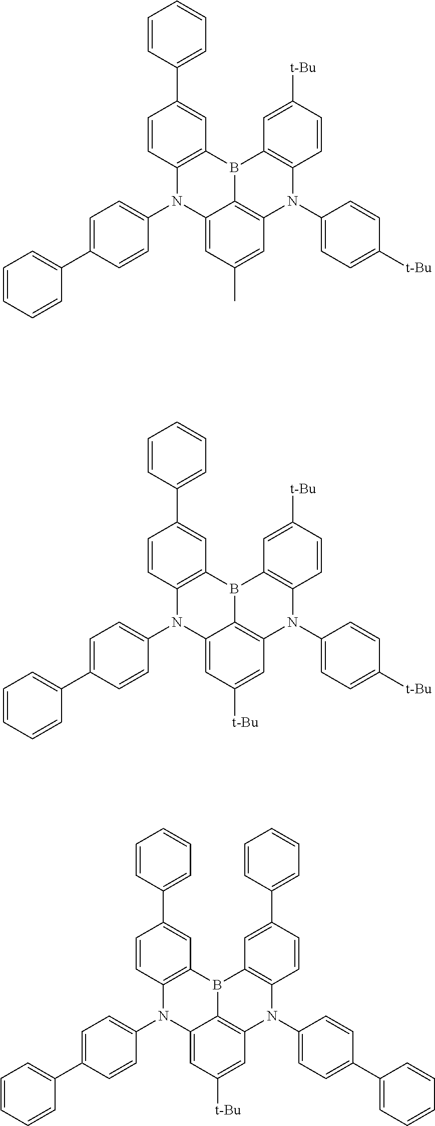 [SMILES: Cc1cc2c3c(c1)N(c1ccc(C(C)(C)C)cc1)c1ccc(C(C)(C)C)cc1B3c1cc(-c3ccccc3)ccc1N2c1ccc(-c2ccccc2)cc1 ; CC(C)(C)c1ccc(N2c3ccc(C(C)(C)C)cc3B3c4cc(-c5ccccc5)ccc4N(c4ccc(-c5ccccc5)cc4)c4cc(C(C)(C)C)cc2c43)cc1 ; CC(C)(C)c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1]

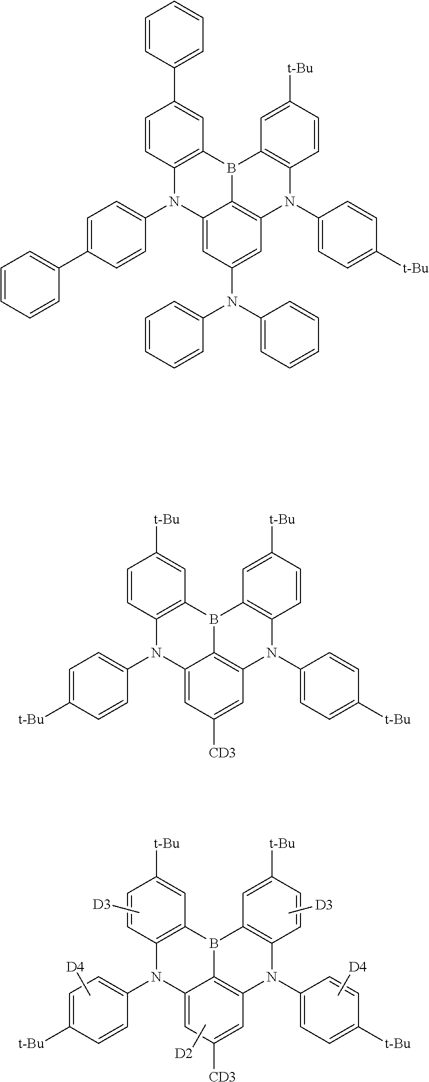

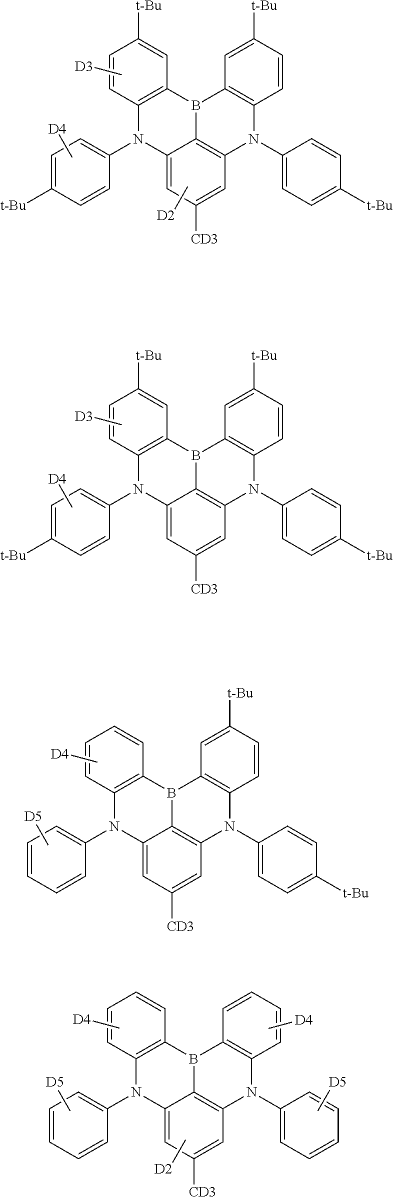

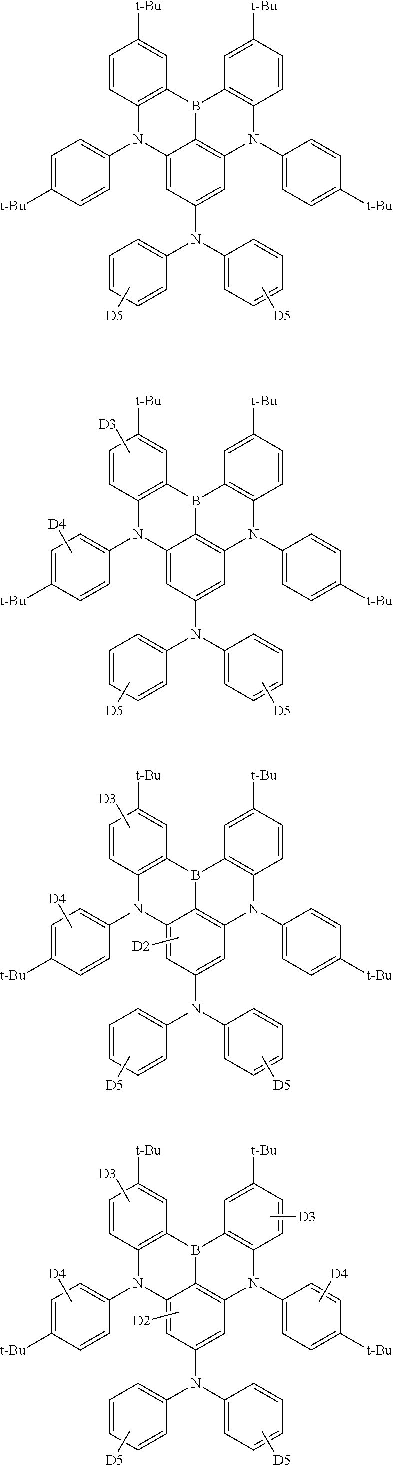

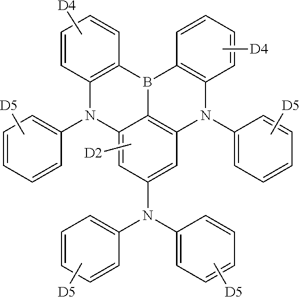

In the compounds above, D2 to D5 mean that two (2) to five (5) hydrogens each have been replaced with deuterium.

The hole blocking layer, the electron buffer layer, or both of them of the present disclosure include at least one compound(s) represented by formula 11 above. That is, the compound represented by formula 11 above may be included in the hole blocking layer, may be included in the electron buffer layer, or may be included in the hole blocking layer and the electron buffer layer, respectively.

In formula 11 above, Z₁ to Z₃ each independently represent CR₁₈.

In formula 11 above, R₁₈ each independently represents hydrogen, deuterium, halogen, cyano, a substituted or unsubstituted (C1-C30)alkyl, a substituted or unsubstituted (C6-C50)aryl, a substituted or unsubstituted (3- to 50-membered)heteroaryl, a substituted or unsubstituted (C3-C30)cycloalkyl, a substituted or unsubstituted (3- to 7-membered)heterocycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N-(Ar₄)(Ar₅), provided that R₁₈ does not include 2-dimethylfluorenyl, quinolinyl, and pyridyl. Preferably R₁₈ each independently may be hydrogen, a substituted or unsubstituted (C6-C40)aryl, or a substituted or unsubstituted (5- to 45-membered)heteroaryl, more preferably hydrogen, a substituted or unsubstituted (C6-C30)aryl, or a substituted or unsubstituted (5- to 40-membered)heteroaryl, for example, hydrogen, a substituted or unsubstituted phenyl, substituted indole, a substituted or unsubstituted naphthyl, a substituted or unsubstituted biphenyl, substituted phenylnaphthyl, substituted biphenylnaphthyl, fluorenyl substituted with dimethyl (2-dimethylfluorenyl is excluded), fluorenyl substituted with diphenyl, benzofluorenyl substituted with dimethyl, a substituted or unsubstituted terphenyl, unsubstituted spirobifluorenyl, a substituted or unsubstituted carbazolyl, substituted benzocarbazolyl, unsubstituted dibenzofuranyl, or a substituted or unsubstituted (16- to 38-membered)heteroaryl containing at least one of N, O, and S.

According to one embodiment of the present disclosure, the compound represented by formula 11 above may be represented by the following formula 11-1.

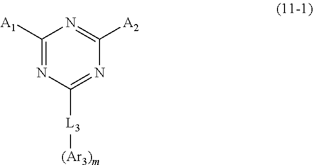

In formula 11-1,

A₁ and A₂ each independently are as defined as R₁₈ in formula 11 above, and

m represents an integer of 1 or 2, and when m is 2, each of Ar₂ may be the same or different.

In formula 11-1 above, L₃ represents a single bond, a substituted or unsubstituted (C6-C50)arylene, or a substituted or unsubstituted (5- to 50-membered)heteroarylene; preferably a single bond, a substituted or unsubstituted (C6-C45)arylene, or a substituted or unsubstituted (5- to 45-membered)heteroarylene; more preferably a single bond, a substituted or unsubstituted (C6-C30)arylene, or a substituted or unsubstituted (5- to 30-membered)heteroarylene. For example, L₃ may be a single bond, phenylene unsubstituted or substituted with pyridinyl, unsubstituted naphthylene, biphenylene unsubstituted or substituted with pyridinyl, unsubstituted terphenylene, unsubstituted phenylnaphthylene, unsubstituted biphenylnaphthylene, indolene substituted with phenyl, carbazolylene unsubstituted or substituted with phenyl, or unsubstituted benzocarbazolylene.

In formula 11-1 above, Ar₃ may be a substituted or unsubstituted (C6-C50)aryl, or a substituted or unsubstituted (5- to 50-membered)heteroaryl, provided that 2-dimethylfluorenyl, quinolinyl, and pyridyl are excluded, preferably a substituted or unsubstituted (C6-C45)aryl, or a substituted or unsubstituted (5- to 45-membered)heteroaryl, more preferably a substituted or unsubstituted (C6-C30)aryl, or a substituted or unsubstituted (5- to 40-membered)heteroaryl. For example, Ar₃ may be unsubstituted naphthyl, fluorenyl substituted with dimethyl (2-dimethylfluorenyl is excluded), fluorenyl substituted with diphenyl, benzofluorenyl substituted with dimethyl, unsubstituted phenanthrenyl, unsubstituted triphenylenyl, unsubstituted spirobifluorenyl, benzoimidazolyl substituted with phenyl, indolyl substituted with phenyl, substituted or unsubstituted carbazolyl, unsubstituted dibenzothiophenyl, unsubstituted dibenzofuranyl, benzocarbazolyl unsubstituted or substituted with phenyl, unsubstituted dibenzocarbazolyl, unsubstituted benzophenanthrothiophenyl, or a substituted or unsubstituted (13- to 38-membered)heteroaryl containing at least one of N, O and S and may be a spiro structure. The substituent of the substituted carbazolyl may be at least one of fluorenyl substituted with phenyl, carbazolyl substituted with phenyl, methyl, phenyl, dibenzothiophenyl, and dibenzofuranyl. The substituent of the substituted (13- to 38-membered)heteroaryl may be at least one of methyl, ter-butyl, phenyl, naphthyl, and biphenyl.

In formulas 1, 2, and 11-1 above, L₄ each independently represent a single bond, a substituted or unsubstituted (C6-C30)arylene, or a substituted or unsubstituted (3- to 30-membered)heteroarylene.

In formulas 1, 2, and 11-1 above, Ar₄ and Ar₅ each independently represent hydrogen, a substituted or unsubstituted (C1-C30)alkyl, a substituted or unsubstituted (C2-C30)alkenyl, a substituted or unsubstituted fused ring of an (C3-C30) aliphatic ring and (C6-C30) aromatic ring, a substituted or unsubstituted (C6-C30)aryl, or a substituted or unsubstituted (3- to 30-membered)heteroaryl.

According to one embodiment, the compound represented by formula 11 above may be more specifically illustrated by the following compounds, but is not limited thereto.

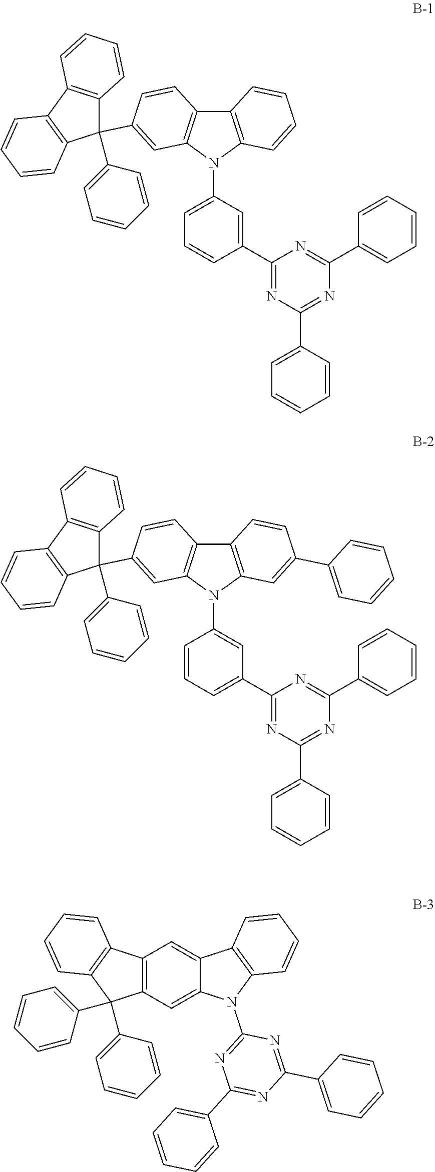 [SMILES: c1ccc(-c2nc(-c3ccccc3)nc(-c3cccc(-n4c5ccccc5c5ccc(C6(c7ccccc7)c7ccccc7-c7ccccc76)cc54)c3)n2)cc1 ; c1ccc(-c2ccc3c4ccc(C5(c6ccccc6)c6ccccc6-c6ccccc65)cc4n(-c4cccc(-c5nc(-c6ccccc6)nc(-c6ccccc6)n5)c4)c3c2)cc1 ; c1ccc(-c2nc(-c3ccccc3)nc(-n3c4ccccc4c4cc5c(cc43)C(c3ccccc3)(c3ccccc3)c3ccccc3-5)n2)cc1]

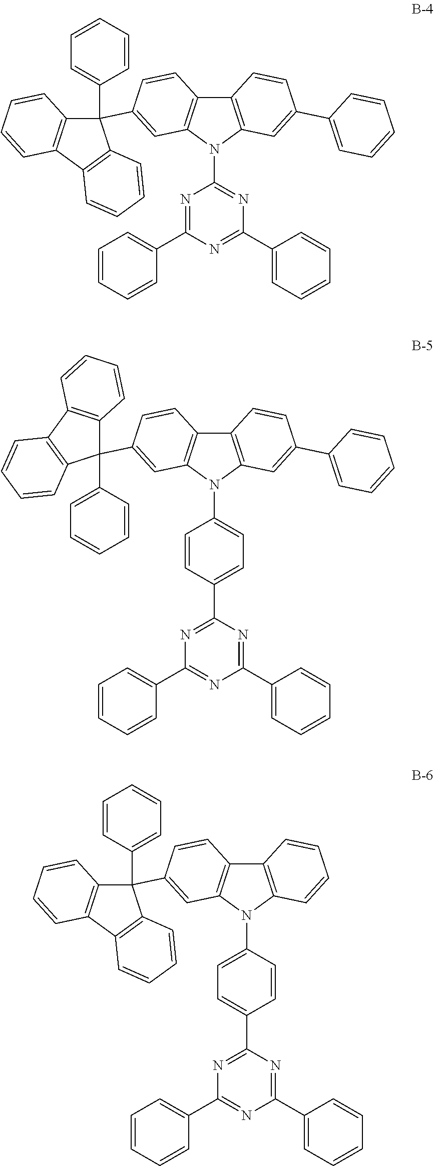 [SMILES: c1ccc(-c2ccc3c4ccc(C5(c6ccccc6)c6ccccc6-c6ccccc65)cc4n(-c4nc(-c5ccccc5)nc(-c5ccccc5)n4)c3c2)cc1 ; c1ccc(-c2ccc3c4ccc(C5(c6ccccc6)c6ccccc6-c6ccccc65)cc4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5ccccc5c5ccc(C6(c7ccccc7)c7ccccc7-c7ccccc76)cc54)cc3)n2)cc1]

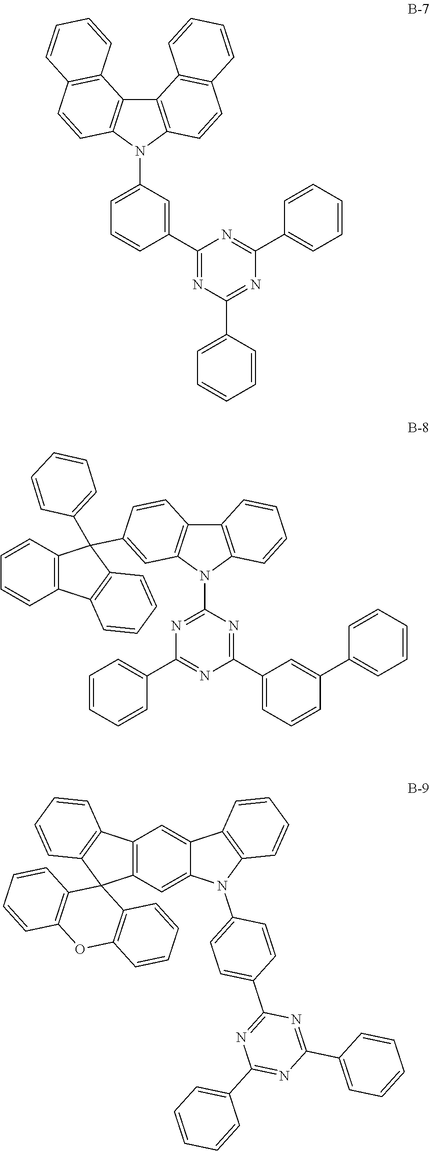 [SMILES: c1ccc(-c2nc(-c3ccccc3)nc(-c3cccc(-n4c5ccc6ccccc6c5c5c6ccccc6ccc54)c3)n2)cc1 ; c1ccc(-c2cccc(-c3nc(-c4ccccc4)nc(-n4c5ccccc5c5ccc(C6(c7ccccc7)c7ccccc7-c7ccccc76)cc54)n3)c2)cc1 ; c1ccc(-c2nc(-c3ccccc3)nc(-c3ccc(-n4c5ccccc5c5cc6c(cc54)C4(c5ccccc5Oc5ccccc54)c4ccccc4-6)cc3)n2)cc1]

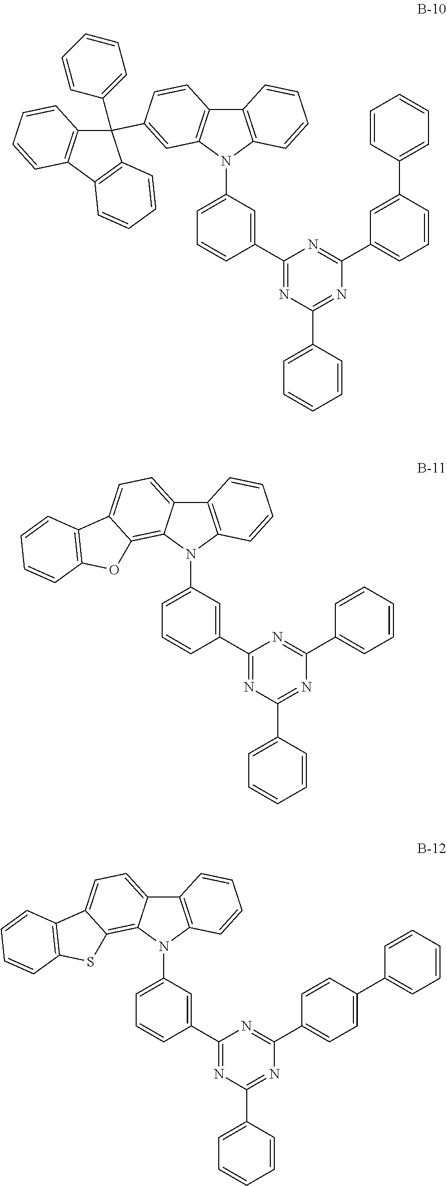 [SMILES: c1ccc(-c2cccc(-c3nc(-c4ccccc4)nc(-c4cccc(-n5c6ccccc6c6ccc(C7(c8ccccc8)c8ccccc8-c8ccccc87)cc65)c4)n3)c2)cc1 ; c1ccc(-c2nc(-c3ccccc3)nc(-c3cccc(-n4c5ccccc5c5ccc6c7ccccc7oc6c54)c3)n2)cc1 ; c1ccc(-c2ccc(-c3nc(-c4ccccc4)nc(-c4cccc(-n5c6ccccc6c6ccc7c8ccccc8sc7c65)c4)n3)cc2)cc1]

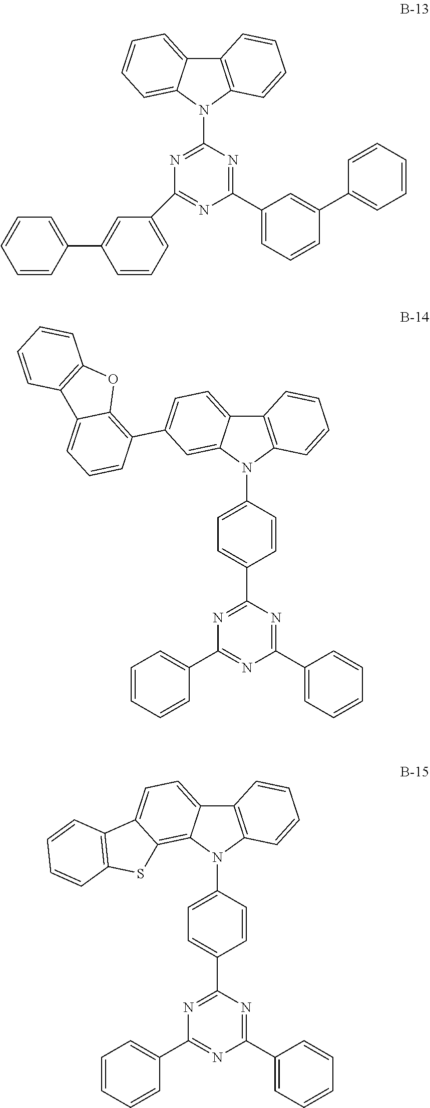 [SMILES: c1ccc(-c2cccc(-c3nc(-c4cccc(-c5ccccc5)c4)nc(-n4c5ccccc5c5ccccc54)n3)c2)cc1 ; c1ccc(-c2nc(-c3ccccc3)nc(-c3ccc(-n4c5ccccc5c5ccc(-c6cccc7c6oc6ccccc67)cc54)cc3)n2)cc1 ; c1ccc(-c2nc(-c3ccccc3)nc(-c3ccc(-n4c5ccccc5c5ccc6c7ccccc7sc6c54)cc3)n2)cc1]

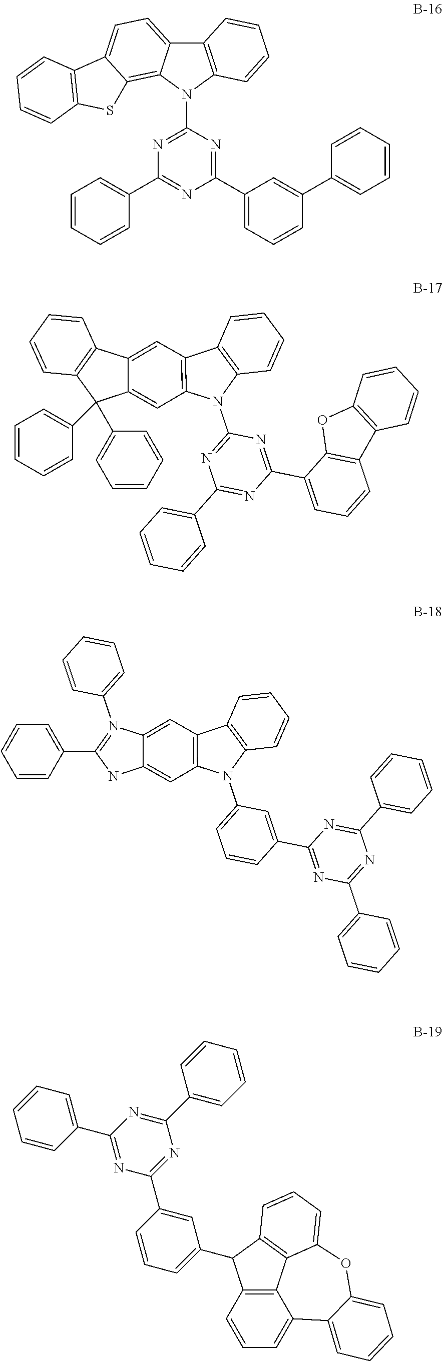

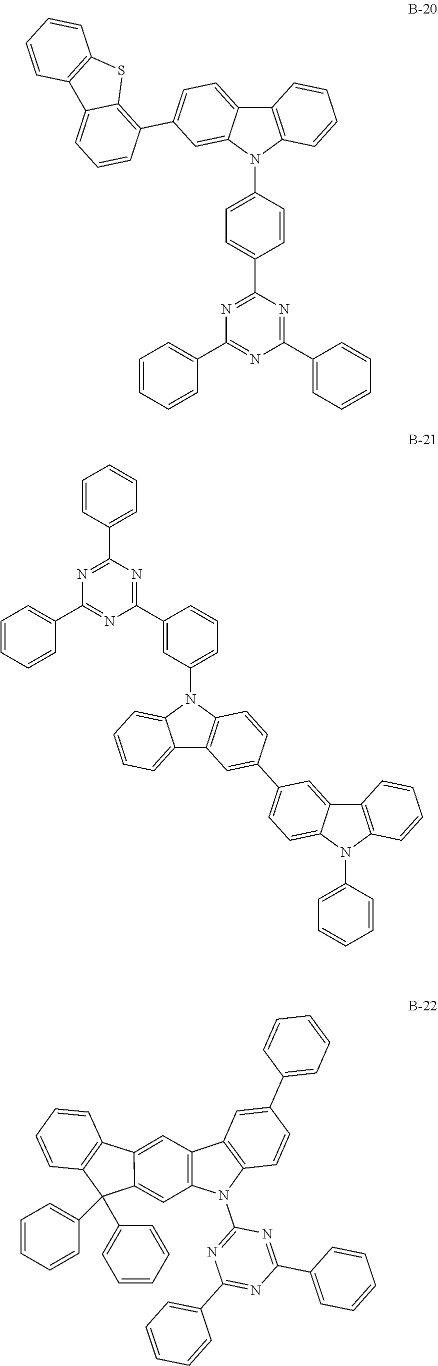 [SMILES: c1ccc(-c2nc(-c3ccccc3)nc(-c3ccc(-n4c5ccccc5c5ccc(-c6cccc7c6sc6ccccc67)cc54)cc3)n2)cc1 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc3c(c2)c2cc4c(cc2n3-c2nc(-c3ccccc3)nc(-c3ccccc3)n2)C(c2ccccc2)(c2ccccc2)c2ccccc2-4)cc1]

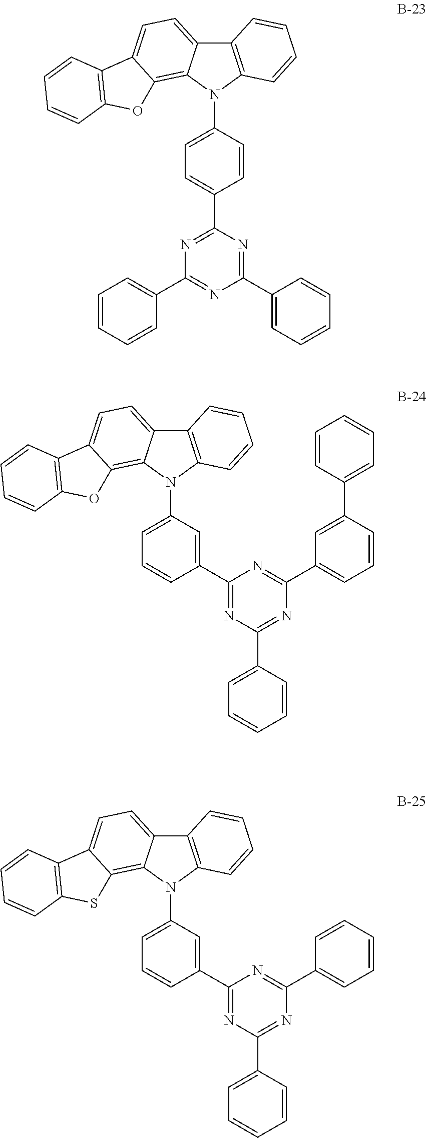 [SMILES: c1ccc(-c2nc(-c3ccccc3)nc(-c3ccc(-n4c5ccccc5c5ccc6c7ccccc7oc6c54)cc3)n2)cc1 ; c1ccc(-c2cccc(-c3nc(-c4ccccc4)nc(-c4cccc(-n5c6ccccc6c6ccc7c8ccccc8oc7c65)c4)n3)c2)cc1 ; c1ccc(-c2nc(-c3ccccc3)nc(-c3cccc(-n4c5ccccc5c5ccc6c7ccccc7sc6c54)c3)n2)cc1]

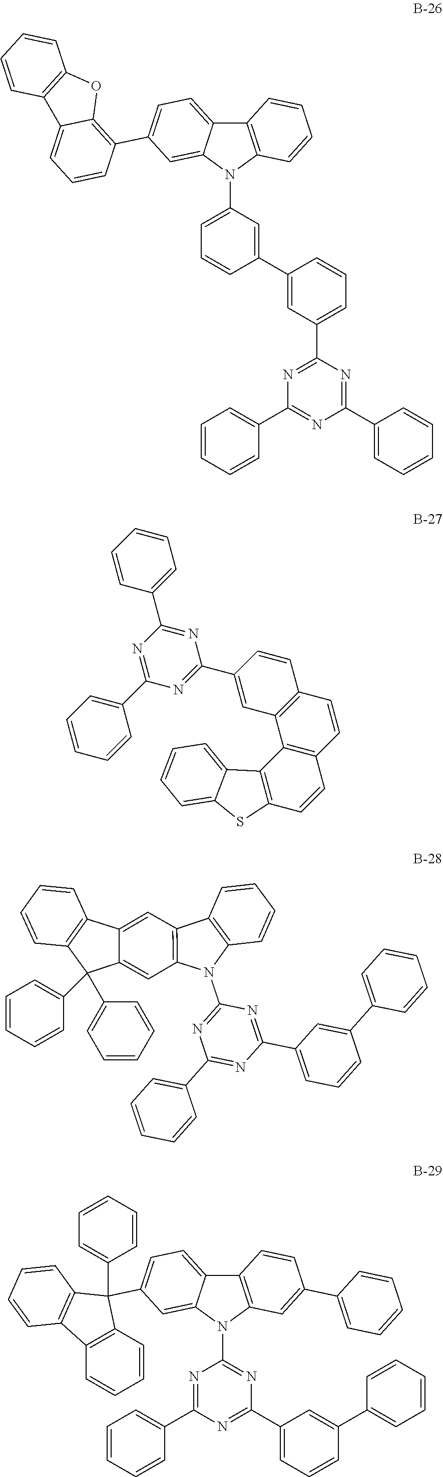 [SMILES: c1ccc(-c2nc(-c3ccccc3)nc(-c3cccc(-c4cccc(-n5c6ccccc6c6ccc(-c7cccc8c7oc7ccccc78)cc65)c4)c3)n2)cc1 ; c1ccc(-c2nc(-c3ccccc3)nc(-c3ccc4ccc5ccc6sc7ccccc7c6c5c4c3)n2)cc1 ; c1ccc(-c2cccc(-c3nc(-c4ccccc4)nc(-n4c5ccccc5c5cc6c(cc54)C(c4ccccc4)(c4ccccc4)c4ccccc4-6)n3)c2)cc1 ; c1ccc(-c2cccc(-c3nc(-c4ccccc4)nc(-n4c5cc(-c6ccccc6)ccc5c5ccc(C6(c7ccccc7)c7ccccc7-c7ccccc76)cc54)n3)c2)cc1]

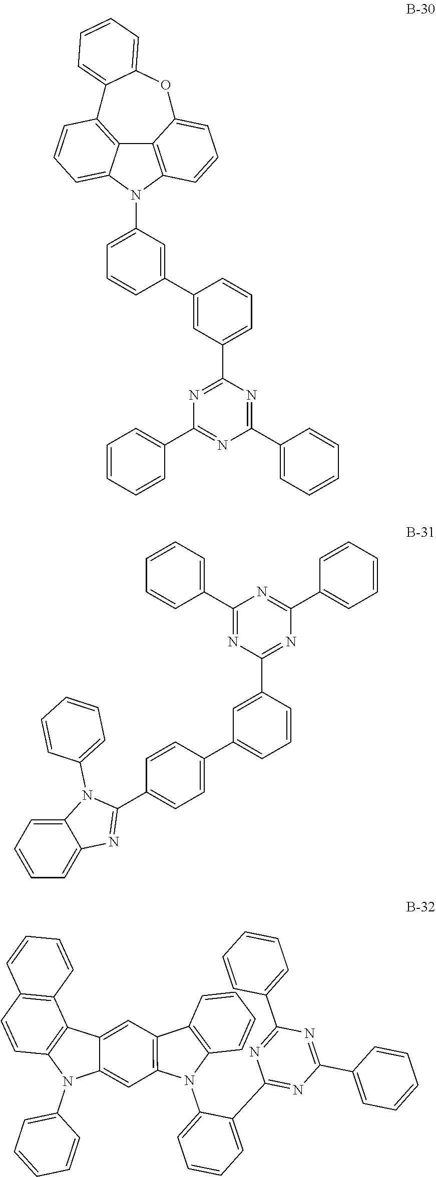 [SMILES: c1ccc(-c2nc(-c3ccccc3)nc(-c3cccc(-c4cccc(-n5c6cccc7oc8ccccc8c8cccc5c8c76)c4)c3)n2)cc1 ; c1ccc(-c2nc(-c3ccccc3)nc(-c3cccc(-c4ccc(-c5nc6ccccc6n5-c5ccccc5)cc4)c3)n2)cc1 ; c1ccc(-c2nc(-c3ccccc3)nc(-c3ccccc3-n3c4ccccc4c4cc5c6c7ccccc7ccc6n(-c6ccccc6)c5cc43)n2)cc1]

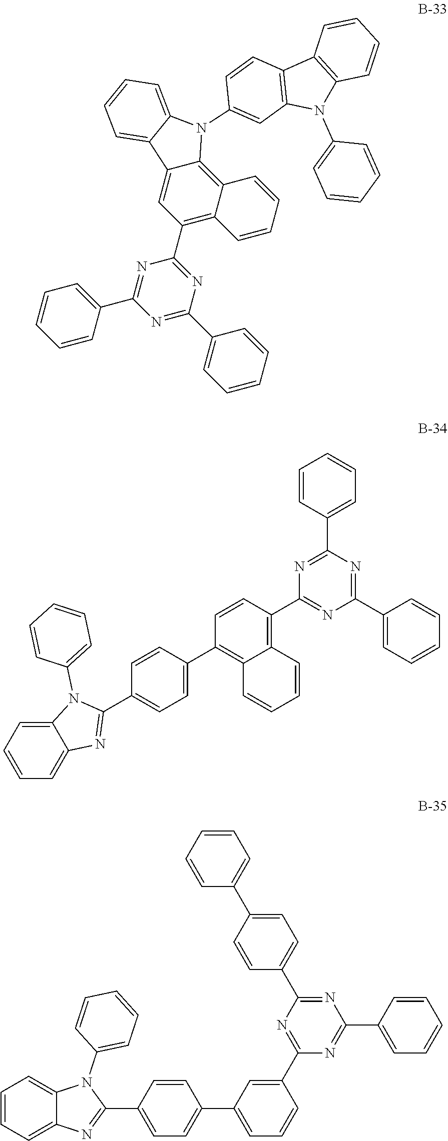 [SMILES: c1ccc(-c2nc(-c3ccccc3)nc(-c3cc4c5ccccc5n(-c5ccc6c7ccccc7n(-c7ccccc7)c6c5)c4c4ccccc34)n2)cc1 ; c1ccc(-c2nc(-c3ccccc3)nc(-c3ccc(-c4ccc(-c5nc6ccccc6n5-c5ccccc5)cc4)c4ccccc34)n2)cc1 ; c1ccc(-c2ccc(-c3nc(-c4ccccc4)nc(-c4cccc(-c5ccc(-c6nc7ccccc7n6-c6ccccc6)cc5)c4)n3)cc2)cc1]

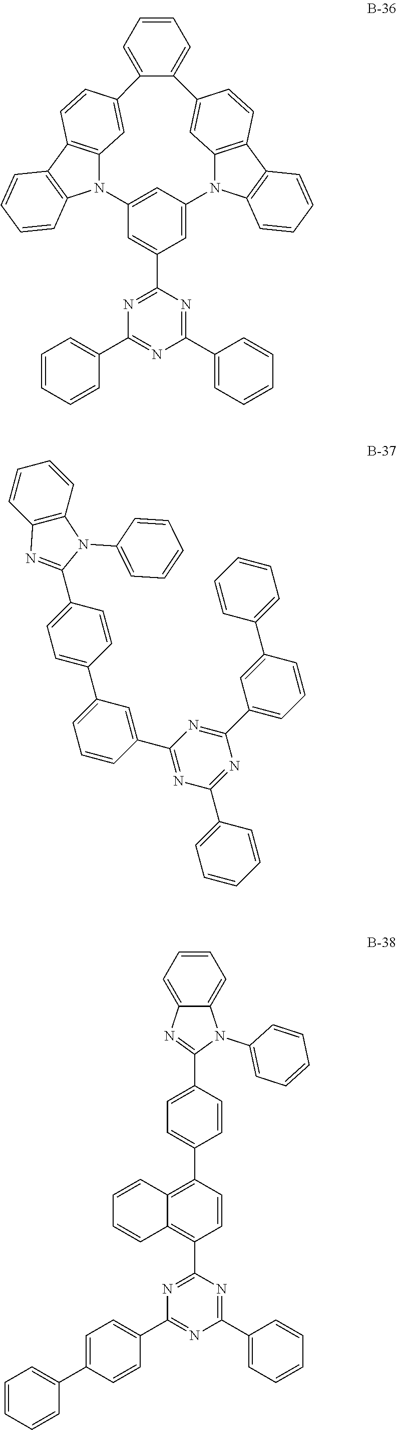 [SMILES: c1ccc(-c2nc(-c3ccccc3)nc(-c3cc4cc(c3)n3c5ccccc5c5ccc(cc53)c3ccccc3c3ccc5c6ccccc6n4c5c3)n2)cc1 ; c1ccc(-c2cccc(-c3nc(-c4ccccc4)nc(-c4cccc(-c5ccc(-c6nc7ccccc7n6-c6ccccc6)cc5)c4)n3)c2)cc1 ; c1ccc(-c2ccc(-c3nc(-c4ccccc4)nc(-c4ccc(-c5ccc(-c6nc7ccccc7n6-c6ccccc6)cc5)c5ccccc45)n3)cc2)cc1]

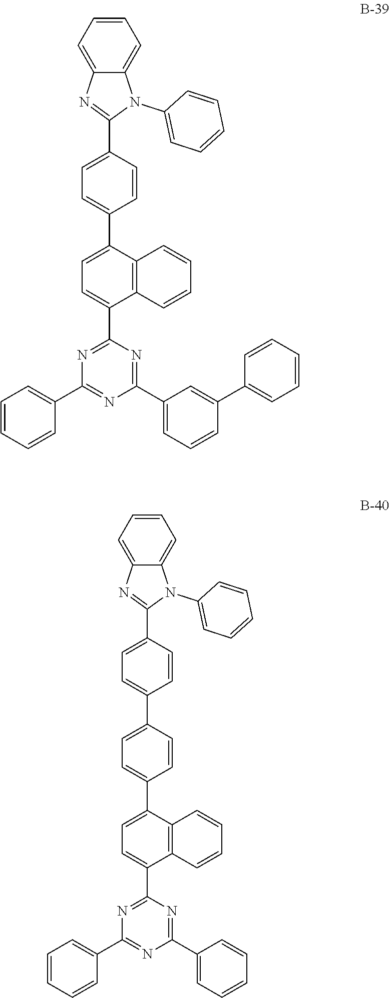 [SMILES: c1ccc(-c2cccc(-c3nc(-c4ccccc4)nc(-c4ccc(-c5ccc(-c6nc7ccccc7n6-c6ccccc6)cc5)c5ccccc45)n3)c2)cc1 ; c1ccc(-c2nc(-c3ccccc3)nc(-c3ccc(-c4ccc(-c5ccc(-c6nc7ccccc7n6-c6ccccc6)cc5)cc4)c4ccccc34)n2)cc1]

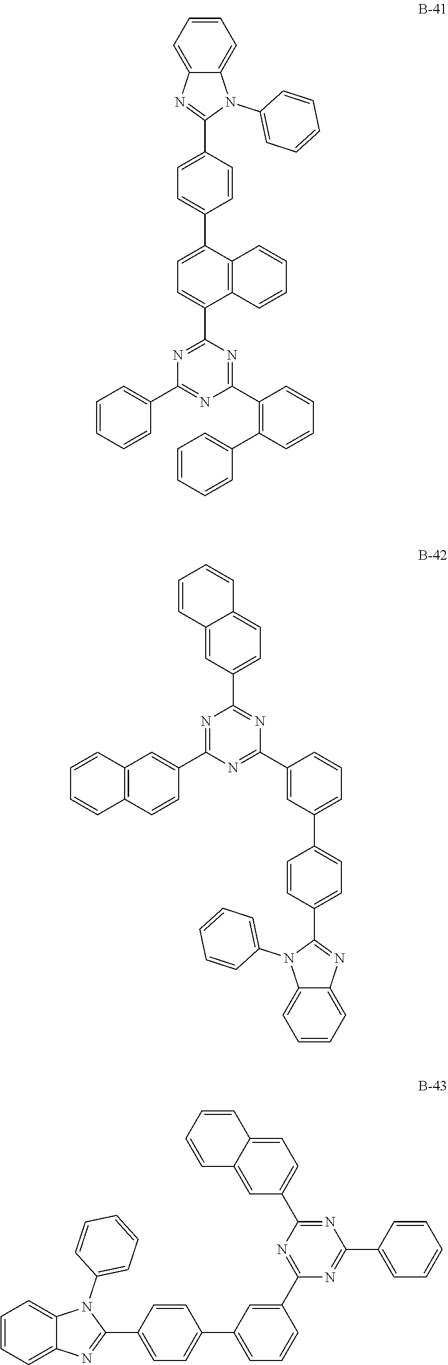 [SMILES: c1ccc(-c2nc(-c3ccccc3-c3ccccc3)nc(-c3ccc(-c4ccc(-c5nc6ccccc6n5-c5ccccc5)cc4)c4ccccc34)n2)cc1 ; c1ccc(-n2c(-c3ccc(-c4cccc(-c5nc(-c6ccc7ccccc7c6)nc(-c6ccc7ccccc7c6)n5)c4)cc3)nc3ccccc32)cc1 ; c1ccc(-c2nc(-c3cccc(-c4ccc(-c5nc6ccccc6n5-c5ccccc5)cc4)c3)nc(-c3ccc4ccccc4c3)n2)cc1]

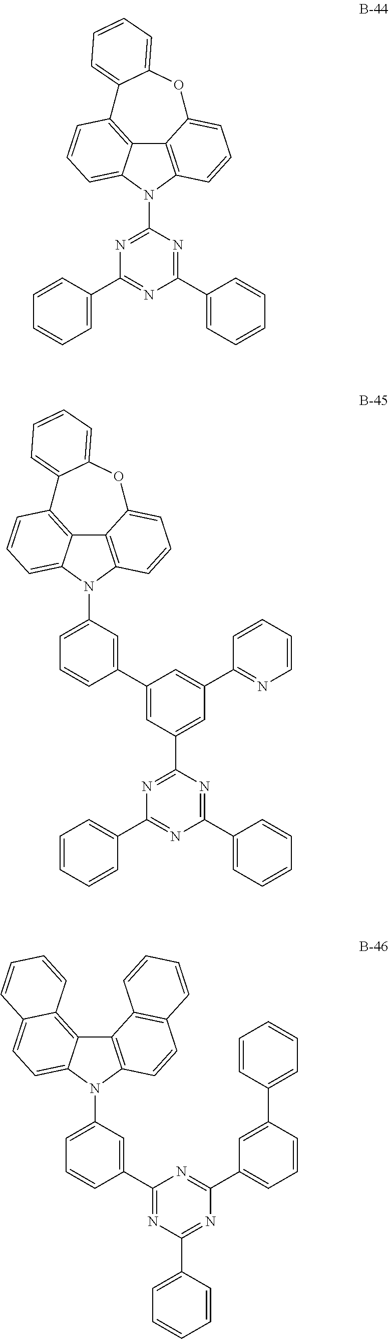 [SMILES: c1ccc(-c2nc(-c3ccccc3)nc(-n3c4cccc5oc6ccccc6c6cccc3c6c54)n2)cc1 ; c1ccc(-c2nc(-c3ccccc3)nc(-c3cc(-c4cccc(-n5c6cccc7oc8ccccc8c8cccc5c8c76)c4)cc(-c4ccccn4)c3)n2)cc1 ; c1ccc(-c2cccc(-c3nc(-c4ccccc4)nc(-c4cccc(-n5c6ccc7ccccc7c6c6c7ccccc7ccc65)c4)n3)c2)cc1]

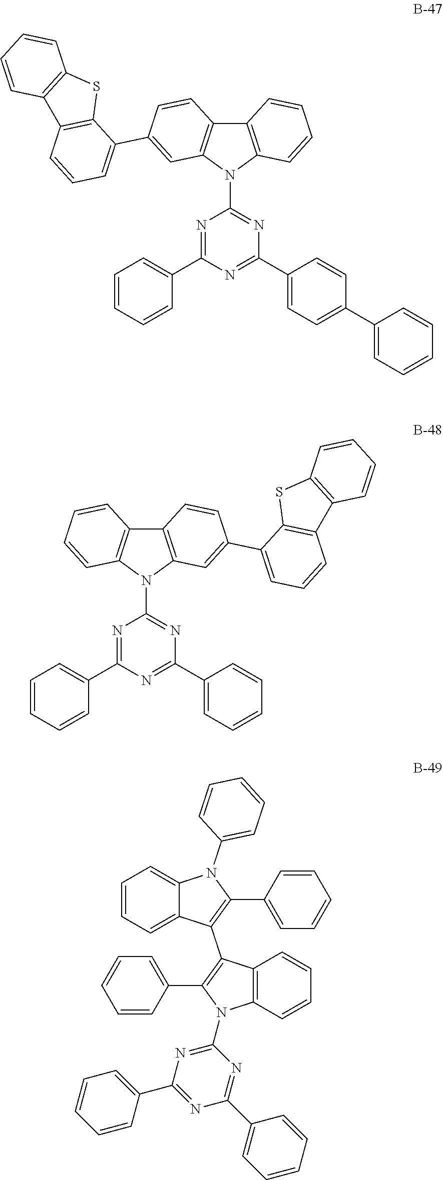 [SMILES: c1ccc(-c2ccc(-c3nc(-c4ccccc4)nc(-n4c5ccccc5c5ccc(-c6cccc7c6sc6ccccc67)cc54)n3)cc2)cc1 ; c1ccc(-c2nc(-c3ccccc3)nc(-n3c4ccccc4c4ccc(-c5cccc6c5sc5ccccc56)cc43)n2)cc1 ; c1ccc(-c2nc(-c3ccccc3)nc(-n3c(-c4ccccc4)c(-c4c(-c5ccccc5)n(-c5ccccc5)c5ccccc45)c4ccccc43)n2)cc1]

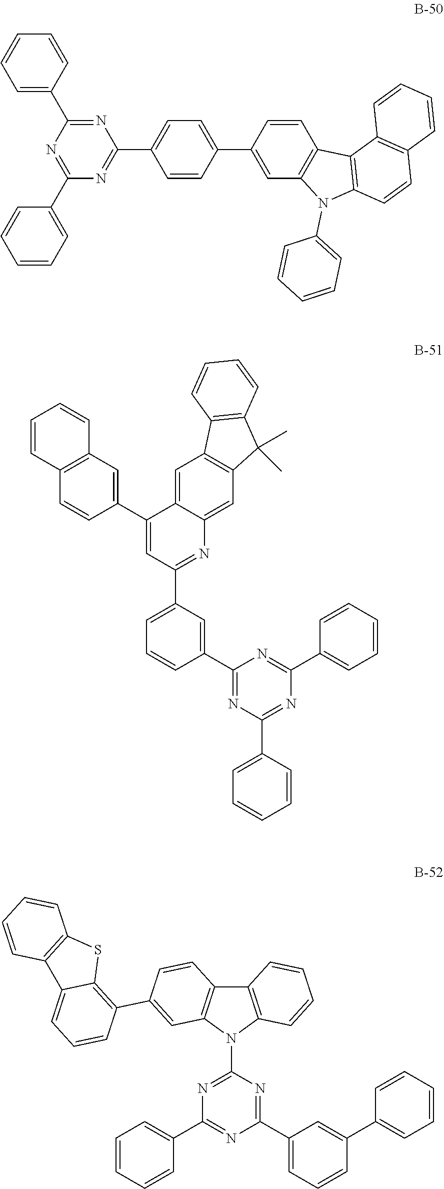 [SMILES: c1ccc(-c2nc(-c3ccccc3)nc(-c3ccc(-c4ccc5c6c7ccccc7ccc6n(-c6ccccc6)c5c4)cc3)n2)cc1 ; CC1(C)c2ccccc2-c2cc3c(-c4ccc5ccccc5c4)cc(-c4cccc(-c5nc(-c6ccccc6)nc(-c6ccccc6)n5)c4)nc3cc21 ; c1ccc(-c2cccc(-c3nc(-c4ccccc4)nc(-n4c5ccccc5c5ccc(-c6cccc7c6sc6ccccc67)cc54)n3)c2)cc1]

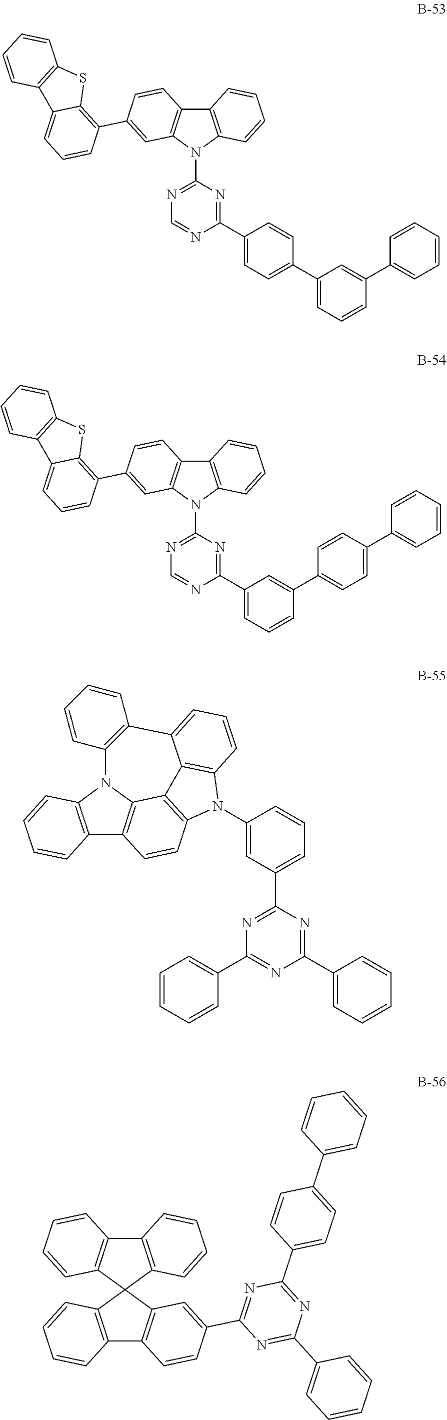 [SMILES: c1ccc(-c2cccc(-c3ccc(-c4ncnc(-n5c6ccccc6c6ccc(-c7cccc8c7sc7ccccc78)cc65)n4)cc3)c2)cc1 ; c1ccc(-c2ccc(-c3cccc(-c4ncnc(-n5c6ccccc6c6ccc(-c7cccc8c7sc7ccccc78)cc65)n4)c3)cc2)cc1 ; c1ccc(-c2nc(-c3ccccc3)nc(-c3cccc(-n4c5cccc6c7ccccc7n7c8ccccc8c8ccc4c(c65)c87)c3)n2)cc1 ; c1ccc(-c2ccc(-c3nc(-c4ccccc4)nc(-c4ccc5c(c4)C4(c6ccccc6-c6ccccc64)c4ccccc4-5)n3)cc2)cc1]

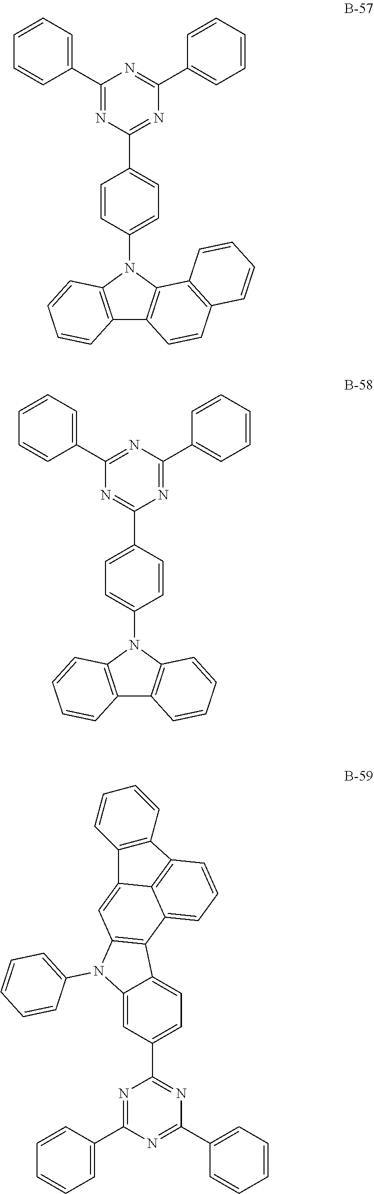 [SMILES: c1ccc(-c2nc(-c3ccccc3)nc(-c3ccc(-n4c5ccccc5c5ccc6ccccc6c54)cc3)n2)cc1 ; c1ccc(-c2nc(-c3ccccc3)nc(-c3ccc(-n4c5ccccc5c5ccccc54)cc3)n2)cc1 ; c1ccc(-c2nc(-c3ccccc3)nc(-c3ccc4c5c6cccc7c6c(cc5n(-c5ccccc5)c4c3)-c3ccccc3-7)n2)cc1]

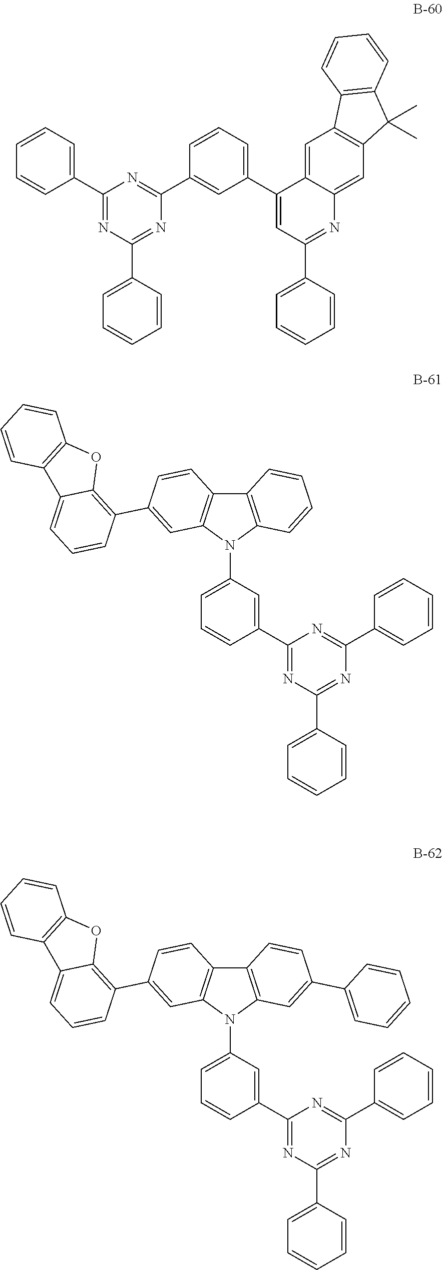 [SMILES: CC1(C)c2ccccc2-c2cc3c(-c4cccc(-c5nc(-c6ccccc6)nc(-c6ccccc6)n5)c4)cc(-c4ccccc4)nc3cc21 ; c1ccc(-c2nc(-c3ccccc3)nc(-c3cccc(-n4c5ccccc5c5ccc(-c6cccc7c6oc6ccccc67)cc54)c3)n2)cc1 ; c1ccc(-c2ccc3c4ccc(-c5cccc6c5oc5ccccc56)cc4n(-c4cccc(-c5nc(-c6ccccc6)nc(-c6ccccc6)n5)c4)c3c2)cc1]

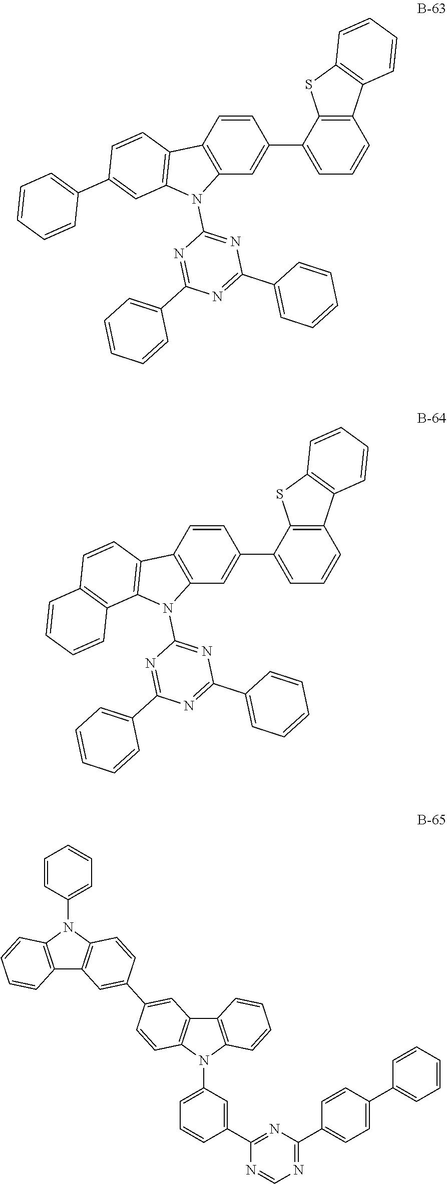 [SMILES: c1ccc(-c2ccc3c4ccc(-c5cccc6c5sc5ccccc56)cc4n(-c4nc(-c5ccccc5)nc(-c5ccccc5)n4)c3c2)cc1 ; c1ccc(-c2nc(-c3ccccc3)nc(-n3c4cc(-c5cccc6c5sc5ccccc56)ccc4c4ccc5ccccc5c43)n2)cc1 ; c1ccc(-c2ccc(-c3ncnc(-c4cccc(-n5c6ccccc6c6cc(-c7ccc8c(c7)c7ccccc7n8-c7ccccc7)ccc65)c4)n3)cc2)cc1]

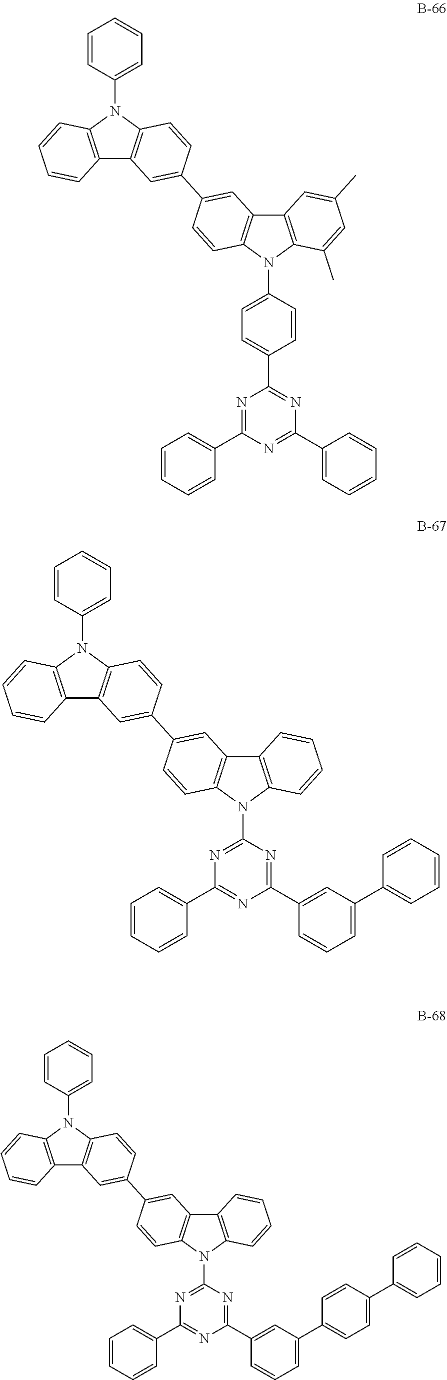 [SMILES: Cc1cc(C)c2c(c1)c1cc(-c3ccc4c(c3)c3ccccc3n4-c3ccccc3)ccc1n2-c1ccc(-c2nc(-c3ccccc3)nc(-c3ccccc3)n2)cc1 ; c1ccc(-c2cccc(-c3nc(-c4ccccc4)nc(-n4c5ccccc5c5cc(-c6ccc7c(c6)c6ccccc6n7-c6ccccc6)ccc54)n3)c2)cc1 ; c1ccc(-c2ccc(-c3cccc(-c4nc(-c5ccccc5)nc(-n5c6ccccc6c6cc(-c7ccc8c(c7)c7ccccc7n8-c7ccccc7)ccc65)n4)c3)cc2)cc1]

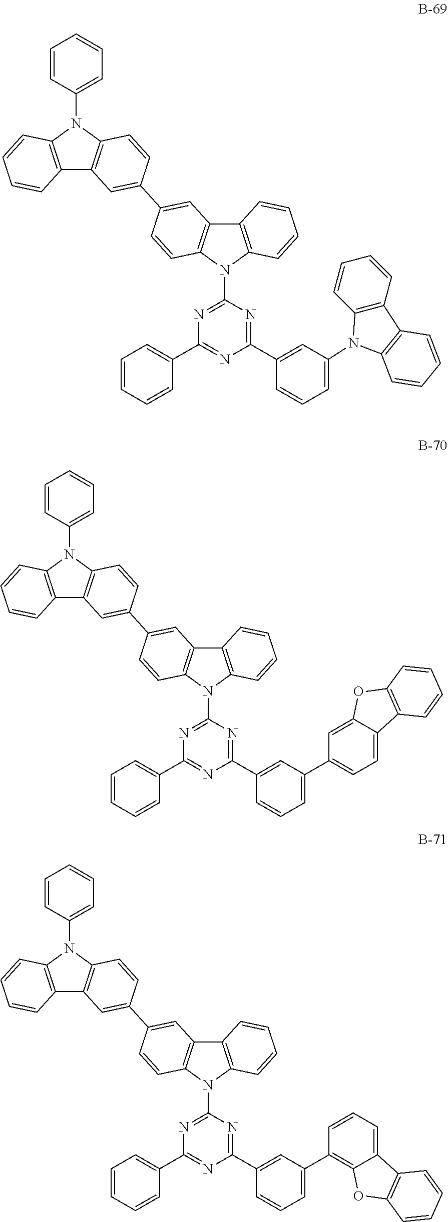 [SMILES: c1ccc(-c2nc(-c3cccc(-n4c5ccccc5c5ccccc54)c3)nc(-n3c4ccccc4c4cc(-c5ccc6c(c5)c5ccccc5n6-c5ccccc5)ccc43)n2)cc1 ; c1ccc(-c2nc(-c3cccc(-c4ccc5c(c4)oc4ccccc45)c3)nc(-n3c4ccccc4c4cc(-c5ccc6c(c5)c5ccccc5n6-c5ccccc5)ccc43)n2)cc1 ; c1ccc(-c2nc(-c3cccc(-c4cccc5c4oc4ccccc45)c3)nc(-n3c4ccccc4c4cc(-c5ccc6c(c5)c5ccccc5n6-c5ccccc5)ccc43)n2)cc1]

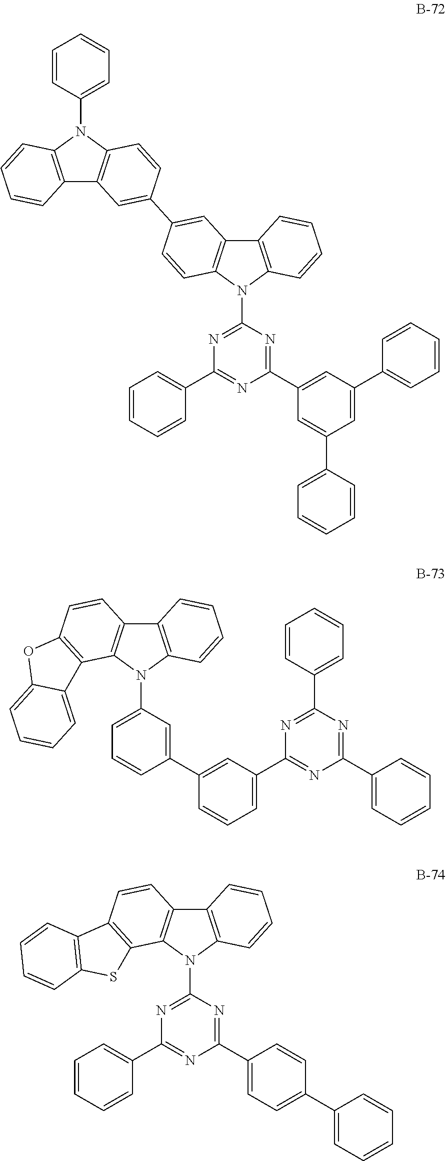 [SMILES: c1ccc(-c2cc(-c3ccccc3)cc(-c3nc(-c4ccccc4)nc(-n4c5ccccc5c5cc(-c6ccc7c(c6)c6ccccc6n7-c6ccccc6)ccc54)n3)c2)cc1 ; c1ccc(-c2nc(-c3ccccc3)nc(-c3cccc(-c4cccc(-n5c6ccccc6c6ccc7oc8ccccc8c7c65)c4)c3)n2)cc1 ; c1ccc(-c2ccc(-c3nc(-c4ccccc4)nc(-n4c5ccccc5c5ccc6c7ccccc7sc6c54)n3)cc2)cc1]

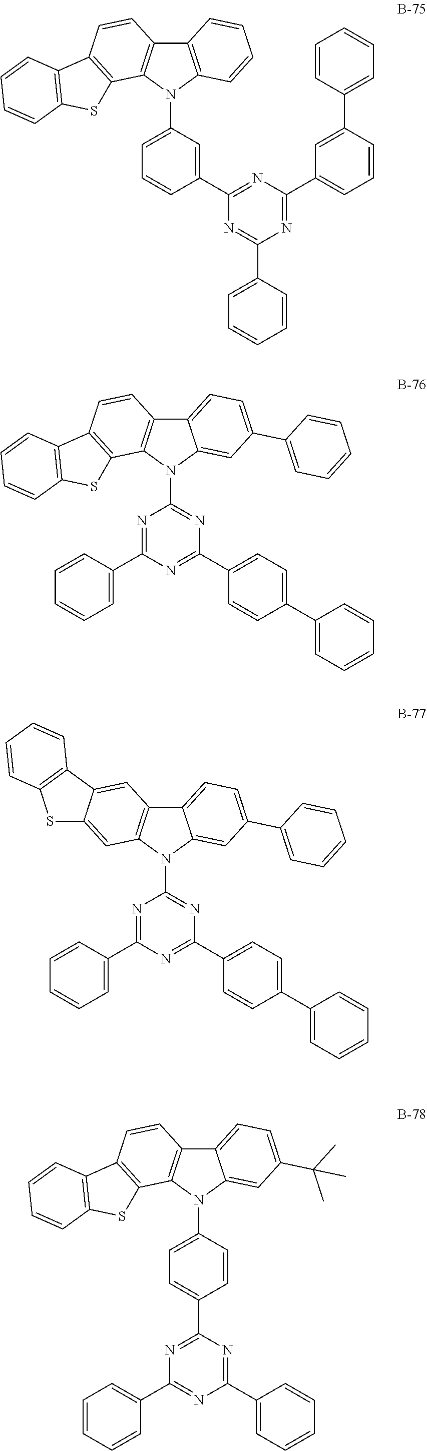 [SMILES: c1ccc(-c2cccc(-c3nc(-c4ccccc4)nc(-c4cccc(-n5c6ccccc6c6ccc7c8ccccc8sc7c65)c4)n3)c2)cc1 ; c1ccc(-c2ccc(-c3nc(-c4ccccc4)nc(-n4c5cc(-c6ccccc6)ccc5c5ccc6c7ccccc7sc6c54)n3)cc2)cc1 ; c1ccc(-c2ccc(-c3nc(-c4ccccc4)nc(-n4c5cc(-c6ccccc6)ccc5c5cc6c(cc54)sc4ccccc46)n3)cc2)cc1 ; CC(C)(C)c1ccc2c3ccc4c5ccccc5sc4c3n(-c3ccc(-c4nc(-c5ccccc5)nc(-c5ccccc5)n4)cc3)c2c1]

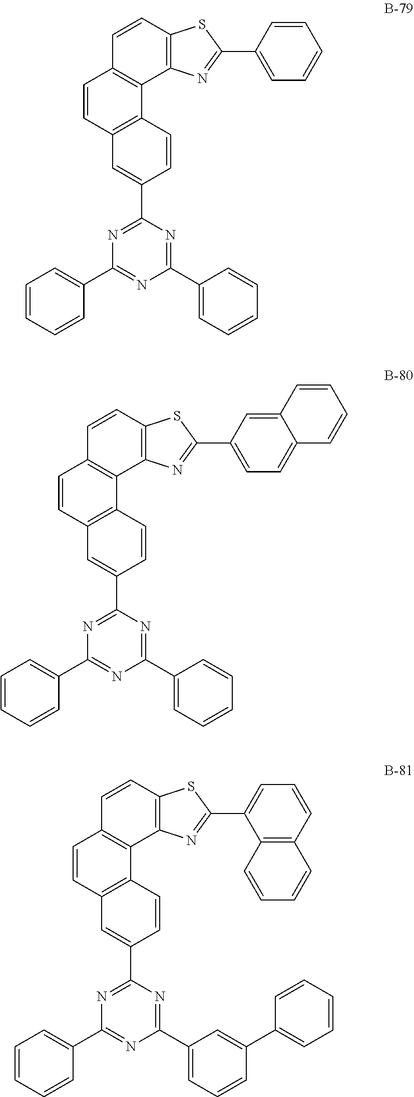 [SMILES: c1ccc(-c2nc(-c3ccccc3)nc(-c3ccc4c(ccc5ccc6sc(-c7ccccc7)nc6c54)c3)n2)cc1 ; c1ccc(-c2nc(-c3ccccc3)nc(-c3ccc4c(ccc5ccc6sc(-c7ccc8ccccc8c7)nc6c54)c3)n2)cc1 ; c1ccc(-c2cccc(-c3nc(-c4ccccc4)nc(-c4ccc5c(ccc6ccc7sc(-c8cccc9ccccc89)nc7c65)c4)n3)c2)cc1]

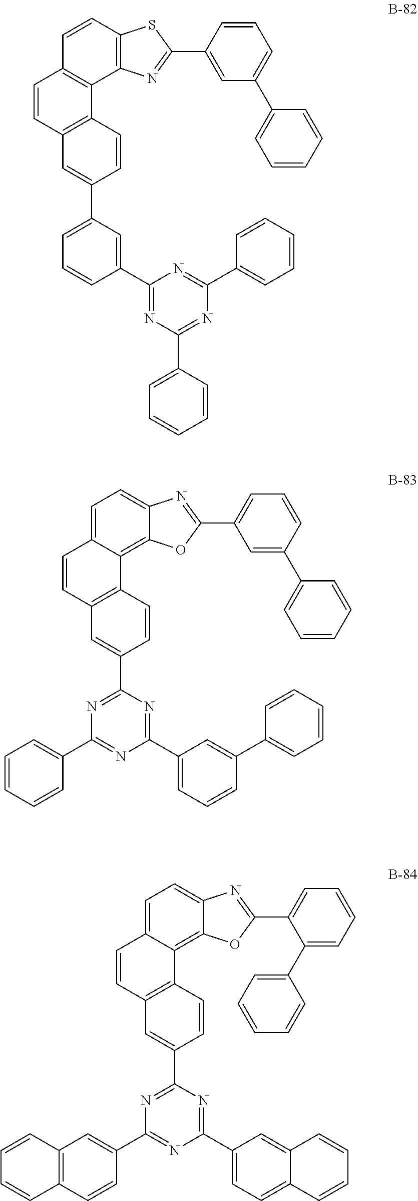 [SMILES: c1ccc(-c2cccc(-c3nc4c(ccc5ccc6cc(-c7cccc(-c8nc(-c9ccccc9)nc(-c9ccccc9)n8)c7)ccc6c54)s3)c2)cc1 ; c1ccc(-c2cccc(-c3nc(-c4ccccc4)nc(-c4ccc5c(ccc6ccc7nc(-c8cccc(-c9ccccc9)c8)oc7c65)c4)n3)c2)cc1 ; c1ccc(-c2ccccc2-c2nc3ccc4ccc5cc(-c6nc(-c7ccc8ccccc8c7)nc(-c7ccc8ccccc8c7)n6)ccc5c4c3o2)cc1]

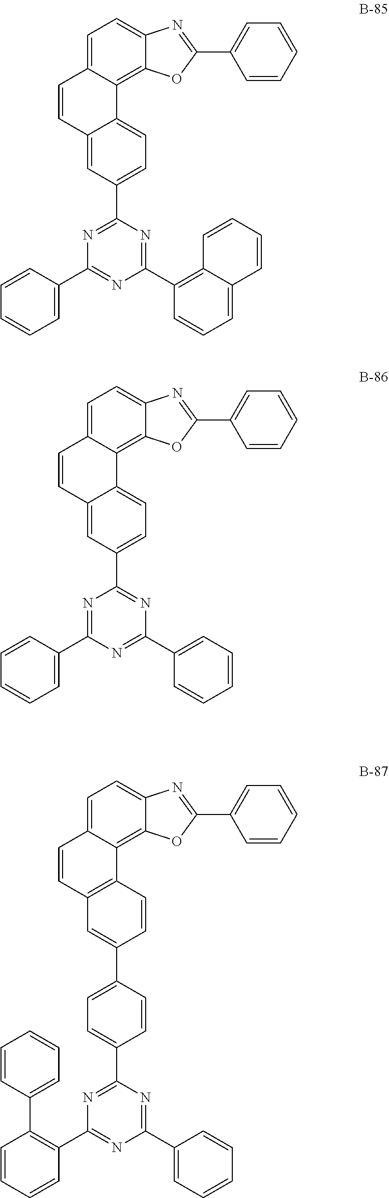 [SMILES: c1ccc(-c2nc(-c3ccc4c(ccc5ccc6nc(-c7ccccc7)oc6c54)c3)nc(-c3cccc4ccccc34)n2)cc1 ; c1ccc(-c2nc(-c3ccccc3)nc(-c3ccc4c(ccc5ccc6nc(-c7ccccc7)oc6c54)c3)n2)cc1 ; c1ccc(-c2nc(-c3ccc(-c4ccc5c(ccc6ccc7nc(-c8ccccc8)oc7c65)c4)cc3)nc(-c3ccccc3-c3ccccc3)n2)cc1]

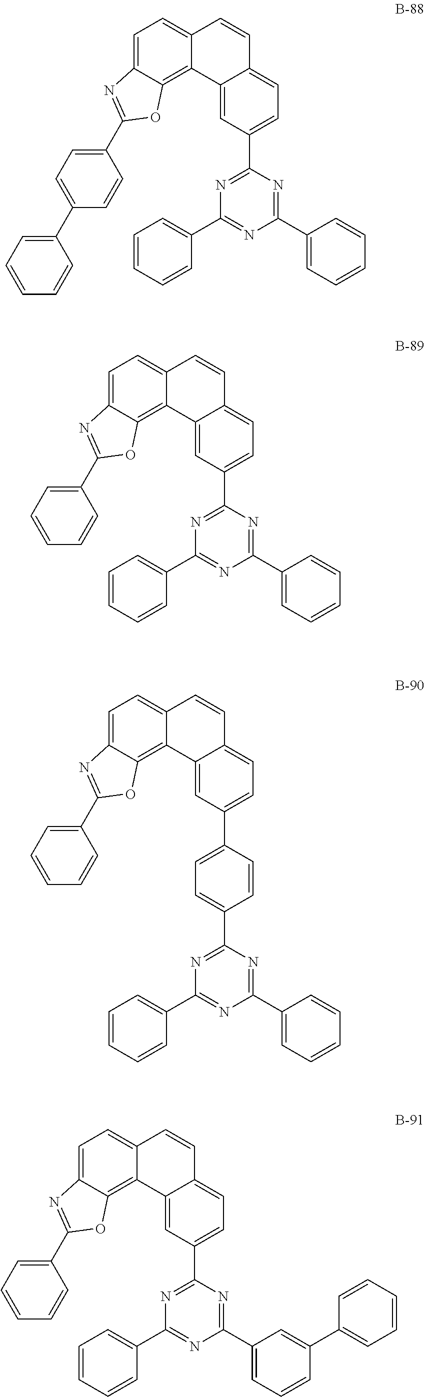 [SMILES: c1ccc(-c2ccc(-c3nc4ccc5ccc6ccc(-c7nc(-c8ccccc8)nc(-c8ccccc8)n7)cc6c5c4o3)cc2)cc1 ; c1ccc(-c2nc(-c3ccccc3)nc(-c3ccc4ccc5ccc6nc(-c7ccccc7)oc6c5c4c3)n2)cc1 ; c1ccc(-c2nc(-c3ccccc3)nc(-c3ccc(-c4ccc5ccc6ccc7nc(-c8ccccc8)oc7c6c5c4)cc3)n2)cc1 ; c1ccc(-c2cccc(-c3nc(-c4ccccc4)nc(-c4ccc5ccc6ccc7nc(-c8ccccc8)oc7c6c5c4)n3)c2)cc1]

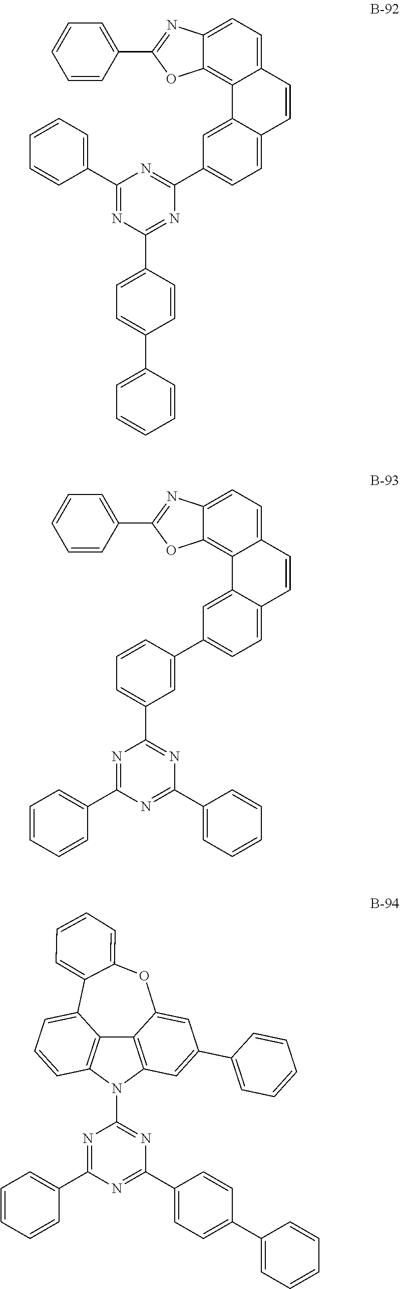 [SMILES: c1ccc(-c2ccc(-c3nc(-c4ccccc4)nc(-c4ccc5ccc6ccc7nc(-c8ccccc8)oc7c6c5c4)n3)cc2)cc1 ; c1ccc(-c2nc(-c3ccccc3)nc(-c3cccc(-c4ccc5ccc6ccc7nc(-c8ccccc8)oc7c6c5c4)c3)n2)cc1 ; c1ccc(-c2ccc(-c3nc(-c4ccccc4)nc(-n4c5cc(-c6ccccc6)cc6oc7ccccc7c7cccc4c7c65)n3)cc2)cc1]

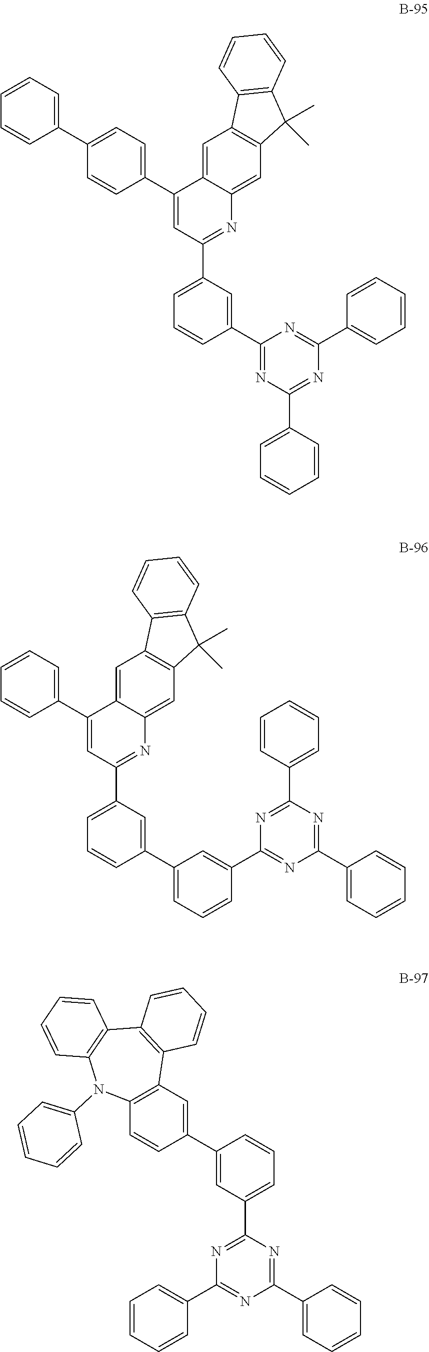 [SMILES: CC1(C)c2ccccc2-c2cc3c(-c4ccc(-c5ccccc5)cc4)cc(-c4cccc(-c5nc(-c6ccccc6)nc(-c6ccccc6)n5)c4)nc3cc21 ; CC1(C)c2ccccc2-c2cc3c(-c4ccccc4)cc(-c4cccc(-c5cccc(-c6nc(-c7ccccc7)nc(-c7ccccc7)n6)c5)c4)nc3cc21 ; c1ccc(-c2nc(-c3ccccc3)nc(-c3cccc(-c4ccc5c(c4)-c4ccccc4-c4ccccc4N5c4ccccc4)c3)n2)cc1]

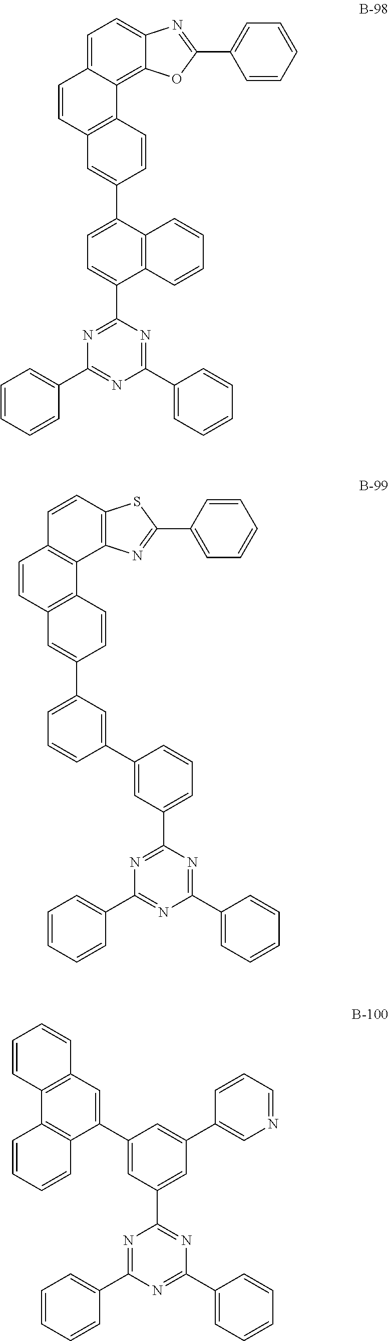 [SMILES: c1ccc(-c2nc(-c3ccccc3)nc(-c3ccc(-c4ccc5c(ccc6ccc7nc(-c8ccccc8)oc7c65)c4)c4ccccc34)n2)cc1 ; c1ccc(-c2nc(-c3ccccc3)nc(-c3cccc(-c4cccc(-c5ccc6c(ccc7ccc8sc(-c9ccccc9)nc8c76)c5)c4)c3)n2)cc1 ; c1ccc(-c2nc(-c3ccccc3)nc(-c3cc(-c4cccnc4)cc(-c4cc5ccccc5c5ccccc45)c3)n2)cc1]

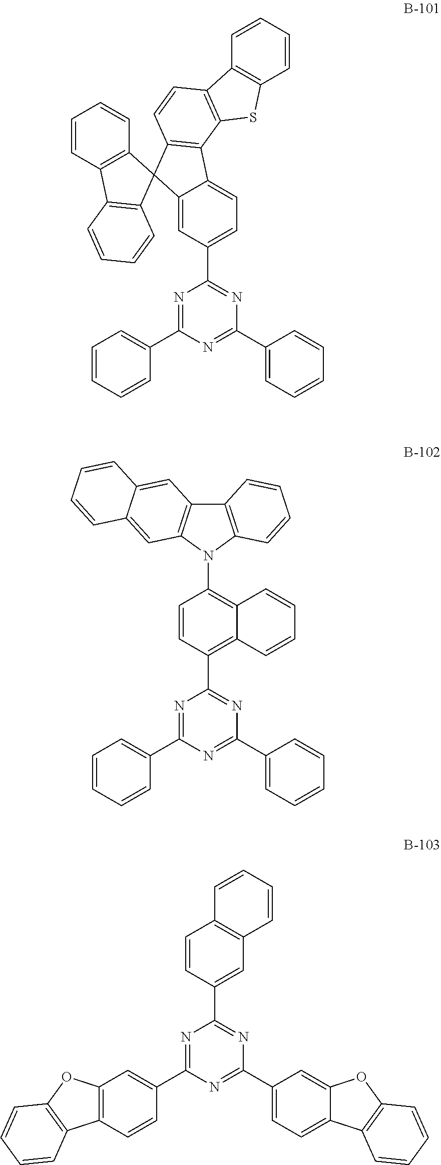 [SMILES: c1ccc(-c2nc(-c3ccccc3)nc(-c3ccc4c(c3)C3(c5ccccc5-c5ccccc53)c3ccc5c(sc6ccccc65)c3-4)n2)cc1 ; c1ccc(-c2nc(-c3ccccc3)nc(-c3ccc(-n4c5ccccc5c5cc6ccccc6cc54)c4ccccc34)n2)cc1 ; c1ccc2cc(-c3nc(-c4ccc5c(c4)oc4ccccc45)nc(-c4ccc5c(c4)oc4ccccc45)n3)ccc2c1]

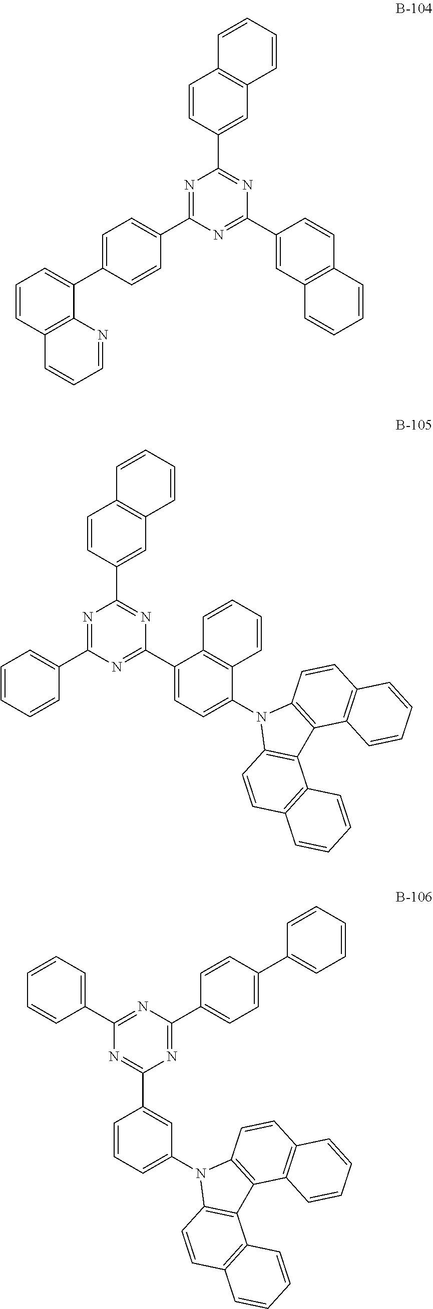 [SMILES: c1ccc2cc(-c3nc(-c4ccc(-c5cccc6cccnc56)cc4)nc(-c4ccc5ccccc5c4)n3)ccc2c1 ; c1ccc(-c2nc(-c3ccc4ccccc4c3)nc(-c3ccc(-n4c5ccc6ccccc6c5c5c6ccccc6ccc54)c4ccccc34)n2)cc1 ; c1ccc(-c2ccc(-c3nc(-c4ccccc4)nc(-c4cccc(-n5c6ccc7ccccc7c6c6c7ccccc7ccc65)c4)n3)cc2)cc1]

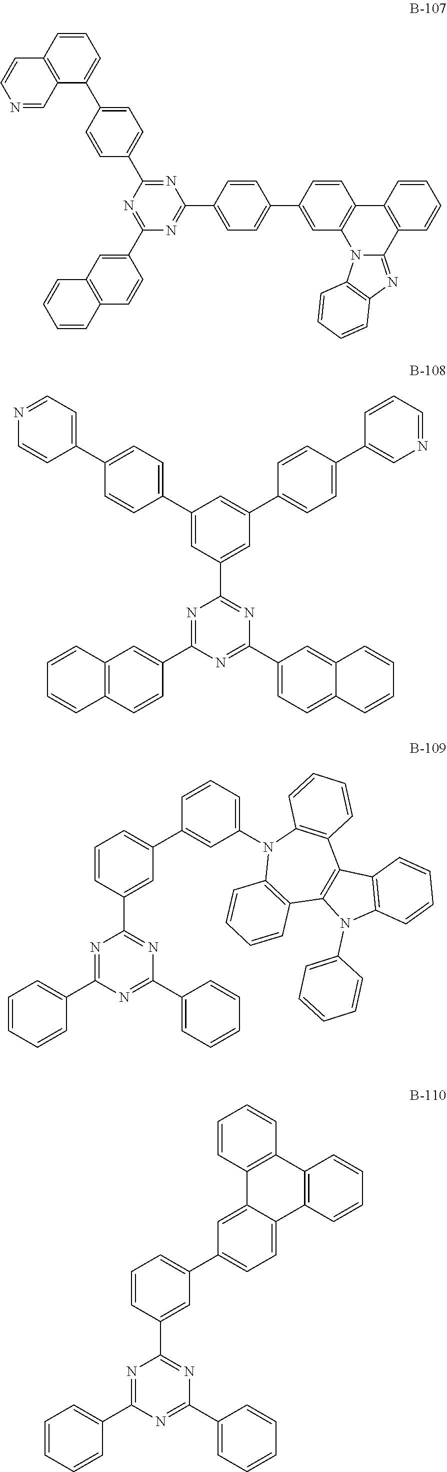 [SMILES: c1ccc2cc(-c3nc(-c4ccc(-c5ccc6c7ccccc7c7nc8ccccc8n7c6c5)cc4)nc(-c4ccc(-c5cccc6ccncc56)cc4)n3)ccc2c1 ; c1cncc(-c2ccc(-c3cc(-c4ccc(-c5ccncc5)cc4)cc(-c4nc(-c5ccc6ccccc6c5)nc(-c5ccc6ccccc6c5)n4)c3)cc2)c1 ; c1ccc(-c2nc(-c3ccccc3)nc(-c3cccc(-c4cccc(N5c6ccccc6-c6c(n(-c7ccccc7)c7ccccc67)-c6ccccc65)c4)c3)n2)cc1 ; c1ccc(-c2nc(-c3ccccc3)nc(-c3cccc(-c4ccc5c6ccccc6c6ccccc6c5c4)c3)n2)cc1]

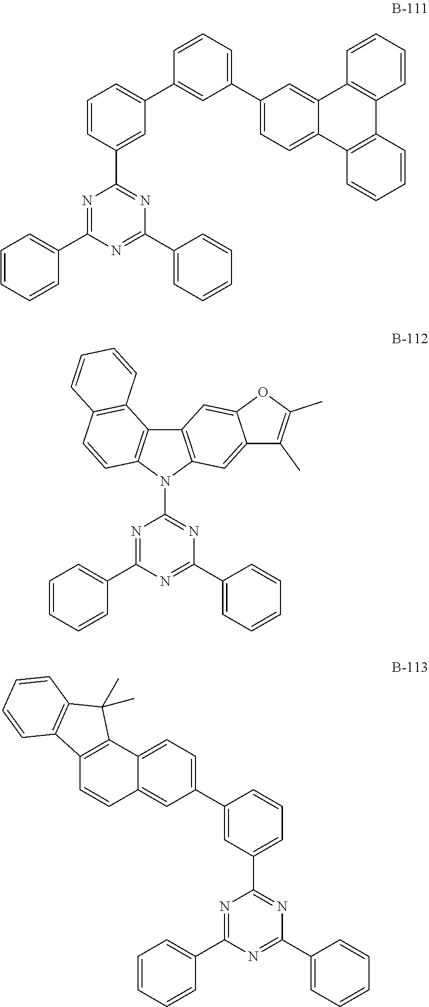 [SMILES: c1ccc(-c2nc(-c3ccccc3)nc(-c3cccc(-c4cccc(-c5ccc6c7ccccc7c7ccccc7c6c5)c4)c3)n2)cc1 ; Cc1oc2cc3c4c5ccccc5ccc4n(-c4nc(-c5ccccc5)nc(-c5ccccc5)n4)c3cc2c1C ; CC1(C)c2ccccc2-c2ccc3cc(-c4cccc(-c5nc(-c6ccccc6)nc(-c6ccccc6)n5)c4)ccc3c21]

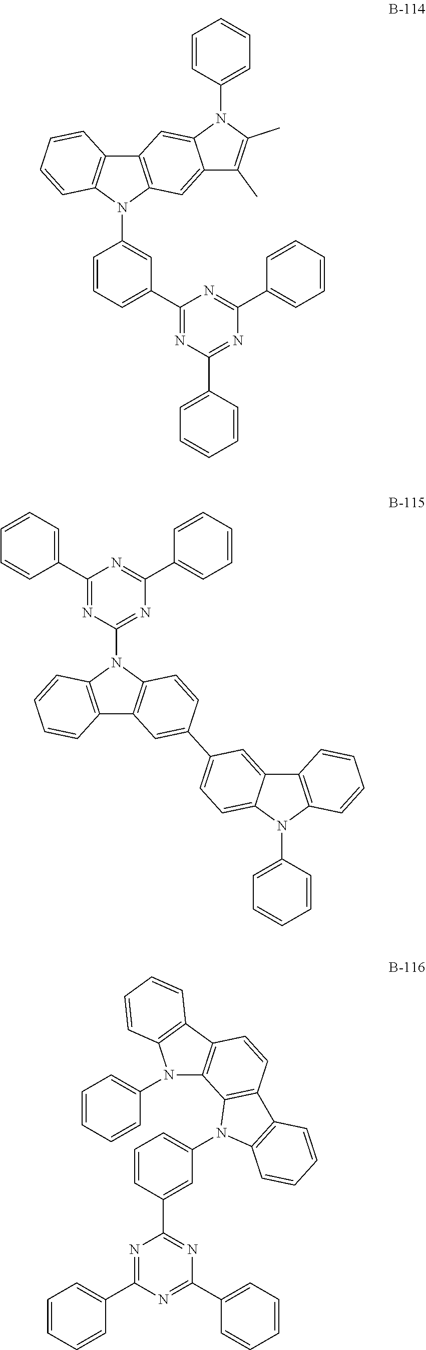 [SMILES: Cc1c(C)n(-c2ccccc2)c2cc3c4ccccc4n(-c4cccc(-c5nc(-c6ccccc6)nc(-c6ccccc6)n5)c4)c3cc12 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3cccc(-n4c5ccccc5c5ccc6c7ccccc7n(-c7ccccc7)c6c54)c3)n2)cc1]

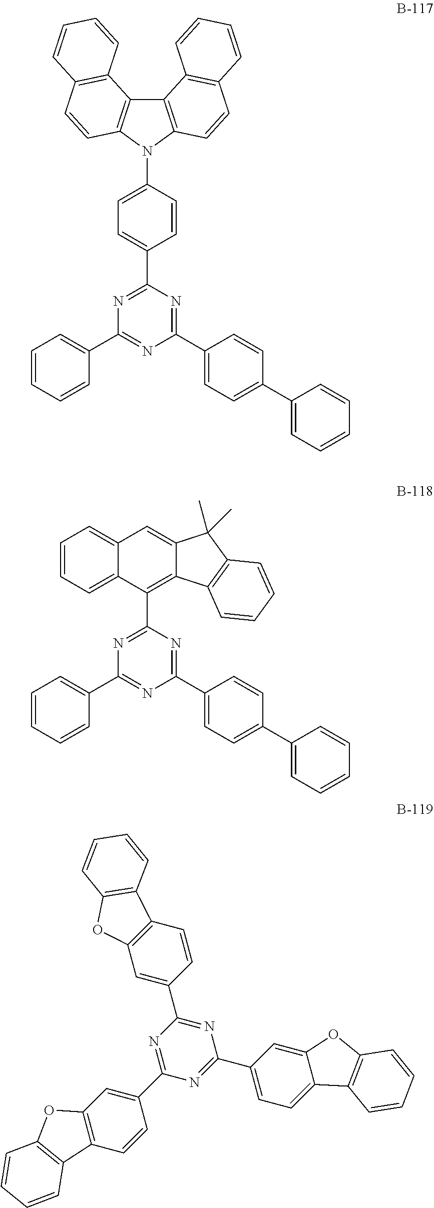 [SMILES: c1ccc(-c2ccc(-c3nc(-c4ccccc4)nc(-c4ccc(-n5c6ccc7ccccc7c6c6c7ccccc7ccc65)cc4)n3)cc2)cc1 ; CC1(C)c2ccccc2-c2c1cc1ccccc1c2-c1nc(-c2ccccc2)nc(-c2ccc(-c3ccccc3)cc2)n1 ; c1ccc2c(c1)oc1cc(-c3nc(-c4ccc5c(c4)oc4ccccc45)nc(-c4ccc5c(c4)oc4ccccc45)n3)ccc12]

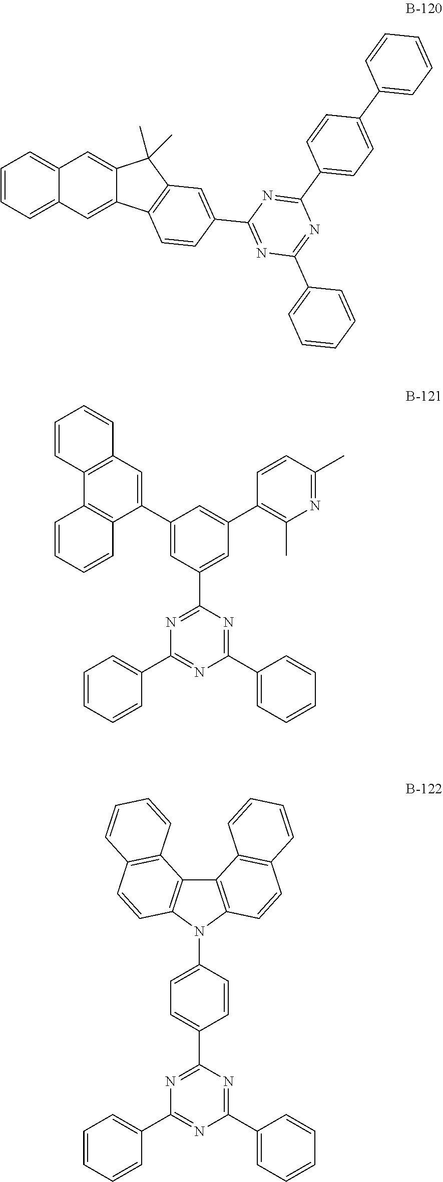 [SMILES: CC1(C)c2cc(-c3nc(-c4ccccc4)nc(-c4ccc(-c5ccccc5)cc4)n3)ccc2-c2cc3ccccc3cc21 ; Cc1ccc(-c2cc(-c3nc(-c4ccccc4)nc(-c4ccccc4)n3)cc(-c3cc4ccccc4c4ccccc34)c2)c(C)n1 ; c1ccc(-c2nc(-c3ccccc3)nc(-c3ccc(-n4c5ccc6ccccc6c5c5c6ccccc6ccc54)cc3)n2)cc1]

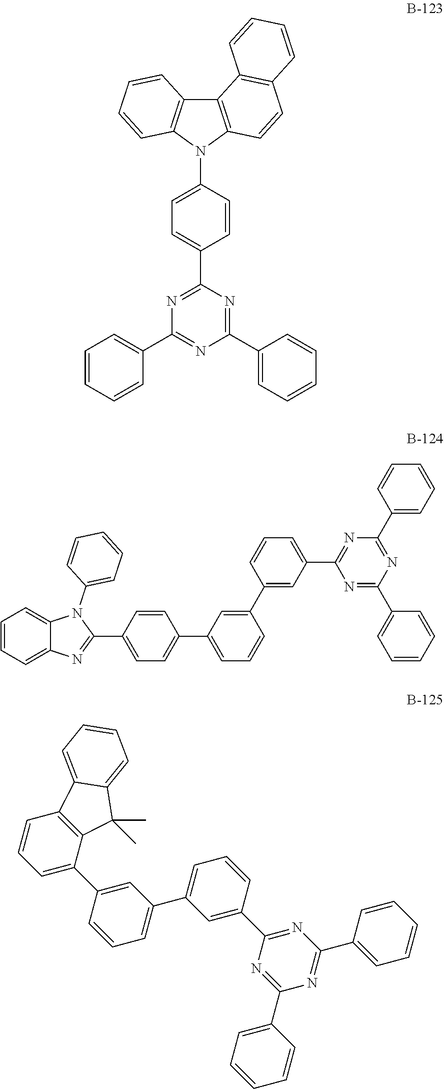 [SMILES: c1ccc(-c2nc(-c3ccccc3)nc(-c3ccc(-n4c5ccccc5c5c6ccccc6ccc54)cc3)n2)cc1 ; c1ccc(-c2nc(-c3ccccc3)nc(-c3cccc(-c4cccc(-c5ccc(-c6nc7ccccc7n6-c6ccccc6)cc5)c4)c3)n2)cc1 ; CC1(C)c2ccccc2-c2cccc(-c3cccc(-c4cccc(-c5nc(-c6ccccc6)nc(-c6ccccc6)n5)c4)c3)c21]

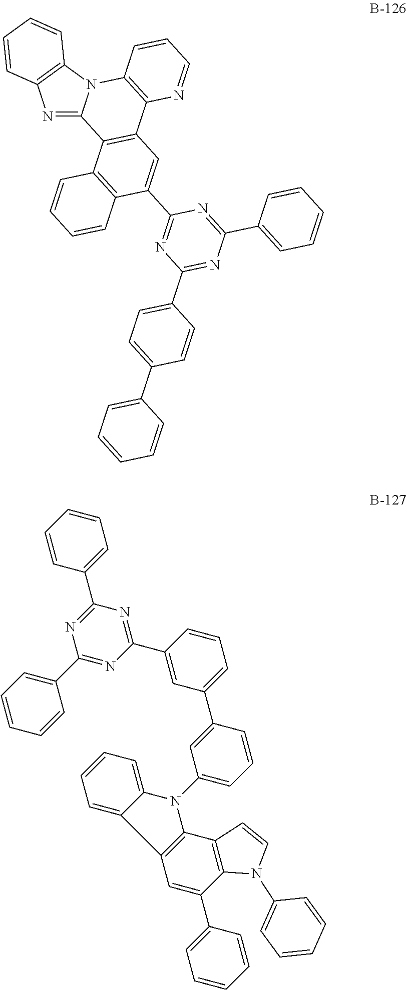 [SMILES: c1ccc(-c2ccc(-c3nc(-c4ccccc4)nc(-c4cc5c6ncccc6n6c7ccccc7nc6c5c5ccccc45)n3)cc2)cc1 ; c1ccc(-c2nc(-c3ccccc3)nc(-c3cccc(-c4cccc(-n5c6ccccc6c6cc(-c7ccccc7)c7c(ccn7-c7ccccc7)c65)c4)c3)n2)cc1]

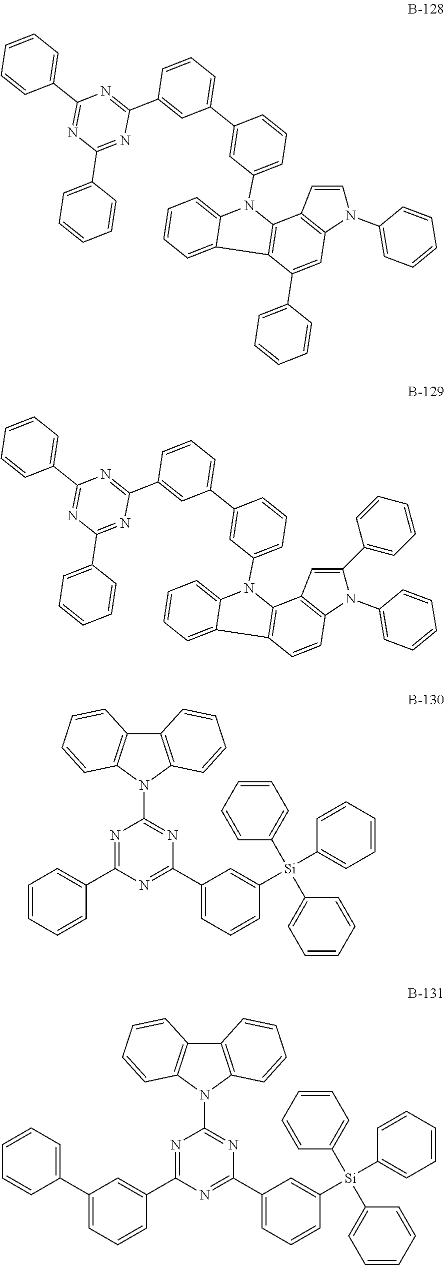 [SMILES: c1ccc(-c2nc(-c3ccccc3)nc(-c3cccc(-c4cccc(-n5c6ccccc6c6c(-c7ccccc7)cc7c(ccn7-c7ccccc7)c65)c4)c3)n2)cc1 ; c1ccc(-c2nc(-c3ccccc3)nc(-c3cccc(-c4cccc(-n5c6ccccc6c6ccc7c(cc(-c8ccccc8)n7-c7ccccc7)c65)c4)c3)n2)cc1 ; c1ccc(-c2nc(-c3cccc([Si](c4ccccc4)(c4ccccc4)c4ccccc4)c3)nc(-n3c4ccccc4c4ccccc43)n2)cc1 ; c1ccc(-c2cccc(-c3nc(-c4cccc([Si](c5ccccc5)(c5ccccc5)c5ccccc5)c4)nc(-n4c5ccccc5c5ccccc54)n3)c2)cc1]

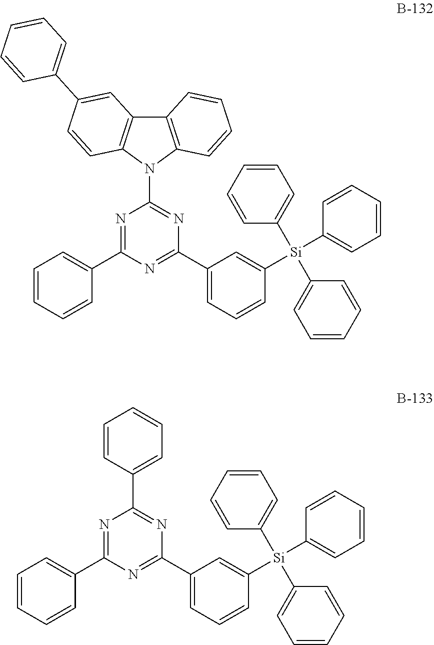 [SMILES: c1ccc(-c2ccc3c(c2)c2ccccc2n3-c2nc(-c3ccccc3)nc(-c3cccc([Si](c4ccccc4)(c4ccccc4)c4ccccc4)c3)n2)cc1 ; c1ccc(-c2nc(-c3ccccc3)nc(-c3cccc([Si](c4ccccc4)(c4ccccc4)c4ccccc4)c3)n2)cc1]

The compound of formula 1 according to the present disclosure can be prepared by a known synthetic method. Specifically, the non-deuteriumated compound of formula 1 can be prepared by known coupling and substitution reactions. For example, the non-deuteriumized compound of formula 1 may be prepared by referring to Korean Patent Application Laid-Open No. 2015-0010016 A (Jan. 28, 2015), etc. The deuteriumated compound of formula 1 can be prepared using a deuteriumized precursor material in a similar manner, or more generally can be prepared by treating a non-deuteriumized compound with a deuteriumized solvent, D6-benzene in the presence of a Lewis acid H/D exchange catalyst such as aluminum trichloride or ethyl aluminum chloride. In addition, the degree of deuteriumization can be controlled by varying reaction conditions such as reaction temperature. For example, the number of deuterium in formula 1 can be adjusted by controlling the reaction temperature and time, the equivalent of acid, etc.

The compound of formula 2 according to the present disclosure can be prepared by a known synthetic method. For example, the compound of formula 2 may be synthesized in the methods described in Korean Patent No. 1876763 (Jul. 11, 2018), Japanese Patent No. 5935199 (May 20, 2016), and Korean Patent Application Laid-Open No. 2017-0130434 A (Nov. 28, 2017), but is not limited thereto.

In addition, the compound of formula 11 or 11-1 may be synthesized by referring to Korean Patent No. 1741415 (May 30, 2017), Korean Patent Application Laid-Open Nos. 2015-0108332 A (Sep. 25, 2015), 2015-0124886 A (Nov. 6, 2015), 2015-0128590 A (Nov. 18, 2015), 2016-0010333 A (Jan. 27, 2016), 2016-0014556 A (Feb. 11, 2016), 2016-0018406 A (Feb. 17, 2016), 2016-0099471 A (Aug. 22, 2016), 2017-0051198 A (May 11, 2017), 2017-0067643 A (Jun. 16, 2017), etc.

The organic electroluminescent device according to one embodiment of the present disclosure includes a substrate, a first electrode formed on the substrate, an organic layer formed on the first electrode, and a second electrode facing the first electrode and formed on the organic layer.

The organic layer includes a hole injection layer, a hole transport layer formed on the hole injection layer, a light-emitting layer formed on the hole transport layer, and an electron transport zone formed on the light-emitting layer, and the electron transport zone includes a hole blocking layer and/or an electron buffer layer formed on the light-emitting layer (if all of the hole blocking layer and the electron buffer layer are formed, the stacking order is irrelevant), an electron transport layer formed on the hole blocking layer or the electron buffer layer, and an electron injection layer formed on the electron transport layer.

The light-emitting layer may be formed using a host compound and a dopant compound. The host compound and dopant compound are as described above. When the light-emitting layer is a layer of two or more layers, each of the light-emitting layers may emit light of different colors. For example, a white light-emitting device can be manufactured by forming three light-emitting layers each emitting blue, red, and green light. In addition, a yellow or orange light-emitting layer may be included, if necessary.

The electron transport zone refers to a zone through which electrons are moved from the second electrode to the light-emitting layer. The electron transport zone may include an electron transporting compound, a reducing dopant, or a combination thereof. The electron transporting compound may be at least one selected from the group consisting of a phenanthrene-based compound, an oxazole-based compound, an isoxazole-based compound, a triazole-based compound, an isothiazole-based compound, an oxadiazole-based compound, a thiadiazole-based compound, a perylene-based compound, an anthracene-based compound, an aluminum complex, and a gallium complex. The reducing dopant may be at least one selected from the group consisting of alkali metals, alkali metal compounds, alkaline earth metals, rare earth metals, halogenated products thereof, oxides thereof, and complexes thereof. Specific examples of the reducing dopant include lithium quinolate, sodium quinolate, cesium quinolate, potassium quinolate, LiF, NaCl, CsF, Li₂O, BaO, and BaF₂, but are not limited thereto.

The hole blocking layer is a layer that blocks the movement of holes injected from the anode toward the cathode since the lifespan and efficiency of the device decreases when holes pass through the light-emitting layer and flow into the cathode. The thickness of the hole blocking layer may be 1 nm to 10 nm. When the thickness is 1 nm or more, the hole blocking role can be basically performed, and when the thickness is less than 10 nm, there is no problem that the driving voltage of the device is increased.

The thickness of the electron buffer layer may be 1 nm or more, and is not particularly limited. Specifically, the thickness of the electron buffer layer may be 2 to 200 nm. The electron buffer layer may be formed on the light-emitting layer by using various known methods such as a vacuum deposition method, a wet process, a laser transfer method, etc. The electron buffer layer refers to a layer that controls charge flow properties. Accordingly, the electron buffer layer may for example, trap electrons, block holes, or lower an energy barrier between the electron transport zone and the light-emitting layer. The electron buffer layer may be included in an organic electroluminescent device emitting all colors such as blue, red, and green, etc.

Each of the electron transport layer and the electron injection layer may be composed of two or more layers.

As a material used for the electron injection layer, a known electron injection material may be used. Examples thereof include lithium quinolate, sodium quinolate, cesium quinolate, potassium quinolate, LiF, NaCl, CsF, Li₂O, BaO, and BaF₂, but are not limited thereto.

The configuration of the organic electroluminescent device described above is only one embodiment provided so that the contents of the present disclosure can be sufficiently delivered to those skilled in the art. The present disclosure is not limited to the corresponding embodiment, and may be embodied in other embodiments.

The present disclosure may include an electron transporting compound, a reducing dopant, or a combination thereof in the electron transport zone.

In addition, the electron transport layer may further include the aforementioned reducing dopant. As a material used for the electron injection layer, a known electron injection material may be used. Examples thereof include lithium quinolate, sodium quinolate, cesium quinolate, potassium quinolate, LiF, NaCl, CsF, Li₂O, BaO, and BaF₂, but are not limited thereto.

A plurality of host materials according to the present disclosure may be used as light-emitting materials for a white organic light-emitting device. The white organic light-emitting device has suggested various structures such as a parallel side-by-side arrangement method, a stacking arrangement method, or color conversion material (CCM) method, etc., according to the arrangement of R (Red), G (Green) or YG (yellowish green), or B (Blue) light-emitting units. In addition, the plurality of host materials according to one embodiment may also be applied to the organic electroluminescent device comprising a QD (quantum dot).

A hole injection layer, a hole transport layer, an electron blocking layer, or a combination thereof may be used between the anode and the light-emitting layer. For the hole injection layer, a plurality of layers may be used for the purpose of lowering a hole injection barrier (or a hole injection voltage) from an anode to a hole transport layer or an electron blocking layer, and two compounds may be used simultaneously for each layer. In addition, the hole injection layer may be doped with p-dopant. The electron blocking layer is located between the hole transport layer (or hole injection layer) and the light-emitting layer, and blocks overflow of electrons from the light-emitting layer to confine excitons in the light-emitting layer to prevent leakage of light. A plurality of layers may be used for the hole transport layer or the electron blocking layer, and a plurality of compounds may be used for each layer.

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

The present disclosure can provide a display device using the organic electroluminescent device comprising the compound represented by formula 1 above, the compound represented by formula 2 above, and the compound represented by formula 11 above. That is, it is possible to manufacture a display device or a lighting device using the organic electroluminescent device of the present disclosure. Specifically, the organic electroluminescent device of the present disclosure can be used for the manufacture of display devices, for example, white organic light-emitting device, smartphones, tablets, notebooks, PCs, TVs, or display devices for vehicles, or lighting devices such as outdoor or indoor lighting.

Hereinafter, the preparation method of compounds according to the present disclosure, and their properties, and the properties of OLED devices of the present disclosure will be explained with reference to representative compounds of the present disclosure in order to understand the present disclosure in detail. However, the following examples are only to describe the properties of the compound according to the present disclosure and the OLED device according to the present disclosure in order to understand the present disclosure in detail, and the present application is not limited to the following examples.

### [Synthesis Example 1] Synthesis of Compound H-190

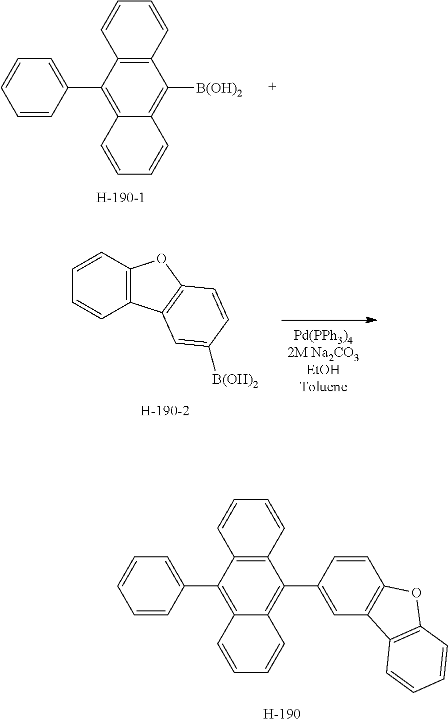 [SMILES: OB(O)c1c2ccccc2c(-c2ccccc2)c2ccccc12 ; OB(O)c1ccc2oc3ccccc3c2c1 ; c1ccc(-c2c3ccccc3c(-c3ccc4oc5ccccc5c4c3)c3ccccc23)cc1]

Compound H-190-1 (32.7 g, 109.8 mmol), compound H-190-2 (22.5 g, 91.5 mmol), tetrakis(triphenylphosphine)palladium (Pd(PPh₃)₄) (5.3 g, 46 mmol), 115 mL of 2 M Na₂CO₃, 460 mL of toluene, and 115 mL of ethanol (EtOH) were added into a flask, and then stirred under reflux at 140° C. for 2 hours. After cooling to room temperature, the organic layer was extracted with ethyl acetate, and the residual water was removed with magnesium sulfate followed by drying. Next it was separated by column chromatography to obtain compound H-190 (21.7 yield: 56.51%).

| | MW | M.P. |
|---|---|---|
| H-190 | 420.15 | 247° C. |

### [Synthesis Example 2] Synthesis of Compound H-206

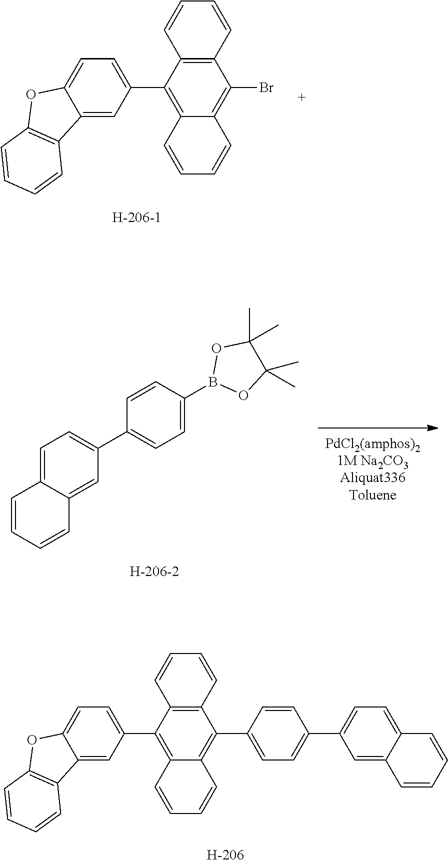 [SMILES: Brc1c2ccccc2c(-c2ccc3oc4ccccc4c3c2)c2ccccc12 ; CC1(C)OB(c2ccc(-c3ccc4ccccc4c3)cc2)OC1(C)C ; c1ccc2cc(-c3ccc(-c4c5ccccc5c(-c5ccc6oc7ccccc7c6c5)c5ccccc45)cc3)ccc2c1]

Compound H-206-1 (10.0 g, 0.024 mol), compound H-206-2 (7.0 g, 0.028 mol), PdCl₂(amphos)₂ (0.84 g, 0.0012 mol), 50 mL of 1 M Na₂CO₃, 150 mL of toluene, and Aliquat336 (0.48 g, 0.0012 mol) were added into a flask, and then stirred at 140° C. for 2 hours. The mixture was cooled to room temperature, and then the organic layer was extracted with ethyl acetate followed by distilling under reduced pressure. Next, it was separated by column chromatography to obtain compound H-206 (10.8 g, yield: 83.72%).

| | MW | M.P. |
|---|---|---|
| H-206 | 546.6 | 310° C. |

### [Synthesis Example 3] Synthesis of Compound B-73

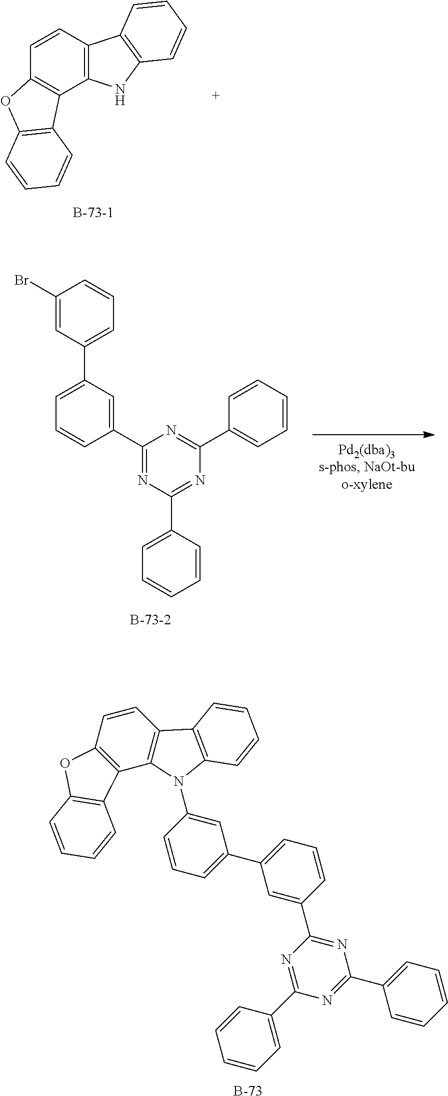

Compound B-73-1 (20 g, 0.078 mol), compound B-73-2 (43.3 g, 0.093 mol), Pd₂(dba)₂ (3.6 g, 0.0039 mol), S-Phos (3.2 g, 0.0078 mol), NaOt-Bu (18.7 g, 0.194 mol), and 520 mL of o-xylene were added into a flask, and then stirred under reflux at 180° C. for 3 hours. The mixture was cooled to room temperature, and then methanol was added thereto followed by filtering the solid under reduced pressure. The obtained solid was dissolved in chloroform, and then separated by column chromatography to obtain compound B-73 (43.6 g, yield: 87.55%).

| | MW | M.P. |
|---|---|---|
| B-73 | 640.7 | 251° C. |

### [Synthesis Example 4] Synthesis of Compound B-113

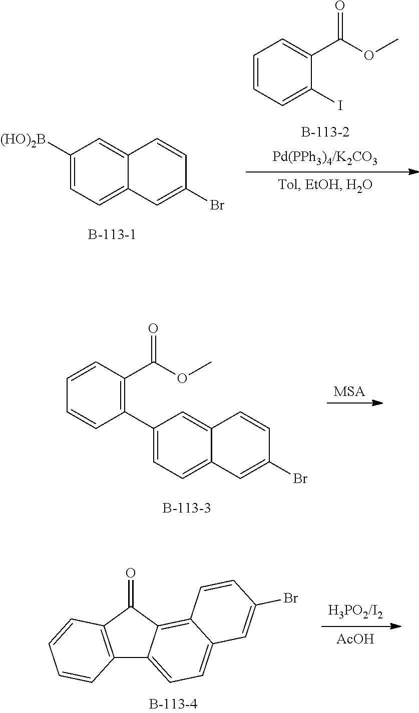

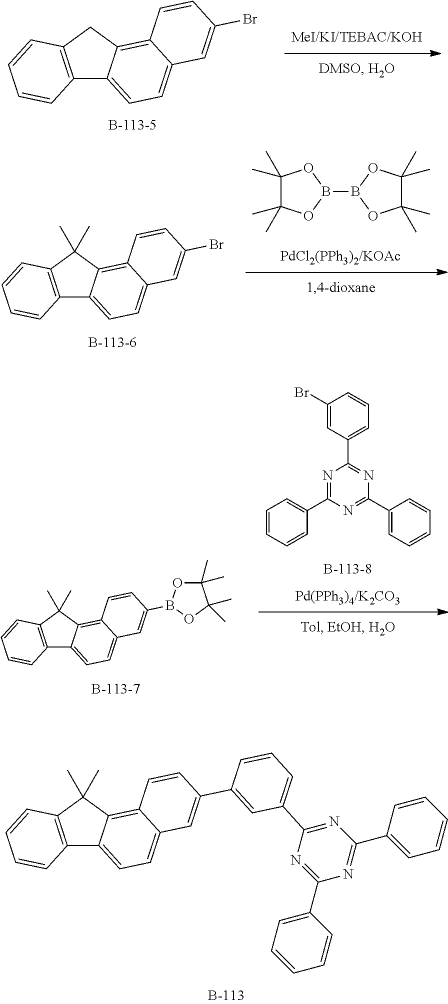

1) Synthesis of Compound B-113-3

Compound B-113-1 (50.3 g, 200.34 mmol), compound B-113-2 (50.0 g, 190.80 mmol), Pd(PPh₃)₄ (6.6 g, 5.72 mmol), potassium carbonate (K₂CO₃) (66.0 g, 477 mmol), 950 mL of toluene, 240 mL of EtOH, and 240 mL of distilled water (H₂O) were added into a flask, and then stirred under reflux for 3 hours. After completion of the reaction, the mixture was extracted with ethyl acetate, and then separated by column chromatography to obtain compound B-113-3 (61.7 g, yield: 85%).

2) Synthesis of Compound B-113-4

Compound B-113-3 (61.7 g, 180.83 mmol) and 720 mL of methanesulfonic acid (MSA) were added into a flask, and then stirred at 70° C. for 2 hours. After completion of the reaction, the mixture was added dropwise to H₂O, and then filtered to obtain compound B-113-4 (40.1 g, yield: 72%).

3) Synthesis of Compound B-113-5

Hypophosphite (H₃PO₂) (22.0 mL, 207.53 mmol), iodine (I₂) (17.1 g, 67.45 mmol), and 650 mL of acetic acid (AcOH) were added into a flask, and then stirred under reflux for 1 hour. Next, compound B-113-4 (40.1 g, 129.71 mmol) was added to the mixture, and then stirred under reflux for 2 hours. After completion of the reaction, the mixture was extracted with ethyl acetate to obtain compound B-113-5 (38.3 g, yield: 100%).

4) Synthesis of Compound B-113-6

Compound B-113-5 (38.3 g, 129.76 mmol), potassium iodide (KI) (2.2 g, 12.98 mmol), potassium hydroxide (KOH) (36.4 g, 648.80 mmol), benzyltriethylammonium chloride (TEBAC) (1.8 g, 6.49 mmol), 650 mL of dimethyl sulfoxide (DMSO), and 65 mL of H₂O were added into a flask, and then stirred at room temperature for 30 minutes. Next, methyl iodide (Mel) (20.2 mL, 324.39 mmol) was added thereto, and then stirred at room temperature for 2 hours. After completion of the reaction, the mixture was extracted with ethyl acetate, and then separated by column chromatography to obtain compound B-113-6 (36.0 g, yield: 86%).

5) Synthesis of Compound B-113-7

Compound B-113-6 (10 g, 30.94 mmol), bis(pinacolato)diborone (11 g, 43.32 mmol), bis(triphenylphosphine)palladium(II)dichloride (PdCl₂(PPh₃)₂) (1.1 g, 1.55 mmol), potassium acetate (KOAc) (6.1 g, 61.88 mmol), and 155 mL of 1,4-dioxane were added into a flask, and then stirred at 130° C. for 6 hours. After completion of the reaction, the mixture was cooled to room temperature and then extracted with ethyl acetate. The extracted organic layer was dried with magnesium sulfate, and then the solvent was removed with a rotary evaporator. Next, it was separated by column chromatography to obtain compound B-113-7 (7.8 g, yield: 68%).

6) Synthesis of compound B-113

Compound B-113-7 (3.0 g, 8.10 mmol), compound B-113-8 (3.0 g, 7.72 mmol), Pd(PPh₃)₄ (0.3 g, 0.23 mmol), K₂CO₃ (2.0 g, 19.30 mmol), 40 mL of toluene, 10 mL of EtOH, and 10 mL of H₂O were added into a flask, and then stirred under reflux for 4 hours. After completion of the reaction, the mixture was extracted with ethyl acetate, and then separated by column chromatography to obtain compound B-113 (2.8 g, yield: 67%).

| | MW | M.P. |
|---|---|---|
| B-113 | 551.69 | 219° C. |

### [Synthesis Example 5] Synthesis of Compound B-93

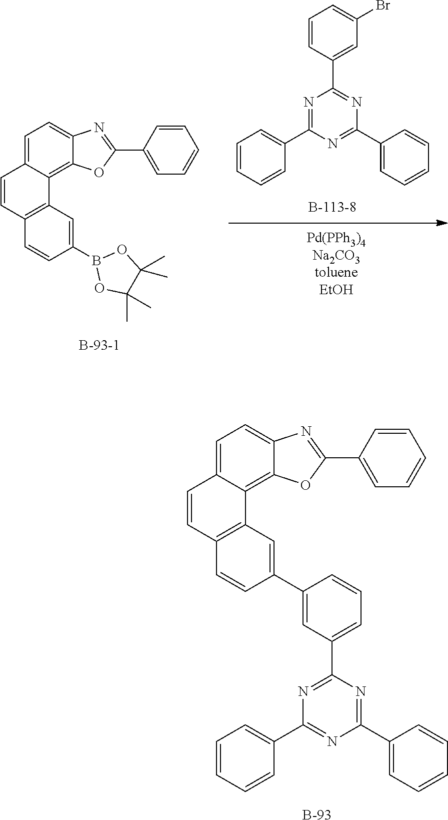

Compound B-93-1 (3 g, 7.1 mmol), compound B-113-8 (3.04 g, 7.8 mmol). Pd(PPh₃)₄ (0.41 g, 0.36 mmol), sodium carbonate (Na₂CO₃) (1.9 g, 17.8 mmol), 24 mL of toluene, 6 mL of EtOH, and 6 mL of H₂O were added into a flask, and then stirred at 120° C. for 4 hours. After completion of the reaction, the mixture was added dropwise to methanol, and then the obtained solid was filtered. The obtained solid was purified by column chromatography and recrystallization to obtain compound B-93 (2.3 g, 53.6%).

| | MW | M.P. |
|---|---|---|
| B-93 | 602.70 | 288° C. |

### [Synthesis Example 6] Synthesis of Compound B-45

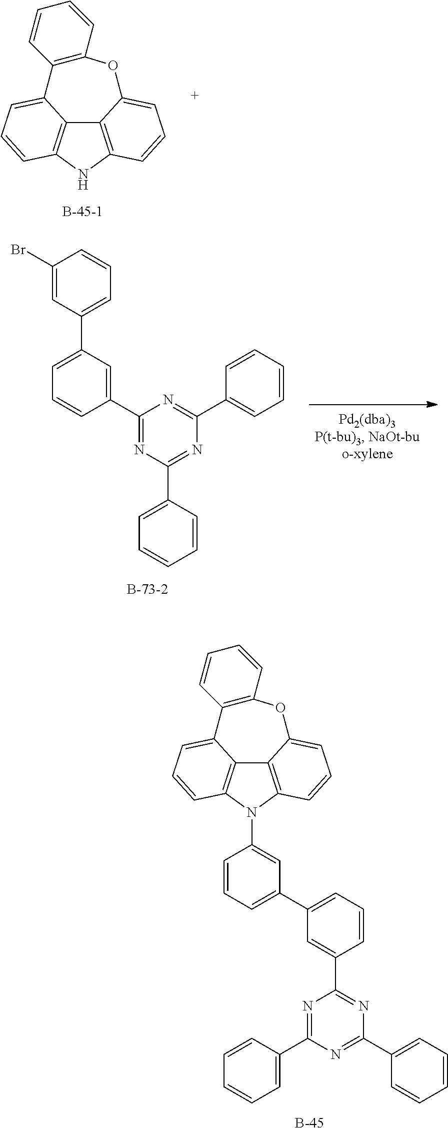

Compound B-45-1 (43.8 g, 0.170 mol), compound B-73-2 (119 g, 0.255 mol), Pd₂(dba)₃ (16 g, 0.017 mol). P(t-bu)₃ (8.5 mL, 0.034 mol), NaOt-Bu (498 g, 0.51 mol), and 900 mL of o-xylene were added to a flask, and then stirred under reflux at 180° C. for 2 hours. The mixture was cooled to room temperature, and then methanol was added thereto. Next, the solid was filtered under reduced pressure. The obtained solid was dissolved in chloroform, and then separated by column chromatography to obtain compound B-45 (38 g, yield: 35.18%).

| | MW | M.P. |
|---|---|---|
| B-45 | 640.7 | 238° C. |

### [Device Example 1-1] Preparation of an OLED According to the Present Disclosure

An OLED according to the present disclosure was produced. First, a transparent electrode indium tin oxide (ITO) thin film (10 A/sq) on a glass substrate for an OLED (GEOMATEC CO., LTD., Japan) was subjected to an ultrasonic washing with acetone, ethanol and distilled water, sequentially, and thereafter was stored in isopropanol and then used. After evacuating until the degree of vacuum in the chamber reaches 10-torr, the ITO substrate was mounted on a substrate holder of a vacuum vapor deposition apparatus. Then, compound HT-1 described in the following Table 1 as a first hole injection compound was introduced into one cell of the vacuum vapor deposition apparatus, and compound HI-1 described in the following Table 1 was introduced into another cell of the vacuum vapor deposition apparatus. The two materials were evaporated at different rates and compound HI-1 was deposited in a doping amount of 3 wt % based on the total amount of compound HT-1 and compound HI-1 to form a hole injection layer having a thickness of 10 nm. Next, compound HT-1 was deposited as a first hole transport layer having a thickness of 75 nm on the hole injection layer. Next, compound HT-2 was then introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a light-emitting layer was formed thereon as follows: Compound H-190 was introduced into one cell of the vacuum vapor deposition apparatus as a host, and compound BD was introduced into another cell as a dopant. The two materials were evaporated at different rates and the dopant was deposited in a doping amount of 2 wt % based on the total amount of the host and dopant to form a light-emitting layer having a thickness of 20 nm on the second hole transport layer. Next, compound B-73 was deposited as a hole blocking layer having a thickness of 5 nm on the light-emitting layer. Next, compound ET-1 and compound EI-1 in another two cells were evaporated at a rate of 1:1 to deposit an electron transport layer having a thickness of 30 nm on the hole blocking layer. Next, after depositing compound EI-1 as an electron injection layer having a thickness of 2 nm, an Al cathode having a thickness of 80 nm was deposited by another vacuum vapor deposition apparatus. Thus, an OLED was produced.

The time taken for luminance to decrease from 100% to 95% at a luminance of 1,400 nits (lifespan; T95) of the organic electroluminescent device according to Device Example 1-1 prepared as described above was 56 hours.

### [Device Example 1-2] Preparation of an OLED According to the Present Disclosure

An OLED was produced in the same manner as in Device Example 1-1, except that compound B-113 was used as a material of the hole blocking layer.

The time taken for luminance to decrease from 100% to 95% at a luminance of 1,400 nits (lifespan; T95) of the organic electroluminescent device according to Device Example 1-2 prepared as described above was 59 hours.

### [Device Example 1-3] Preparation of an OLED According to the Present Disclosure

An OLED was produced in the same manner as in Device Example 1-1, except that compound B-93 was used as a material of the hole blocking layer.

The time taken for luminance to decrease from 100% to 95% at a luminance of 1,400 nits (lifespan; T95) of the organic electroluminescent device according to Device Example 1-3 prepared as described above was 60 hours.

### [Device Example 1-4] Preparation of an OLED According to the Present Disclosure

An OLED was produced in the same manner as in Device Example 1-1, except that compound B-45 was used as a material of the hole blocking layer.

The time taken for luminance to decrease from 100% to 95% at a luminance of 1,400 nits (lifespan; T95) of the organic electroluminescent device according to Device Example 1-4 prepared as described above was 52 hours.

### [Device Example 1-5] Preparation of an OLED According to the Present Disclosure

An OLED was produced in the same manner as in Device Example 1-1, except that compound H-206 was used as a host material of the light-emitting layer and compound B-73 was used as a material of the hole blocking layer.

The driving voltage and current efficiency (cd/A) at a luminance of 1,000 nits of the organic electroluminescent device according to Device Example 1-5 prepared as described above were 4.0 V and 5.7 cd/A, respectively.

### [Device Example 1-6] Preparation of an OLED According to the Present Disclosure

An OLED was produced in the same manner as in Device Example 1-1, except that compound H-206 was used as a host material of the light-emitting layer and compound B-113 was used as a material of the hole blocking layer.

The driving voltage and current efficiency (cd/A) at a luminance of 1,000 nits of the organic electroluminescent device according to Device Example 1-6 prepared as described above were 3.9 V and 5.7 cd/A, respectively.

### [Device Example 1-7] Preparation of an OLED According to the Present Disclosure

An OLED was produced in the same manner as in Device Example 1-1, except that compound H-206 was used as a host material of the light-emitting layer and compound B-93 was used as a material of the hole blocking layer.

The driving voltage and current efficiency (cd/A) at a luminance of 1,000 nits of the organic electroluminescent device according to Device Example 1-8 prepared as described above were 4.0 V and 5.5 cd/A, respectively.

### [Device Example 1-8] Preparation of an OLED According to the Present Disclosure

An OLED was produced in the same manner as in Device Example 1-1, except that compound H-206 was used as a host material of the light-emitting layer and compound B-45 was used as a material of the hole blocking layer.

The driving voltage and current efficiency (cd/A) at a luminance of 1,000 nits of the organic electroluminescent device according to Device Example 1-7 prepared as described above were 4.0 V and 5.7 cd/A, respectively.

### [Comparative Example 1-1] Preparation of an OLED Using the Conventional Compound

An OLED was produced in the same manner as in Device Example 1-1, except that compound Balq was used as a material of the hole blocking layer.

The time taken for luminance to decrease from 100% to 95% at a luminance of 1,400 nits (lifespan; T95) of the organic electroluminescent device according to Comparative Example 1-1 prepared as described above was 29 hours.

### [Comparative Example 1-2] Preparation of an OLED Using the Conventional Compound

An OLED was produced in the same manner as in Device Example 1-1, except that compound H-206 was used as a host material of the light-emitting layer and compound Balq was used as a material of the hole blocking layer.

The driving voltage and current efficiency (cd/A) at a luminance of 1,000 nits of the organic electroluminescent device according to Comparative Example 1-2 prepared as described above were 4.3 V and 5.5 cd/A, respectively.

It was confirmed that the organic electroluminescent device prepared by using a combination of host and dopant in a light-emitting layer and a hole blocking material specified in the present disclosure exhibits properties such as long lifespan, low driving voltage, and/or high current efficiency compared to the conventional organic electroluminescent device. In addition, due to the combination of the present disclosure, the hole blocking layer can provide a long-lifespan organic electroluminescent device emitting blue light by blocking the holes and controlling the property of the injected electrons, thereby maintaining the lifespan balance with the organic electroluminescent devices emitting red and green light. Although not limited as a theory, in general, when implementing a display, a high driving voltage in a blue light-emitting device among red light-, green light-, and blue light-emitting devices is an obstacle in terms of white light implementation and power consumption saving in relation to the red light and green light devices. Therefore, it can be a very important point to lower the driving voltage of the blue device even a little. A device having a lower driving voltage can be implemented through the manufacture of a device to which a combination of compounds of the present disclosure is applied.

The compounds used in Device Examples and Comparative Examples above are shown in the following Table 1.

TABLE 1

| | |
|---|---|
| Hole Injection Layer/ Hole Transport Layer | 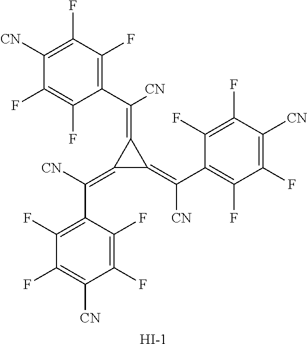 |
| | 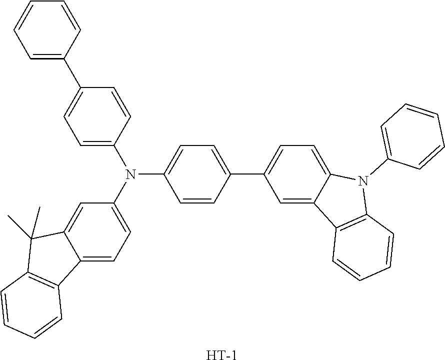 |
| | 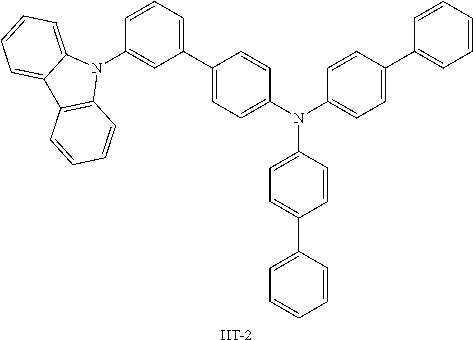 |
| Light-Emitting Layer | 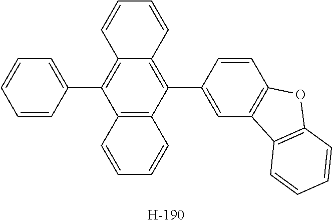 [SMILES: c1ccc(-c2c3ccccc3c(-c3ccc4oc5ccccc5c4c3)c3ccccc23)cc1] |
| | 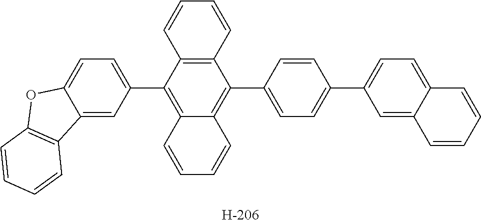 [SMILES: c1ccc2cc(-c3ccc(-c4c5ccccc5c(-c5ccc6oc7ccccc7c6c5)c5ccccc45)cc3)ccc2c1] |
| | 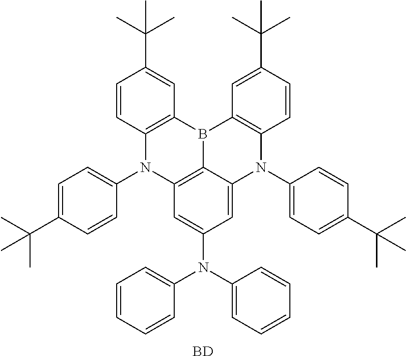 |
| Hole Blocking Layer | 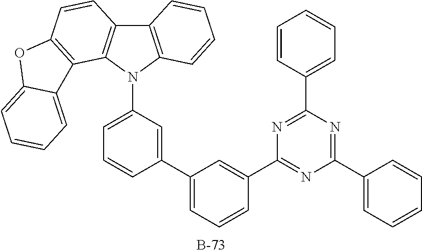 |
| | 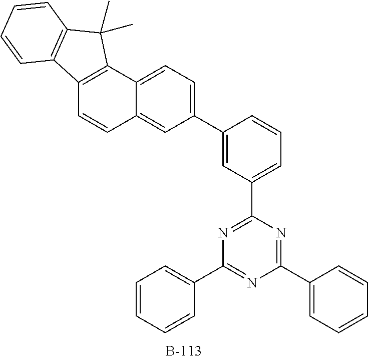 |
| | 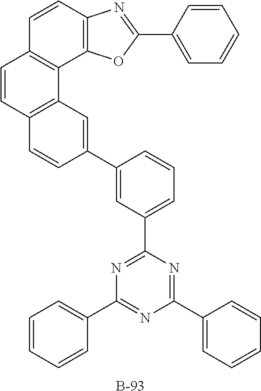 |
| | 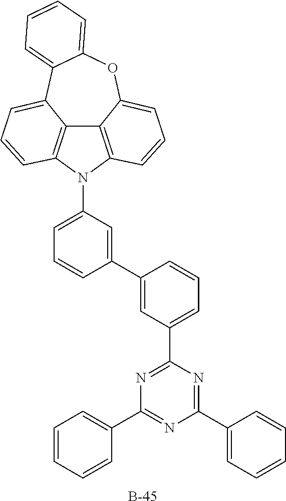 |
| | 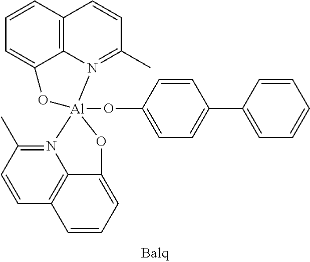 |
| Electron transport Layer/ Electron Injection Layer | 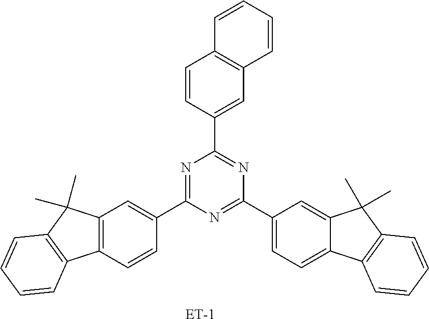 [SMILES: CC1(C)c2ccccc2-c2ccc(-c3nc(-c4ccc5c(c4)C(C)(C)c4ccccc4-5)nc(-c4ccc5ccccc5c4)n3)cc21 ; "ET-1"] |
| | 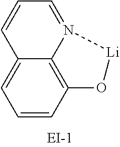 |

1. An organic electroluminescent device comprising a first electrode; a second electrode facing the first electrode; a light-emitting layer between the first electrode and the second electrode; and an electron transport zone between the light-emitting layer and the second electrode,
wherein the electron transport zone includes a hole blocking layer, an electron buffer layer, or both of them, and an electron transport layer and an electron injection layer,
wherein the electron transport layer is formed between the hole blocking layer or the electron buffer layer and the second electrode,
wherein the electron injection layer is formed between the electron transport layer and the second electrode,
wherein the light-emitting layer comprises a compound represented by the following formula 1 and a compound represented by the following formula 2, and
wherein the hole blocking layer, the electron buffer layer, or both of them comprises a compound represented by the following formula 11; 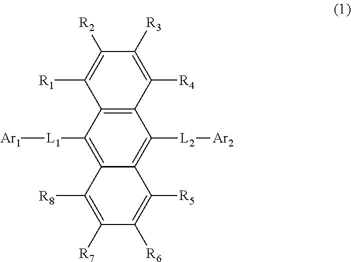
wherein
L₁ and L₂ each independently represent a single bond, a substituted or unsubstituted (C6-C30)arylene, or a substituted or unsubstituted (5- to 30-membered)heteroarylene;
Ar₁ and Ar₂ each independently represent a substituted or unsubstituted (C6-C30)aryl, or a substituted or unsubstituted (5- to 30-membered)heteroaryl; and
R₁ to R₈ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N-(Ar₄)(Ar₅); 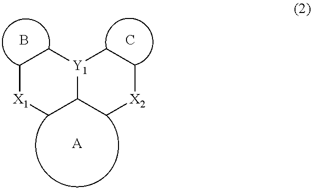
wherein
Ring A, Ring B and Ring C each independently represent a substituted or unsubstituted (C6-C30)aryl, or a substituted or unsubstituted (3- to 50-membered)heteroaryl;
Y₁ represents B;
X₁ and X₂ each independently represent NR; and
R represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N-(Ar₄)(Ar₅); or may be linked to at least one of Ring A, Ring B and Ring C to form a ring(s); 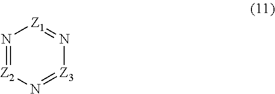
wherein
Z₁ to Z₃ each independently represent CR₁a;
R₁₈ each independently represents hydrogen, deuterium, halogen, cyano, a substituted or unsubstituted (C1-C30)alkyl, a substituted or unsubstituted (C6-C50)aryl, a substituted or unsubstituted (3- to 50-membered)heteroaryl, a substituted or unsubstituted (C3-C30)cycloalkyl, a substituted or unsubstituted (3- to 7-membered)heterocycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N-(Ar)(Ar₅), provided that R₁₈ does not include 2-dimethylfluorenyl, quinolinyl, and pyridyl;
L₄ each independently represents a single bond, a substituted or unsubstituted (C6-C30)arylene, or a substituted or unsubstituted (3- to 30-membered)heteroarylene;
Ar₄ and Ar₅ each independently represent hydrogen, a substituted or unsubstituted (C1-C30)alkyl, a substituted or unsubstituted (C2-C30)alkenyl, a substituted or unsubstituted fused ring of an (C3-C30) aliphatic ring and an (C6-C30) aromatic ring, a substituted or unsubstituted (C6-C30)aryl, or a substituted or unsubstituted (3- to 30-membered)heteroaryl; and
when a plurality of substituents represented by the same symbol are present, each of the substituents represented by the same symbol may be the same or different.

2. The organic electroluminescent device according to claim 1, wherein the substituent of the substituted alkyl, the substituted alkenyl, the substituted aryl, substituted arylene, the substituted heteroaryl, the substituted heteroarylene, the substituted cycloalkyl, the substituted alkoxy, the substituted heterocycloalkyl, the substituted trialkylsilyl, the substituted dialkylarylsilyl, the substituted alkyldiarylsilyl, the substituted triarylsilyl and the substituted fused ring of aliphatic ring and aromatic ring each independently represents at least one selected from the group consisting of deuterium; halogen; cyano; carboxyl; nitro; hydroxy; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (5- to 50-membered)heteroaryl unsubstituted or substituted with at least one of (C1-C30)alkyl, (C6-C30)aryl, and di(C6-C30)arylamino; (C6-C30)aryl unsubstituted or substituted with at least one of (C1-C30)alkyl, (3- to 50-membered)heteroaryl, and mono- or di-(C6-C30)arylamino; tri(C1-C30)alkylsilyl; tri(C6-C30)arylsilyl; di(C1-C30)alkyl(C6-C30)arylsilyl; (C1-C30)alkyldi(C6-C30)arylsilyl; amino; mono- or di-(C1-C30)alkylamino; mono- or di-(C2-C30)alkenylamino; mono- or di-(C6-C30)arylamino unsubstituted or substituted with at least one of (C1-C30)alkyl, (5- to 30-membered)heteroaryl, and di(C6-C30)arylamino; mono- or di-(3- to 30-membered)heteroarylamino; (C1-C30)alkyl(C2-C30)alkenylamino; (C1-C30)alkyl(C6-C30)arylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl.

3. The organic electroluminescent device according to claim 1, wherein L₁ and L₂ in the formula 1 each independently represent a single bond, or are selected from any one of the substituents listed in the following Group 1: 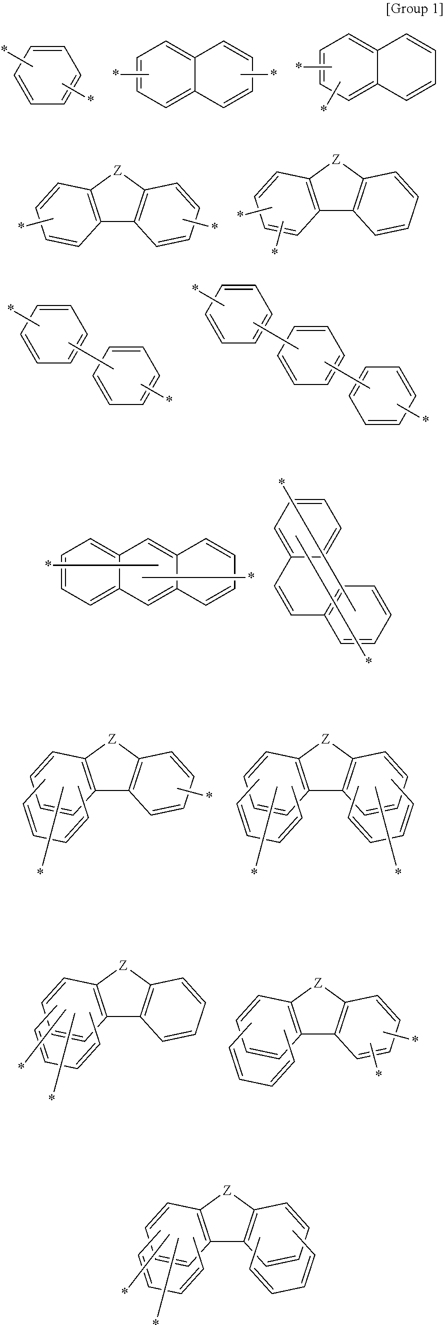
wherein
Z represents O, S, NR₁₀₁, CR₁₀₂R₁₀₃ or SiR₁₀₄R₁₀₅;
R₁₀₁ to R₁₀₅ each independently represent a substituted or unsubstituted (C1-C30)alkyl, a substituted or unsubstituted (C6-C30)aryl, or a substituted or unsubstituted (3- to 30-membered)heteroaryl; or may be linked to the adjacent substituent to form a ring(s); and
each of the two * in any one of the Group 1 represents a bonding site to the anthracene skeleton, and a bonding site to Ar₁ or Ar₂.

4. The organic electroluminescent device according to claim 1, wherein Ar₁ and Ar₂ in the formula 1 are each independently selected from any one of the substituents listed in the following Group 2: 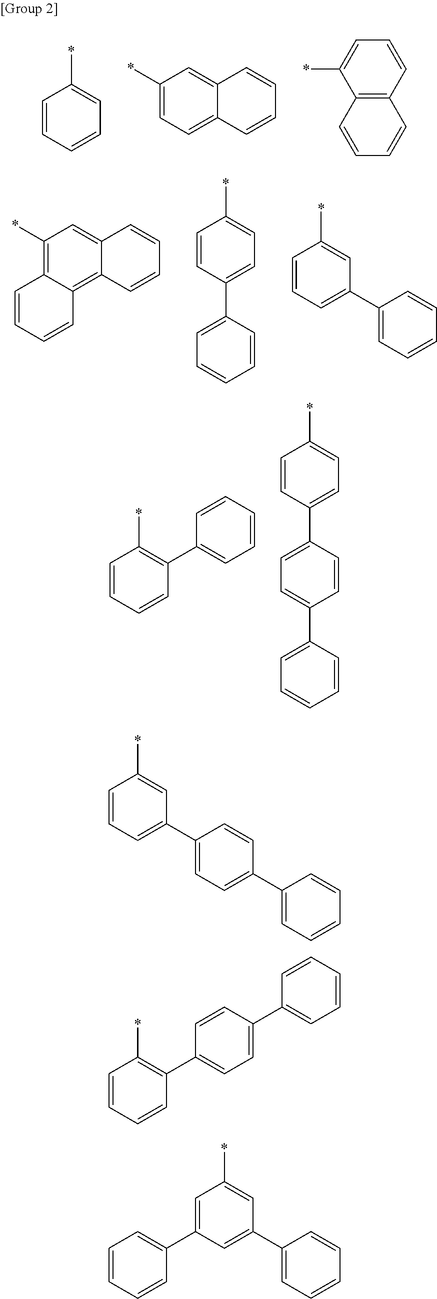 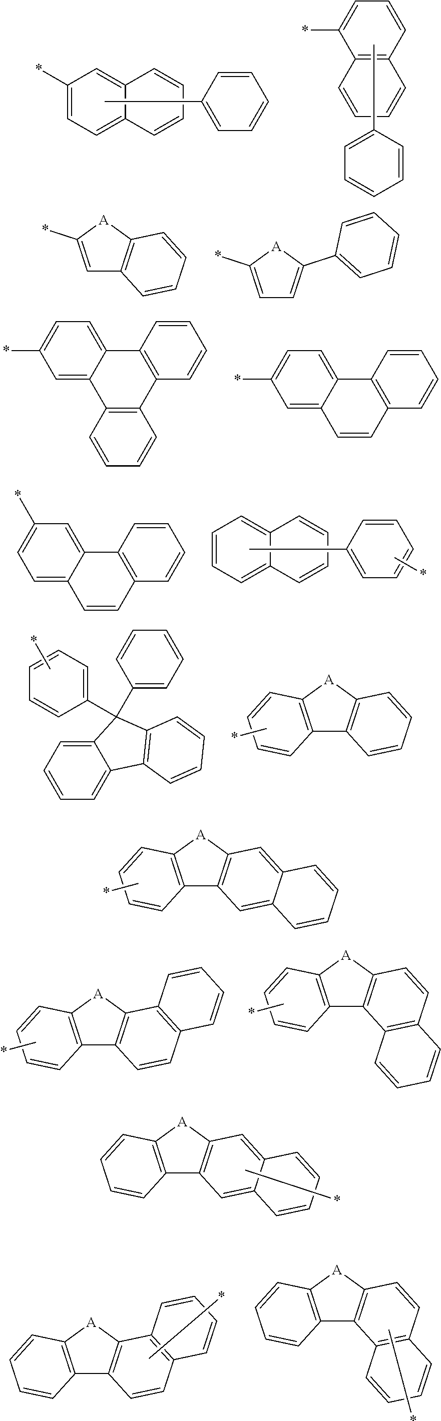 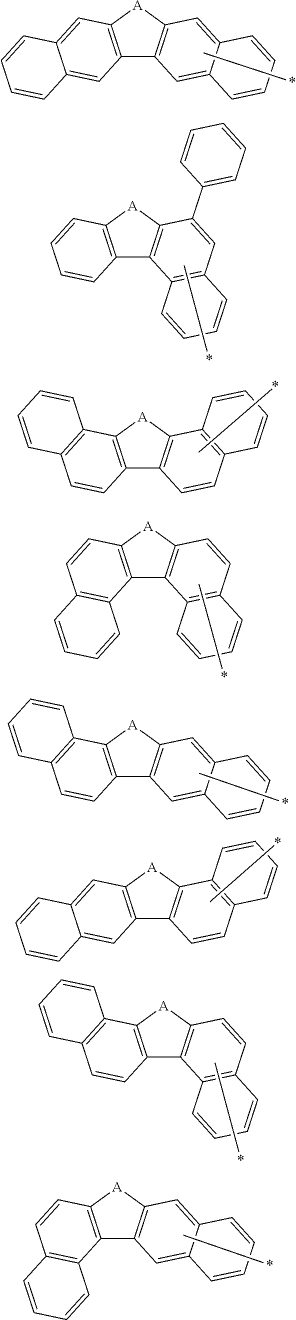 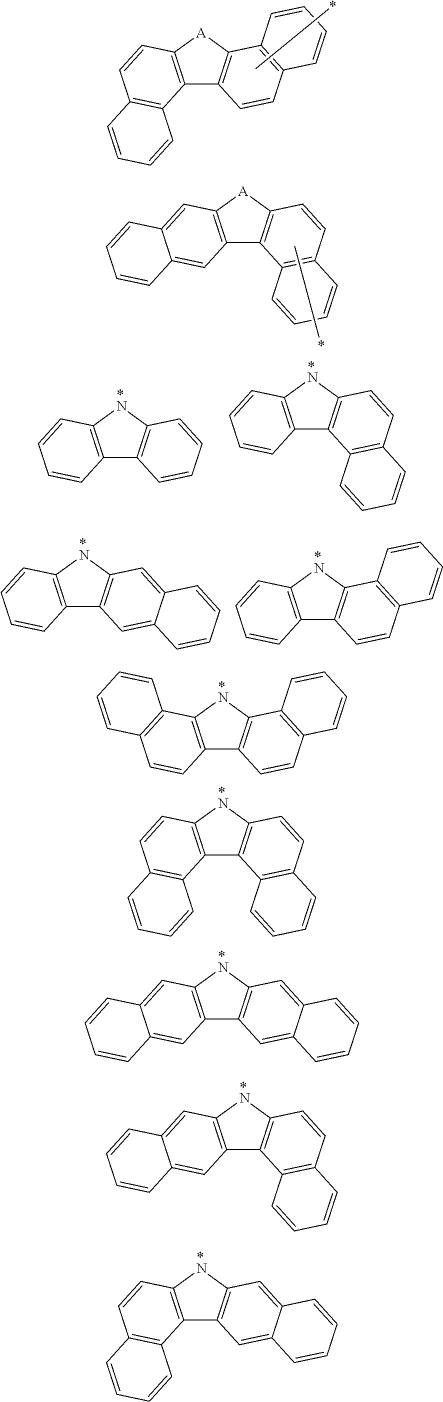 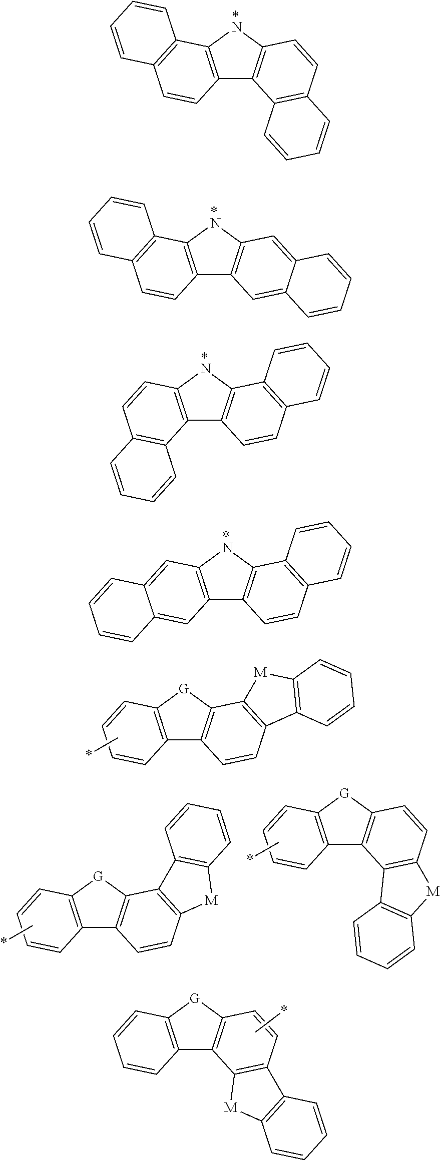 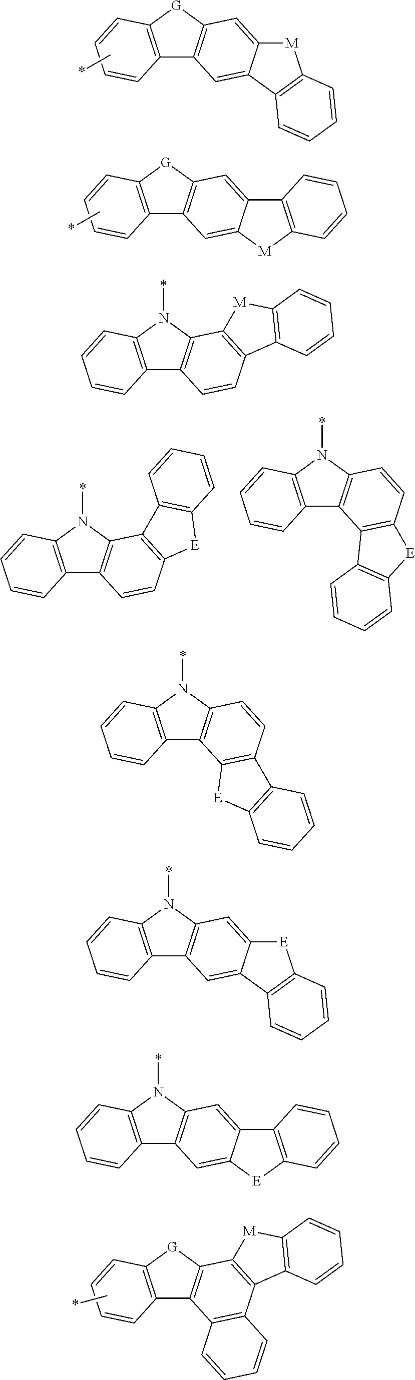 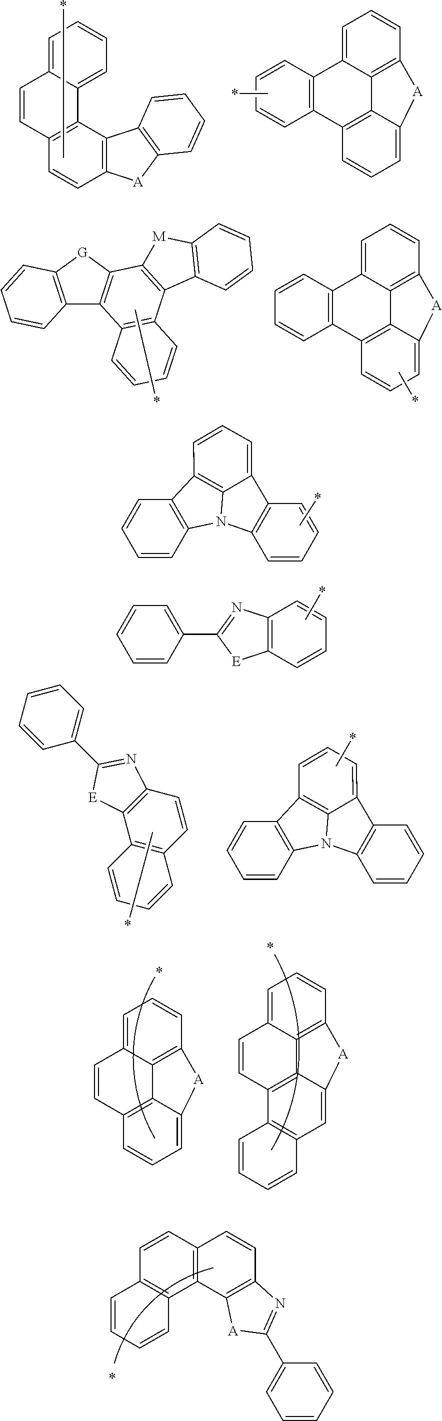
wherein
A, G, E and M each independently represent O, S, NR₁₀₆, CR₁₀₇R₁₀₈ or SiR₁₀₉R₁₁₀;
R₁₀₆ to R₁₁₀ each independently represent a substituted or unsubstituted (C1-C30)alkyl, a substituted or unsubstituted (C6-C30)aryl, or a substituted or unsubstituted (3- to 30-membered)heteroaryl; or may be linked to the adjacent substituent to form a ring(s); and
* represents a bonding site to the anthracene skeleton, L₁, or L₂.

5. The organic electroluminescent device according to claim 1, wherein the compound represented by formula 1 is represented by the following formula 1-1: 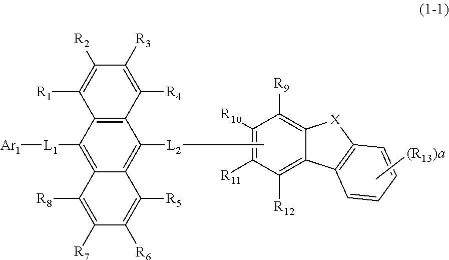
wherein
X represents O or S;
R₉ to R₁₂ each independently represent hydrogen or deuterium, or a bonding site to L₂;
R₁₃ each independently represents hydrogen, deuterium, a substituted or unsubstituted (C6-C50)aryl, or a substituted or unsubstituted (3- to 50-membered)heteroaryl;
a represents an integer of 1 to 4, and when a is an integer of 2 or more, each of R₁₃ may be the same or different; and
L₁, L₂, Ar₁ and R₁ to R₈ are as defined in claim 1.

6. The organic electroluminescent device according to claim 1, wherein the compound represented by formula 2 is represented by the following formula 2-1: 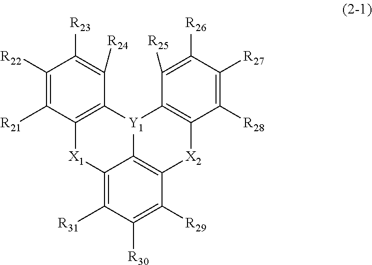
wherein
R₂₁ to R₃₁ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₄-N-(Ar₄)(Ar₅); or may be linked to the adjacent substituent to form a ring(s); and
Y₁, X₁, X₂, L₄, Ar₄, and Ar₅ are as defined in claim 1.

7. The organic electroluminescent device according to claim 1, wherein the compound represented by formula 11 is represented by the following formula 11-1: 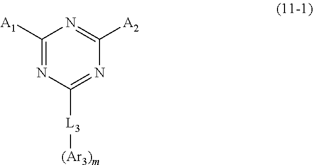
wherein
A₁ and A₂ each independently are as defined as R₁ in formula 11;
L₃ represents a single bond, a substituted or unsubstituted (C6-C50)arylene, or a substituted or unsubstituted (5- to 50-membered)heteroarylene;
Ar₃ represents a substituted or unsubstituted (C6-C50)aryl, or a substituted or unsubstituted (5- to 50-membered)heteroaryl, provided that Ar₃ is not 2-dimethylfluorenyl, quinolinyl, and pyridyl; and
m represents an integer of 1 or 2, and when m is 2, each of Ar₃ may be the same or different.

8. The organic electroluminescent device according to claim 1, wherein the compound represented by formula 1 is selected from the following compounds: 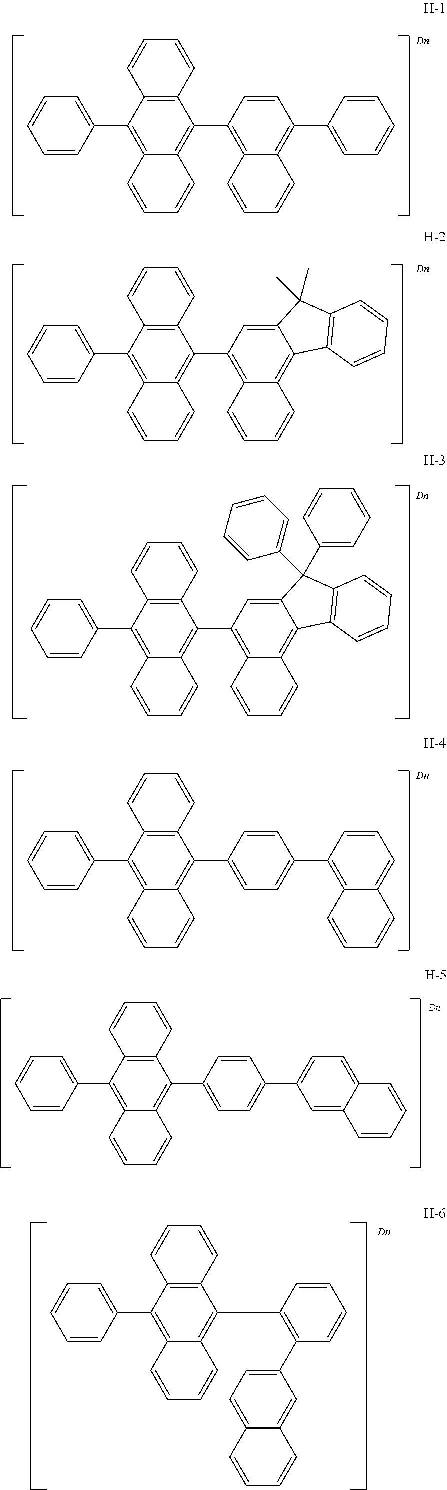 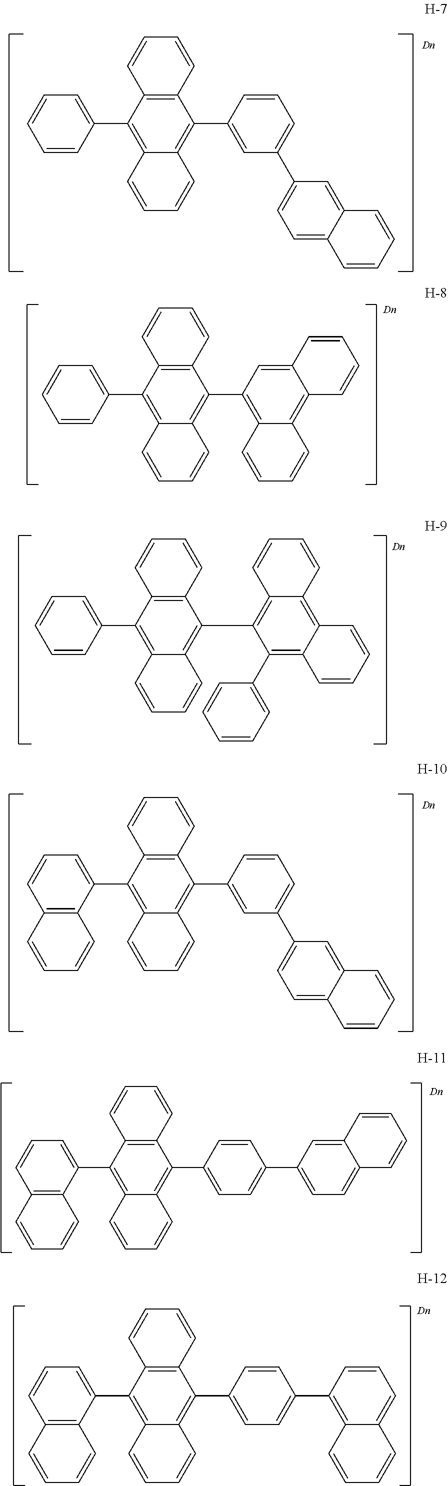 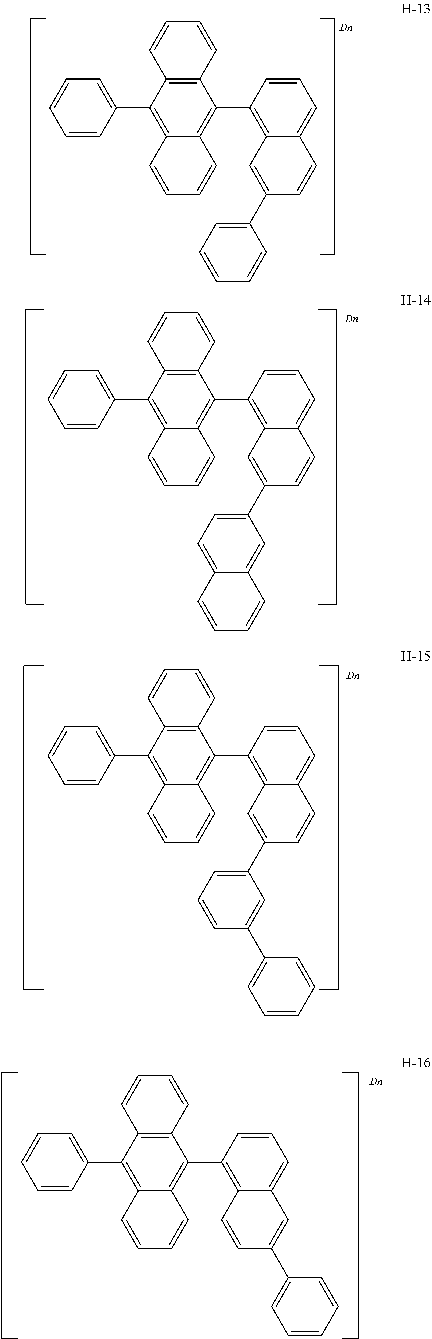 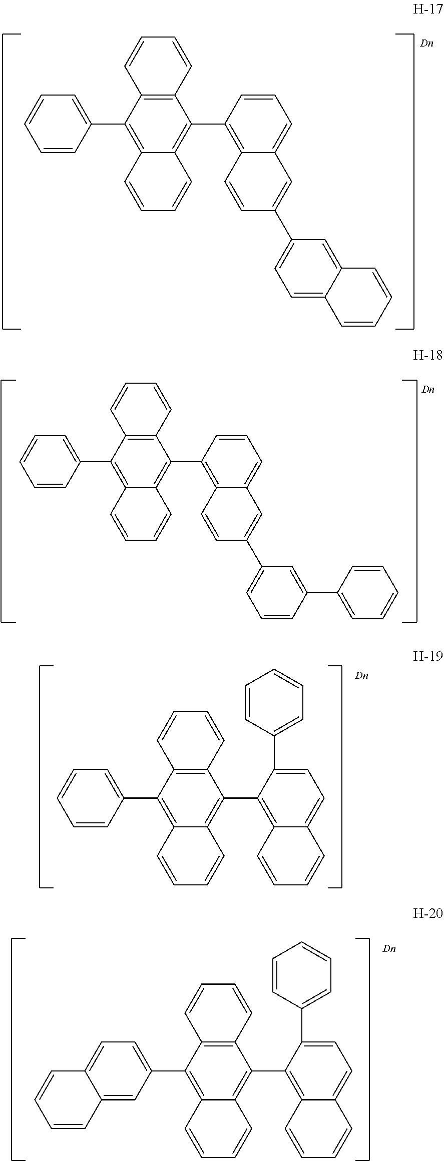 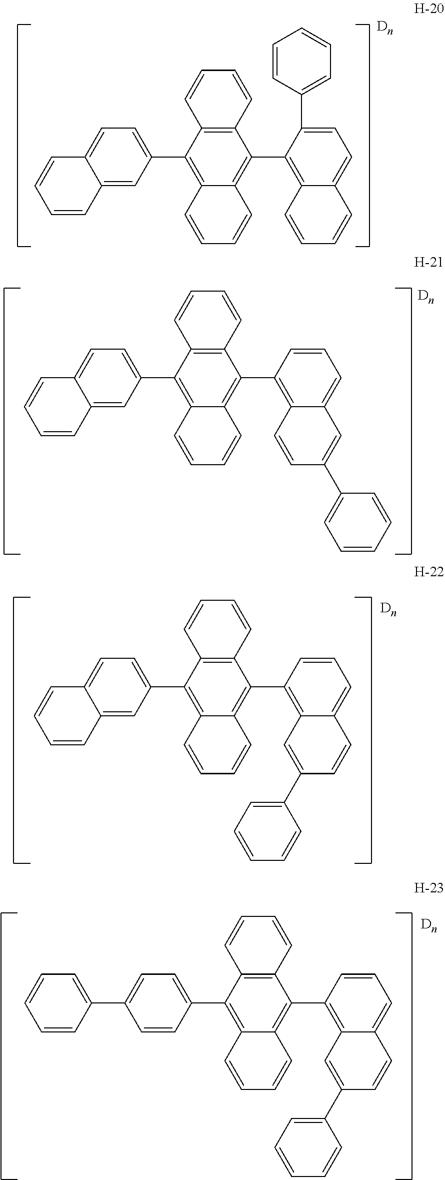 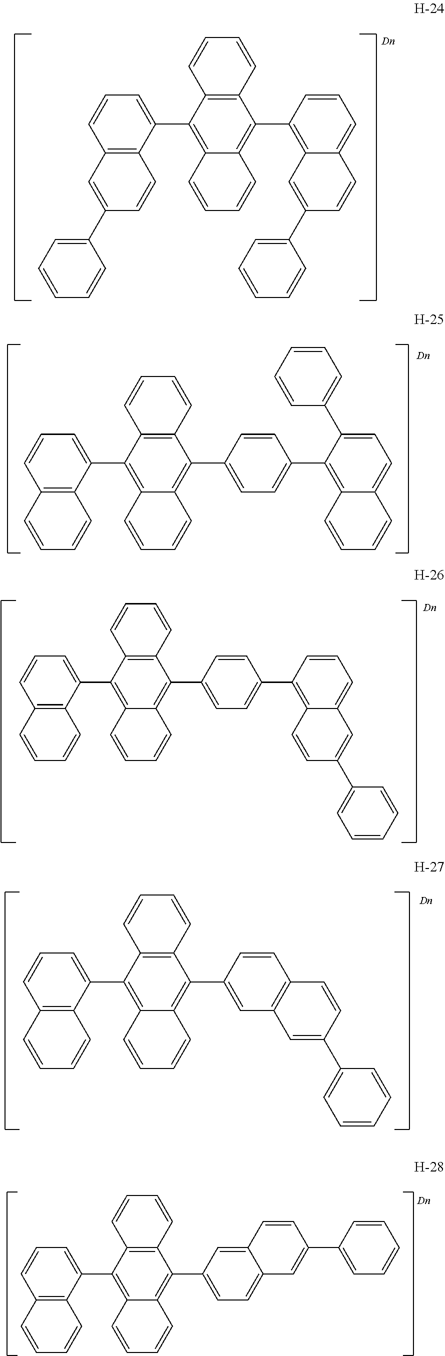 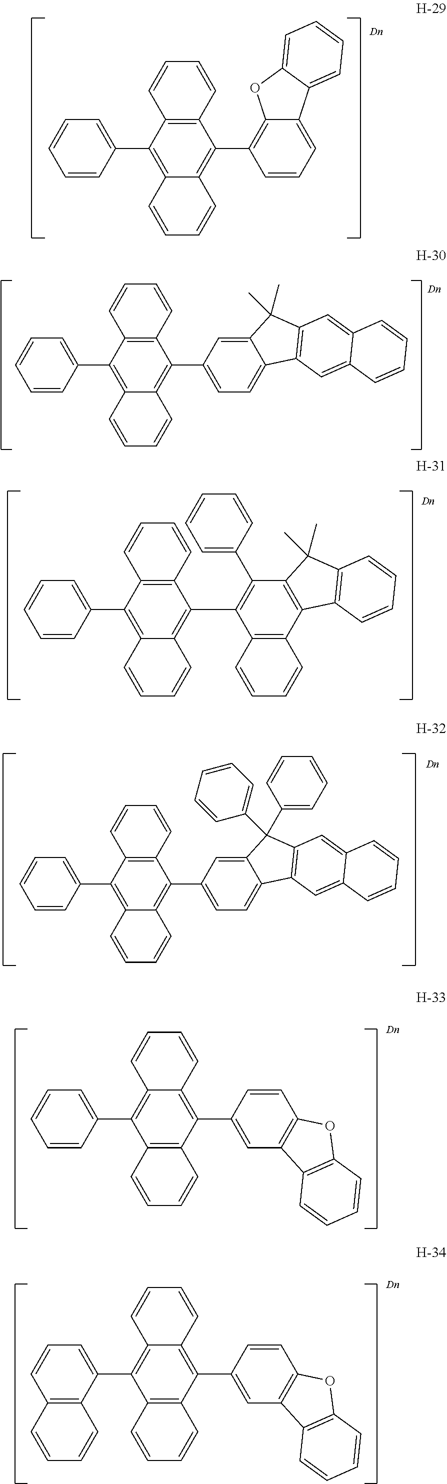 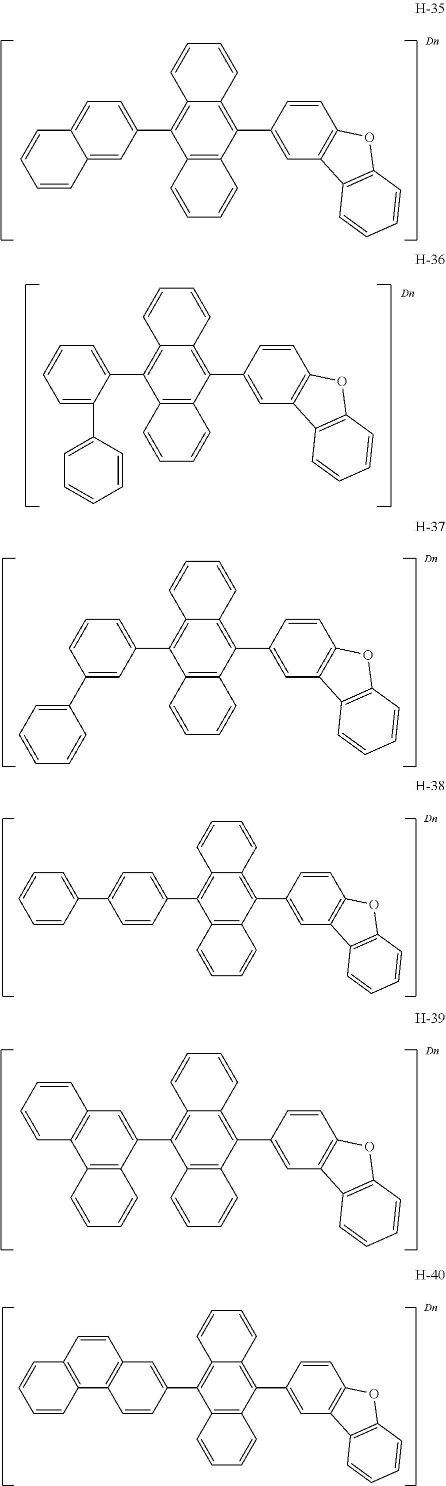 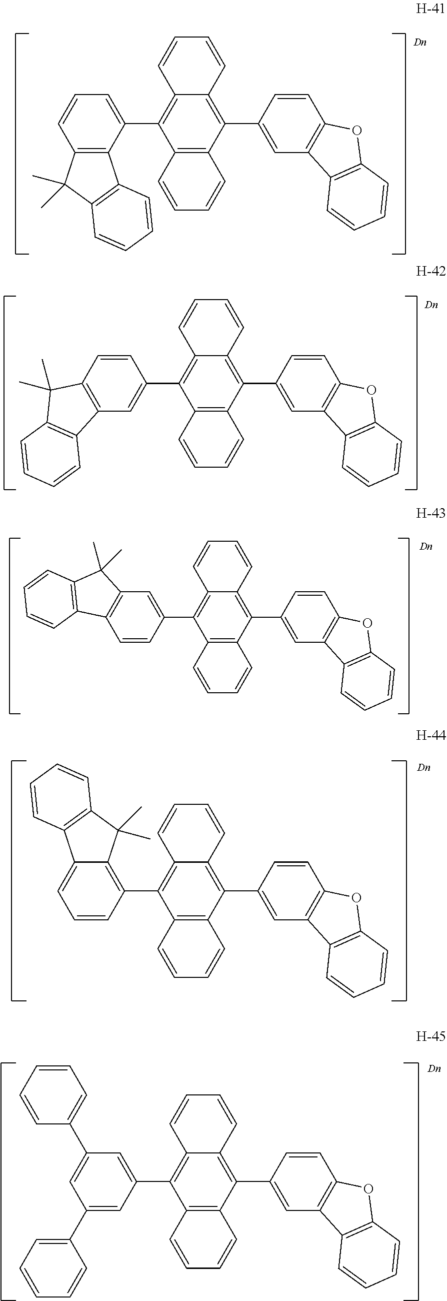 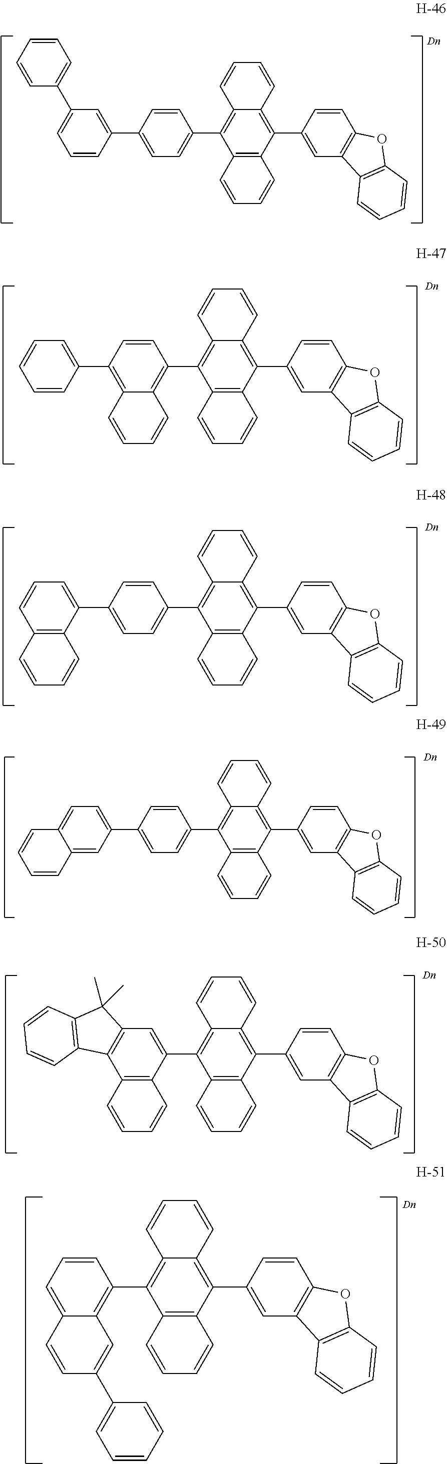 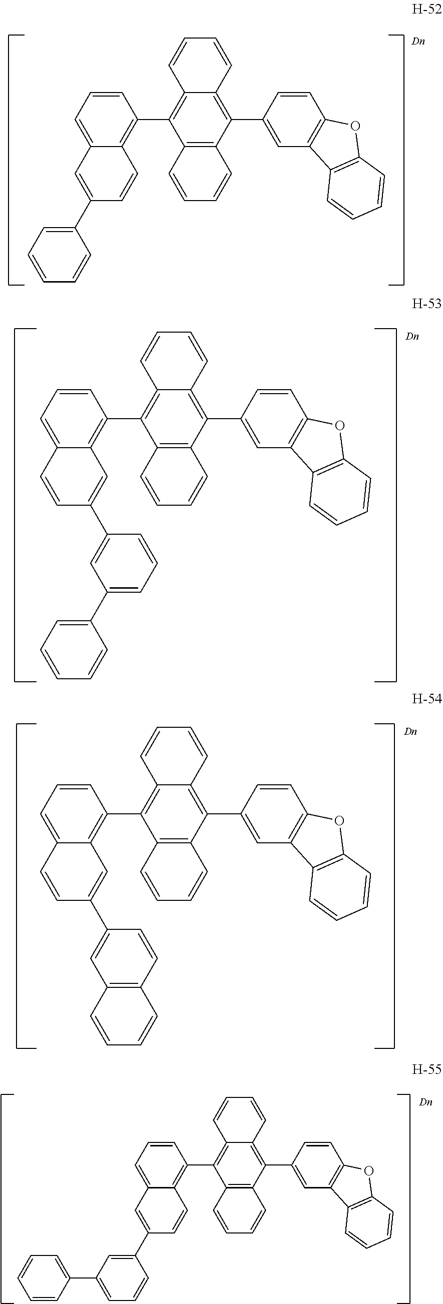 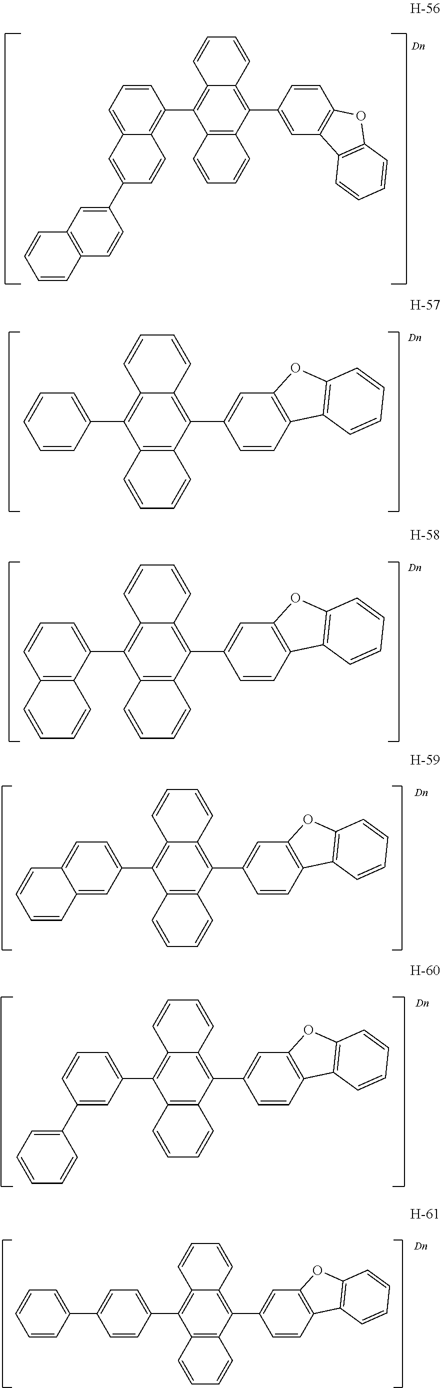 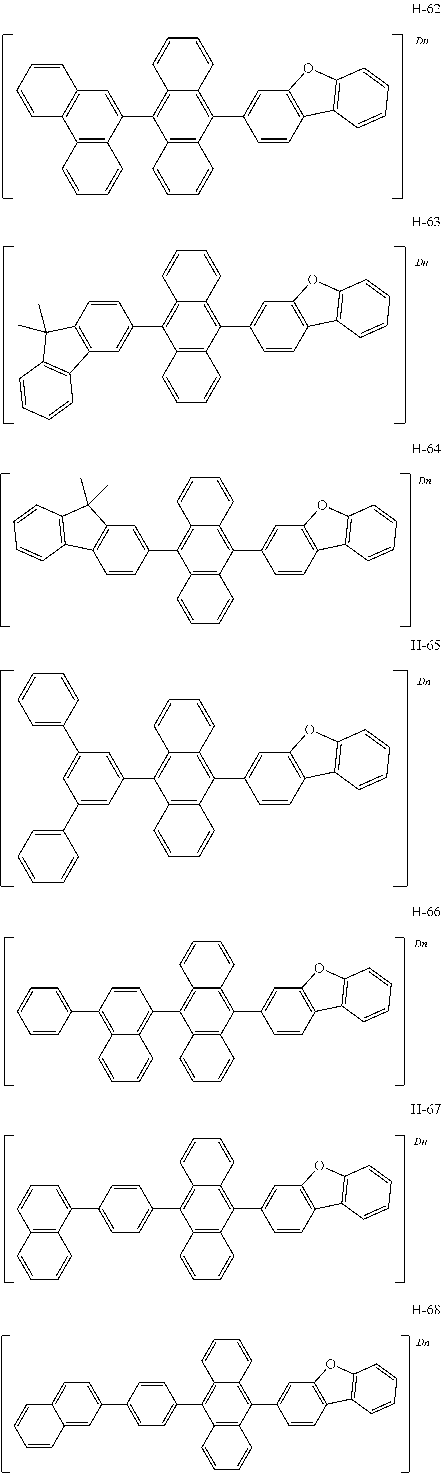 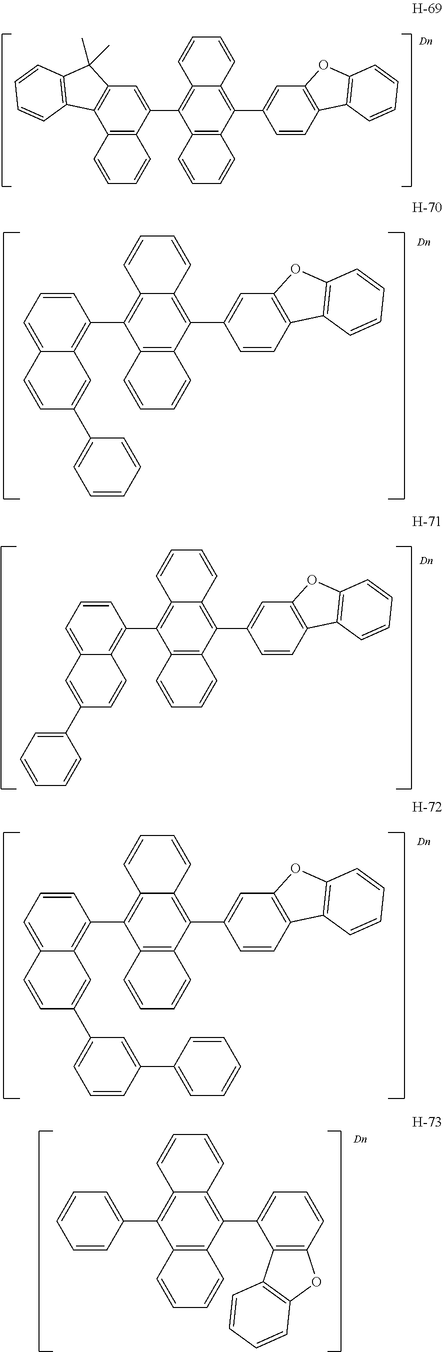 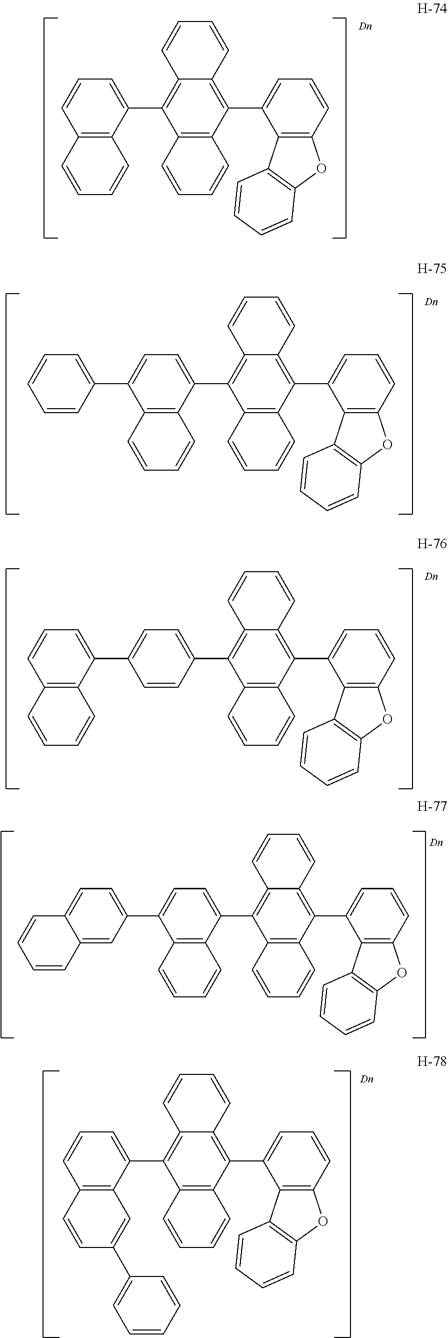 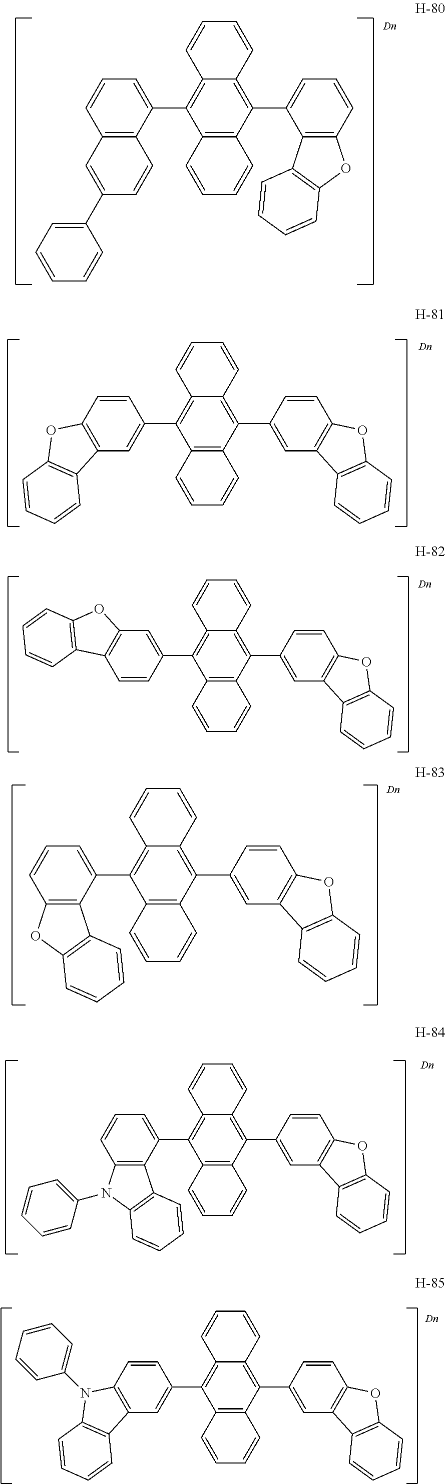 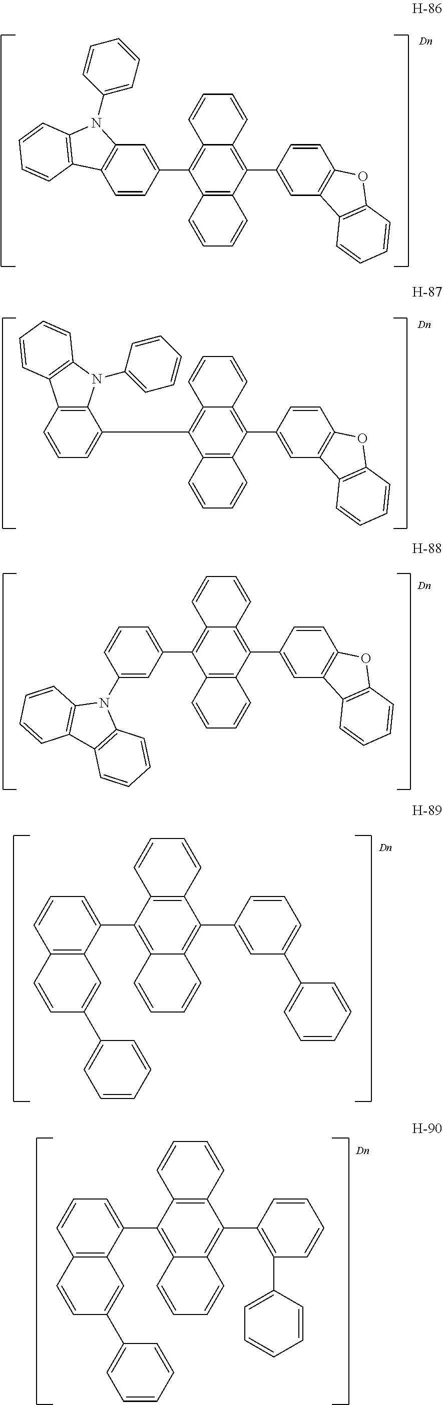 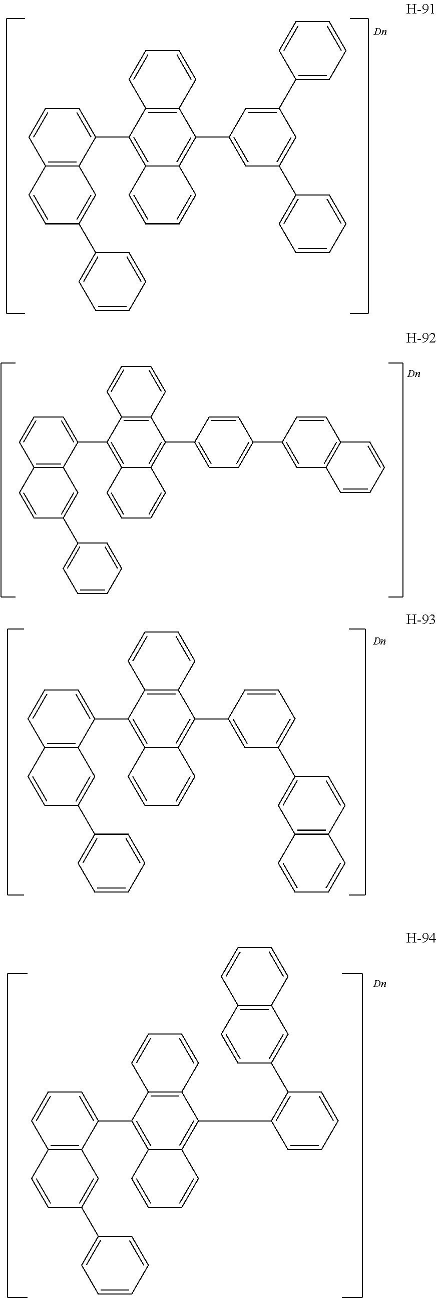 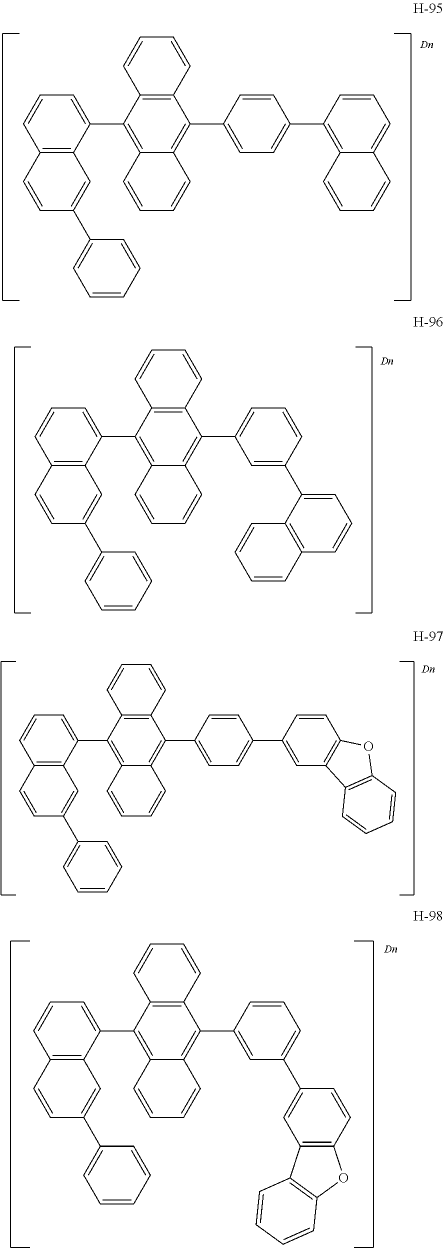 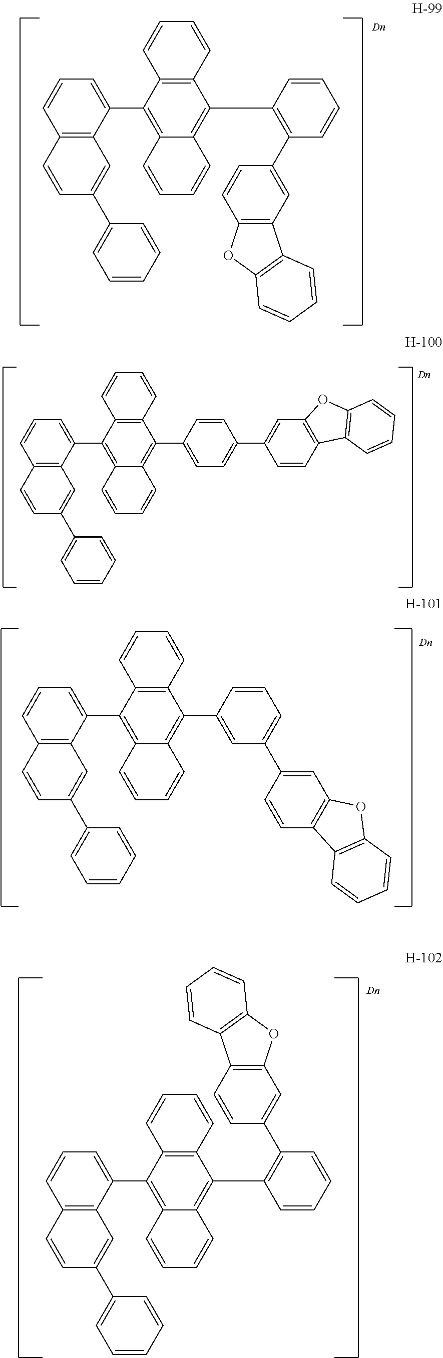 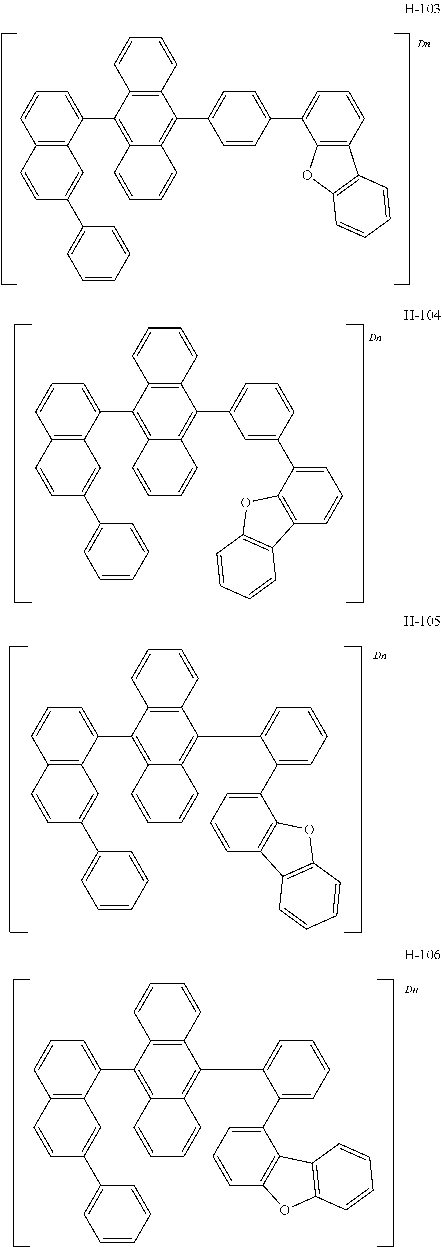 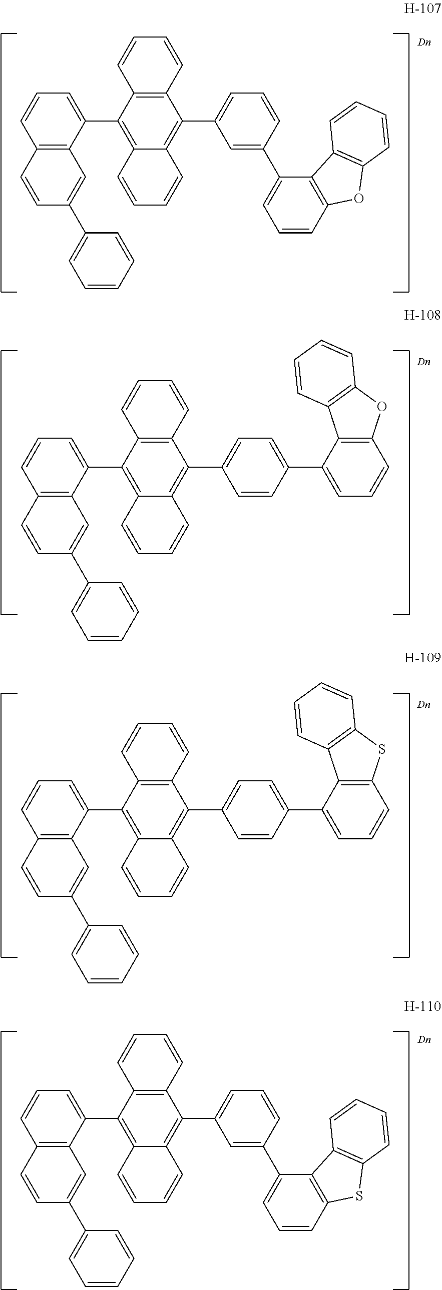 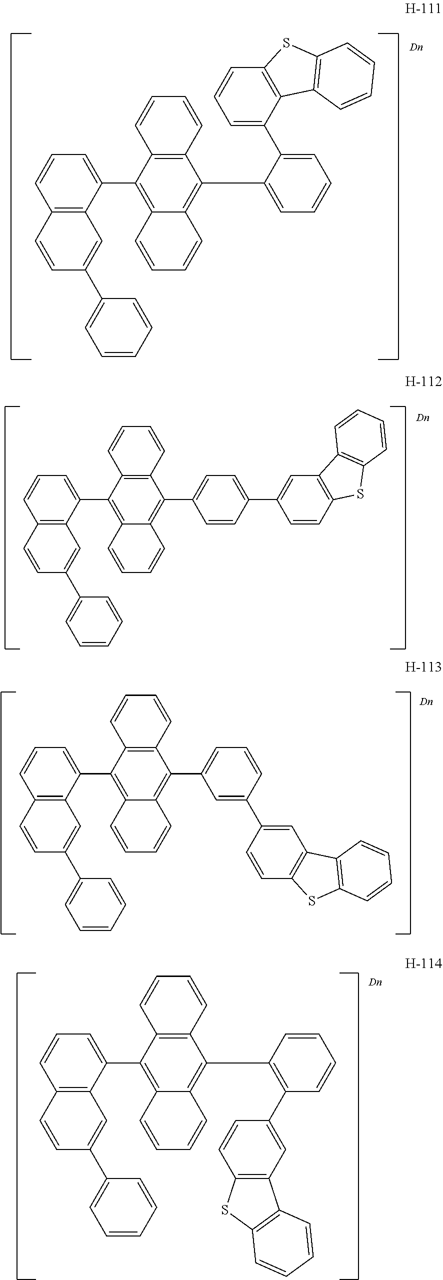 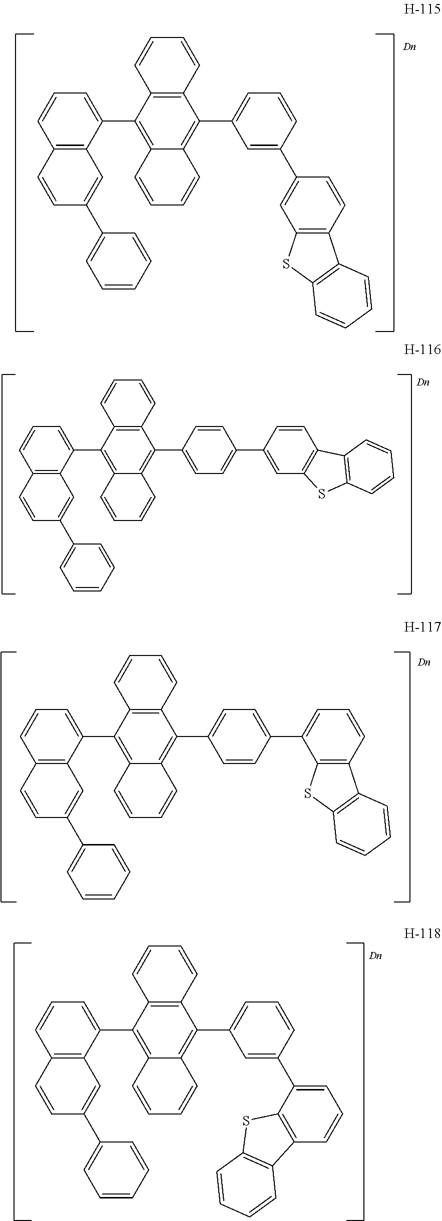 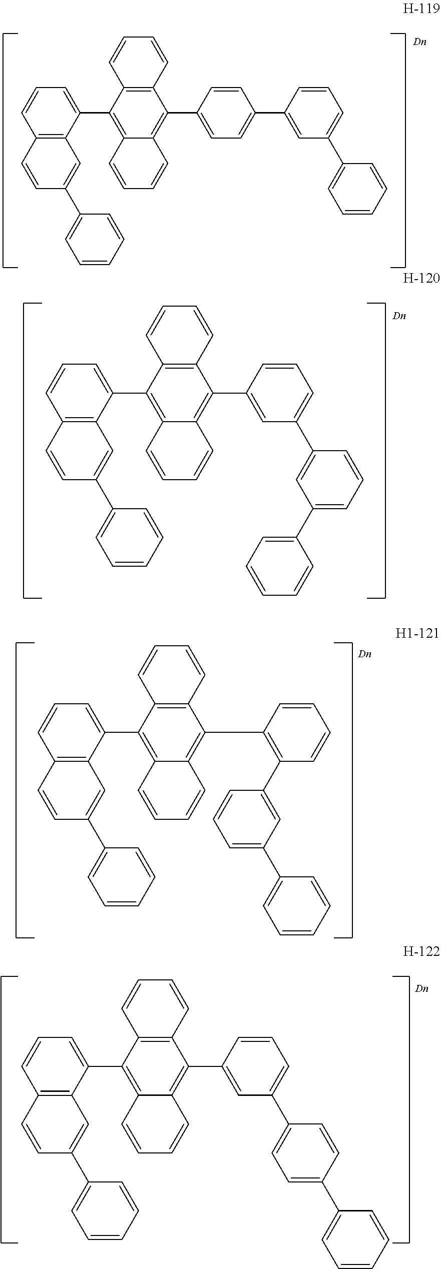 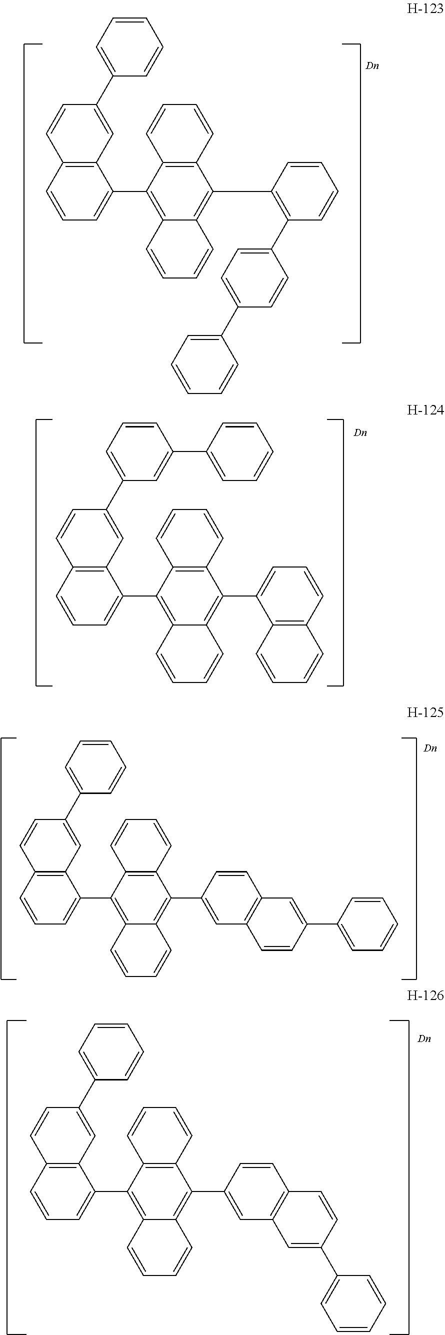 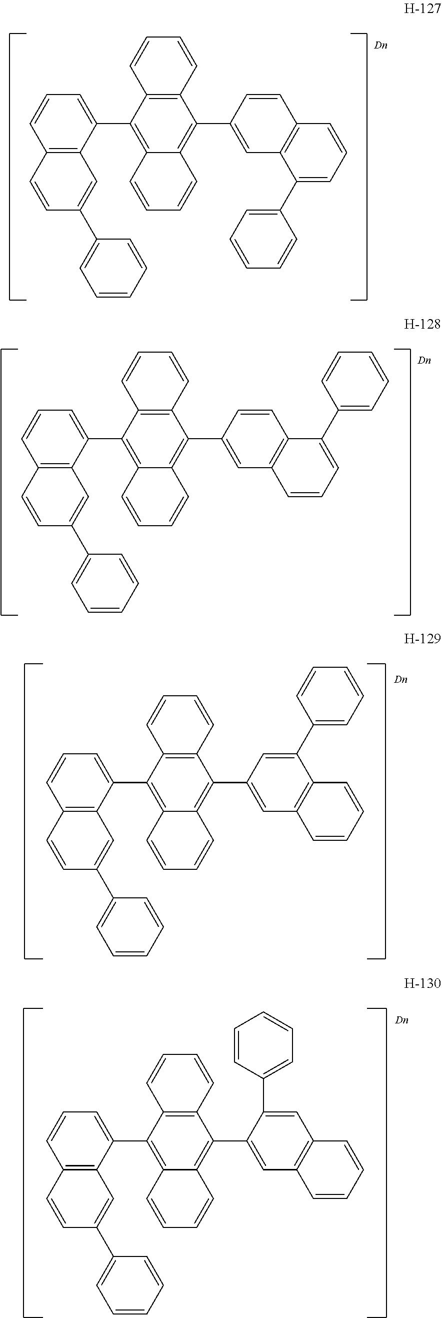 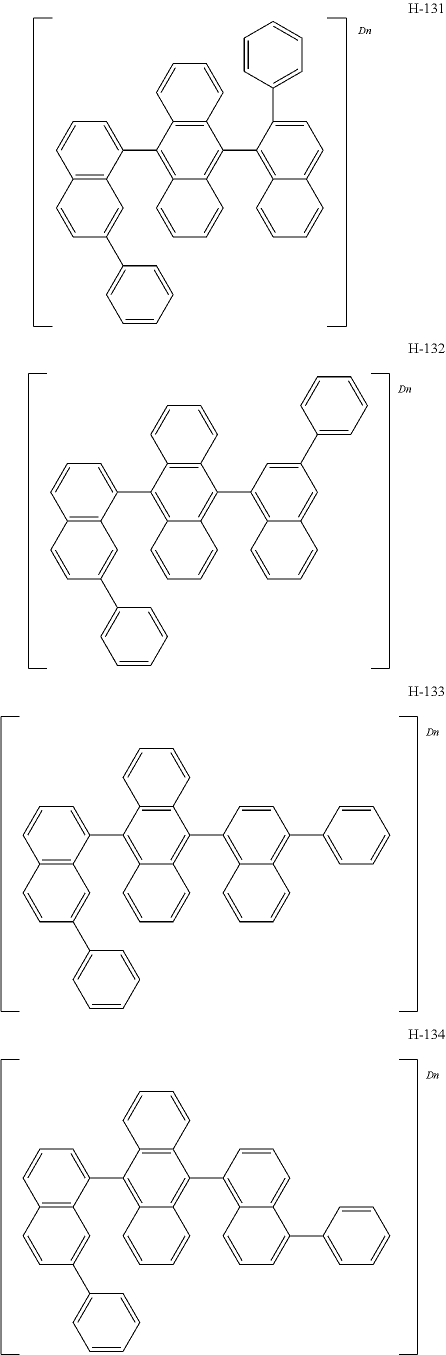 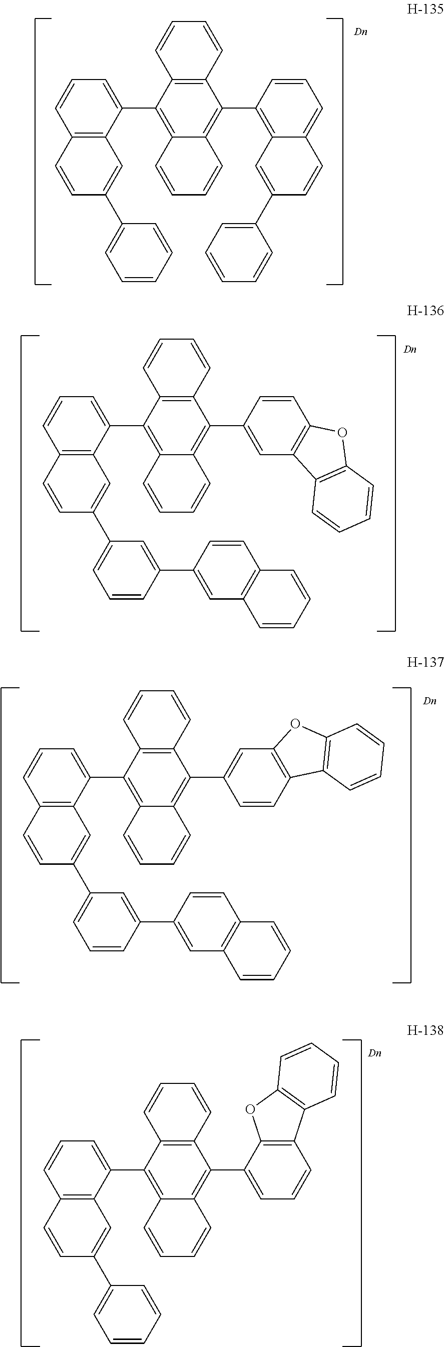 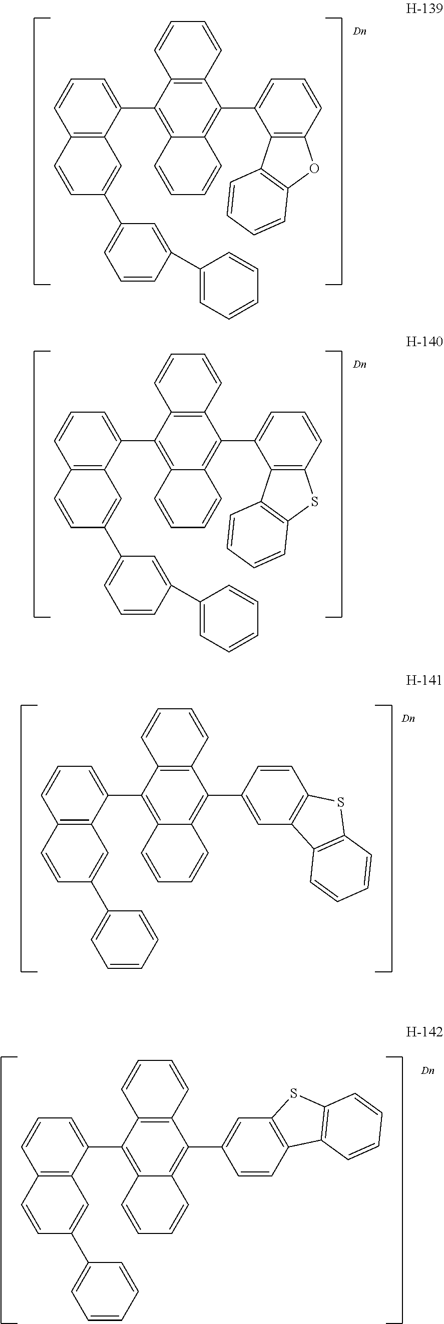 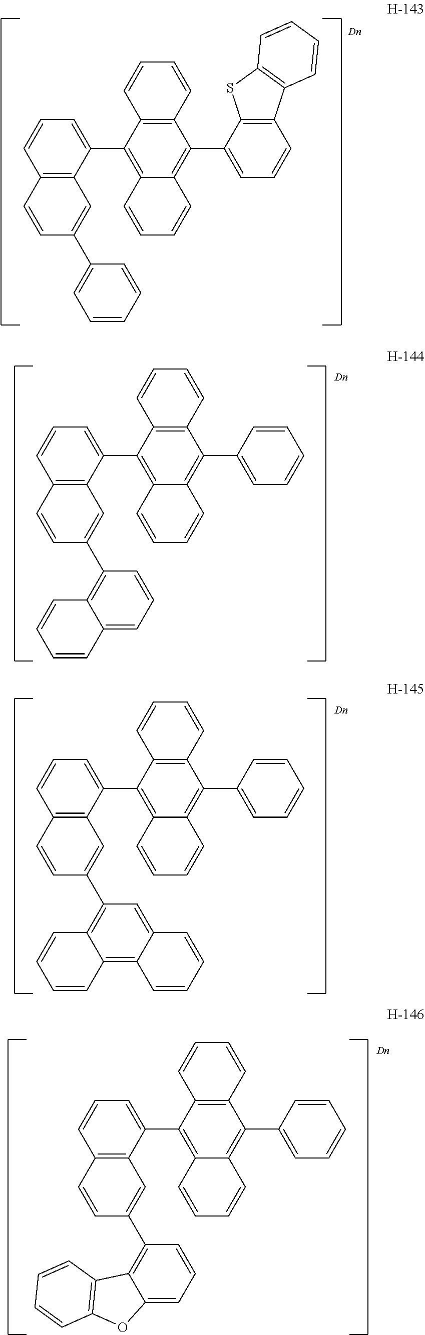 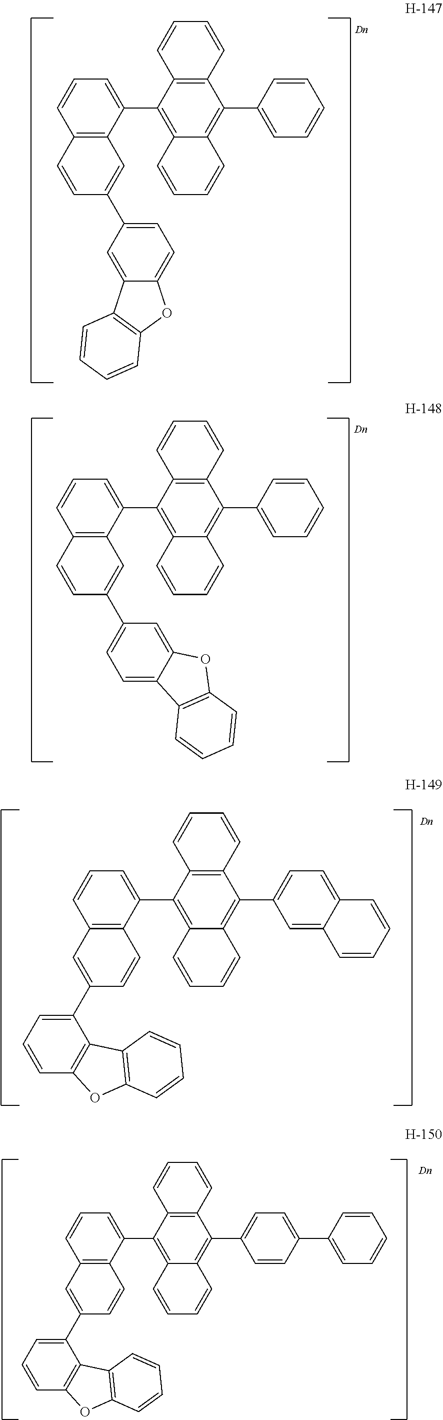 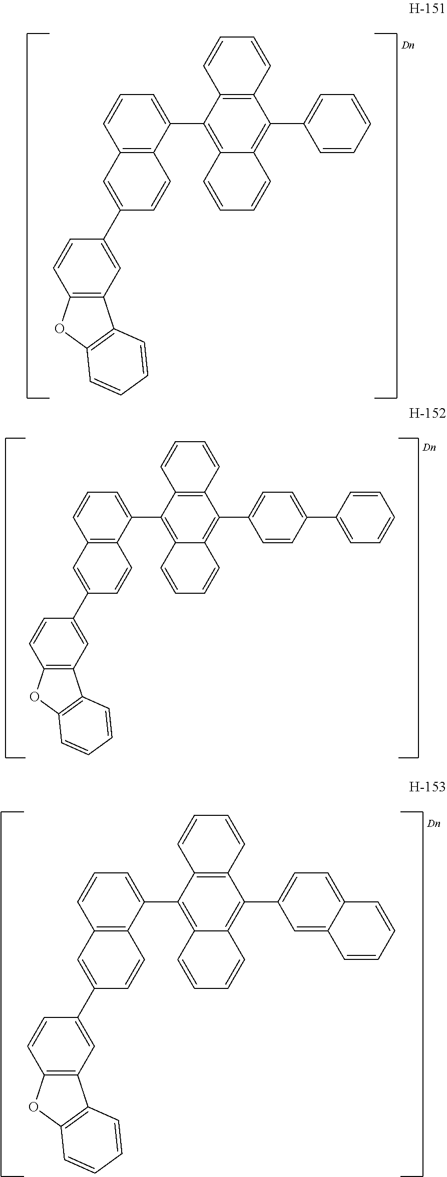 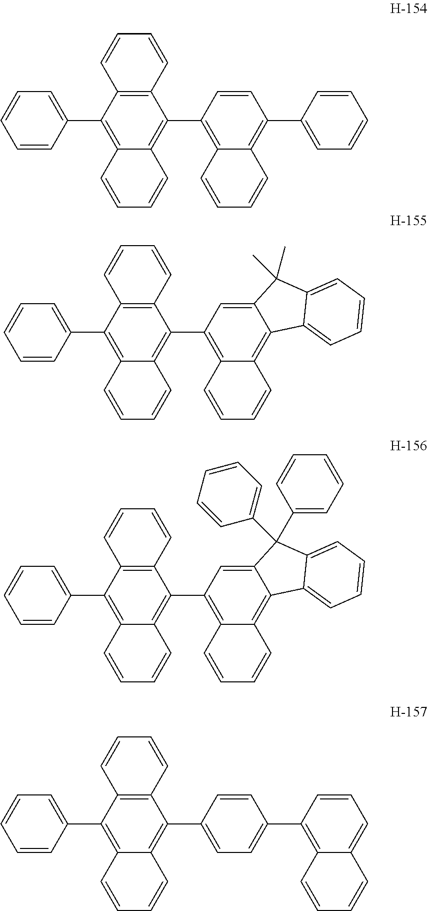 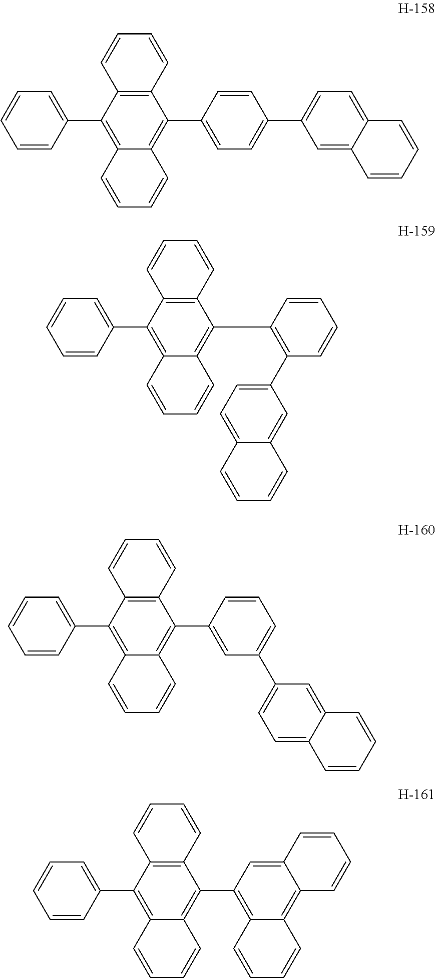 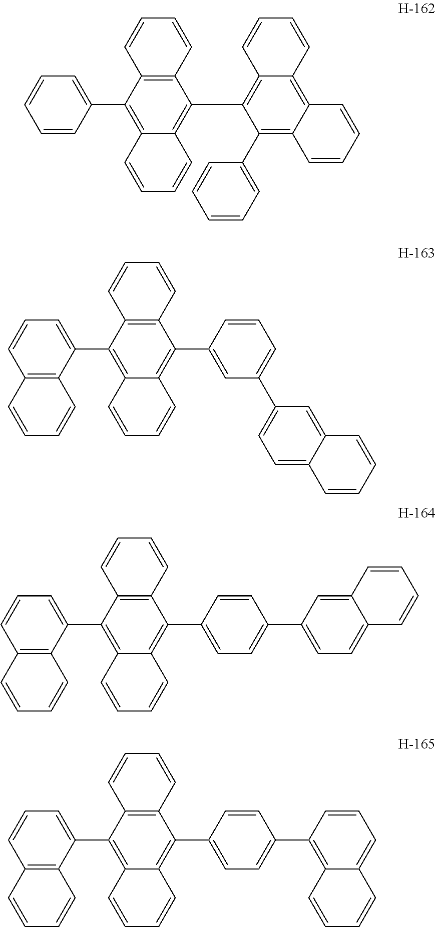 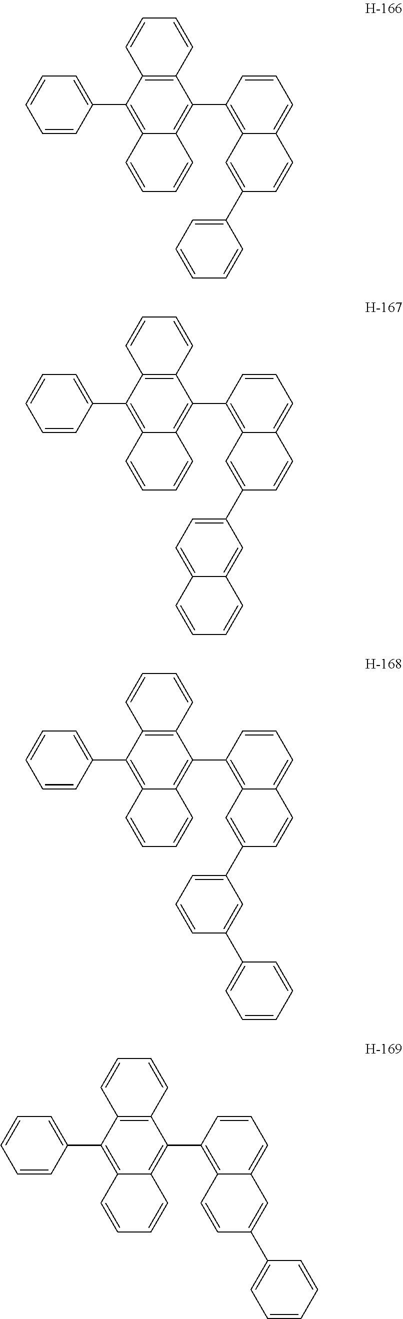 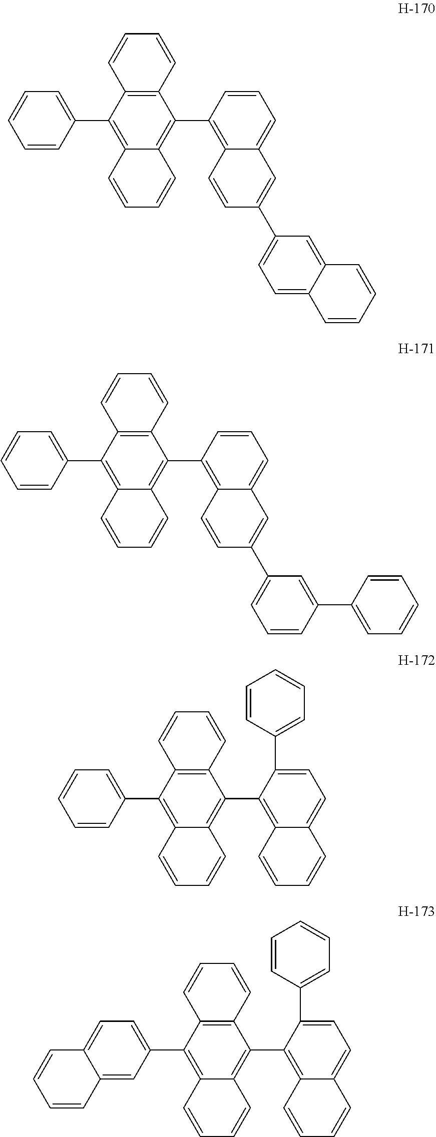 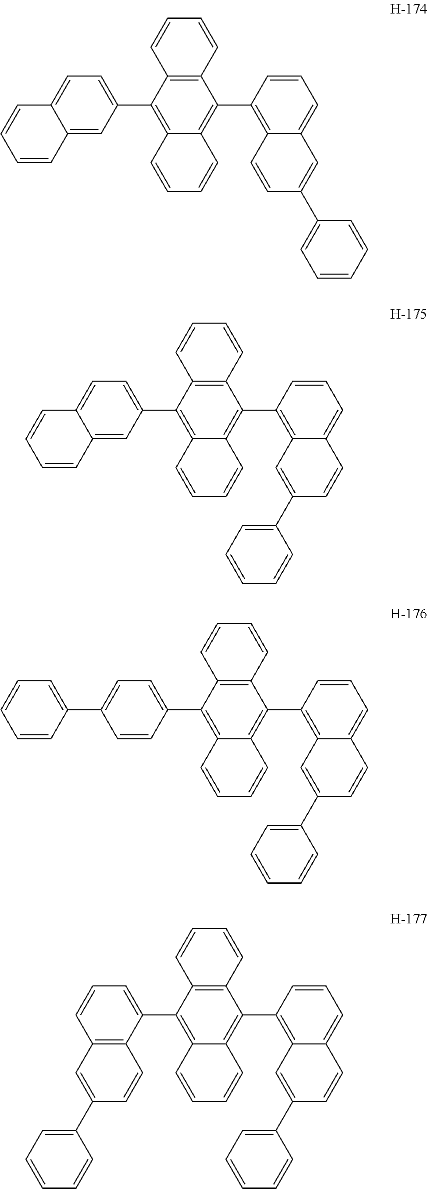 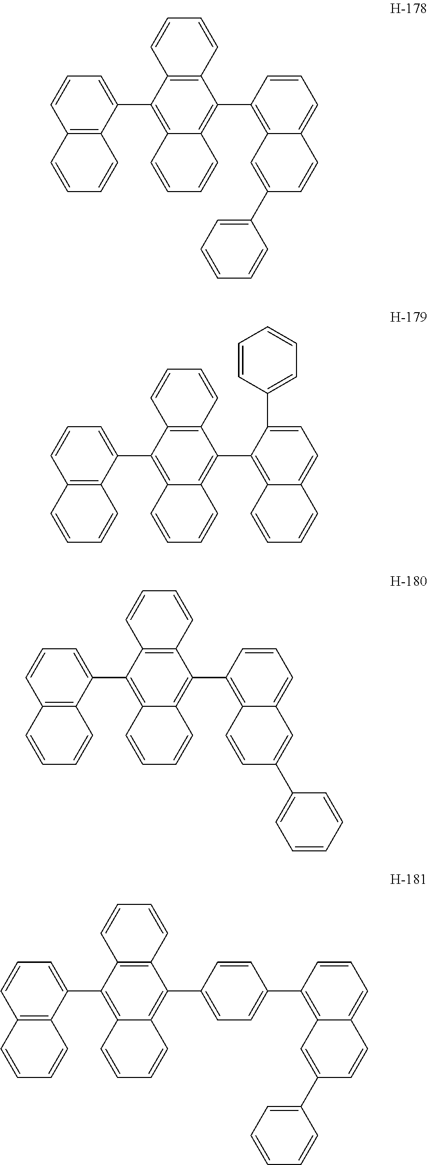 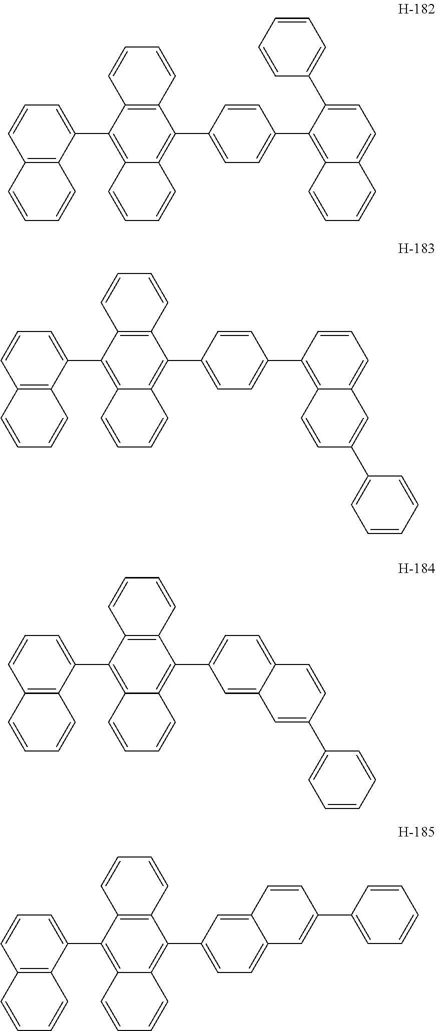 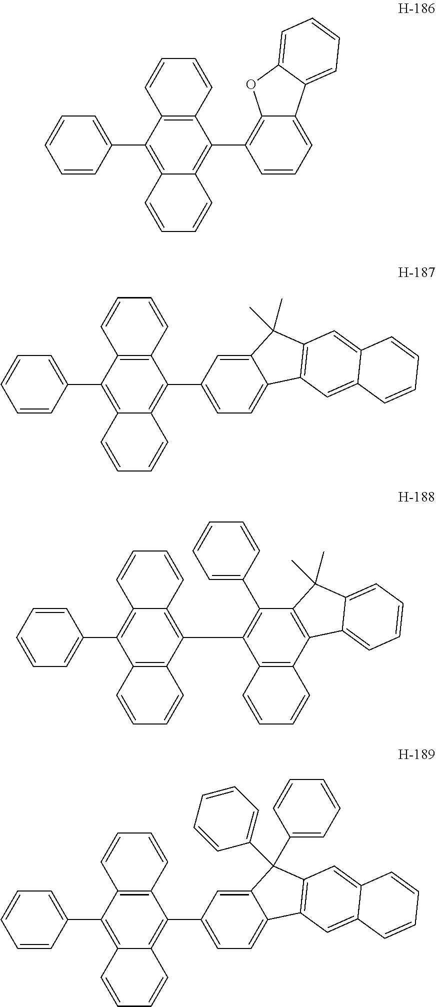 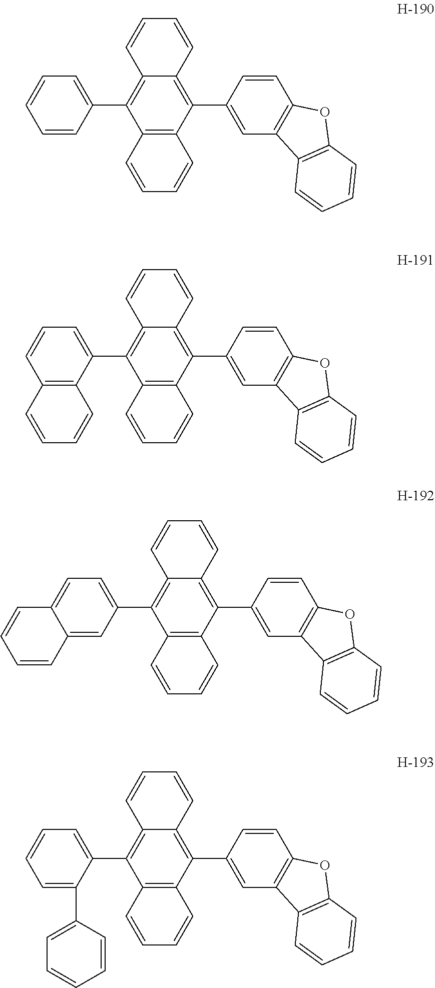 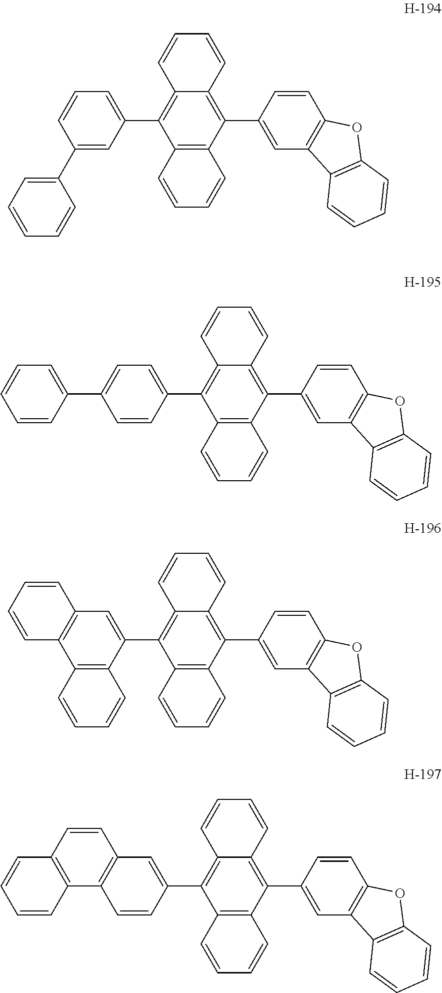 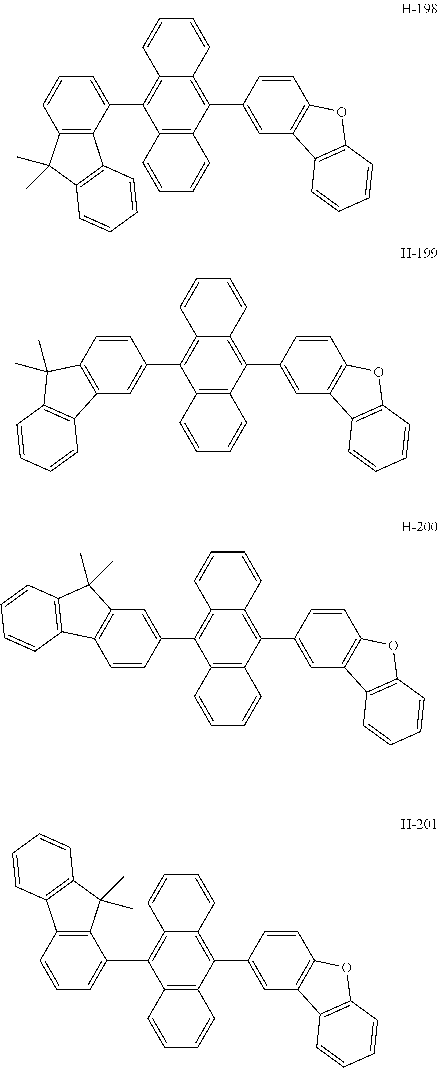 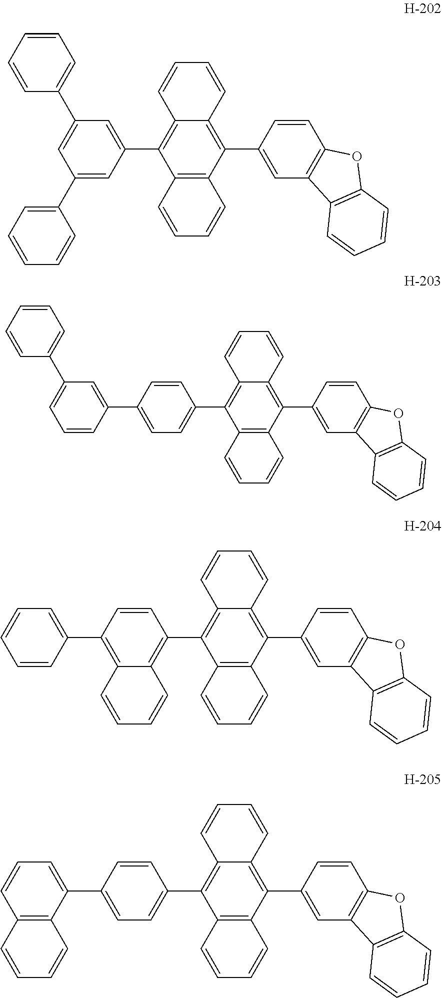 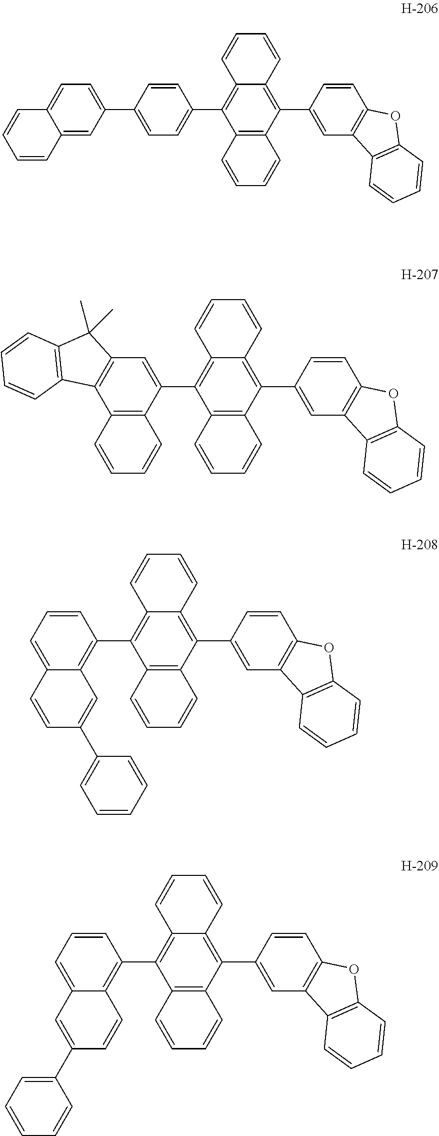 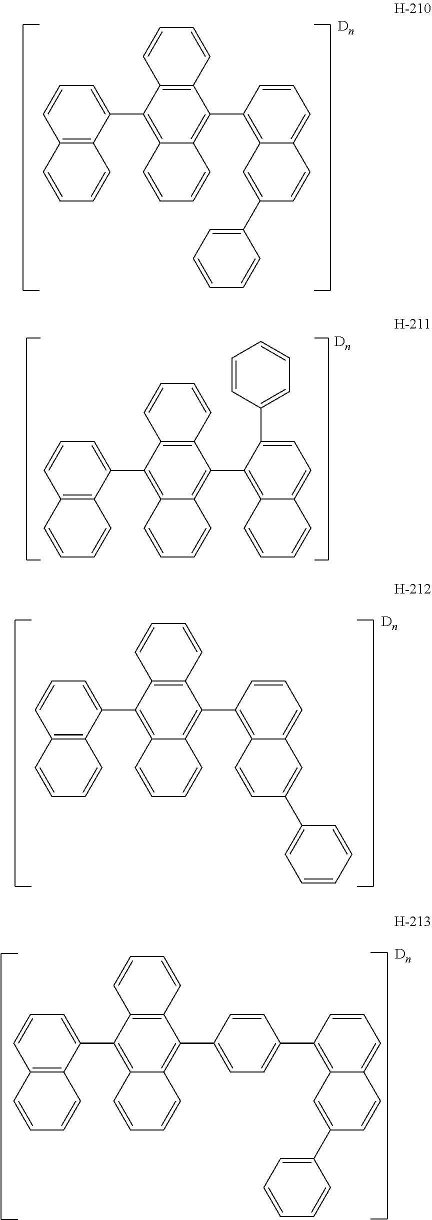 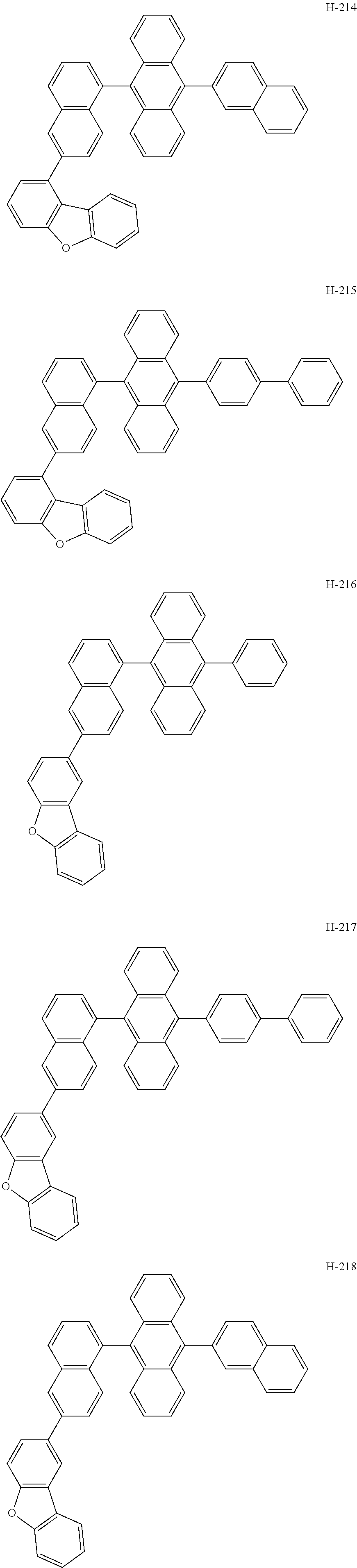 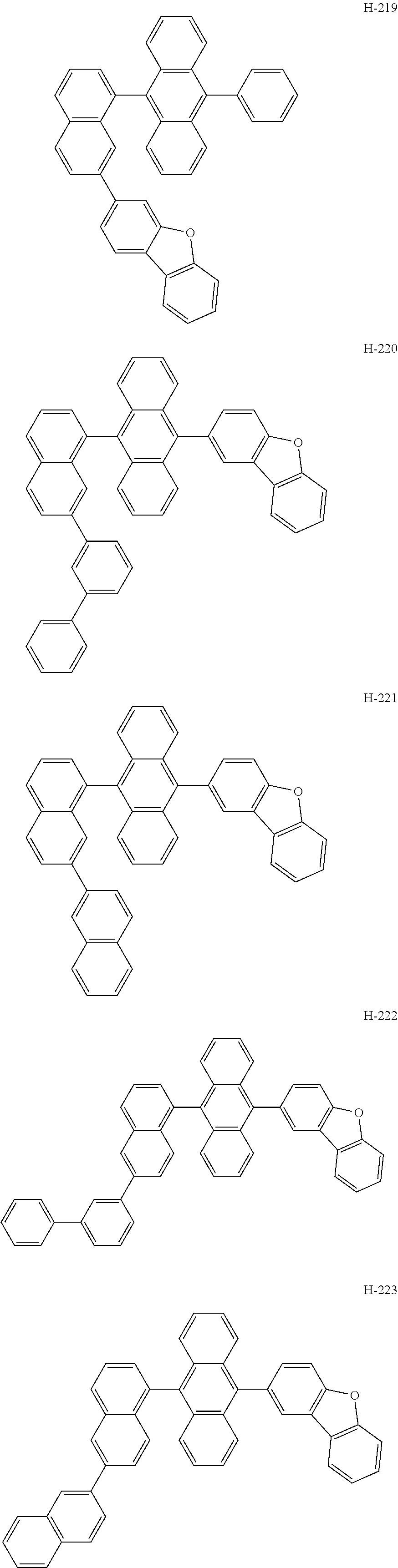 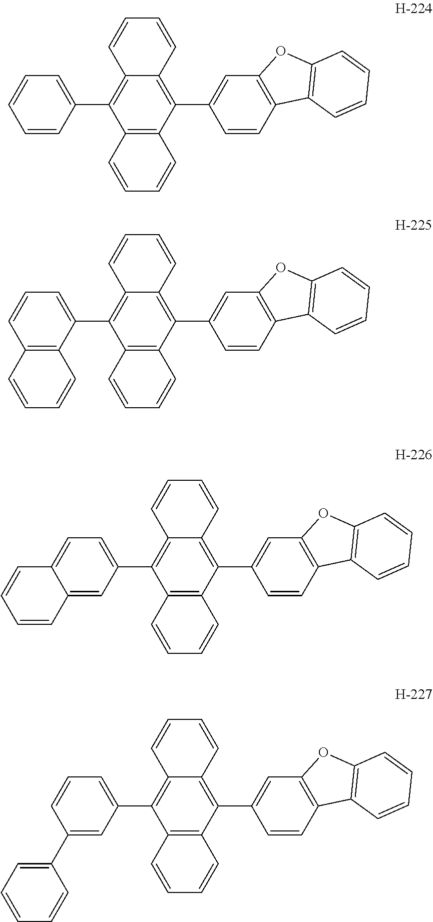 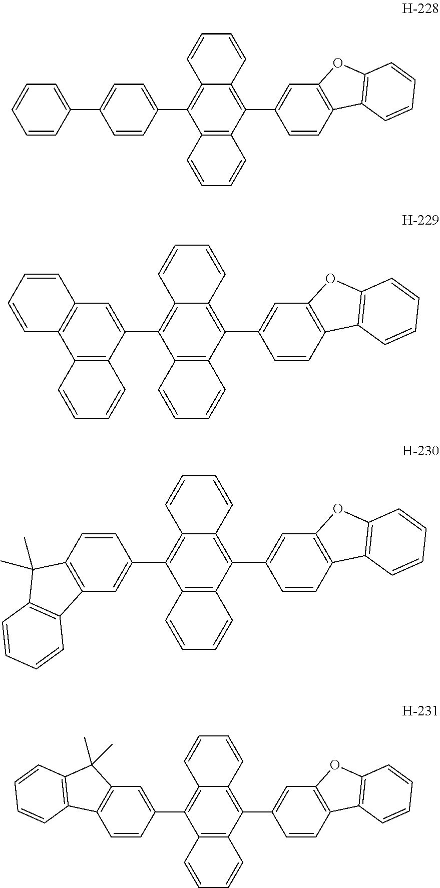 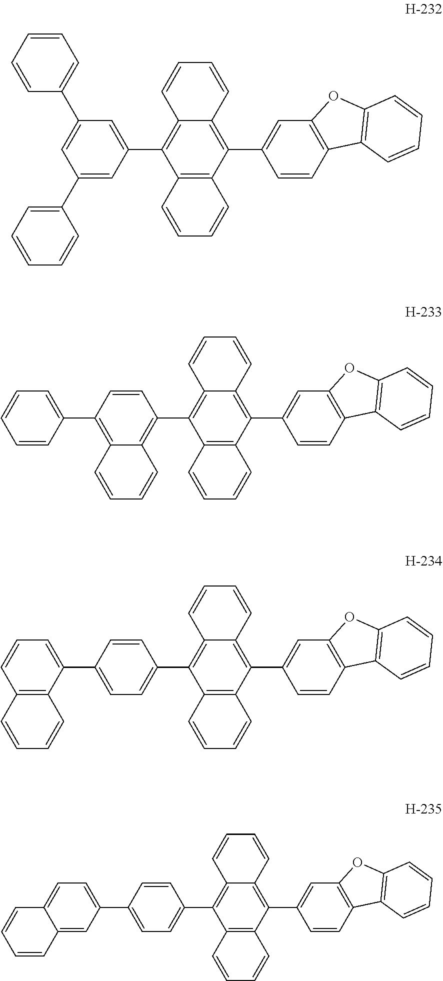 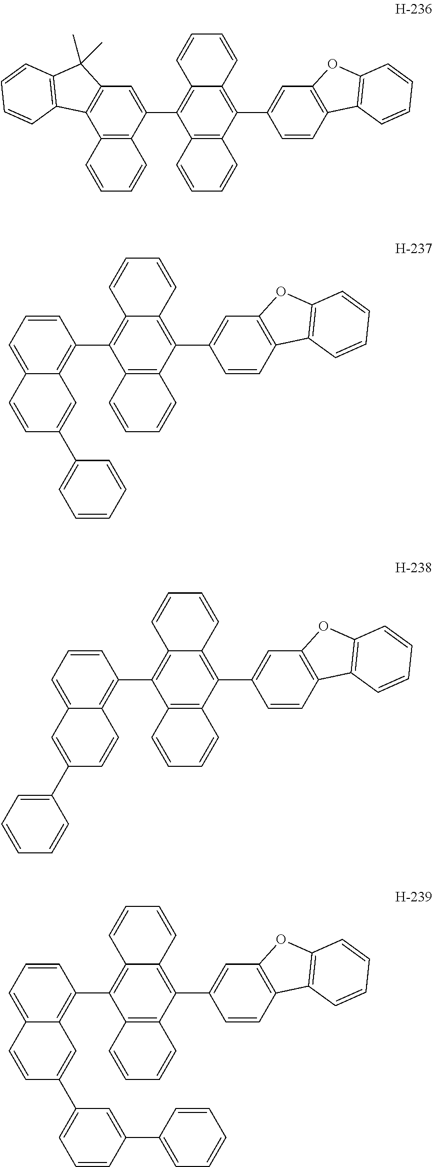 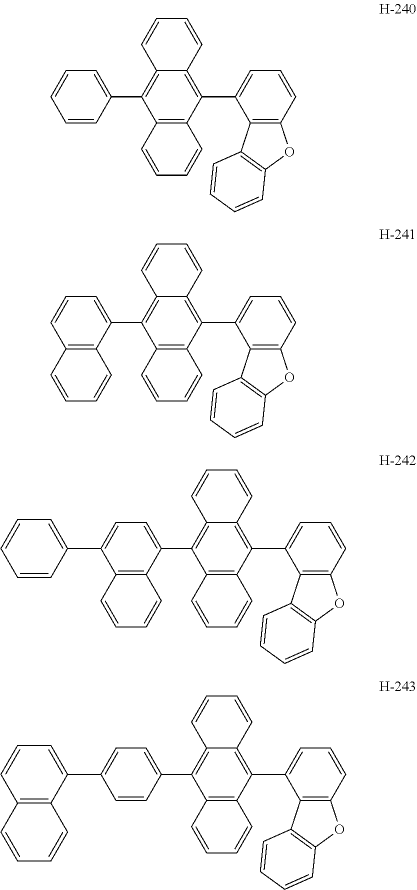 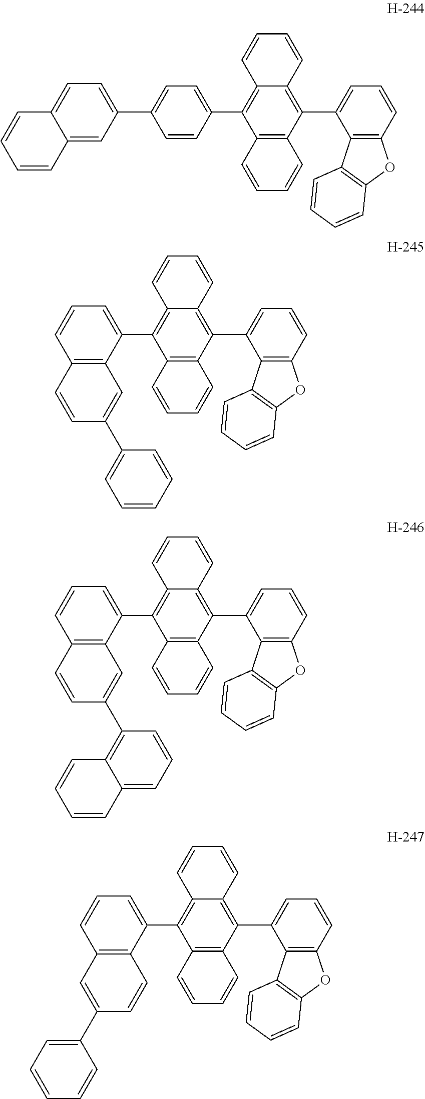 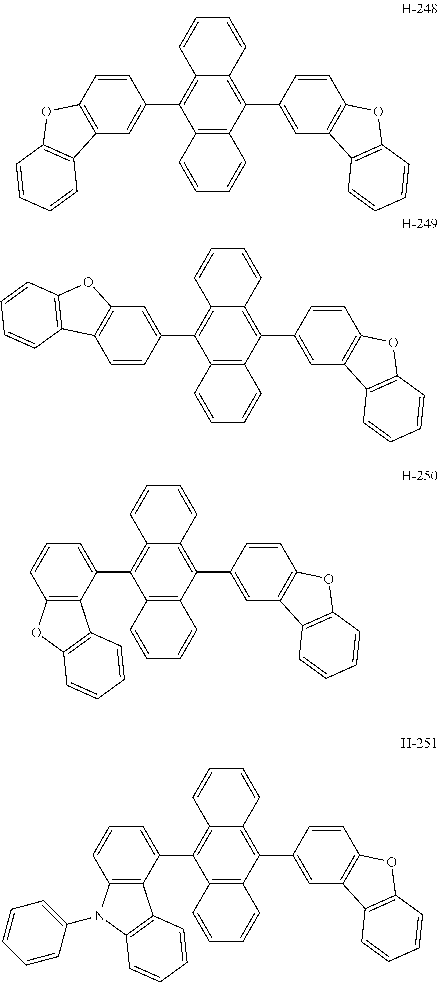 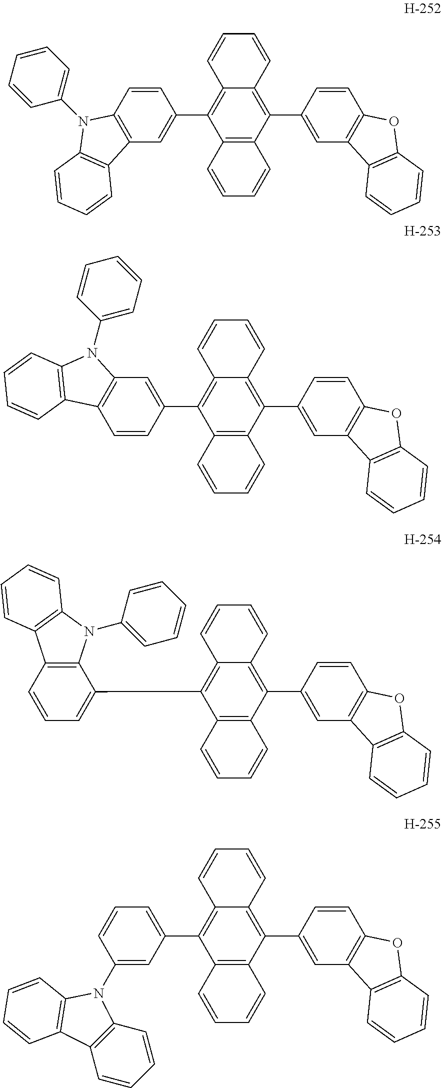 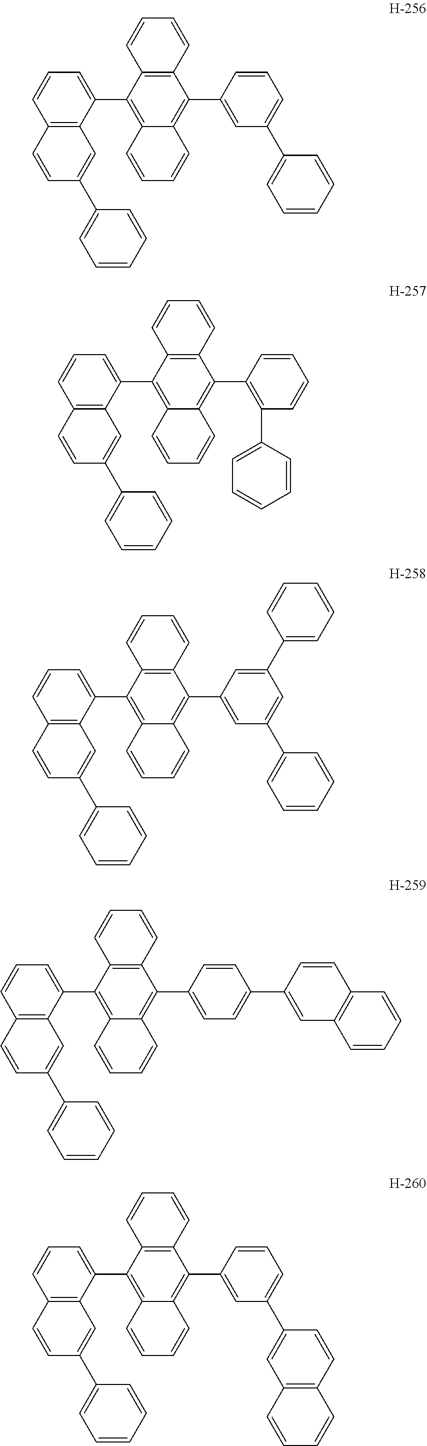 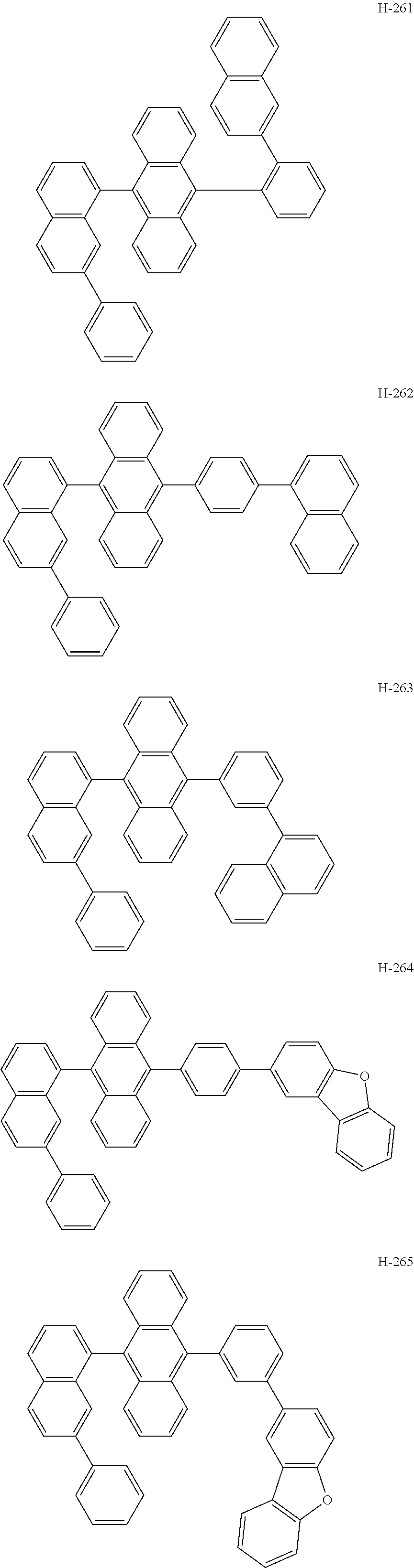 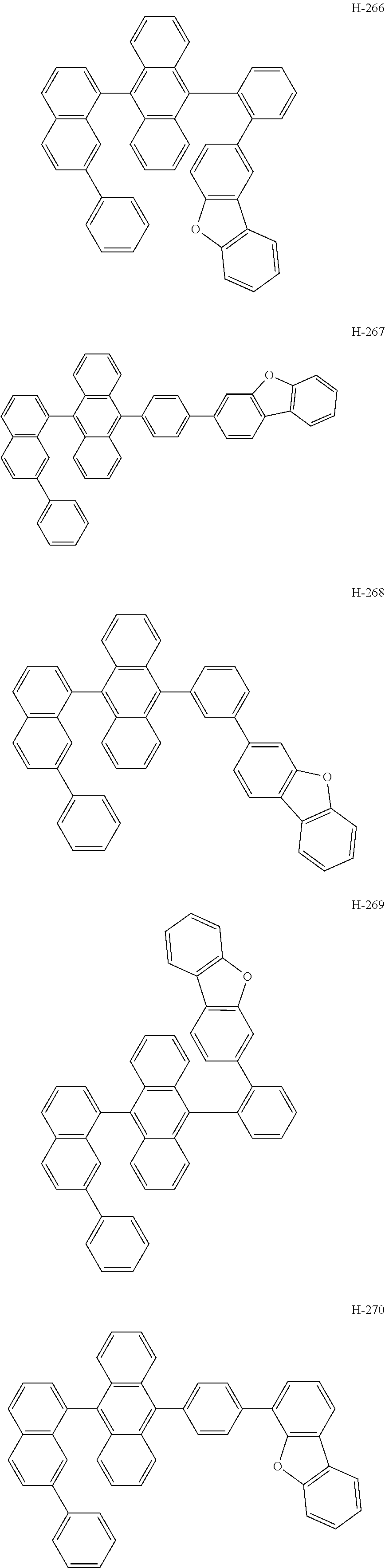 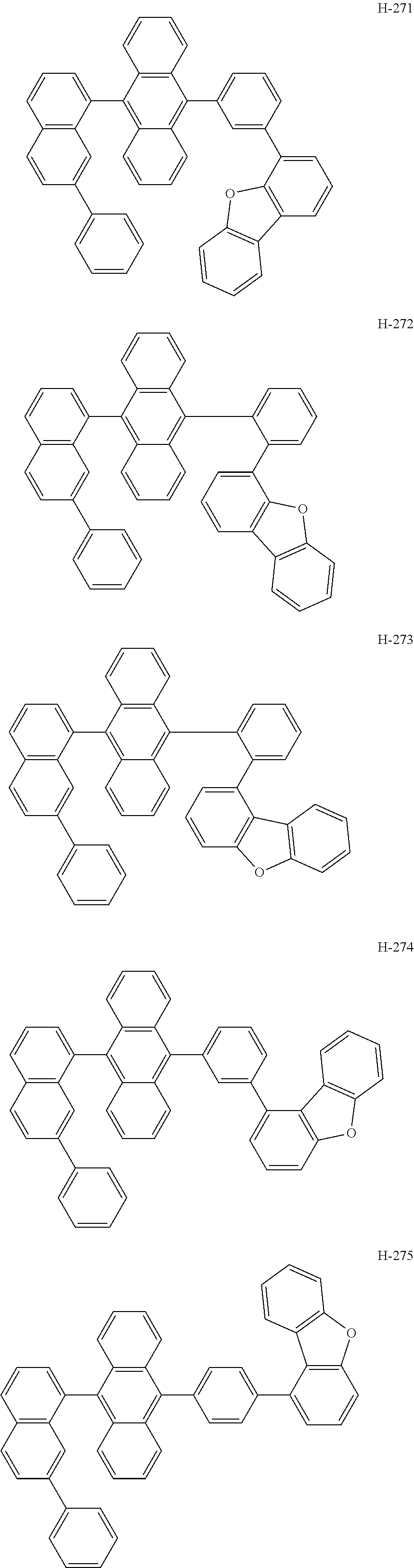 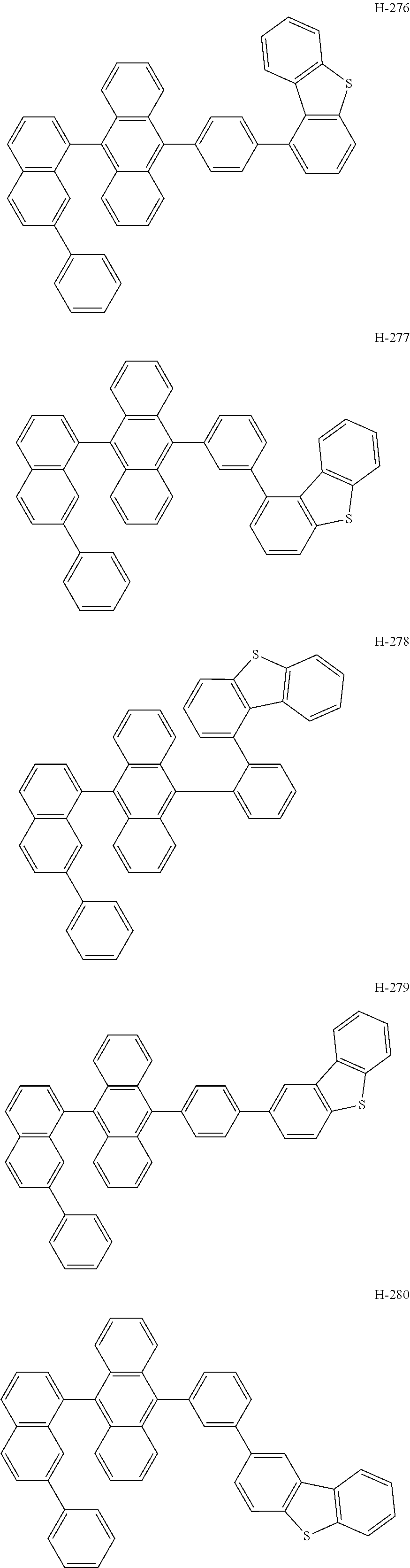 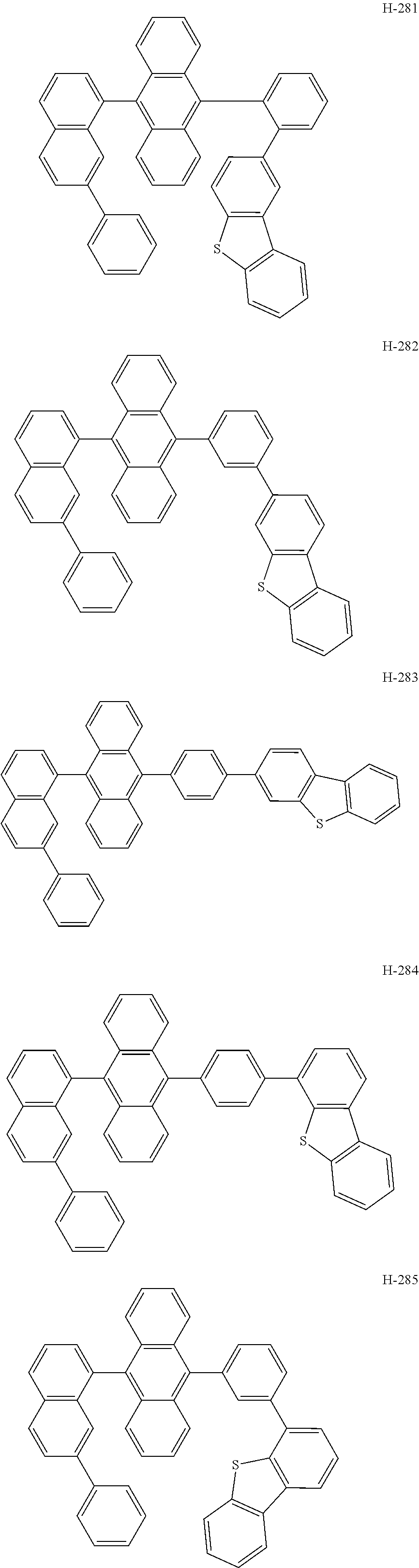 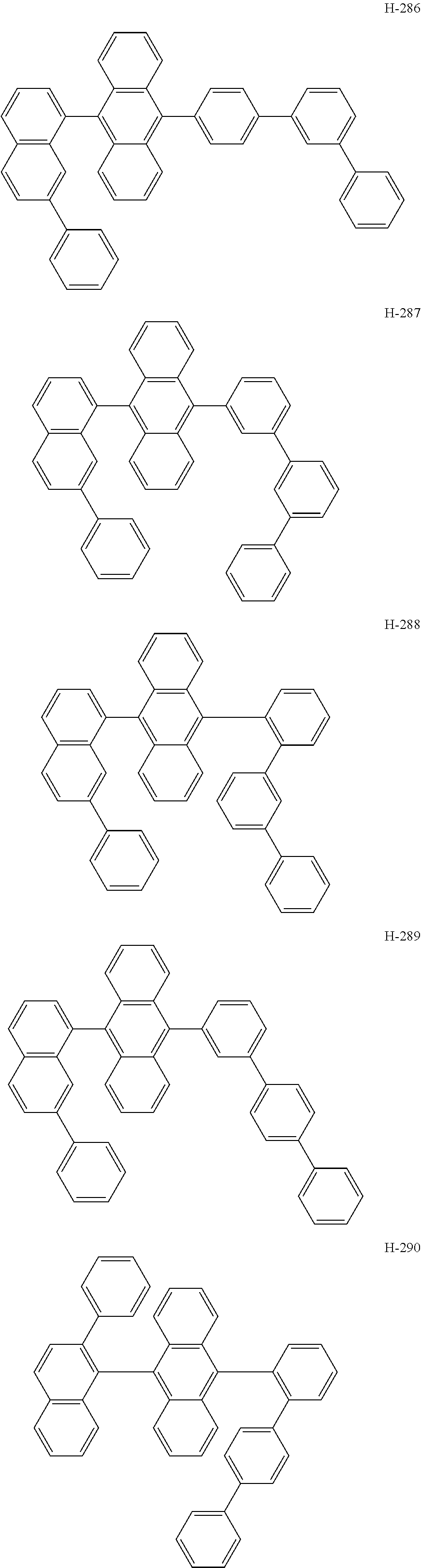 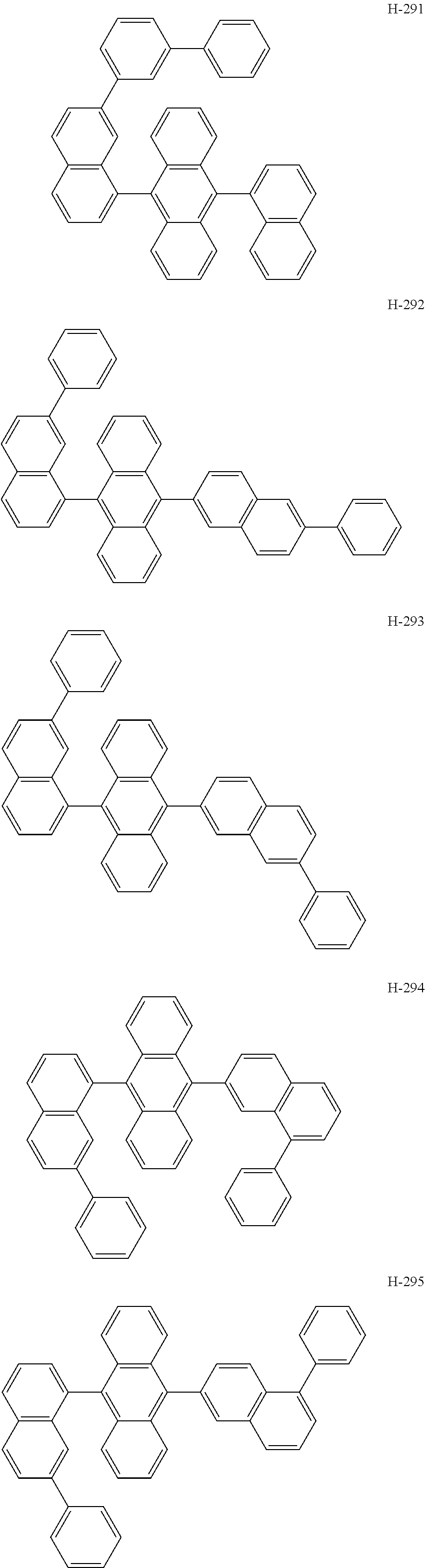 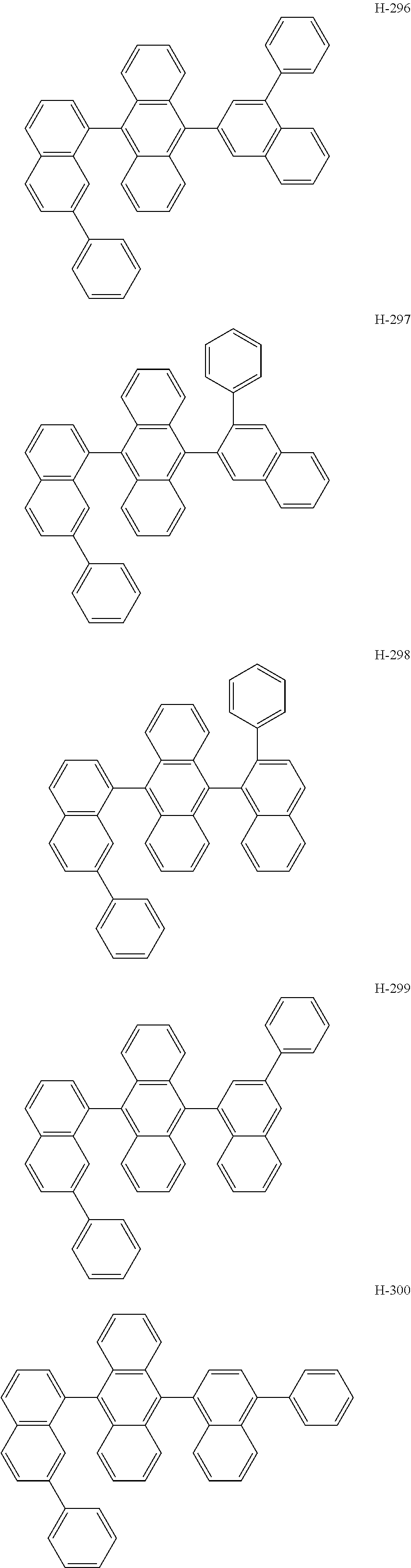 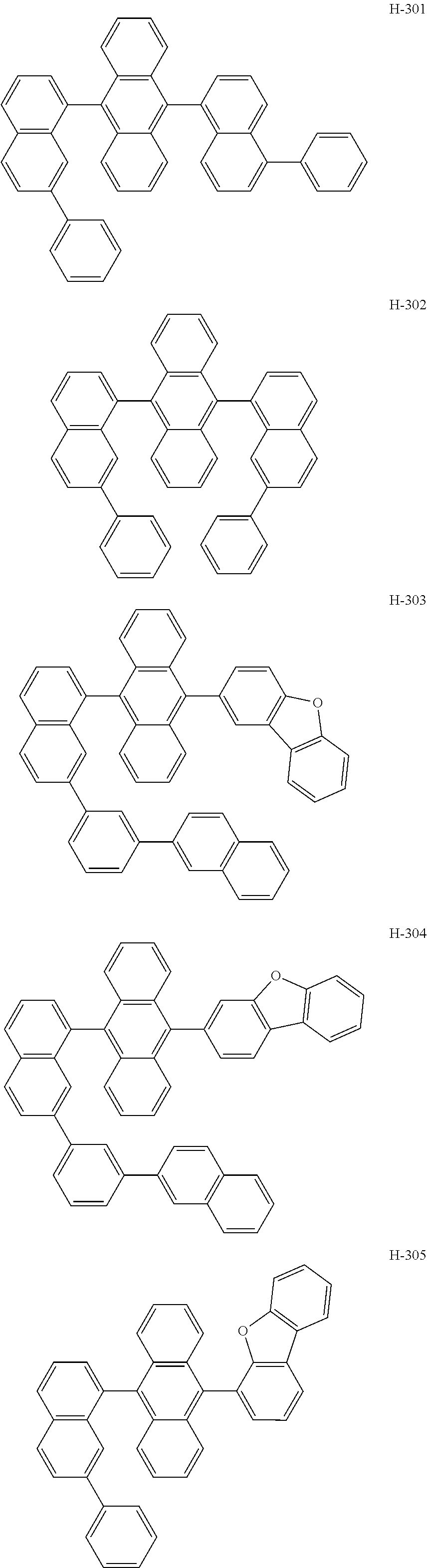 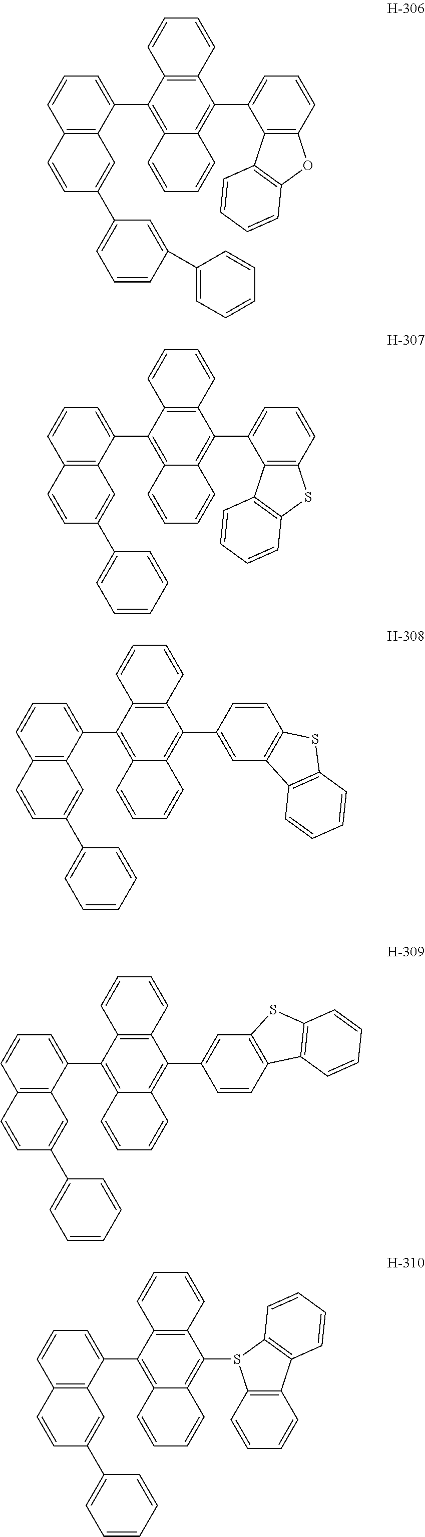 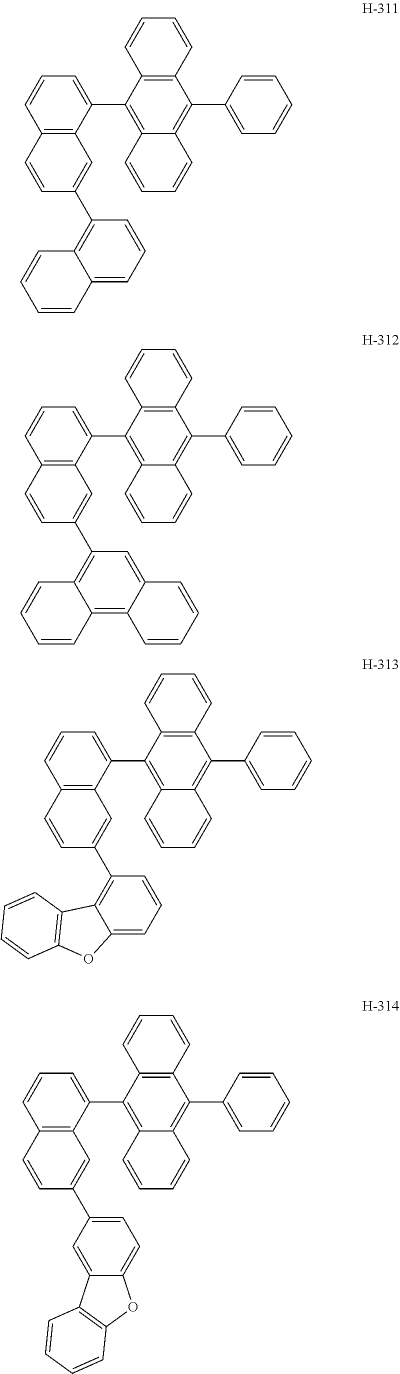 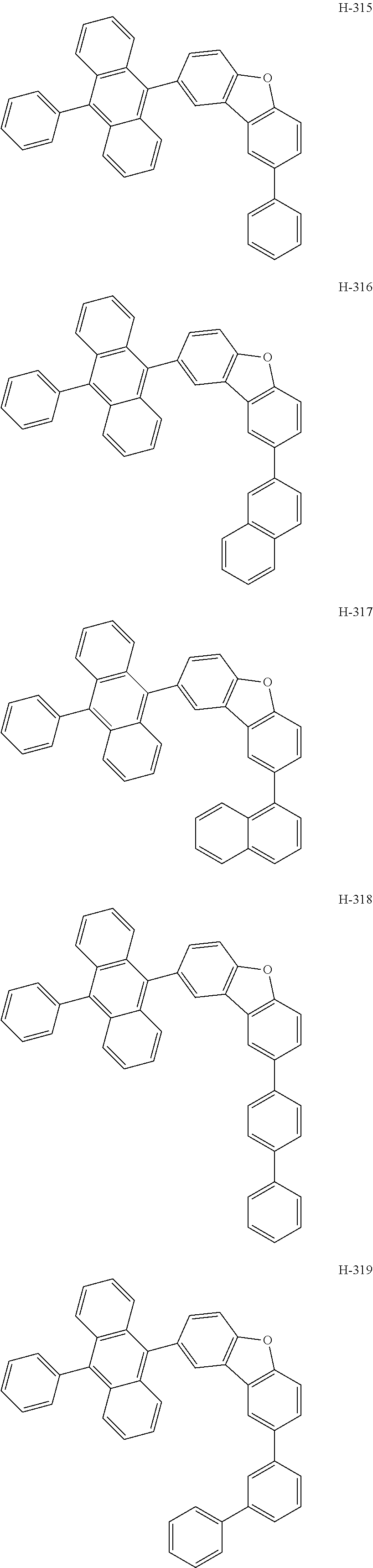 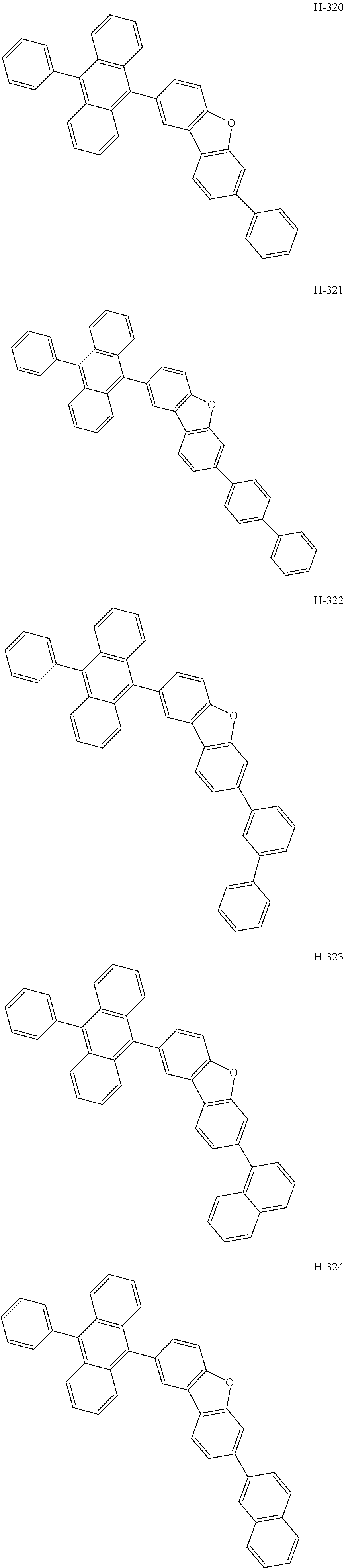 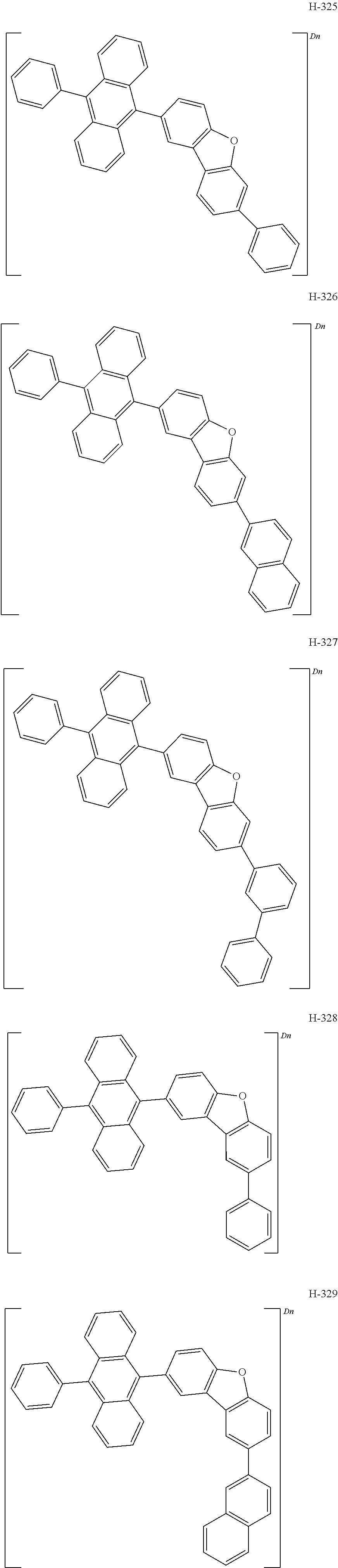 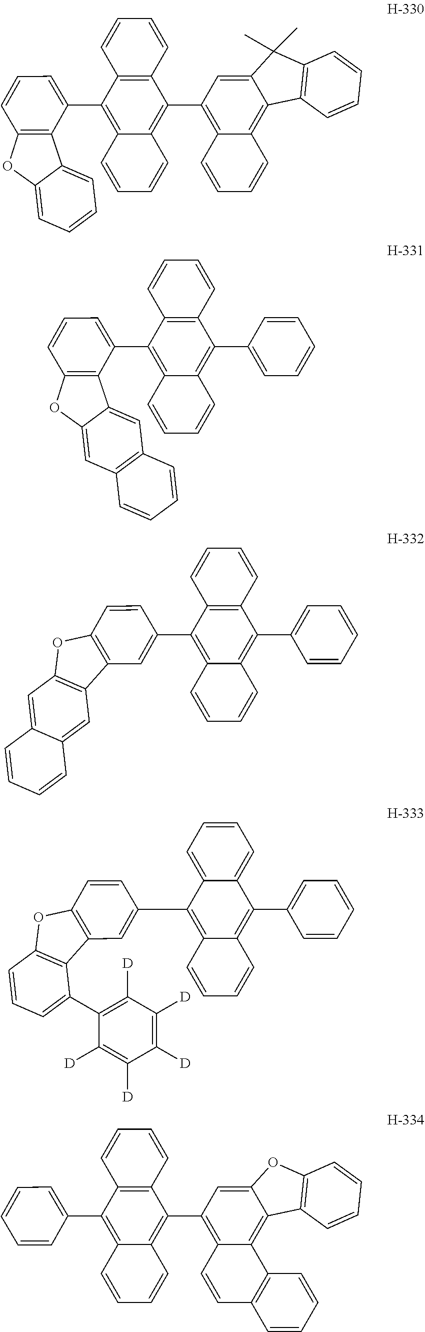 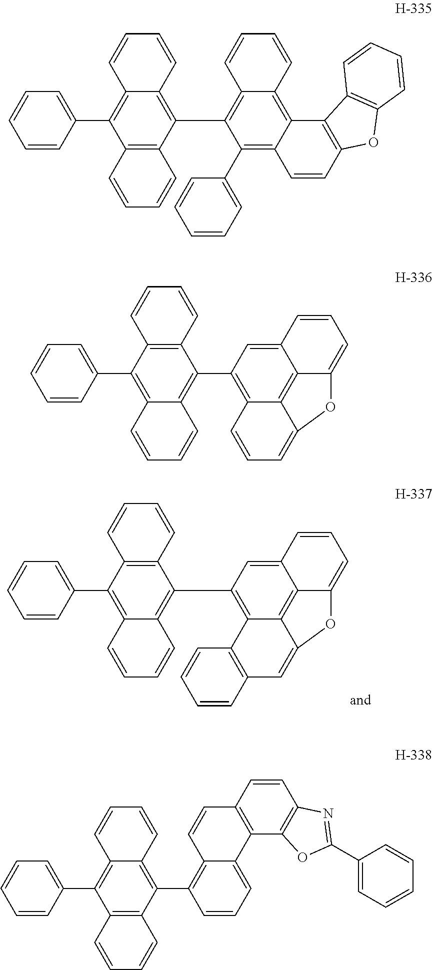
wherein
Dn means that n of the hydrogens are replaced with deuterium, wherein n represents an integer from 1 to 50.

9. The organic electroluminescent device according to claim 1, wherein the compound represented by formula 2 is selected from the following compounds: 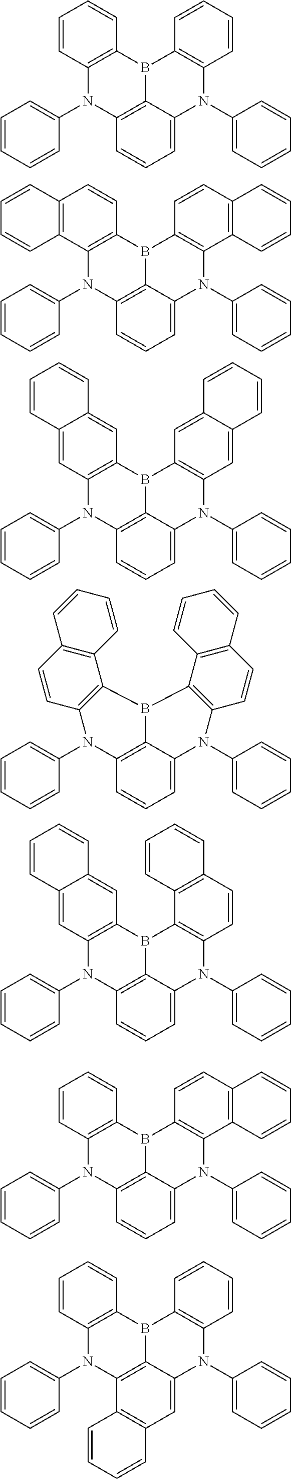 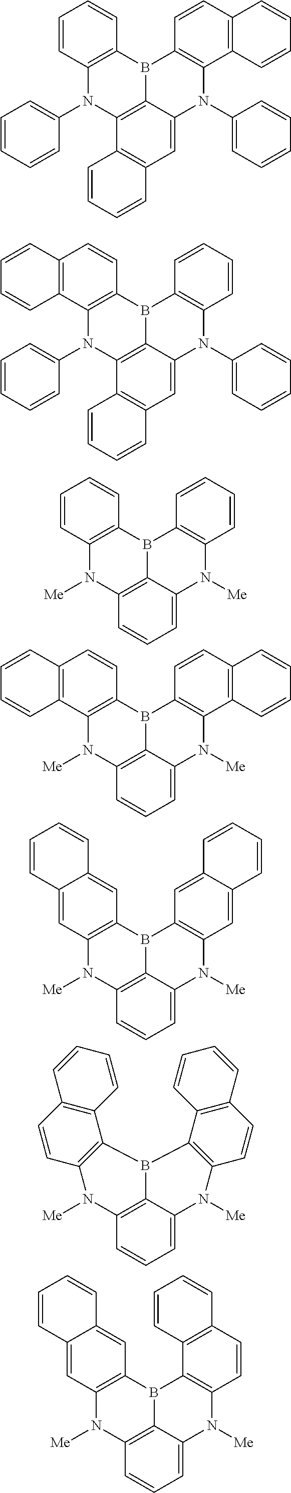 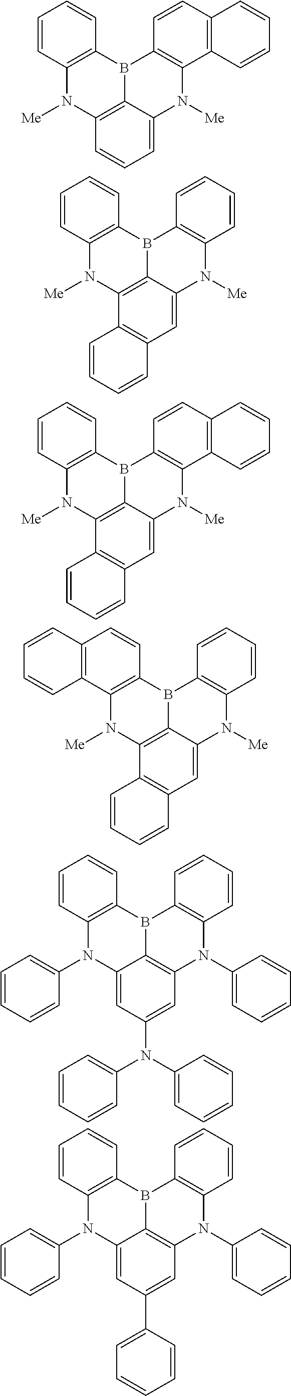 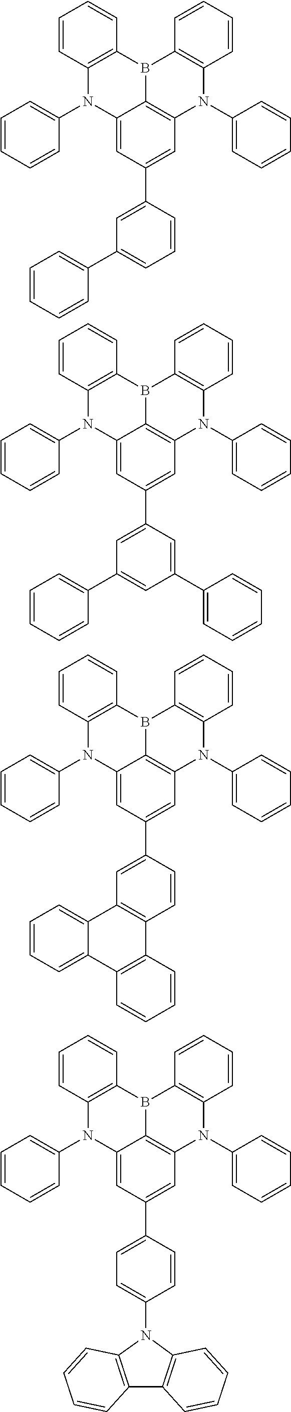 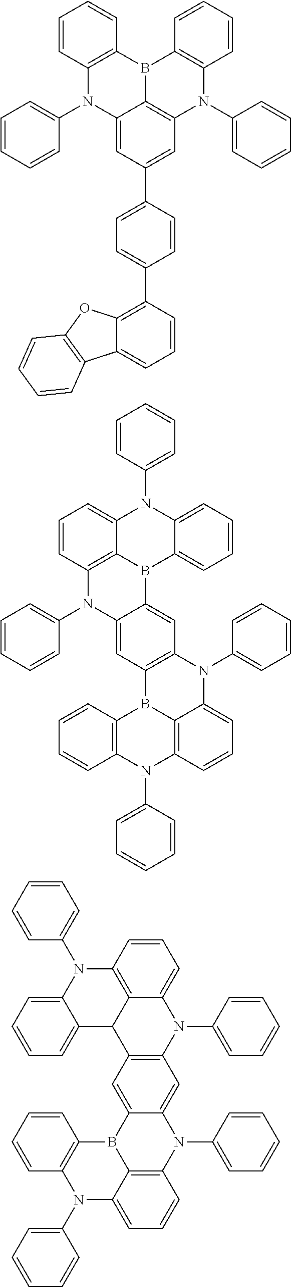 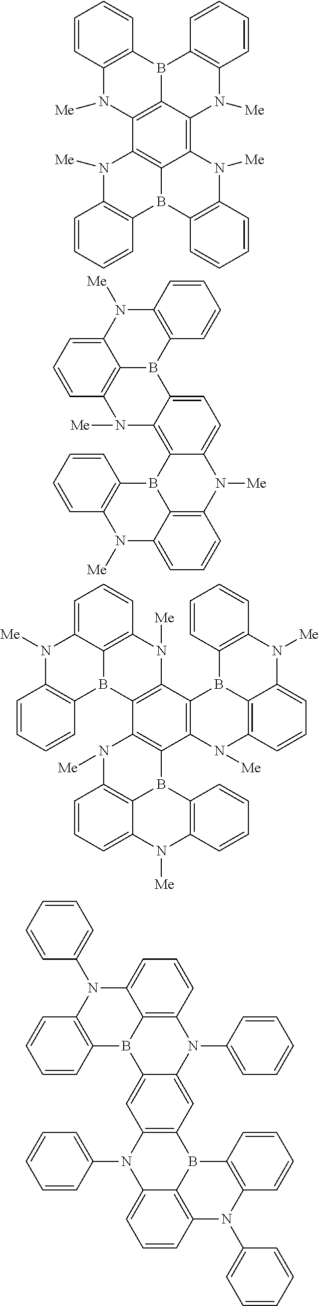 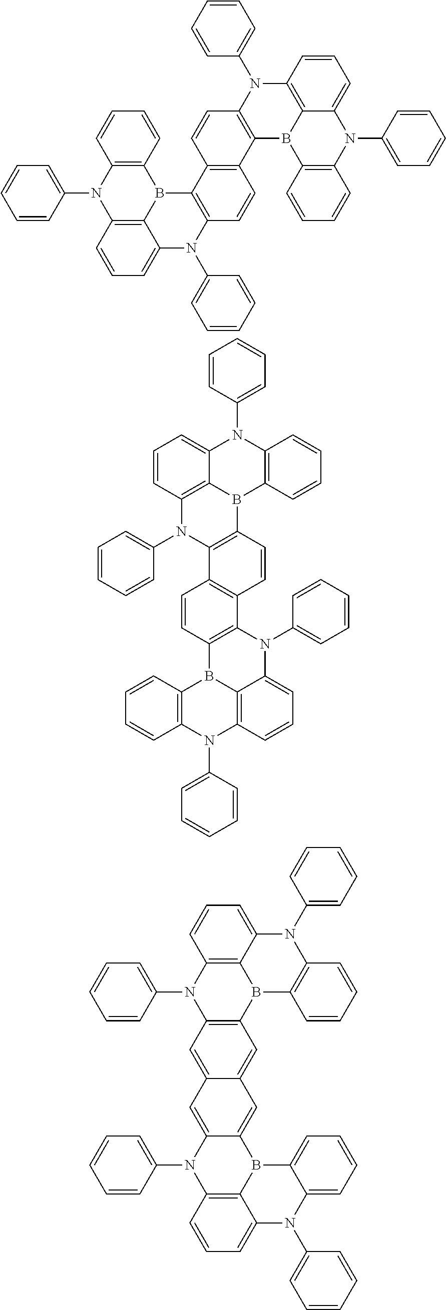 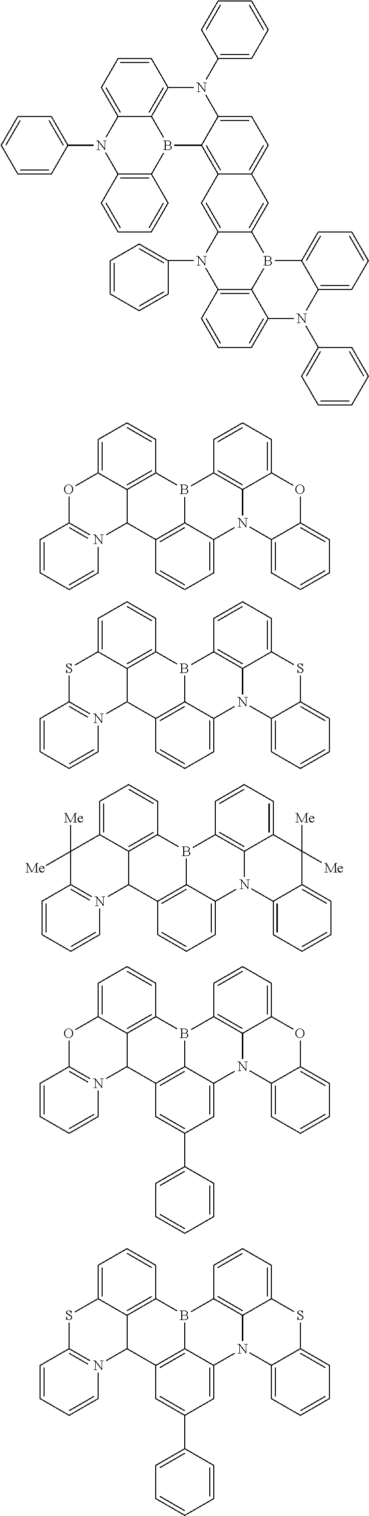 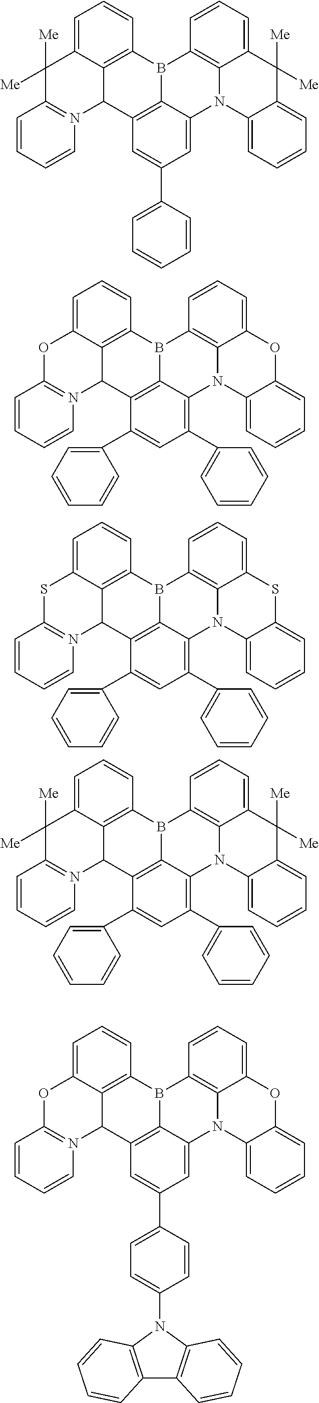 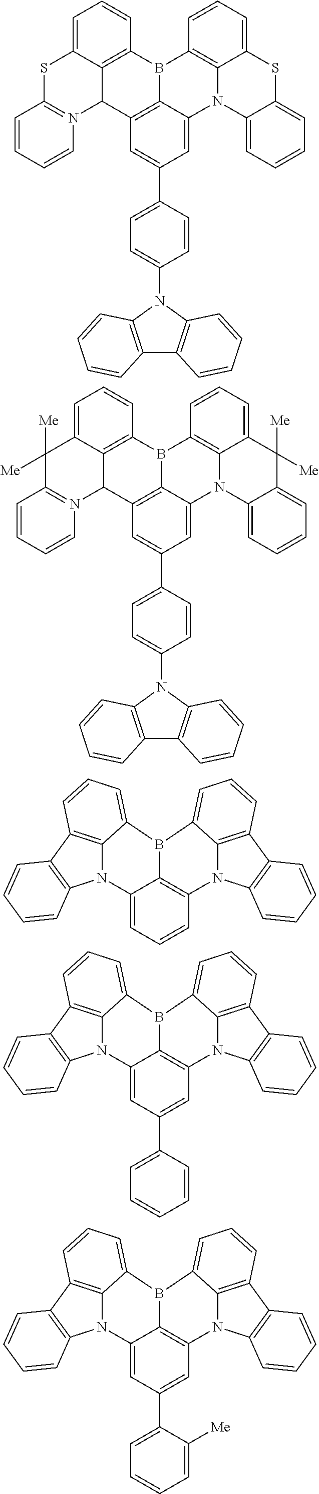 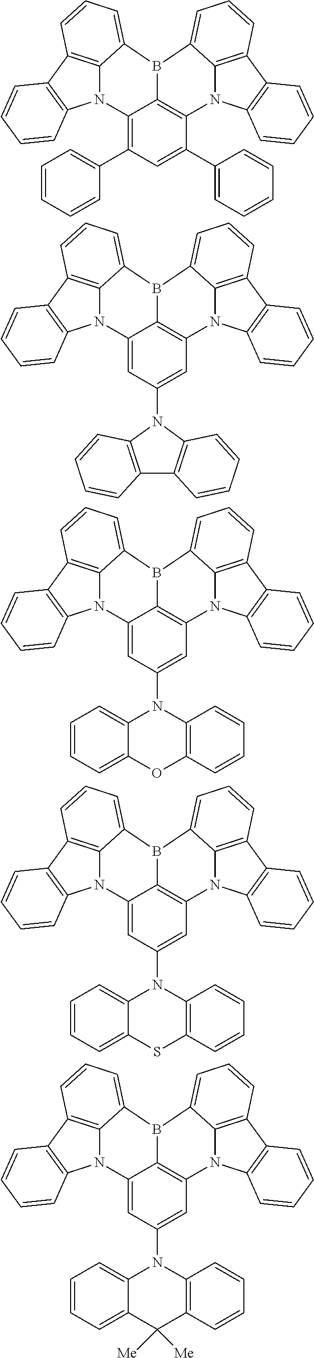 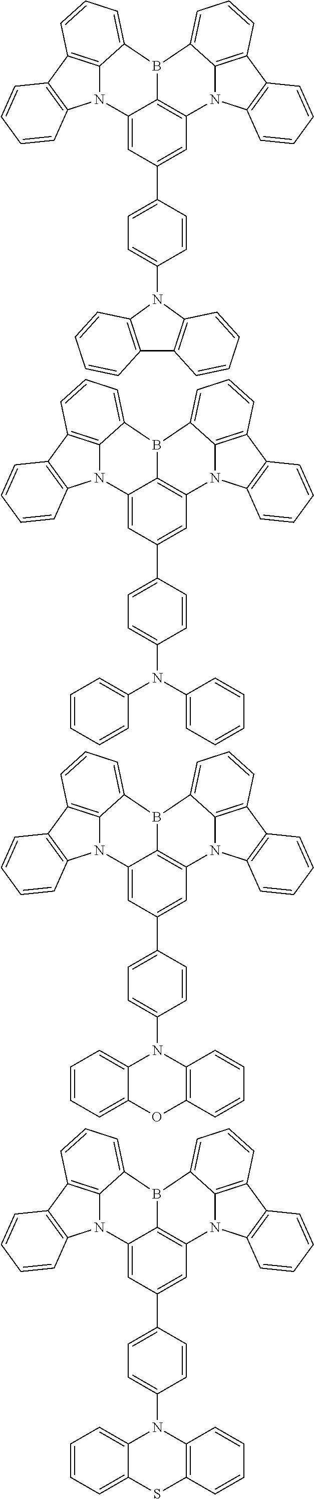 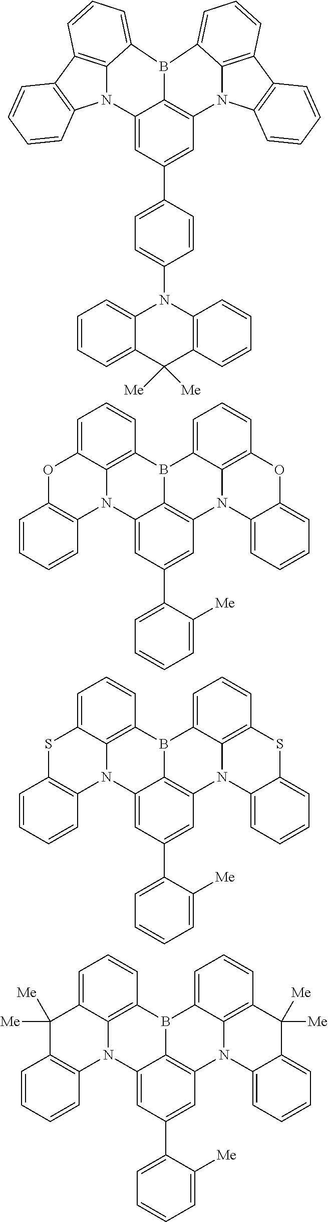 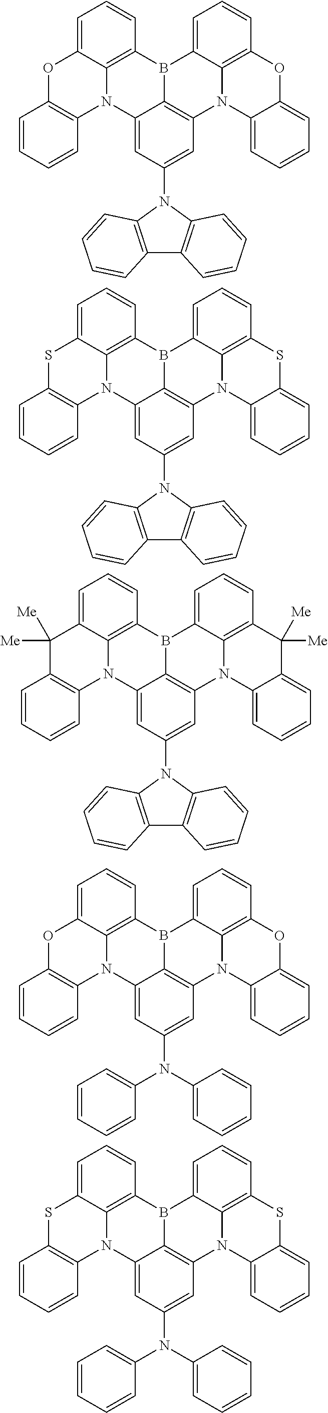 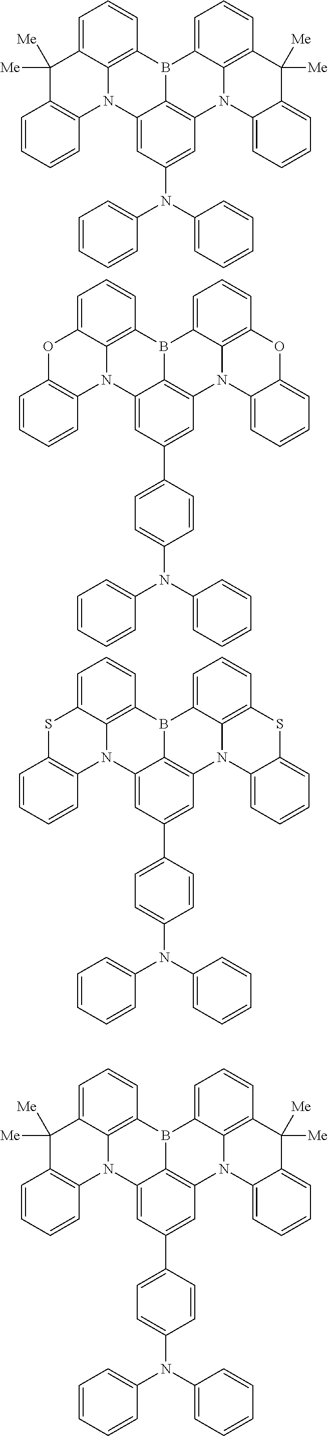 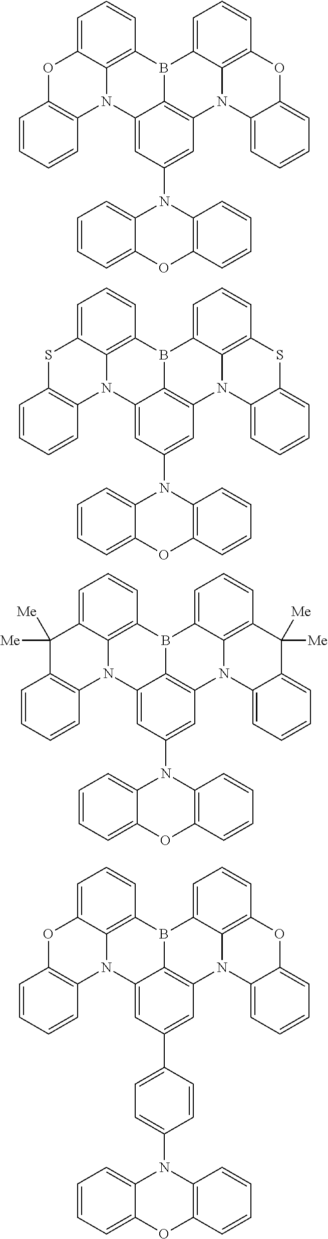 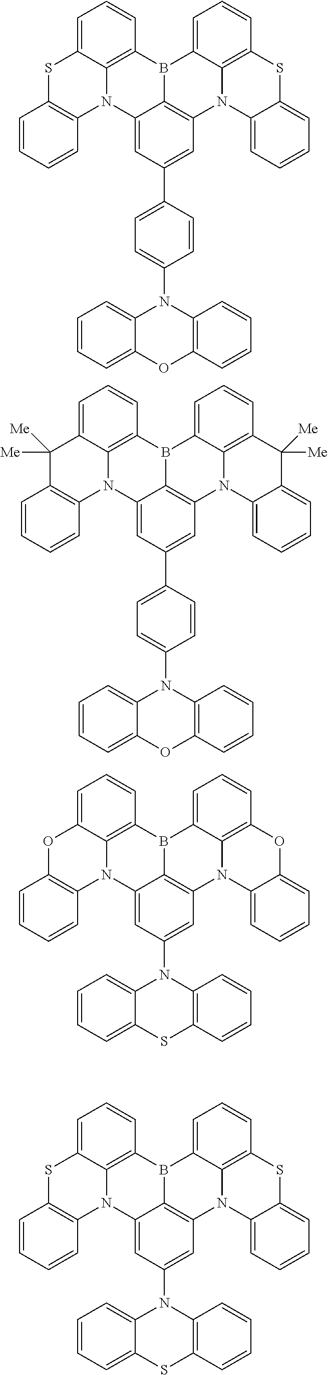 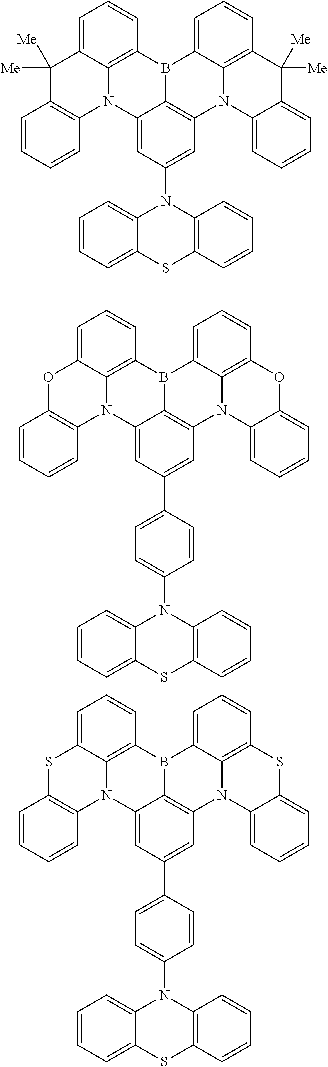 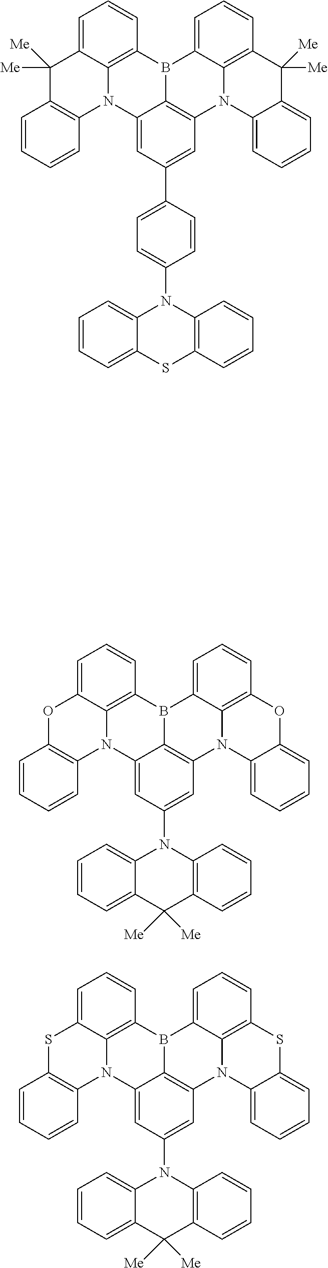 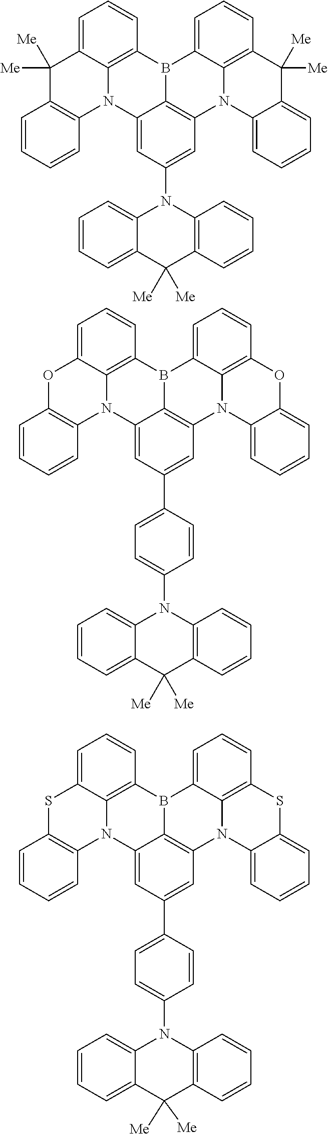 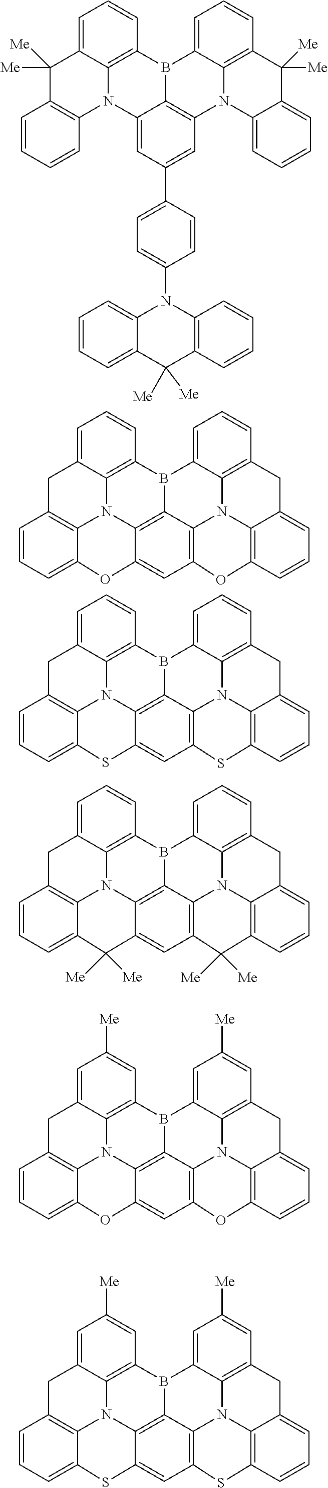 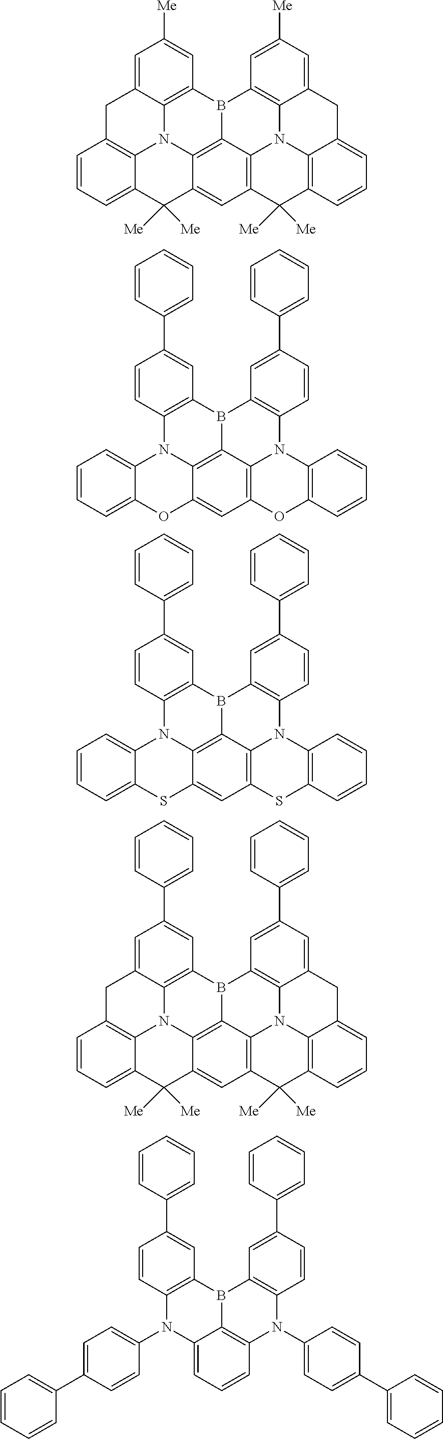 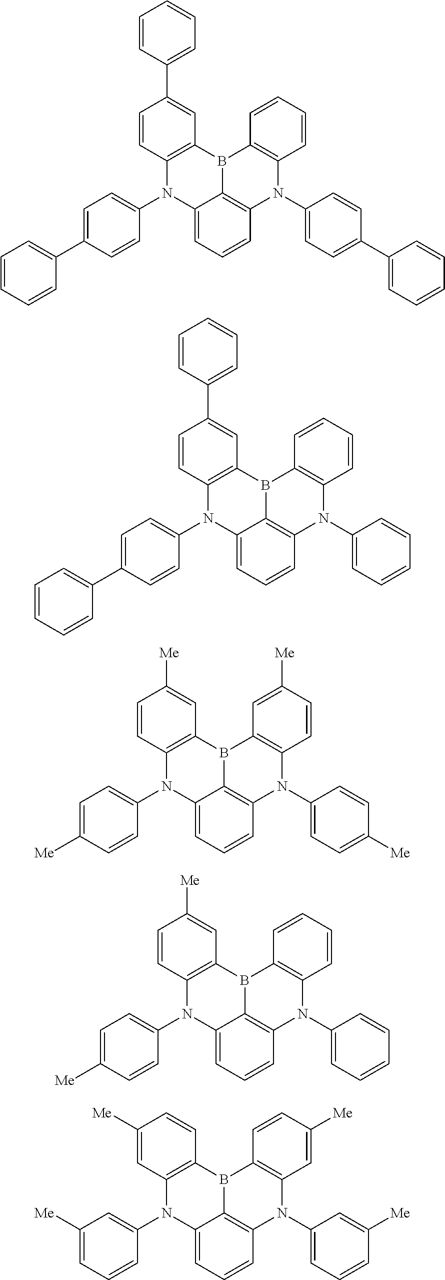 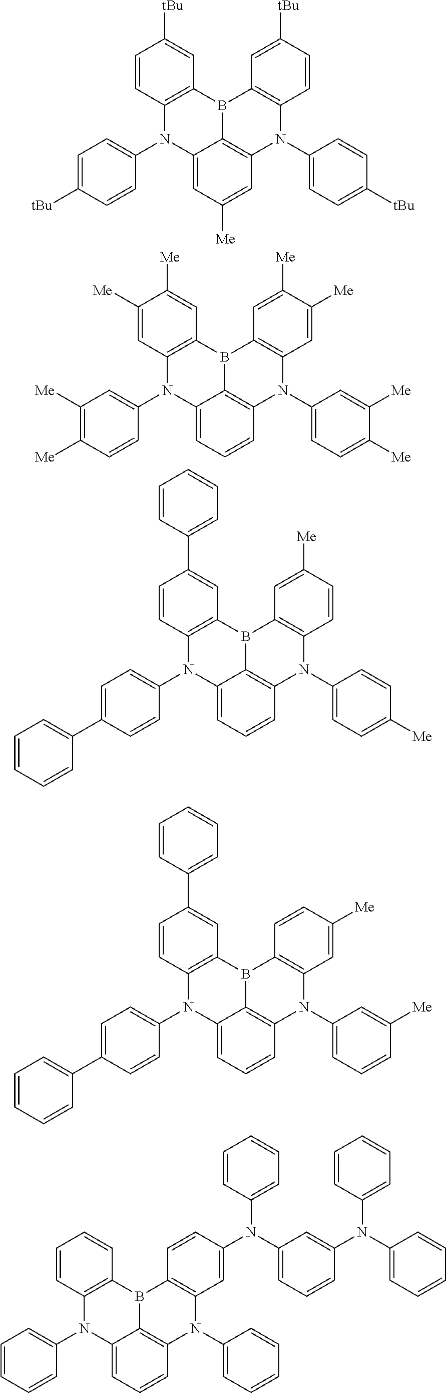 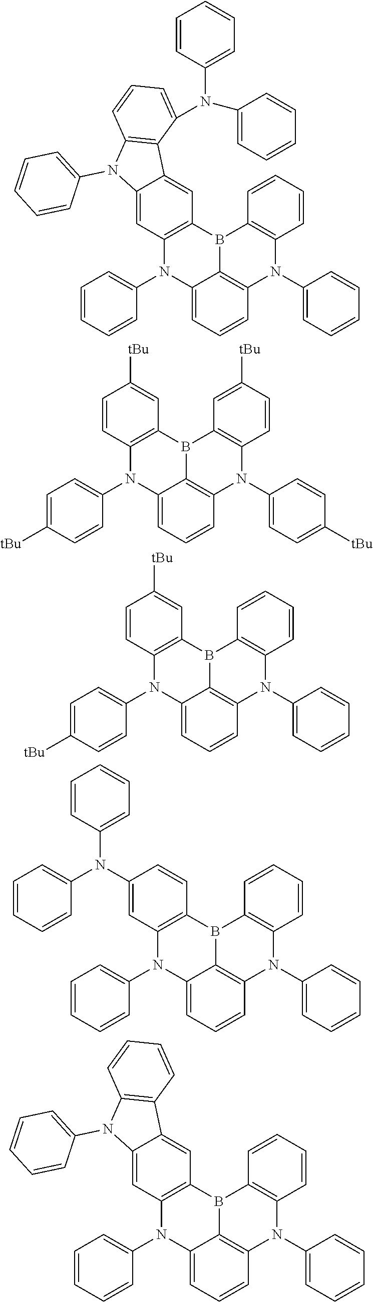 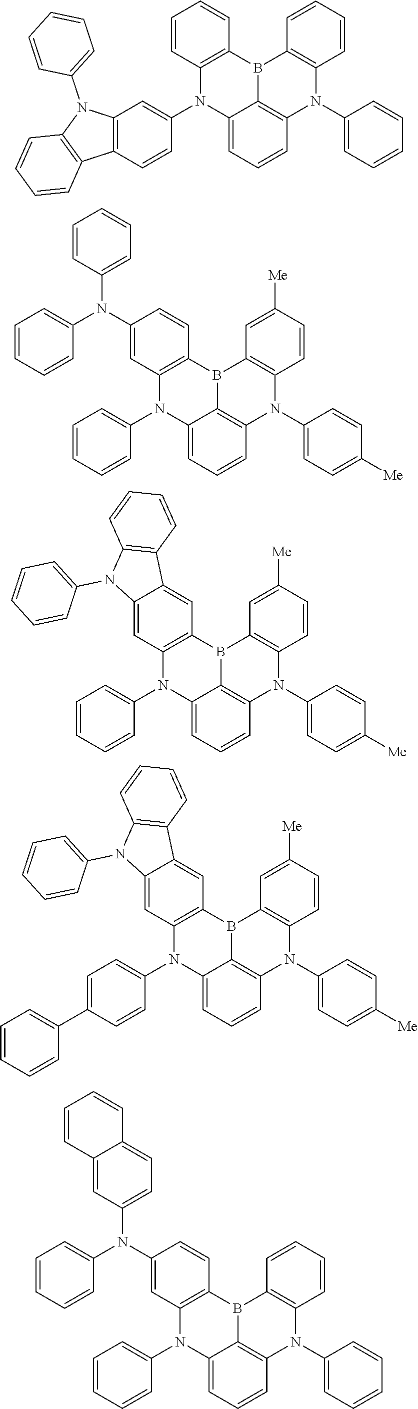 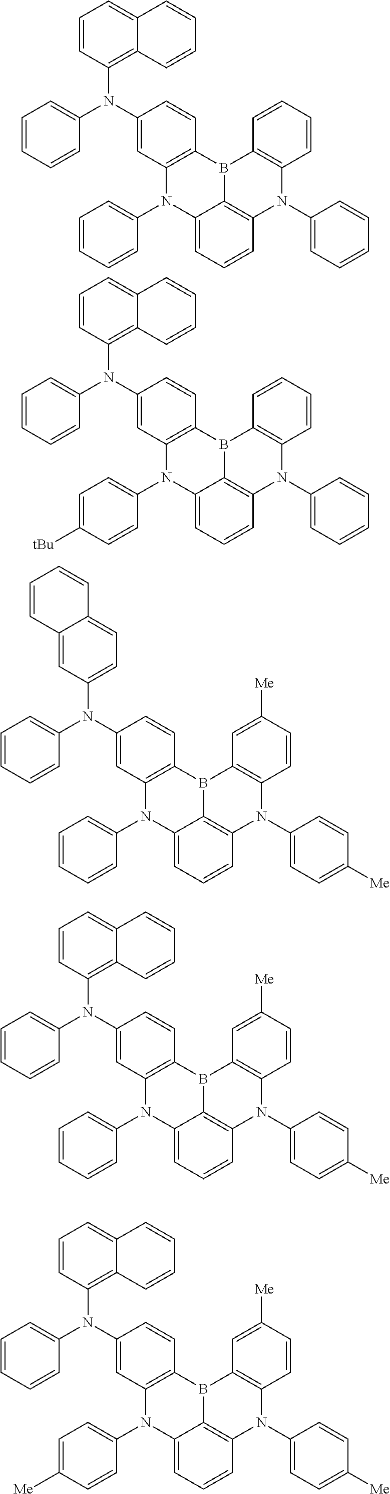 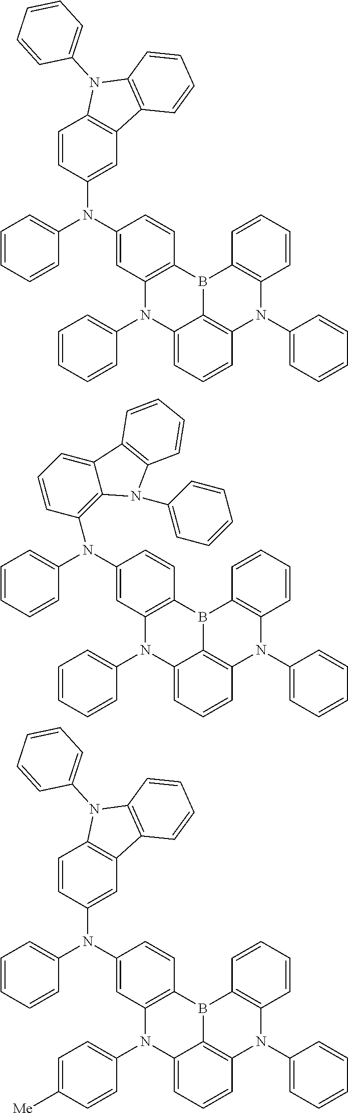 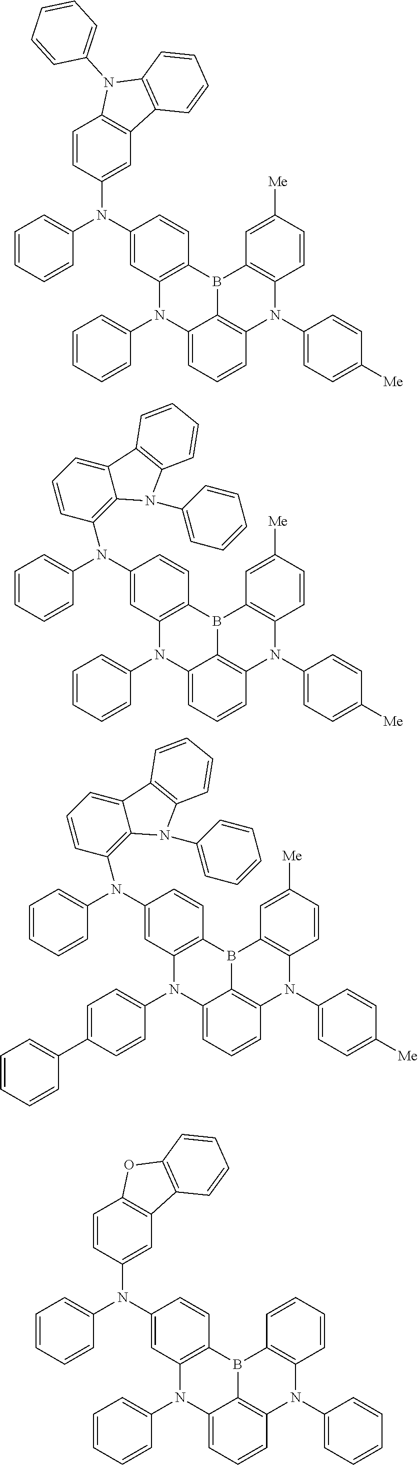 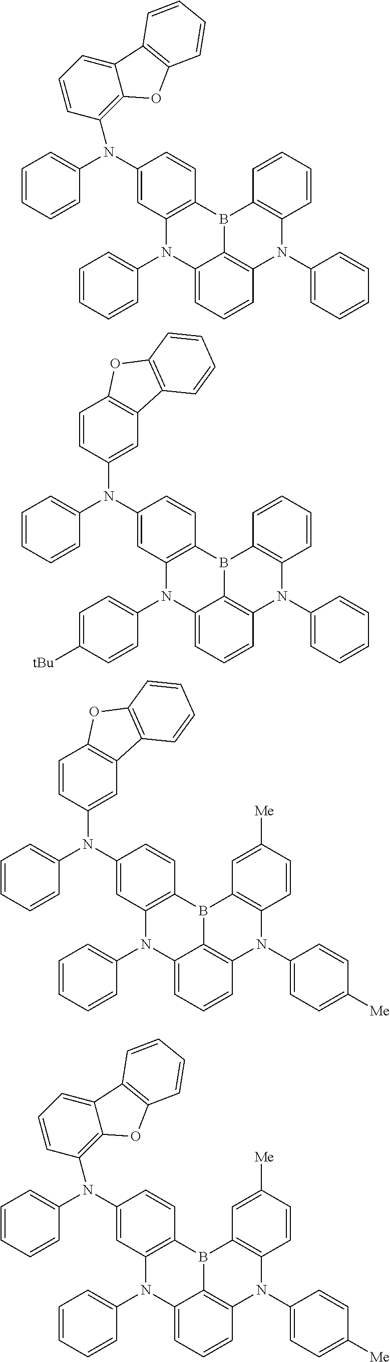 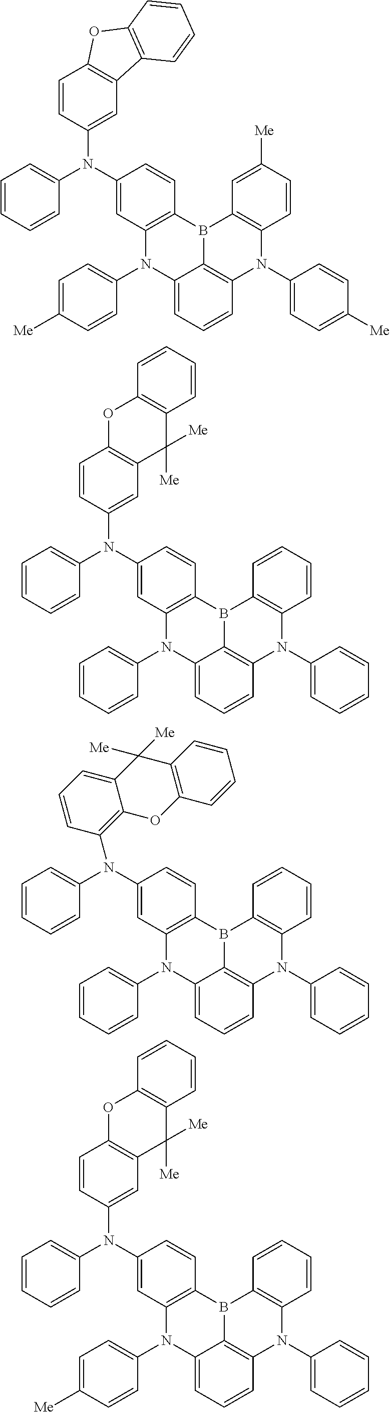 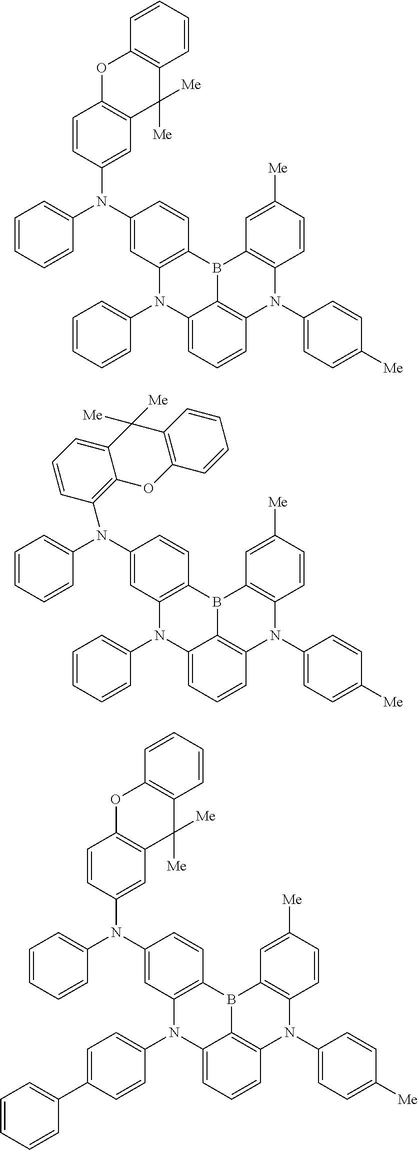 [SMILES: Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1 ; Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1] 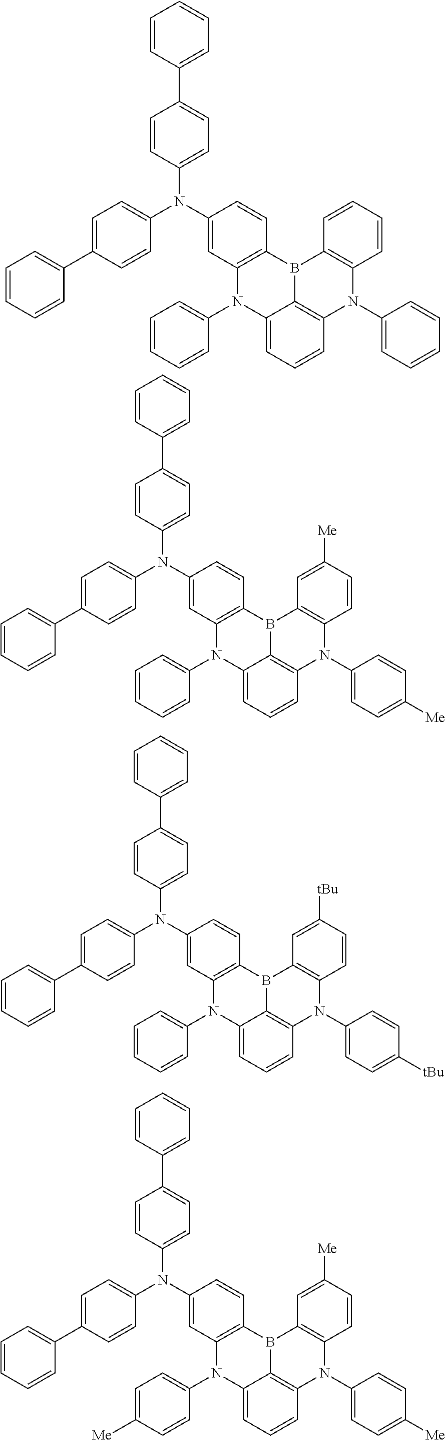 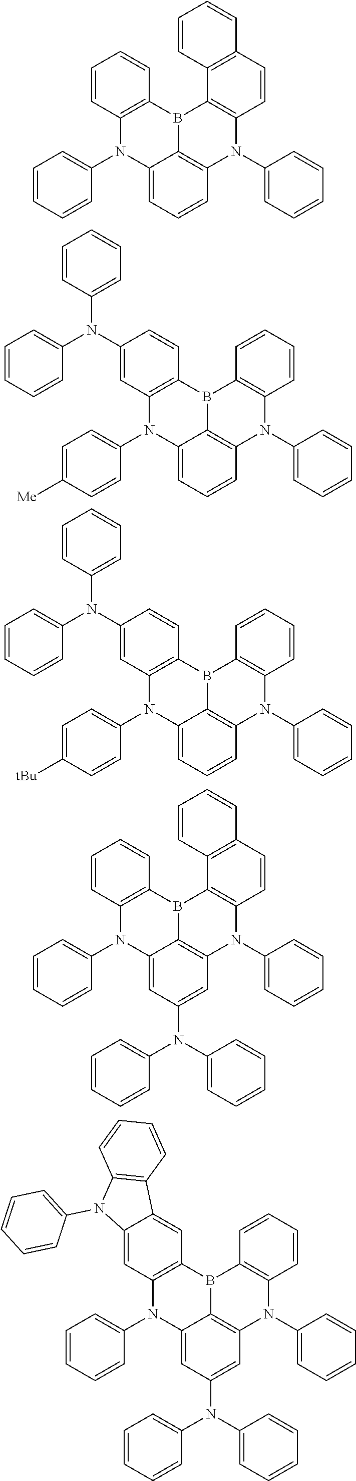 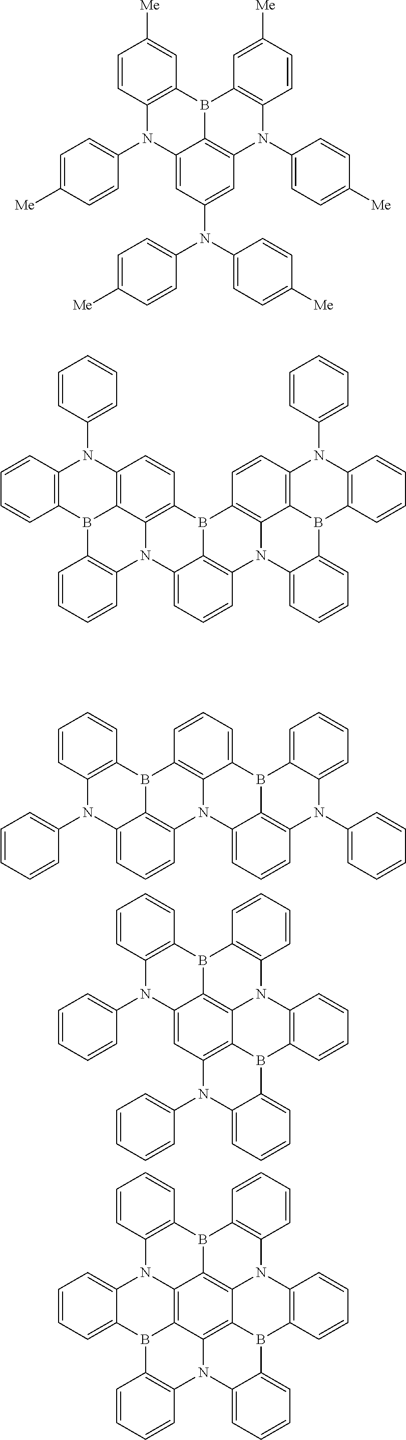 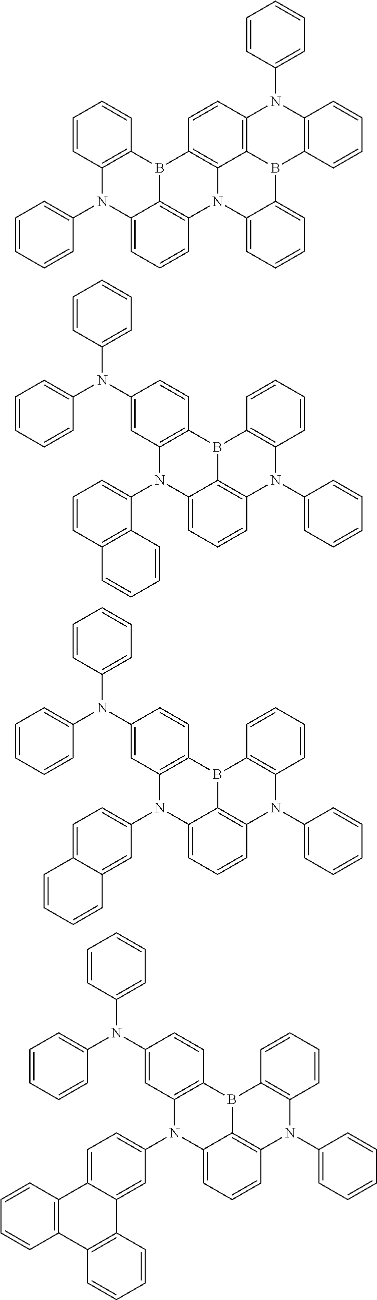 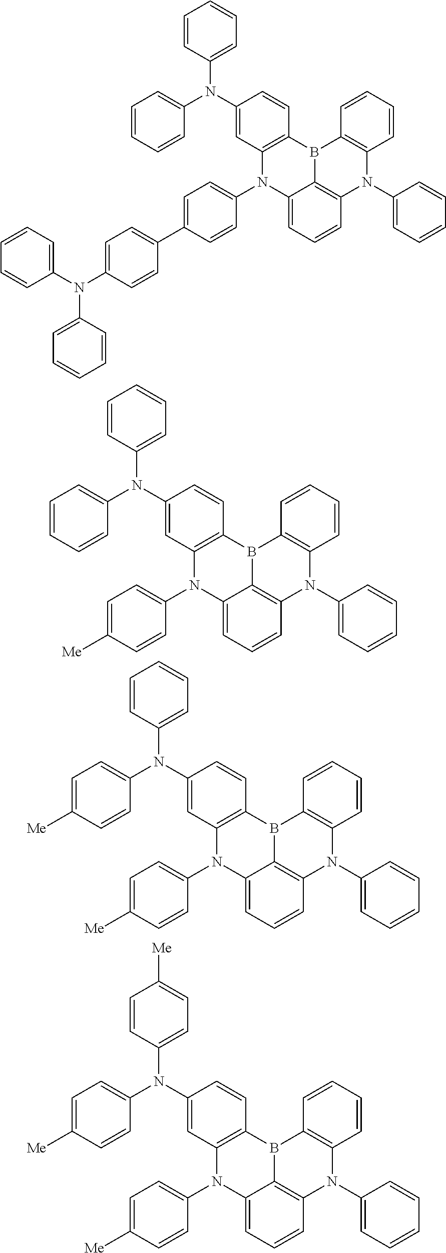 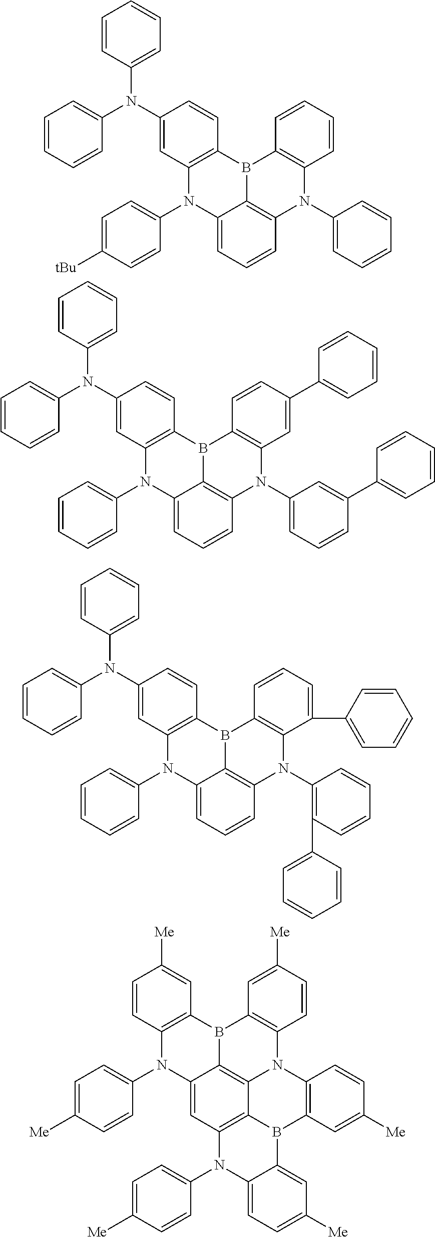 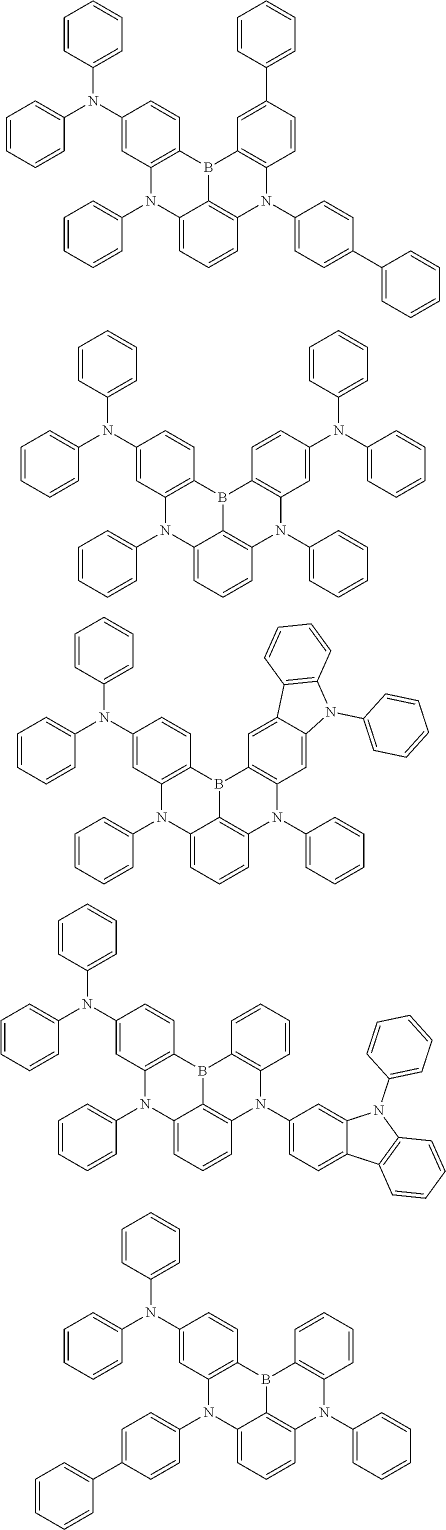 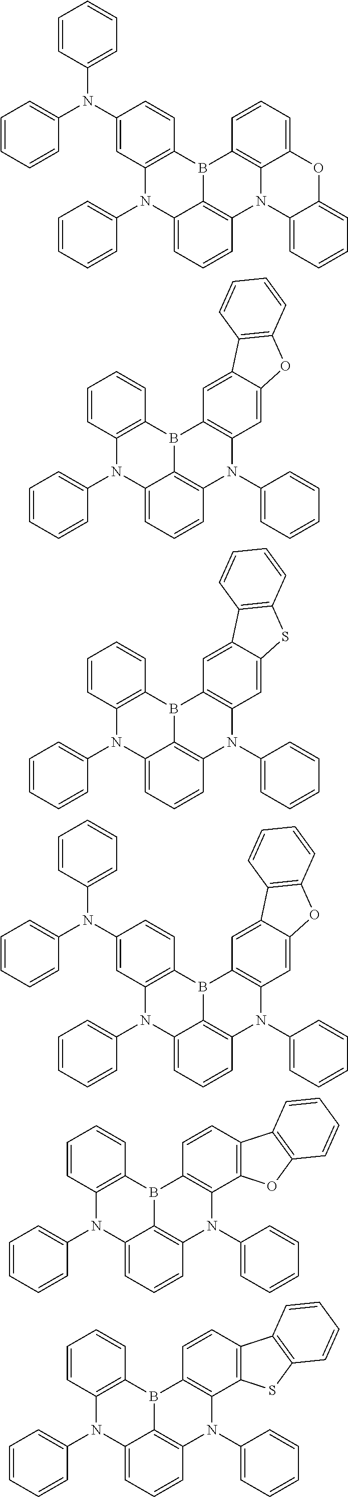 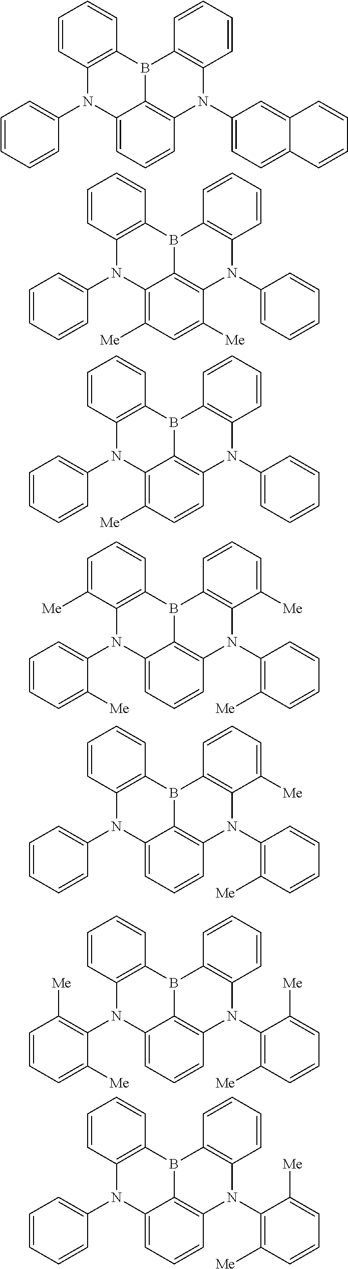 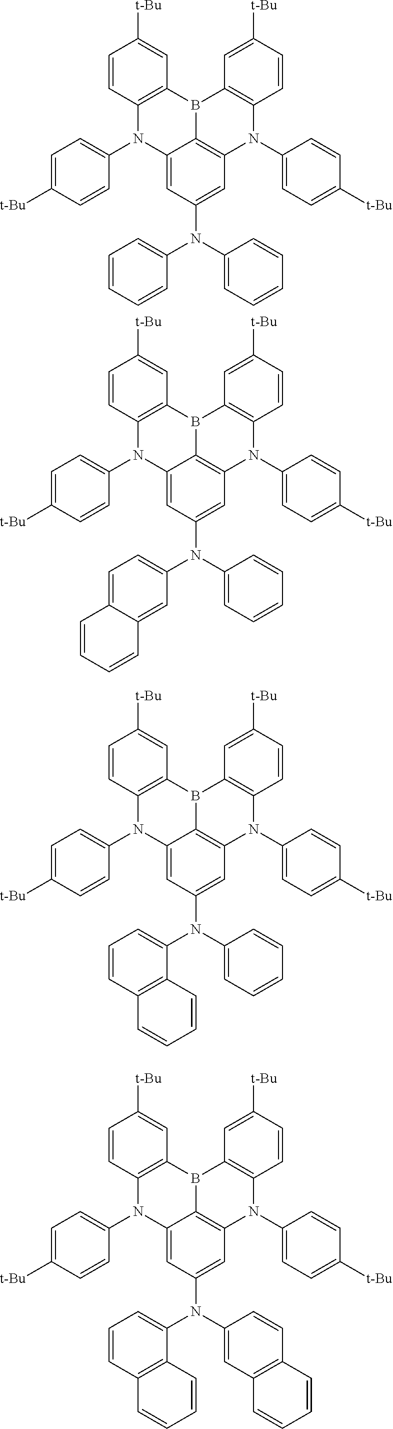 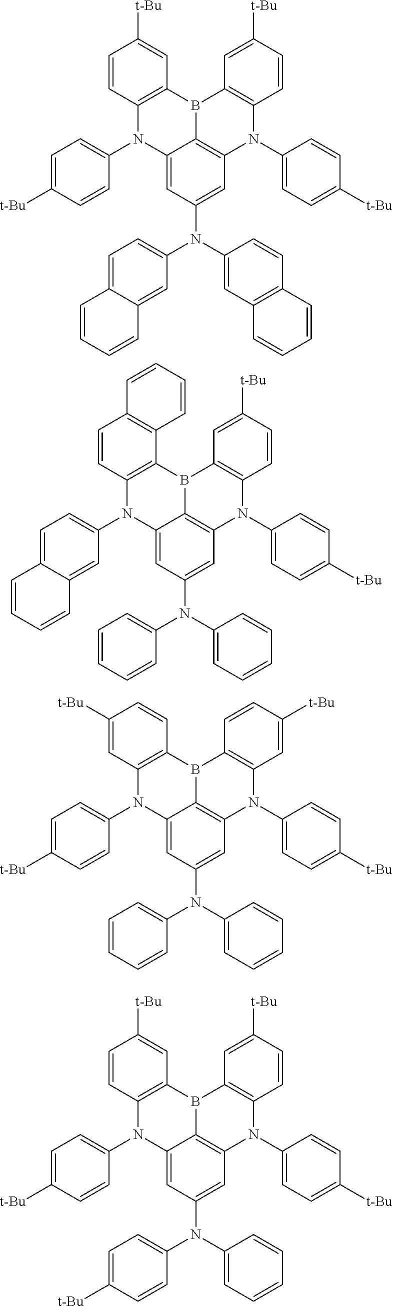 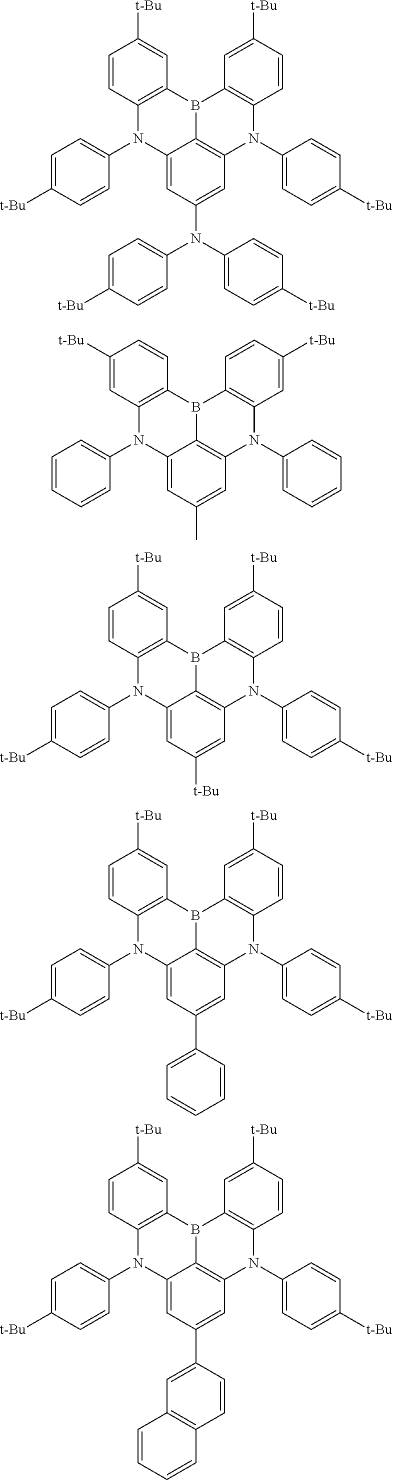 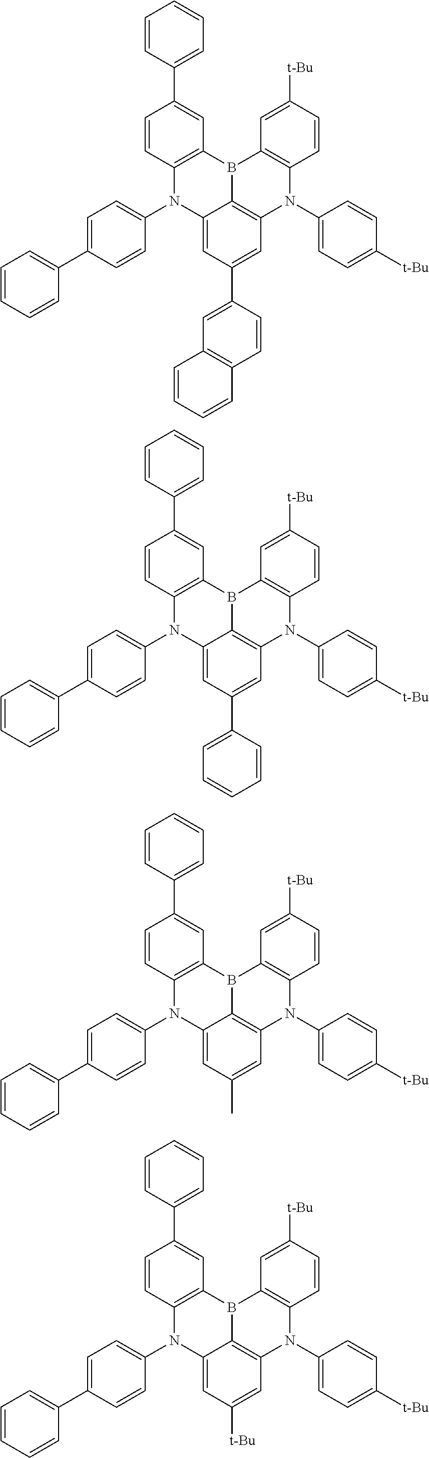 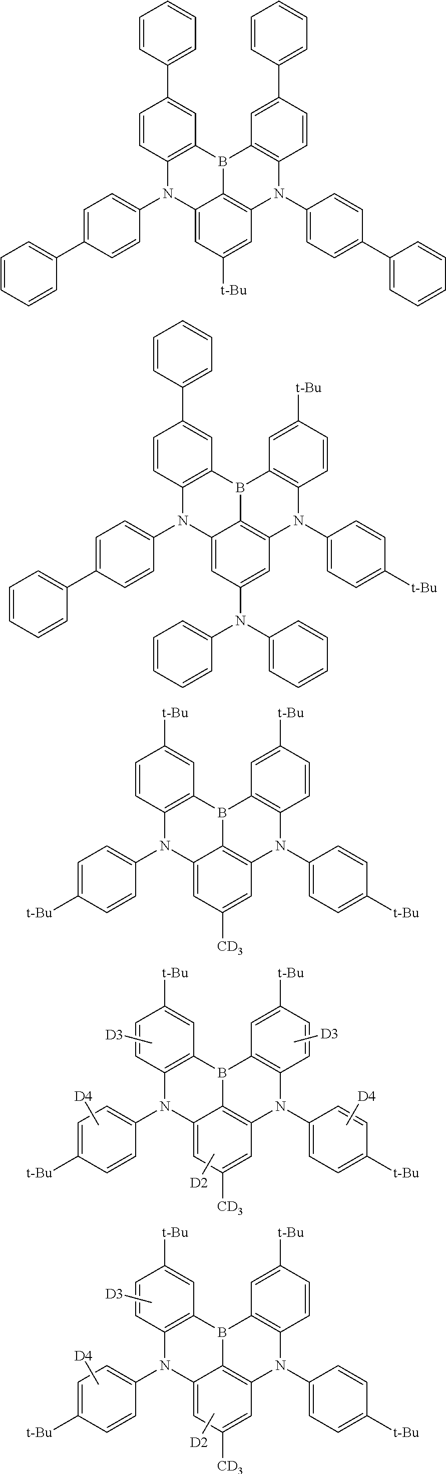 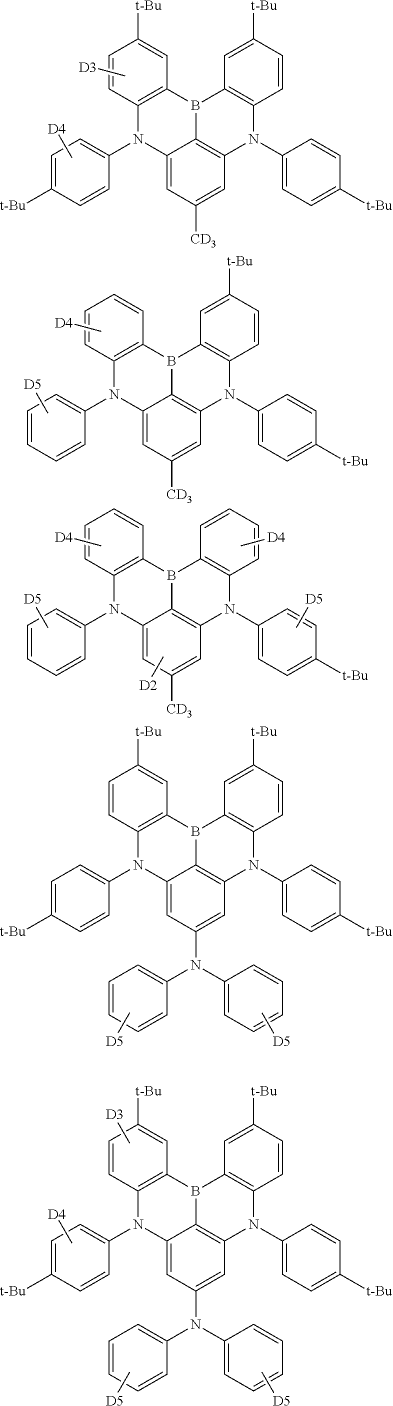 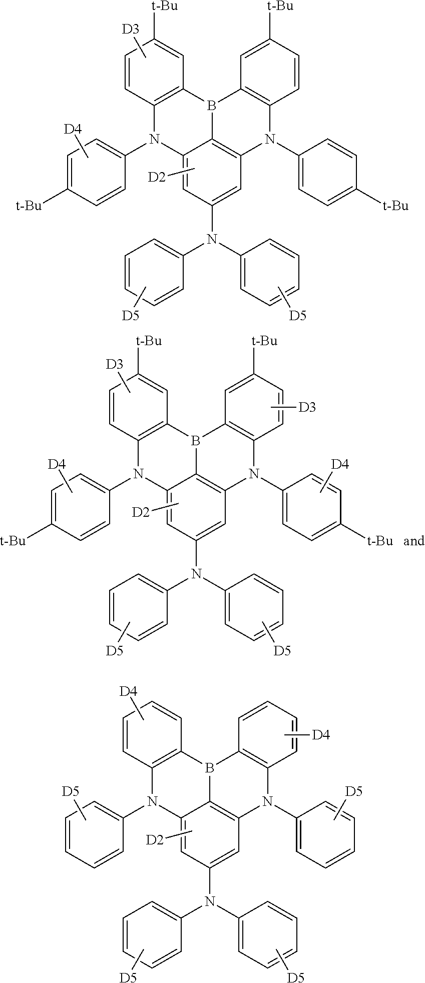
wherein
D2 to D5 represent that two to five hydrogens each have been replaced with deuterium.

10. The organic electroluminescent device according to claim 1, wherein the compound represented by formula 11 is selected from the following compounds: 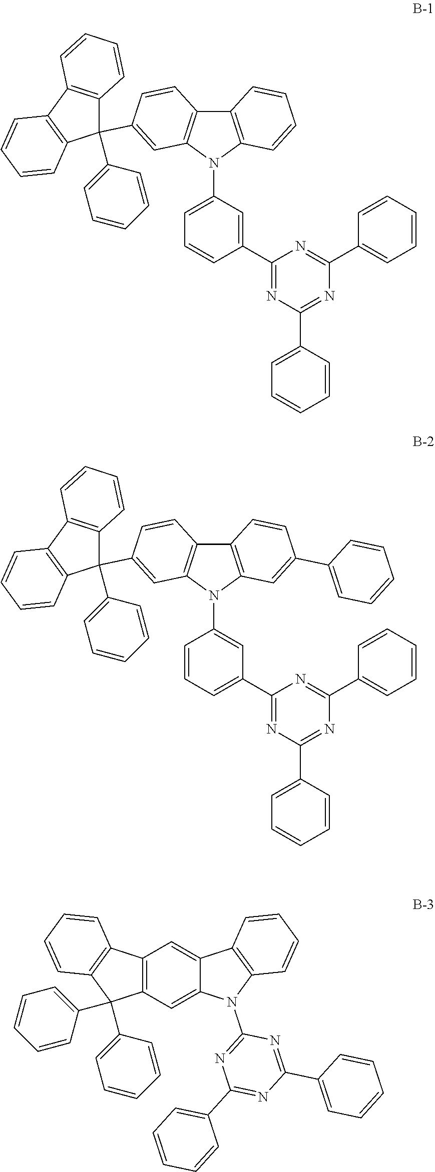 [SMILES: c1ccc(-c2nc(-c3ccccc3)nc(-c3cccc(-n4c5ccccc5c5ccc(C6(c7ccccc7)c7ccccc7-c7ccccc76)cc54)c3)n2)cc1 ; c1ccc(-c2ccc3c4ccc(C5(c6ccccc6)c6ccccc6-c6ccccc65)cc4n(-c4cccc(-c5nc(-c6ccccc6)nc(-c6ccccc6)n5)c4)c3c2)cc1 ; c1ccc(-c2nc(-c3ccccc3)nc(-n3c4ccccc4c4cc5c(cc43)C(c3ccccc3)(c3ccccc3)c3ccccc3-5)n2)cc1] 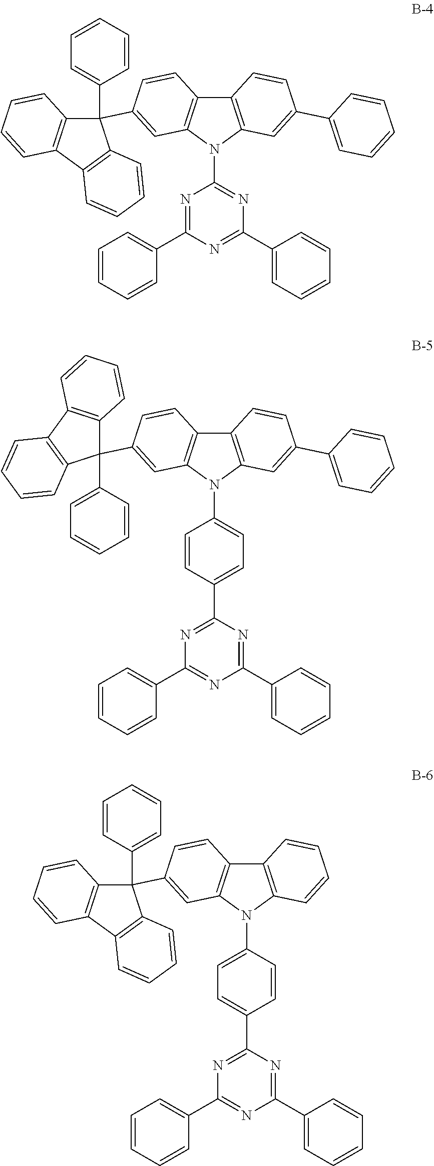 [SMILES: c1ccc(-c2ccc3c4ccc(C5(c6ccccc6)c6ccccc6-c6ccccc65)cc4n(-c4nc(-c5ccccc5)nc(-c5ccccc5)n4)c3c2)cc1 ; c1ccc(-c2ccc3c4ccc(C5(c6ccccc6)c6ccccc6-c6ccccc65)cc4n(-c4ccc(-c5nc(-c6ccccc6)nc(-c6ccccc6)n5)cc4)c3c2)cc1 ; c1ccc(-c2nc(-c3ccccc3)nc(-c3ccc(-n4c5ccccc5c5ccc(C6(c7ccccc7)c7ccccc7-c7ccccc76)cc54)cc3)n2)cc1] 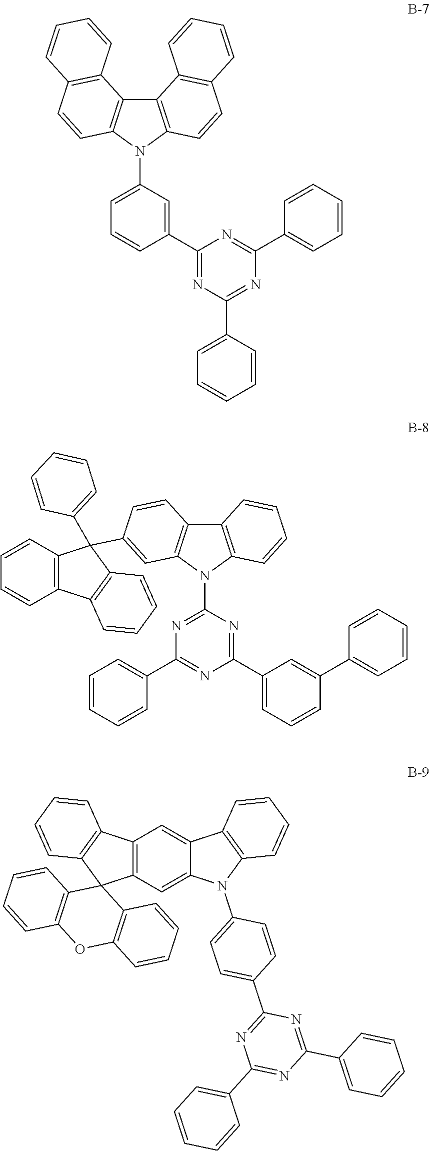 [SMILES: c1ccc(-c2nc(-c3ccccc3)nc(-c3cccc(-n4c5ccc6ccccc6c5c5c6ccccc6ccc54)c3)n2)cc1 ; c1ccc(-c2cccc(-c3nc(-c4ccccc4)nc(-n4c5ccccc5c5ccc(C6(c7ccccc7)c7ccccc7-c7ccccc76)cc54)n3)c2)cc1 ; c1ccc(-c2nc(-c3ccccc3)nc(-c3ccc(-n4c5ccccc5c5cc6c(cc54)C4(c5ccccc5Oc5ccccc54)c4ccccc4-6)cc3)n2)cc1] 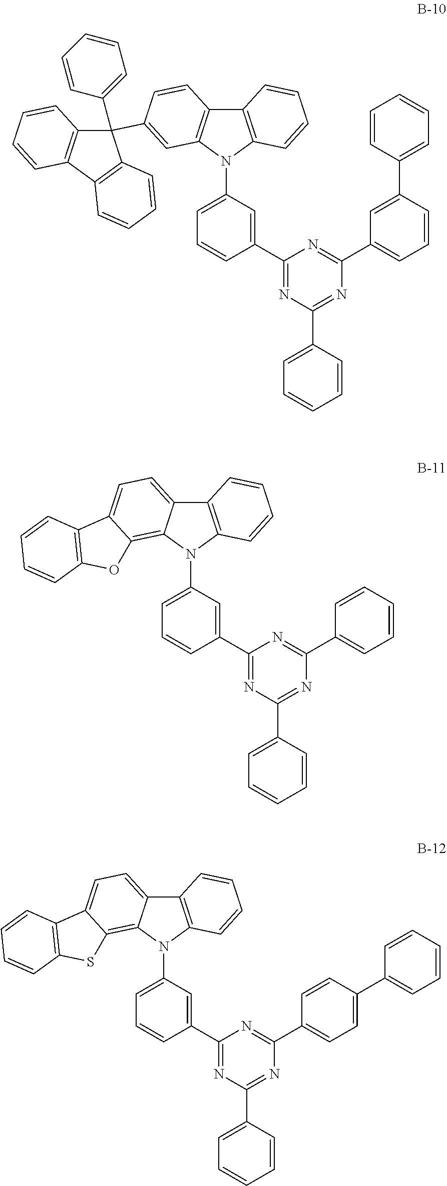 [SMILES: c1ccc(-c2cccc(-c3nc(-c4ccccc4)nc(-c4cccc(-n5c6ccccc6c6ccc(C7(c8ccccc8)c8ccccc8-c8ccccc87)cc65)c4)n3)c2)cc1 ; c1ccc(-c2nc(-c3ccccc3)nc(-c3cccc(-n4c5ccccc5c5ccc6c7ccccc7oc6c54)c3)n2)cc1 ; c1ccc(-c2ccc(-c3nc(-c4ccccc4)nc(-c4cccc(-n5c6ccccc6c6ccc7c8ccccc8sc7c65)c4)n3)cc2)cc1] 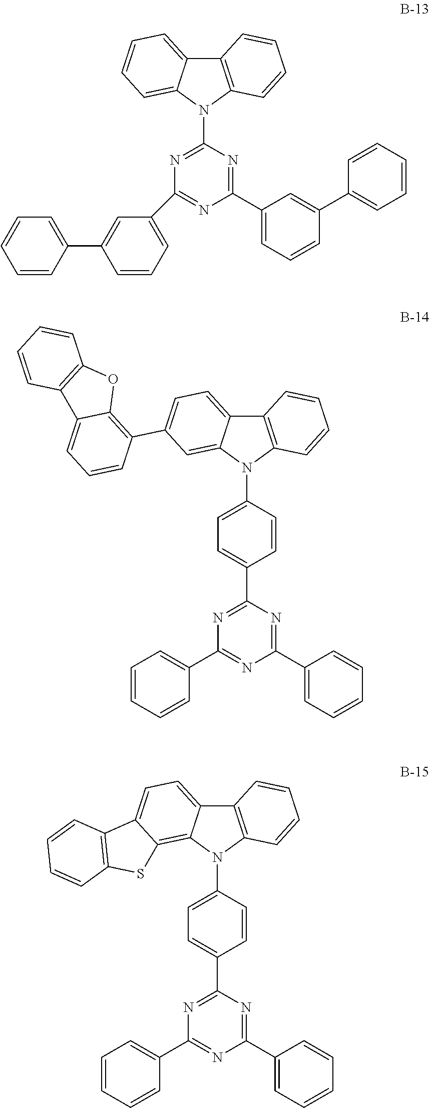 [SMILES: c1ccc(-c2cccc(-c3nc(-c4cccc(-c5ccccc5)c4)nc(-n4c5ccccc5c5ccccc54)n3)c2)cc1 ; c1ccc(-c2nc(-c3ccccc3)nc(-c3ccc(-n4c5ccccc5c5ccc(-c6cccc7c6oc6ccccc67)cc54)cc3)n2)cc1 ; c1ccc(-c2nc(-c3ccccc3)nc(-c3ccc(-n4c5ccccc5c5ccc6c7ccccc7sc6c54)cc3)n2)cc1] 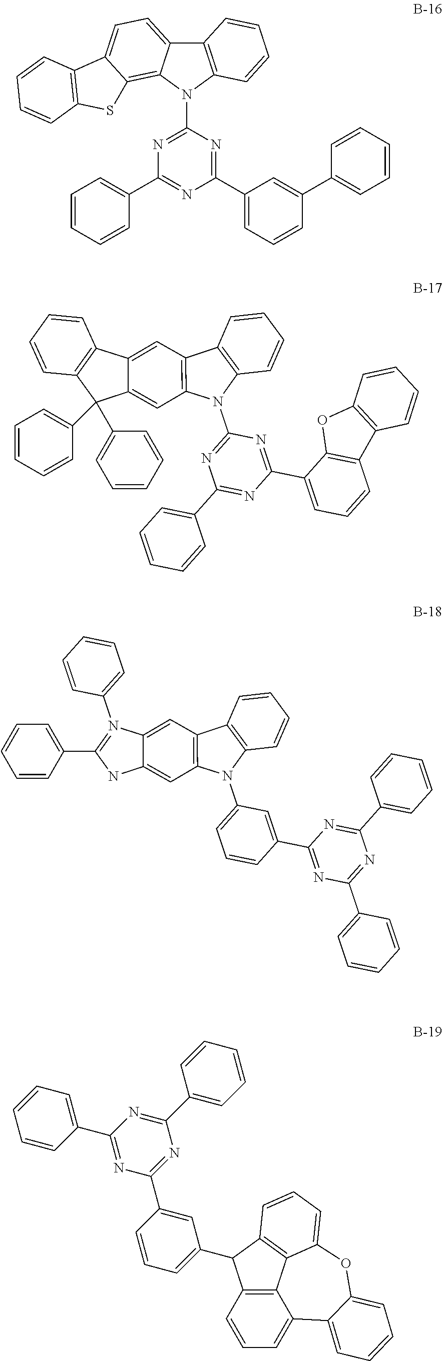 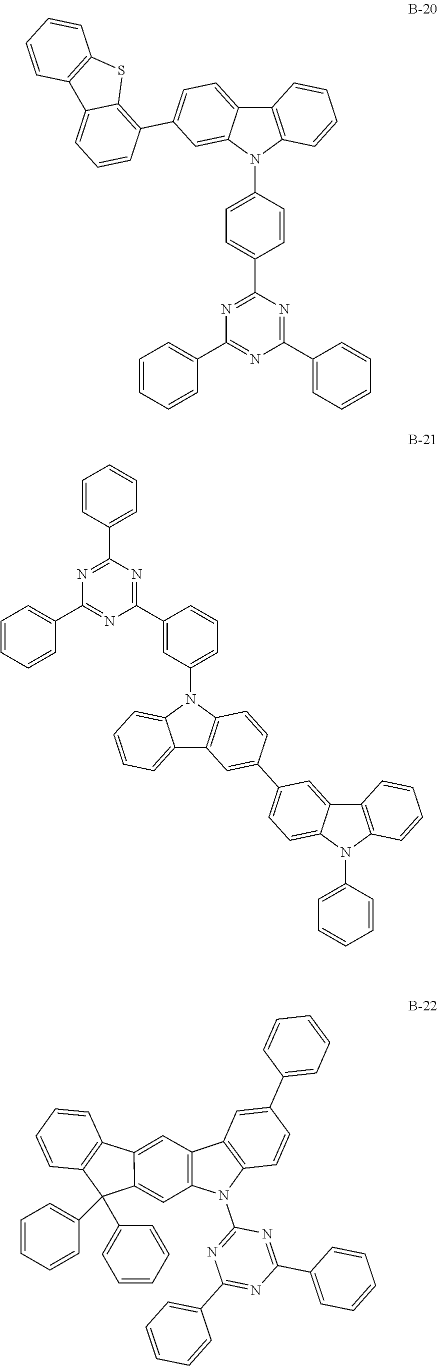 [SMILES: c1ccc(-c2nc(-c3ccccc3)nc(-c3ccc(-n4c5ccccc5c5ccc(-c6cccc7c6sc6ccccc67)cc54)cc3)n2)cc1 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc3c(c2)c2cc4c(cc2n3-c2nc(-c3ccccc3)nc(-c3ccccc3)n2)C(c2ccccc2)(c2ccccc2)c2ccccc2-4)cc1] 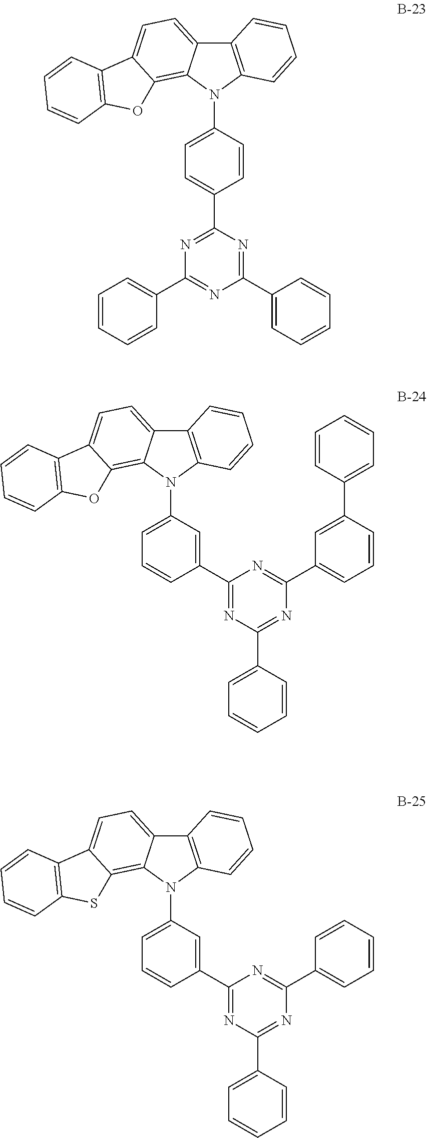 [SMILES: c1ccc(-c2nc(-c3ccccc3)nc(-c3ccc(-n4c5ccccc5c5ccc6c7ccccc7oc6c54)cc3)n2)cc1 ; c1ccc(-c2cccc(-c3nc(-c4ccccc4)nc(-c4cccc(-n5c6ccccc6c6ccc7c8ccccc8oc7c65)c4)n3)c2)cc1 ; c1ccc(-c2nc(-c3ccccc3)nc(-c3cccc(-n4c5ccccc5c5ccc6c7ccccc7sc6c54)c3)n2)cc1] 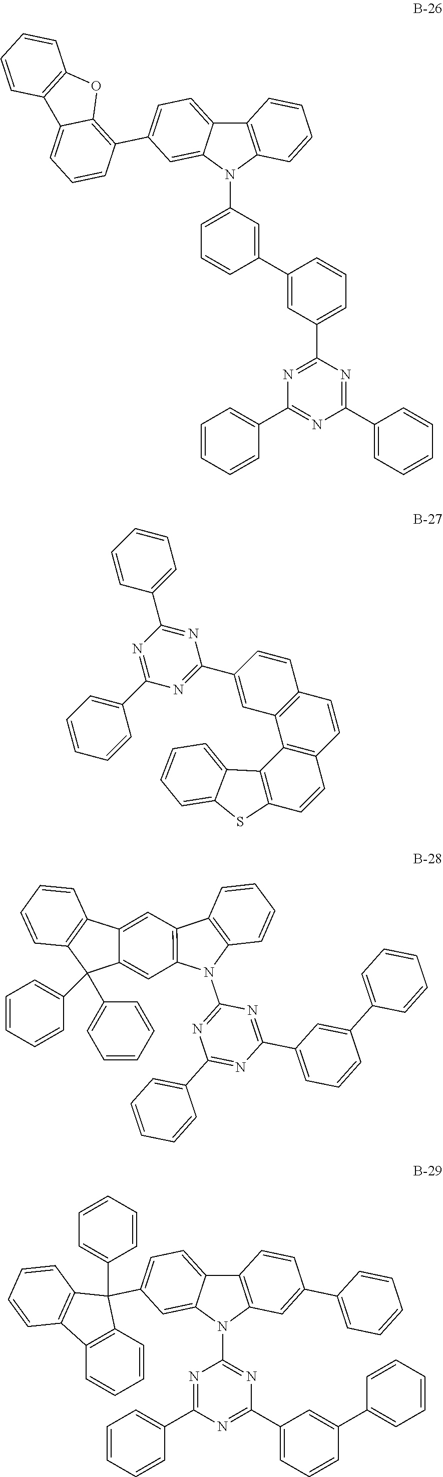 [SMILES: c1ccc(-c2nc(-c3ccccc3)nc(-c3cccc(-c4cccc(-n5c6ccccc6c6ccc(-c7cccc8c7oc7ccccc78)cc65)c4)c3)n2)cc1 ; c1ccc(-c2nc(-c3ccccc3)nc(-c3ccc4ccc5ccc6sc7ccccc7c6c5c4c3)n2)cc1 ; c1ccc(-c2cccc(-c3nc(-c4ccccc4)nc(-n4c5ccccc5c5cc6c(cc54)C(c4ccccc4)(c4ccccc4)c4ccccc4-6)n3)c2)cc1 ; c1ccc(-c2cccc(-c3nc(-c4ccccc4)nc(-n4c5cc(-c6ccccc6)ccc5c5ccc(C6(c7ccccc7)c7ccccc7-c7ccccc76)cc54)n3)c2)cc1] 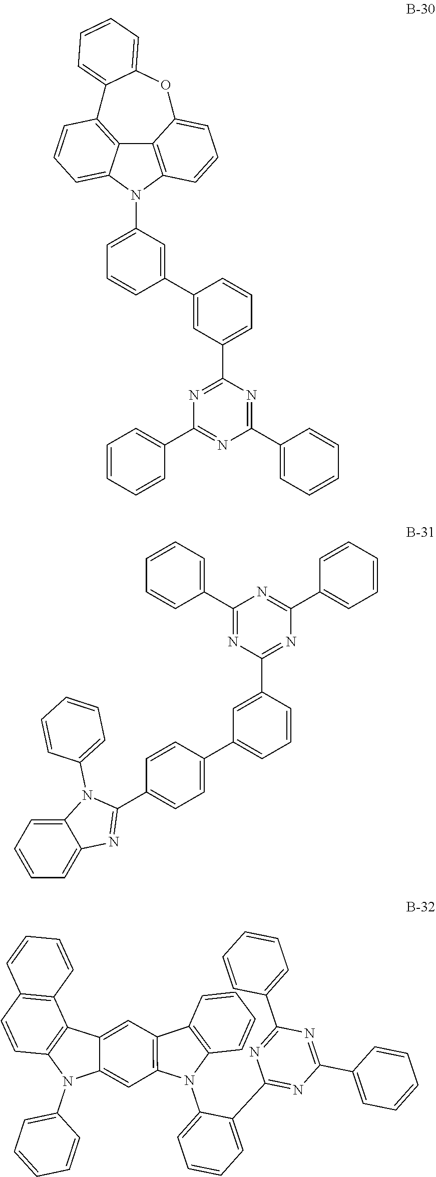 [SMILES: c1ccc(-c2nc(-c3ccccc3)nc(-c3cccc(-c4cccc(-n5c6cccc7oc8ccccc8c8cccc5c8c76)c4)c3)n2)cc1 ; c1ccc(-c2nc(-c3ccccc3)nc(-c3cccc(-c4ccc(-c5nc6ccccc6n5-c5ccccc5)cc4)c3)n2)cc1 ; c1ccc(-c2nc(-c3ccccc3)nc(-c3ccccc3-n3c4ccccc4c4cc5c6c7ccccc7ccc6n(-c6ccccc6)c5cc43)n2)cc1] 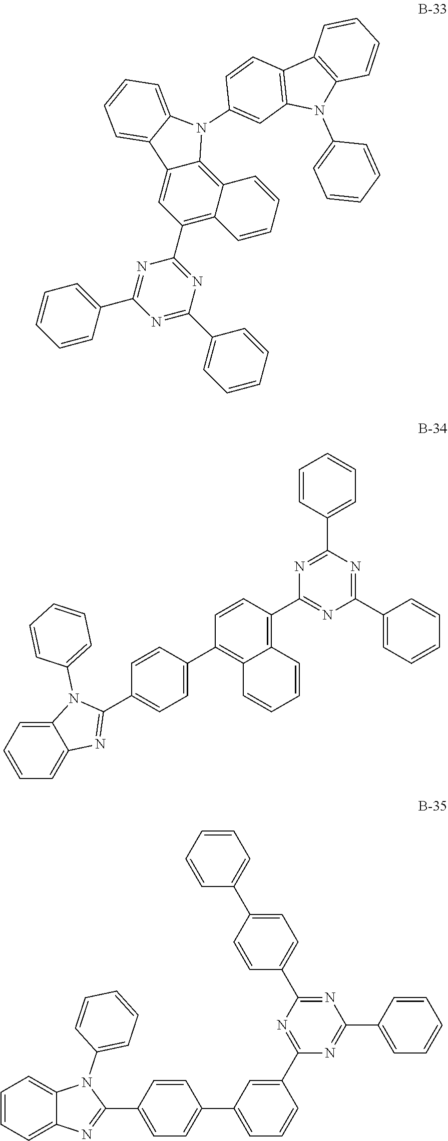 [SMILES: c1ccc(-c2nc(-c3ccccc3)nc(-c3cc4c5ccccc5n(-c5ccc6c7ccccc7n(-c7ccccc7)c6c5)c4c4ccccc34)n2)cc1 ; c1ccc(-c2nc(-c3ccccc3)nc(-c3ccc(-c4ccc(-c5nc6ccccc6n5-c5ccccc5)cc4)c4ccccc34)n2)cc1 ; c1ccc(-c2ccc(-c3nc(-c4ccccc4)nc(-c4cccc(-c5ccc(-c6nc7ccccc7n6-c6ccccc6)cc5)c4)n3)cc2)cc1] 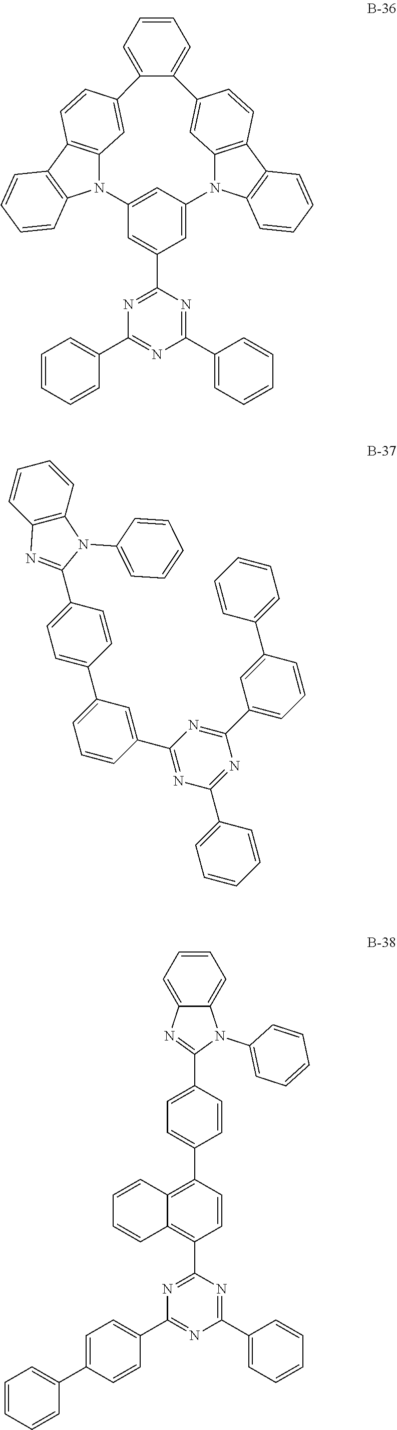 [SMILES: c1ccc(-c2nc(-c3ccccc3)nc(-c3cc4cc(c3)n3c5ccccc5c5ccc(cc53)c3ccccc3c3ccc5c6ccccc6n4c5c3)n2)cc1 ; c1ccc(-c2cccc(-c3nc(-c4ccccc4)nc(-c4cccc(-c5ccc(-c6nc7ccccc7n6-c6ccccc6)cc5)c4)n3)c2)cc1 ; c1ccc(-c2ccc(-c3nc(-c4ccccc4)nc(-c4ccc(-c5ccc(-c6nc7ccccc7n6-c6ccccc6)cc5)c5ccccc45)n3)cc2)cc1] 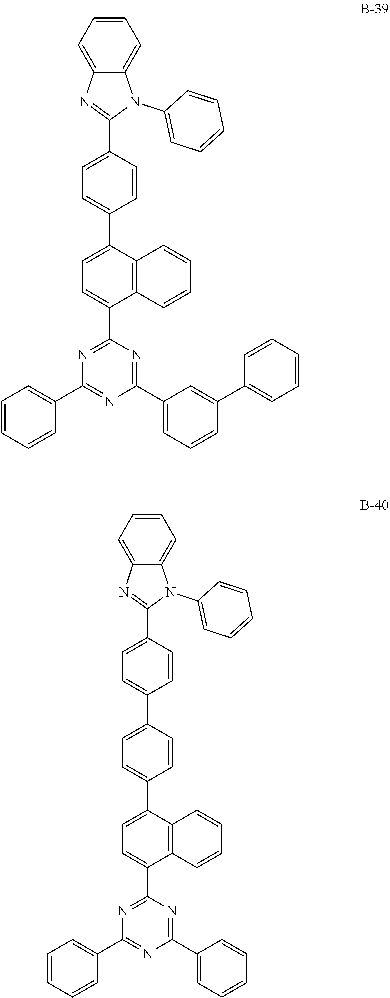 [SMILES: c1ccc(-c2cccc(-c3nc(-c4ccccc4)nc(-c4ccc(-c5ccc(-c6nc7ccccc7n6-c6ccccc6)cc5)c5ccccc45)n3)c2)cc1 ; c1ccc(-c2nc(-c3ccccc3)nc(-c3ccc(-c4ccc(-c5ccc(-c6nc7ccccc7n6-c6ccccc6)cc5)cc4)c4ccccc34)n2)cc1] 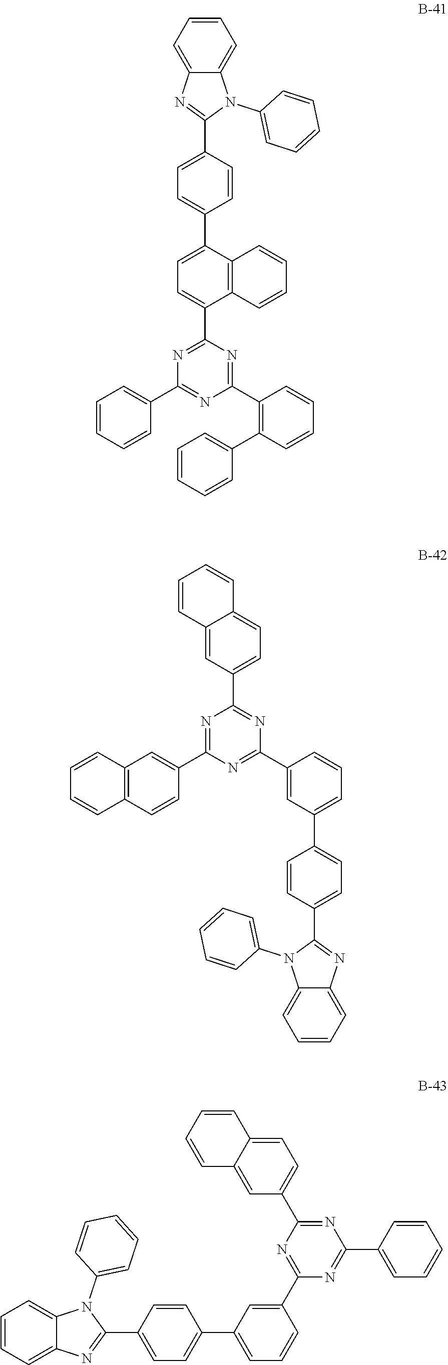 [SMILES: c1ccc(-c2nc(-c3ccccc3-c3ccccc3)nc(-c3ccc(-c4ccc(-c5nc6ccccc6n5-c5ccccc5)cc4)c4ccccc34)n2)cc1 ; c1ccc(-n2c(-c3ccc(-c4cccc(-c5nc(-c6ccc7ccccc7c6)nc(-c6ccc7ccccc7c6)n5)c4)cc3)nc3ccccc32)cc1 ; c1ccc(-c2nc(-c3cccc(-c4ccc(-c5nc6ccccc6n5-c5ccccc5)cc4)c3)nc(-c3ccc4ccccc4c3)n2)cc1] 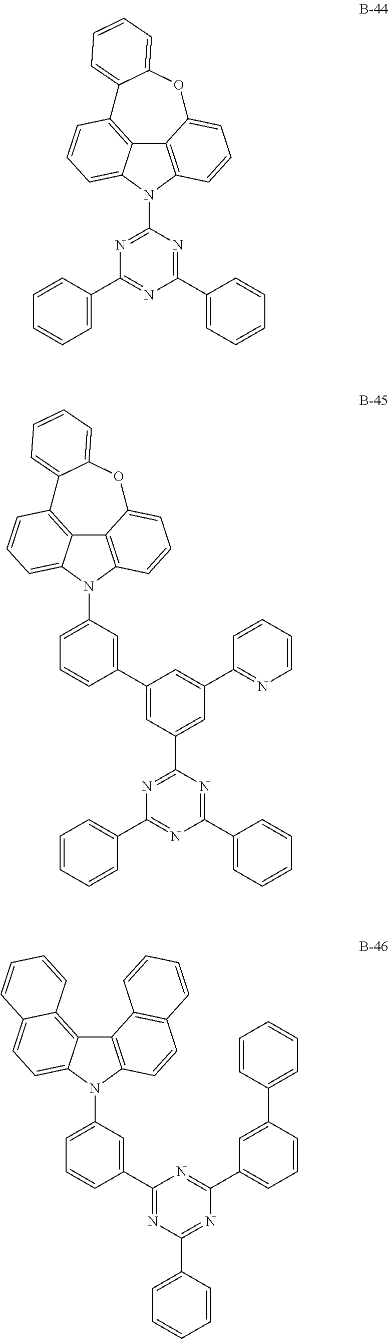 [SMILES: c1ccc(-c2nc(-c3ccccc3)nc(-n3c4cccc5oc6ccccc6c6cccc3c6c54)n2)cc1 ; c1ccc(-c2nc(-c3ccccc3)nc(-c3cc(-c4cccc(-n5c6cccc7oc8ccccc8c8cccc5c8c76)c4)cc(-c4ccccn4)c3)n2)cc1 ; c1ccc(-c2cccc(-c3nc(-c4ccccc4)nc(-c4cccc(-n5c6ccc7ccccc7c6c6c7ccccc7ccc65)c4)n3)c2)cc1] 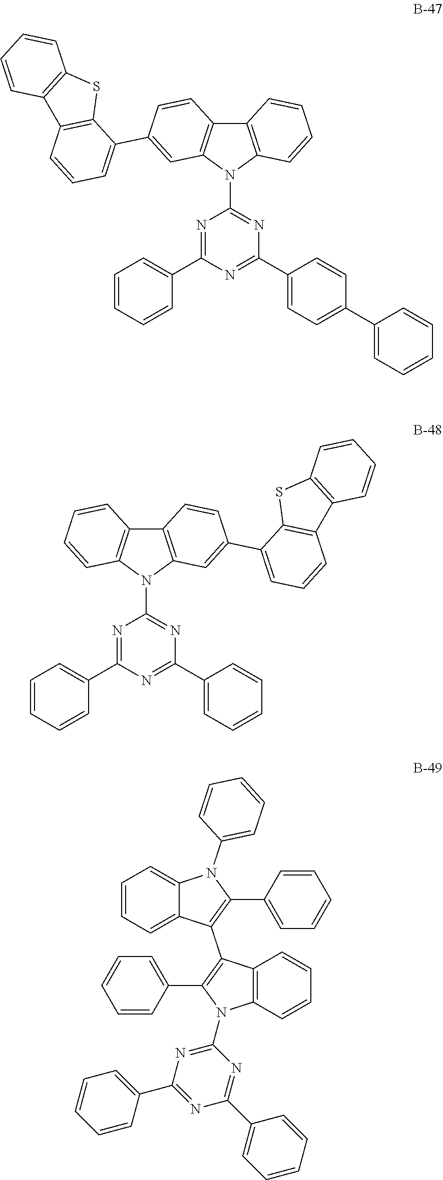 [SMILES: c1ccc(-c2ccc(-c3nc(-c4ccccc4)nc(-n4c5ccccc5c5ccc(-c6cccc7c6sc6ccccc67)cc54)n3)cc2)cc1 ; c1ccc(-c2nc(-c3ccccc3)nc(-n3c4ccccc4c4ccc(-c5cccc6c5sc5ccccc56)cc43)n2)cc1 ; c1ccc(-c2nc(-c3ccccc3)nc(-n3c(-c4ccccc4)c(-c4c(-c5ccccc5)n(-c5ccccc5)c5ccccc45)c4ccccc43)n2)cc1] 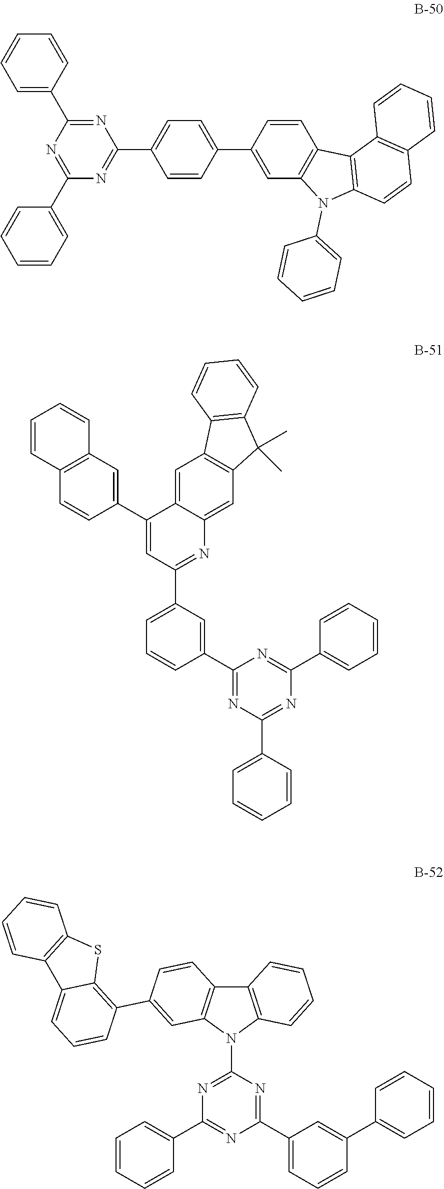 [SMILES: c1ccc(-c2nc(-c3ccccc3)nc(-c3ccc(-c4ccc5c6c7ccccc7ccc6n(-c6ccccc6)c5c4)cc3)n2)cc1 ; CC1(C)c2ccccc2-c2cc3c(-c4ccc5ccccc5c4)cc(-c4cccc(-c5nc(-c6ccccc6)nc(-c6ccccc6)n5)c4)nc3cc21 ; c1ccc(-c2cccc(-c3nc(-c4ccccc4)nc(-n4c5ccccc5c5ccc(-c6cccc7c6sc6ccccc67)cc54)n3)c2)cc1] 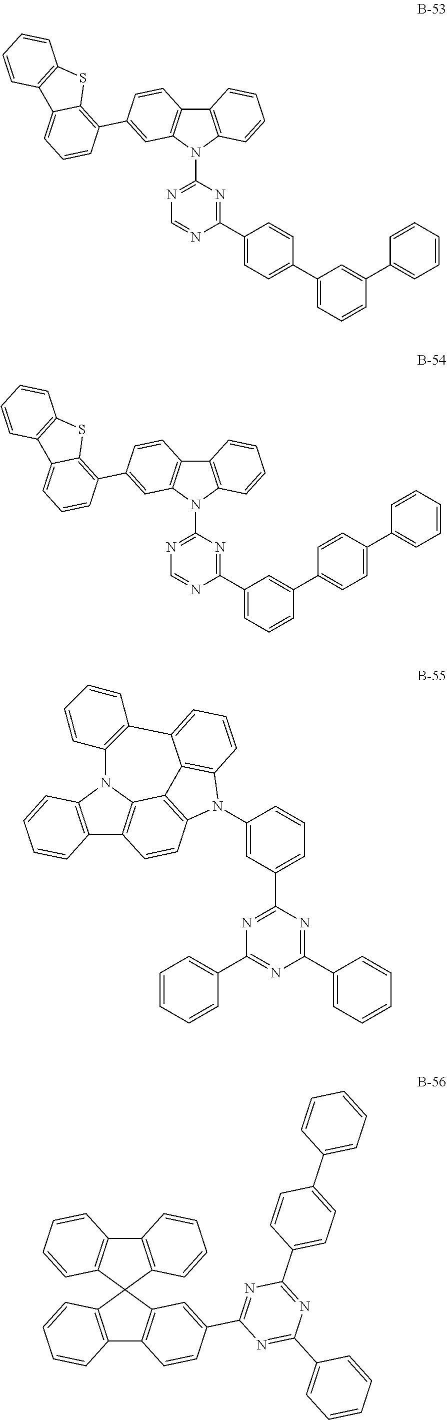 [SMILES: c1ccc(-c2cccc(-c3ccc(-c4ncnc(-n5c6ccccc6c6ccc(-c7cccc8c7sc7ccccc78)cc65)n4)cc3)c2)cc1 ; c1ccc(-c2ccc(-c3cccc(-c4ncnc(-n5c6ccccc6c6ccc(-c7cccc8c7sc7ccccc78)cc65)n4)c3)cc2)cc1 ; c1ccc(-c2nc(-c3ccccc3)nc(-c3cccc(-n4c5cccc6c7ccccc7n7c8ccccc8c8ccc4c(c65)c87)c3)n2)cc1 ; c1ccc(-c2ccc(-c3nc(-c4ccccc4)nc(-c4ccc5c(c4)C4(c6ccccc6-c6ccccc64)c4ccccc4-5)n3)cc2)cc1] 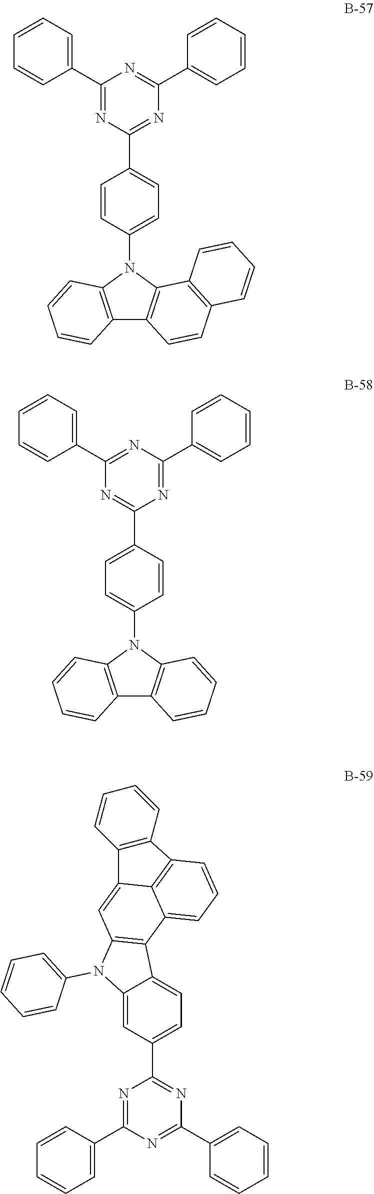 [SMILES: c1ccc(-c2nc(-c3ccccc3)nc(-c3ccc(-n4c5ccccc5c5ccc6ccccc6c54)cc3)n2)cc1 ; c1ccc(-c2nc(-c3ccccc3)nc(-c3ccc(-n4c5ccccc5c5ccccc54)cc3)n2)cc1 ; c1ccc(-c2nc(-c3ccccc3)nc(-c3ccc4c5c6cccc7c6c(cc5n(-c5ccccc5)c4c3)-c3ccccc3-7)n2)cc1] 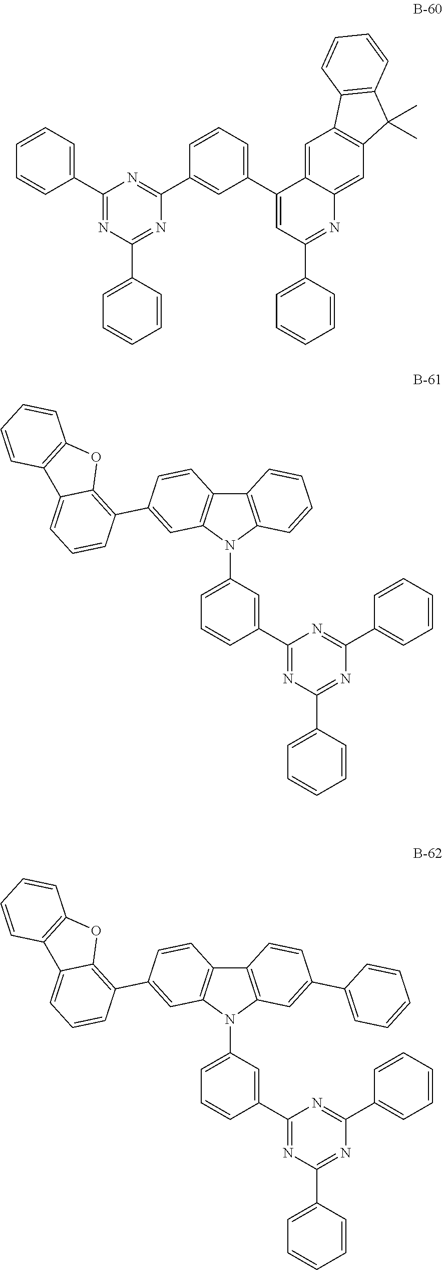 [SMILES: CC1(C)c2ccccc2-c2cc3c(-c4cccc(-c5nc(-c6ccccc6)nc(-c6ccccc6)n5)c4)cc(-c4ccccc4)nc3cc21 ; c1ccc(-c2nc(-c3ccccc3)nc(-c3cccc(-n4c5ccccc5c5ccc(-c6cccc7c6oc6ccccc67)cc54)c3)n2)cc1 ; c1ccc(-c2ccc3c4ccc(-c5cccc6c5oc5ccccc56)cc4n(-c4cccc(-c5nc(-c6ccccc6)nc(-c6ccccc6)n5)c4)c3c2)cc1] 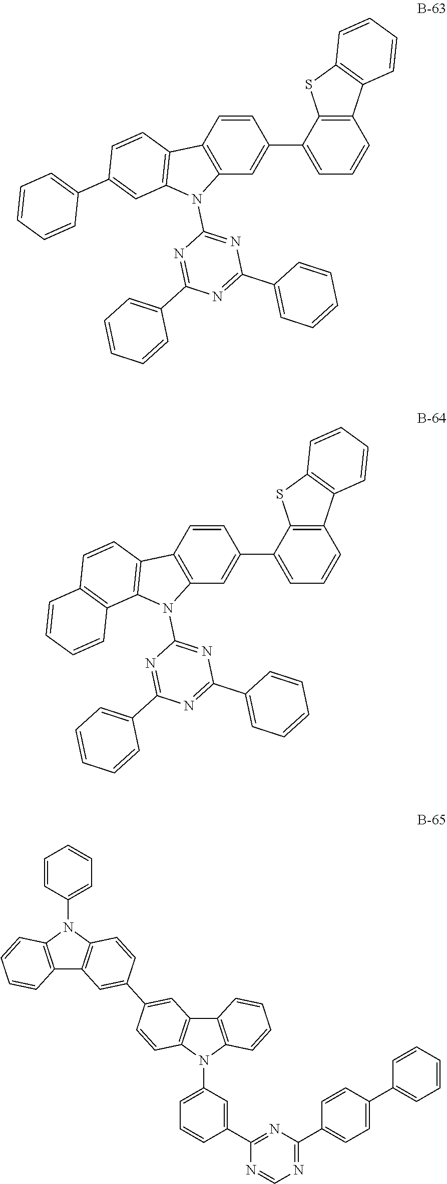 [SMILES: c1ccc(-c2ccc3c4ccc(-c5cccc6c5sc5ccccc56)cc4n(-c4nc(-c5ccccc5)nc(-c5ccccc5)n4)c3c2)cc1 ; c1ccc(-c2nc(-c3ccccc3)nc(-n3c4cc(-c5cccc6c5sc5ccccc56)ccc4c4ccc5ccccc5c43)n2)cc1 ; c1ccc(-c2ccc(-c3ncnc(-c4cccc(-n5c6ccccc6c6cc(-c7ccc8c(c7)c7ccccc7n8-c7ccccc7)ccc65)c4)n3)cc2)cc1] 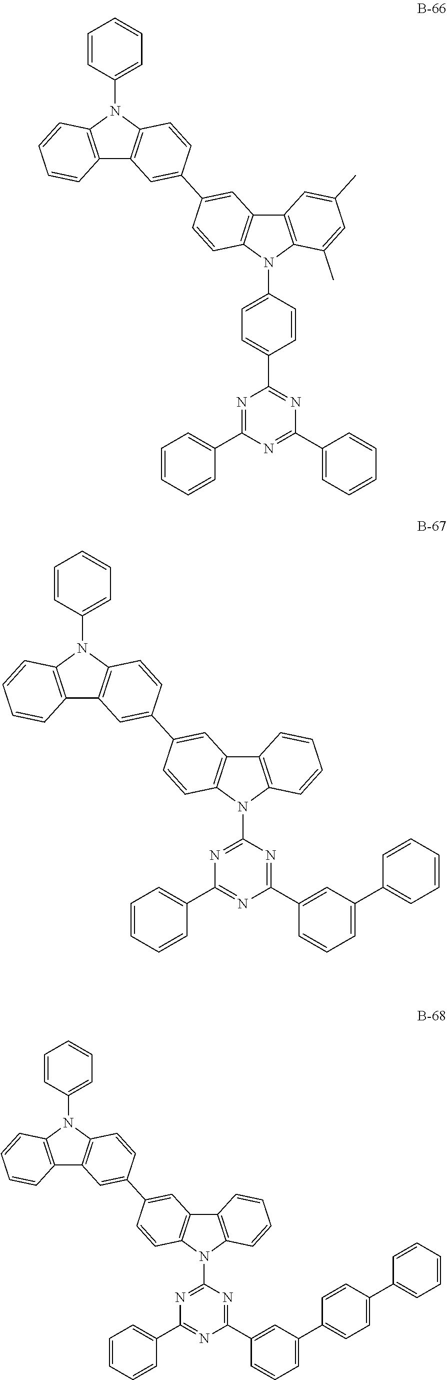 [SMILES: Cc1cc(C)c2c(c1)c1cc(-c3ccc4c(c3)c3ccccc3n4-c3ccccc3)ccc1n2-c1ccc(-c2nc(-c3ccccc3)nc(-c3ccccc3)n2)cc1 ; c1ccc(-c2cccc(-c3nc(-c4ccccc4)nc(-n4c5ccccc5c5cc(-c6ccc7c(c6)c6ccccc6n7-c6ccccc6)ccc54)n3)c2)cc1 ; c1ccc(-c2ccc(-c3cccc(-c4nc(-c5ccccc5)nc(-n5c6ccccc6c6cc(-c7ccc8c(c7)c7ccccc7n8-c7ccccc7)ccc65)n4)c3)cc2)cc1] 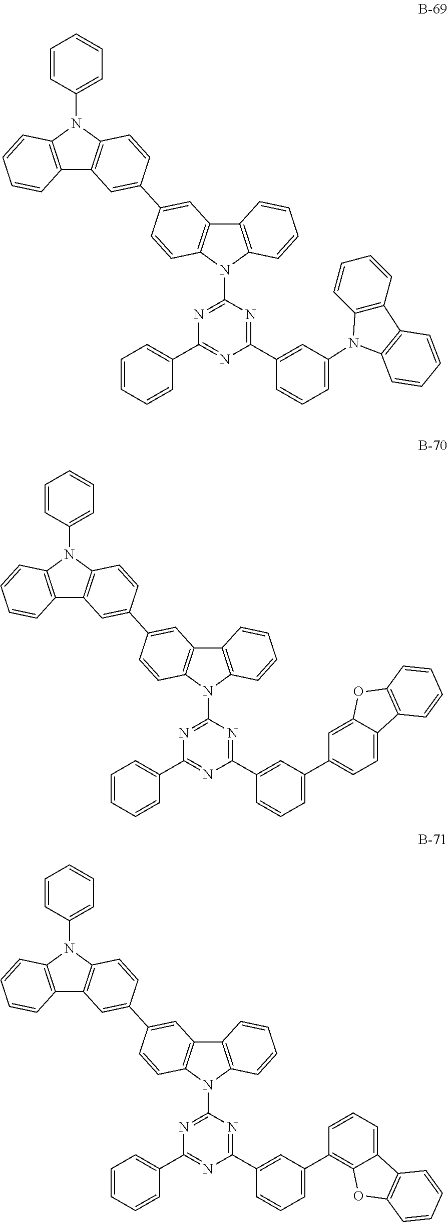 [SMILES: c1ccc(-c2nc(-c3cccc(-n4c5ccccc5c5ccccc54)c3)nc(-n3c4ccccc4c4cc(-c5ccc6c(c5)c5ccccc5n6-c5ccccc5)ccc43)n2)cc1 ; c1ccc(-c2nc(-c3cccc(-c4ccc5c(c4)oc4ccccc45)c3)nc(-n3c4ccccc4c4cc(-c5ccc6c(c5)c5ccccc5n6-c5ccccc5)ccc43)n2)cc1 ; c1ccc(-c2nc(-c3cccc(-c4cccc5c4oc4ccccc45)c3)nc(-n3c4ccccc4c4cc(-c5ccc6c(c5)c5ccccc5n6-c5ccccc5)ccc43)n2)cc1] 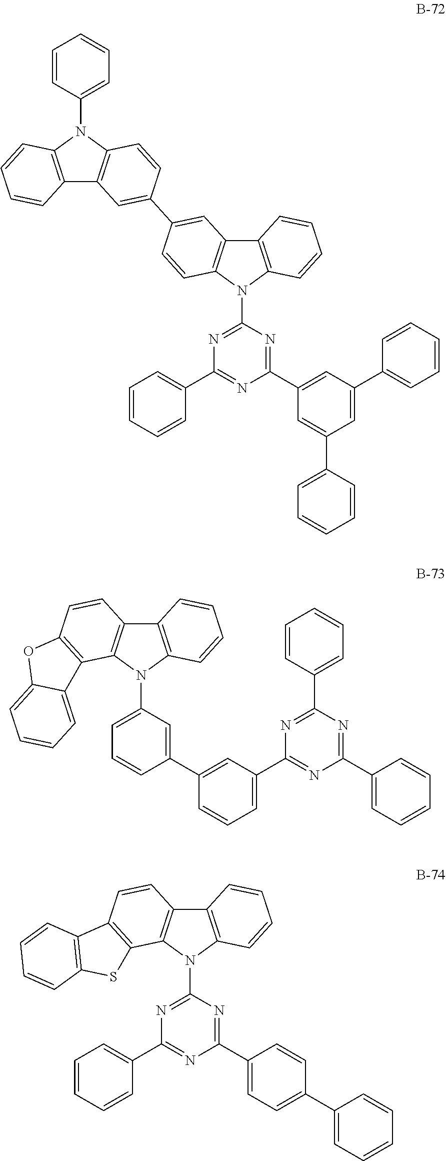 [SMILES: c1ccc(-c2cc(-c3ccccc3)cc(-c3nc(-c4ccccc4)nc(-n4c5ccccc5c5cc(-c6ccc7c(c6)c6ccccc6n7-c6ccccc6)ccc54)n3)c2)cc1 ; c1ccc(-c2nc(-c3ccccc3)nc(-c3cccc(-c4cccc(-n5c6ccccc6c6ccc7oc8ccccc8c7c65)c4)c3)n2)cc1 ; c1ccc(-c2ccc(-c3nc(-c4ccccc4)nc(-n4c5ccccc5c5ccc6c7ccccc7sc6c54)n3)cc2)cc1] 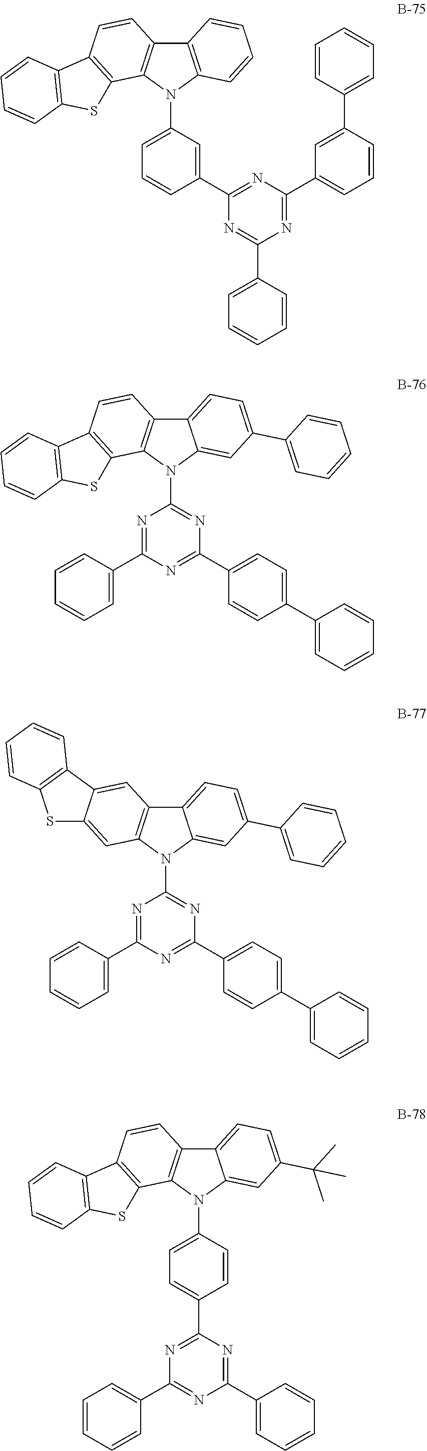 [SMILES: c1ccc(-c2cccc(-c3nc(-c4ccccc4)nc(-c4cccc(-n5c6ccccc6c6ccc7c8ccccc8sc7c65)c4)n3)c2)cc1 ; c1ccc(-c2ccc(-c3nc(-c4ccccc4)nc(-n4c5cc(-c6ccccc6)ccc5c5ccc6c7ccccc7sc6c54)n3)cc2)cc1 ; c1ccc(-c2ccc(-c3nc(-c4ccccc4)nc(-n4c5cc(-c6ccccc6)ccc5c5cc6c(cc54)sc4ccccc46)n3)cc2)cc1 ; CC(C)(C)c1ccc2c3ccc4c5ccccc5sc4c3n(-c3ccc(-c4nc(-c5ccccc5)nc(-c5ccccc5)n4)cc3)c2c1] 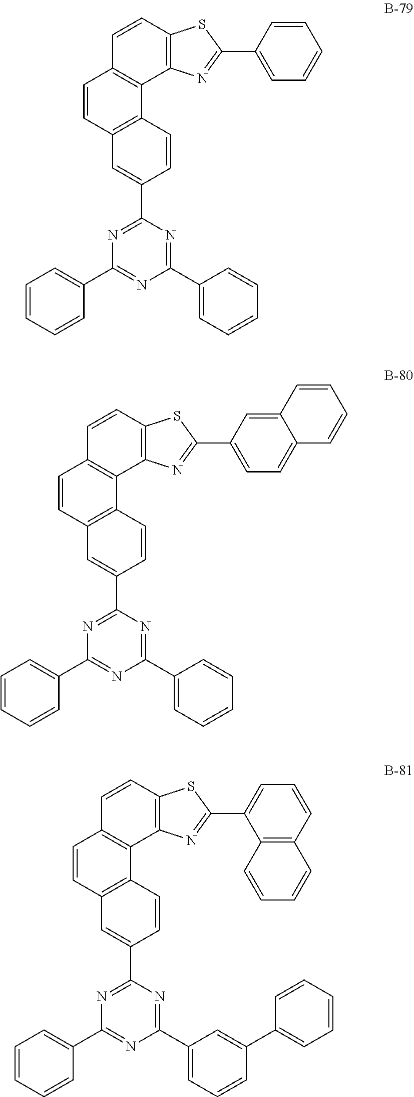 [SMILES: c1ccc(-c2nc(-c3ccccc3)nc(-c3ccc4c(ccc5ccc6sc(-c7ccccc7)nc6c54)c3)n2)cc1 ; c1ccc(-c2nc(-c3ccccc3)nc(-c3ccc4c(ccc5ccc6sc(-c7ccc8ccccc8c7)nc6c54)c3)n2)cc1 ; c1ccc(-c2cccc(-c3nc(-c4ccccc4)nc(-c4ccc5c(ccc6ccc7sc(-c8cccc9ccccc89)nc7c65)c4)n3)c2)cc1] 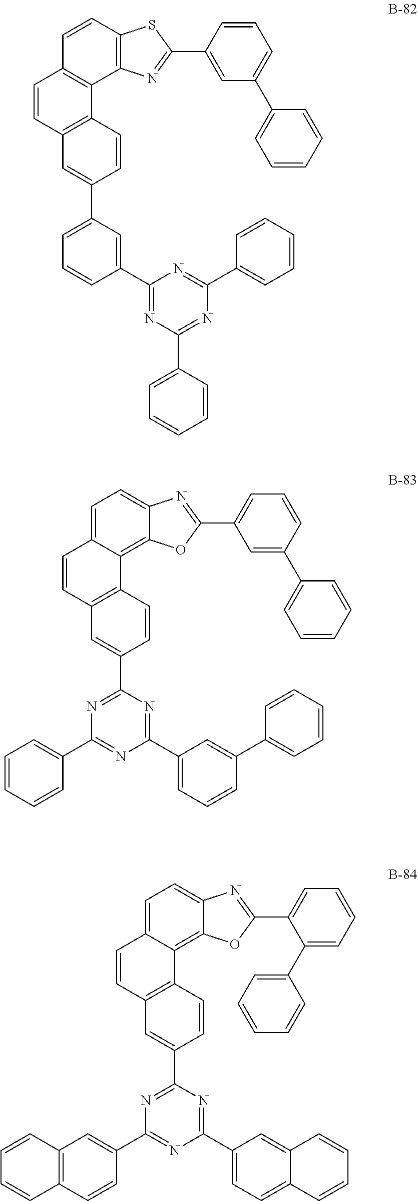 [SMILES: c1ccc(-c2cccc(-c3nc4c(ccc5ccc6cc(-c7cccc(-c8nc(-c9ccccc9)nc(-c9ccccc9)n8)c7)ccc6c54)s3)c2)cc1 ; c1ccc(-c2cccc(-c3nc(-c4ccccc4)nc(-c4ccc5c(ccc6ccc7nc(-c8cccc(-c9ccccc9)c8)oc7c65)c4)n3)c2)cc1 ; c1ccc(-c2ccccc2-c2nc3ccc4ccc5cc(-c6nc(-c7ccc8ccccc8c7)nc(-c7ccc8ccccc8c7)n6)ccc5c4c3o2)cc1] 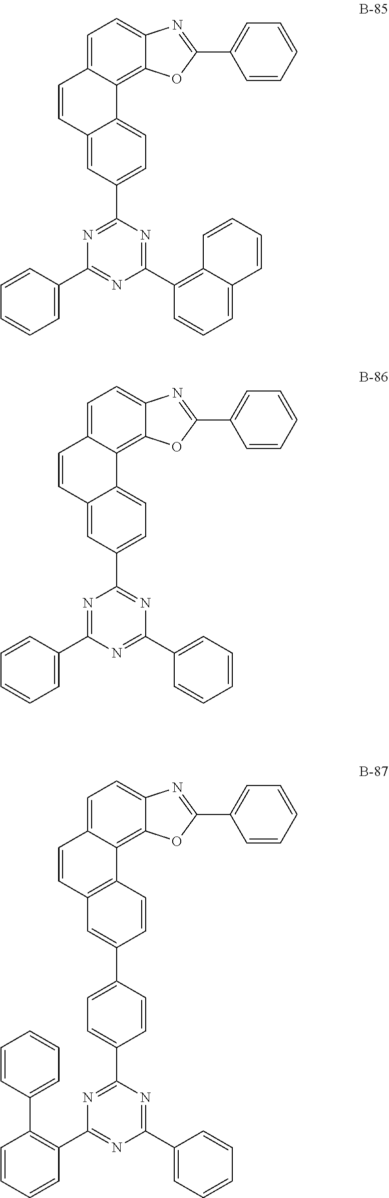 [SMILES: c1ccc(-c2nc(-c3ccc4c(ccc5ccc6nc(-c7ccccc7)oc6c54)c3)nc(-c3cccc4ccccc34)n2)cc1 ; c1ccc(-c2nc(-c3ccccc3)nc(-c3ccc4c(ccc5ccc6nc(-c7ccccc7)oc6c54)c3)n2)cc1 ; c1ccc(-c2nc(-c3ccc(-c4ccc5c(ccc6ccc7nc(-c8ccccc8)oc7c65)c4)cc3)nc(-c3ccccc3-c3ccccc3)n2)cc1] 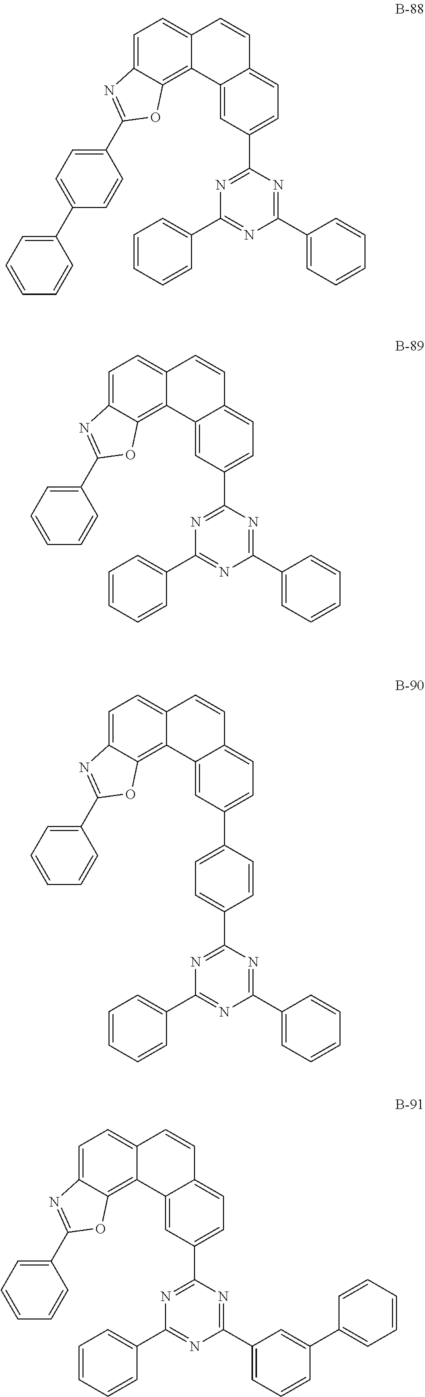 [SMILES: c1ccc(-c2ccc(-c3nc4ccc5ccc6ccc(-c7nc(-c8ccccc8)nc(-c8ccccc8)n7)cc6c5c4o3)cc2)cc1 ; c1ccc(-c2nc(-c3ccccc3)nc(-c3ccc4ccc5ccc6nc(-c7ccccc7)oc6c5c4c3)n2)cc1 ; c1ccc(-c2nc(-c3ccccc3)nc(-c3ccc(-c4ccc5ccc6ccc7nc(-c8ccccc8)oc7c6c5c4)cc3)n2)cc1 ; c1ccc(-c2cccc(-c3nc(-c4ccccc4)nc(-c4ccc5ccc6ccc7nc(-c8ccccc8)oc7c6c5c4)n3)c2)cc1] 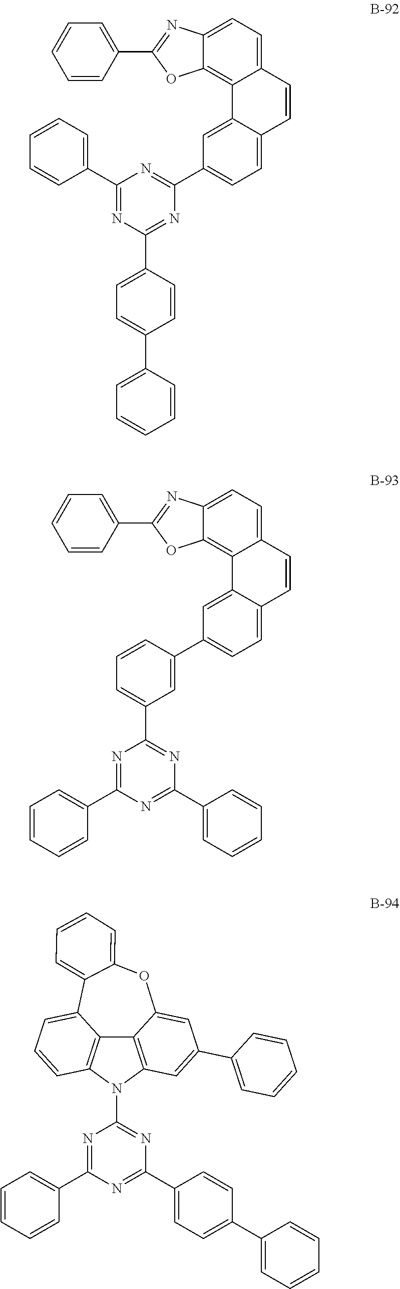 [SMILES: c1ccc(-c2ccc(-c3nc(-c4ccccc4)nc(-c4ccc5ccc6ccc7nc(-c8ccccc8)oc7c6c5c4)n3)cc2)cc1 ; c1ccc(-c2nc(-c3ccccc3)nc(-c3cccc(-c4ccc5ccc6ccc7nc(-c8ccccc8)oc7c6c5c4)c3)n2)cc1 ; c1ccc(-c2ccc(-c3nc(-c4ccccc4)nc(-n4c5cc(-c6ccccc6)cc6oc7ccccc7c7cccc4c7c65)n3)cc2)cc1] 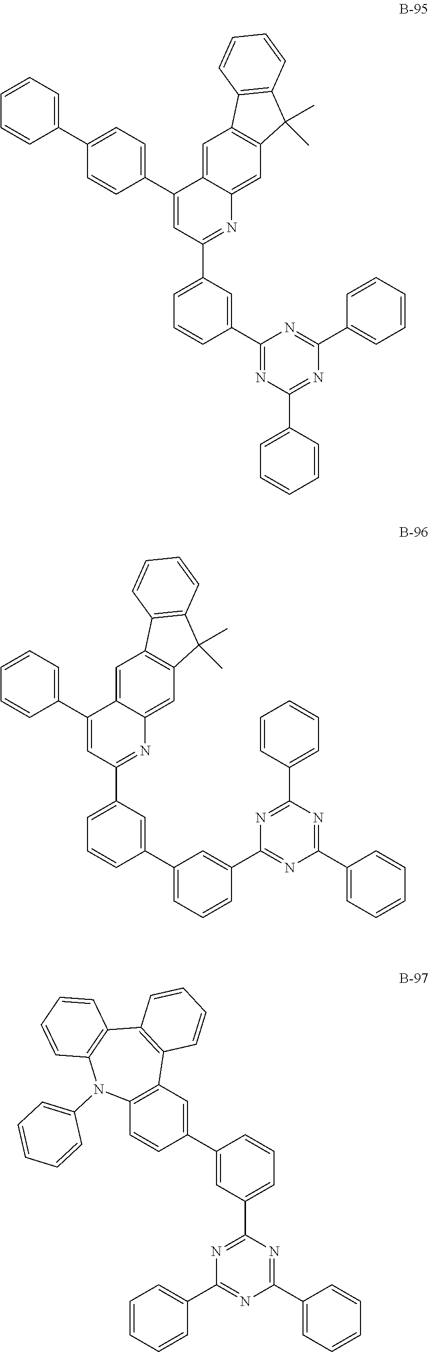 [SMILES: CC1(C)c2ccccc2-c2cc3c(-c4ccc(-c5ccccc5)cc4)cc(-c4cccc(-c5nc(-c6ccccc6)nc(-c6ccccc6)n5)c4)nc3cc21 ; CC1(C)c2ccccc2-c2cc3c(-c4ccccc4)cc(-c4cccc(-c5cccc(-c6nc(-c7ccccc7)nc(-c7ccccc7)n6)c5)c4)nc3cc21 ; c1ccc(-c2nc(-c3ccccc3)nc(-c3cccc(-c4ccc5c(c4)-c4ccccc4-c4ccccc4N5c4ccccc4)c3)n2)cc1] 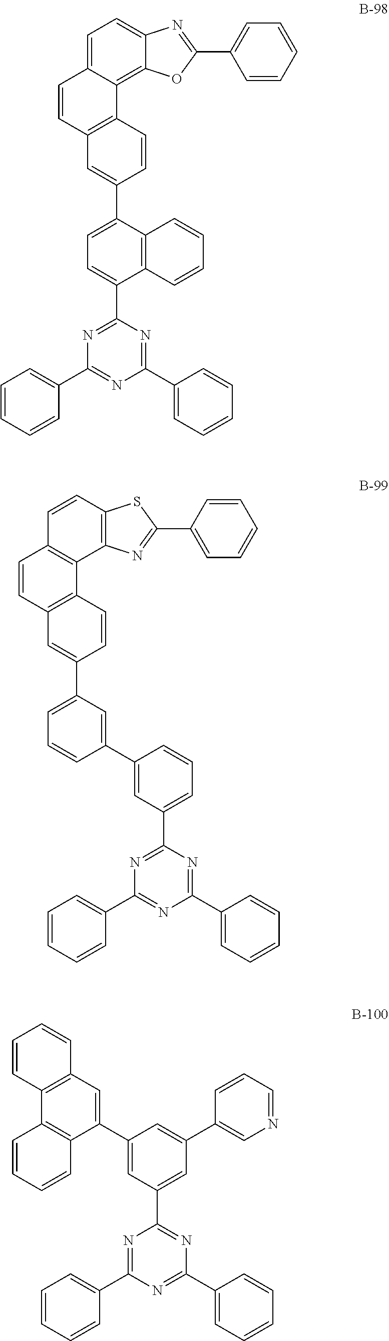 [SMILES: c1ccc(-c2nc(-c3ccccc3)nc(-c3ccc(-c4ccc5c(ccc6ccc7nc(-c8ccccc8)oc7c65)c4)c4ccccc34)n2)cc1 ; c1ccc(-c2nc(-c3ccccc3)nc(-c3cccc(-c4cccc(-c5ccc6c(ccc7ccc8sc(-c9ccccc9)nc8c76)c5)c4)c3)n2)cc1 ; c1ccc(-c2nc(-c3ccccc3)nc(-c3cc(-c4cccnc4)cc(-c4cc5ccccc5c5ccccc45)c3)n2)cc1] 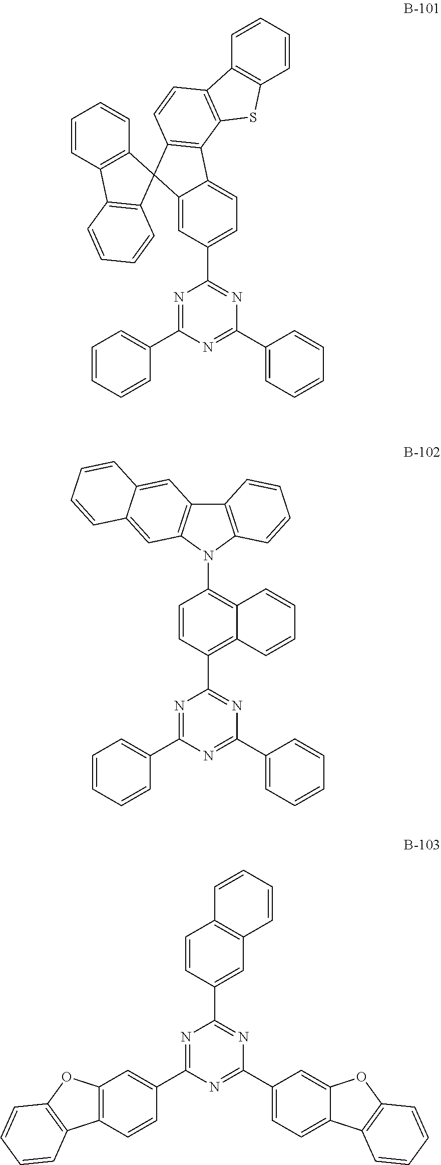 [SMILES: c1ccc(-c2nc(-c3ccccc3)nc(-c3ccc4c(c3)C3(c5ccccc5-c5ccccc53)c3ccc5c(sc6ccccc65)c3-4)n2)cc1 ; c1ccc(-c2nc(-c3ccccc3)nc(-c3ccc(-n4c5ccccc5c5cc6ccccc6cc54)c4ccccc34)n2)cc1 ; c1ccc2cc(-c3nc(-c4ccc5c(c4)oc4ccccc45)nc(-c4ccc5c(c4)oc4ccccc45)n3)ccc2c1] 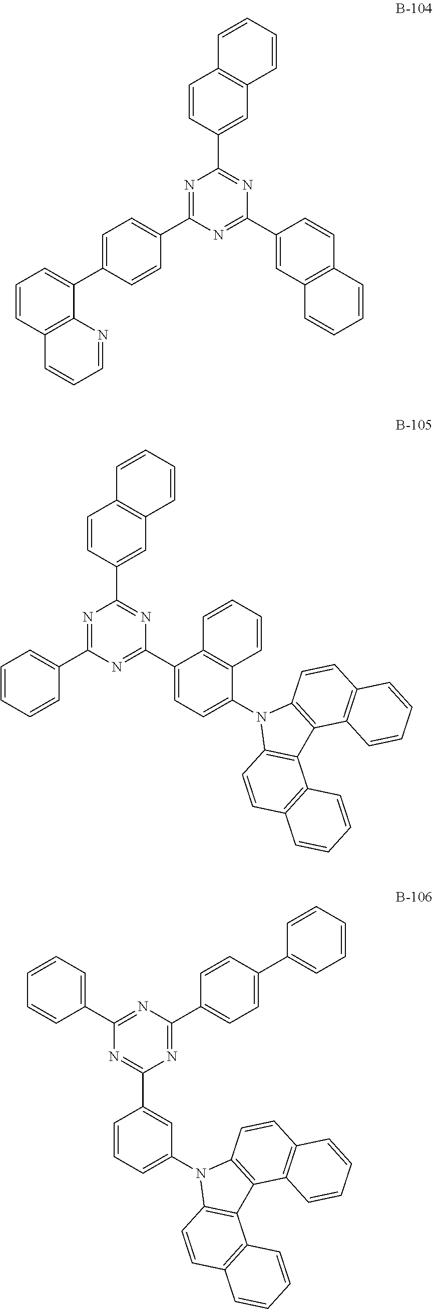 [SMILES: c1ccc2cc(-c3nc(-c4ccc(-c5cccc6cccnc56)cc4)nc(-c4ccc5ccccc5c4)n3)ccc2c1 ; c1ccc(-c2nc(-c3ccc4ccccc4c3)nc(-c3ccc(-n4c5ccc6ccccc6c5c5c6ccccc6ccc54)c4ccccc34)n2)cc1 ; c1ccc(-c2ccc(-c3nc(-c4ccccc4)nc(-c4cccc(-n5c6ccc7ccccc7c6c6c7ccccc7ccc65)c4)n3)cc2)cc1] 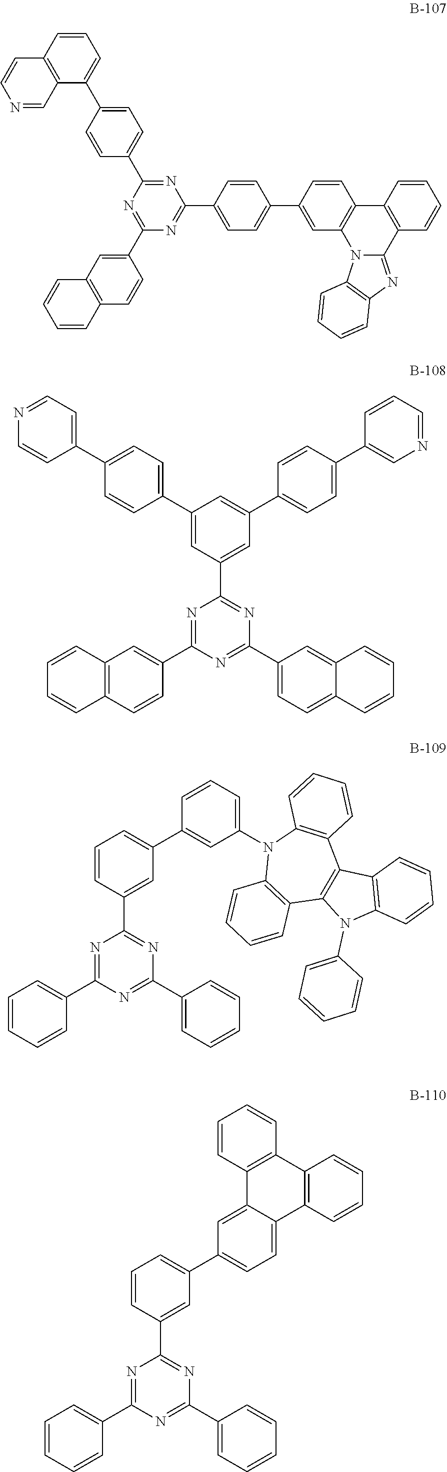 [SMILES: c1ccc2cc(-c3nc(-c4ccc(-c5ccc6c7ccccc7c7nc8ccccc8n7c6c5)cc4)nc(-c4ccc(-c5cccc6ccncc56)cc4)n3)ccc2c1 ; c1cncc(-c2ccc(-c3cc(-c4ccc(-c5ccncc5)cc4)cc(-c4nc(-c5ccc6ccccc6c5)nc(-c5ccc6ccccc6c5)n4)c3)cc2)c1 ; c1ccc(-c2nc(-c3ccccc3)nc(-c3cccc(-c4cccc(N5c6ccccc6-c6c(n(-c7ccccc7)c7ccccc67)-c6ccccc65)c4)c3)n2)cc1 ; c1ccc(-c2nc(-c3ccccc3)nc(-c3cccc(-c4ccc5c6ccccc6c6ccccc6c5c4)c3)n2)cc1] 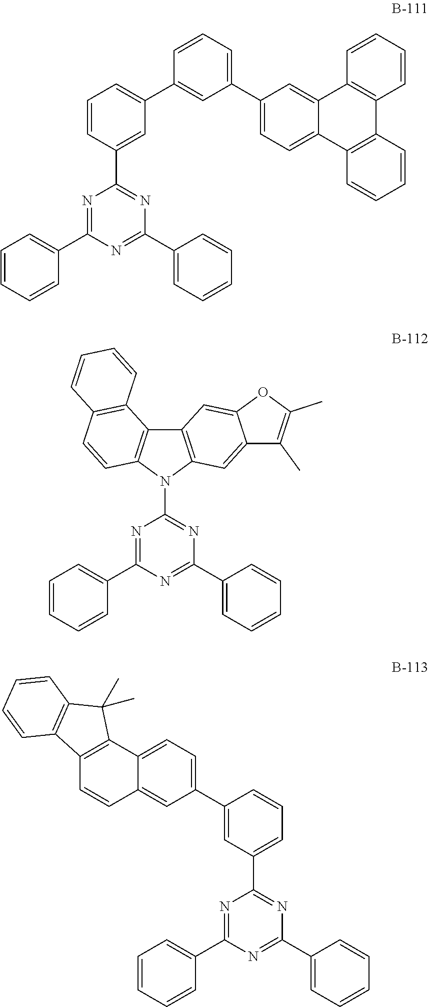 [SMILES: c1ccc(-c2nc(-c3ccccc3)nc(-c3cccc(-c4cccc(-c5ccc6c7ccccc7c7ccccc7c6c5)c4)c3)n2)cc1 ; Cc1oc2cc3c4c5ccccc5ccc4n(-c4nc(-c5ccccc5)nc(-c5ccccc5)n4)c3cc2c1C ; CC1(C)c2ccccc2-c2ccc3cc(-c4cccc(-c5nc(-c6ccccc6)nc(-c6ccccc6)n5)c4)ccc3c21] 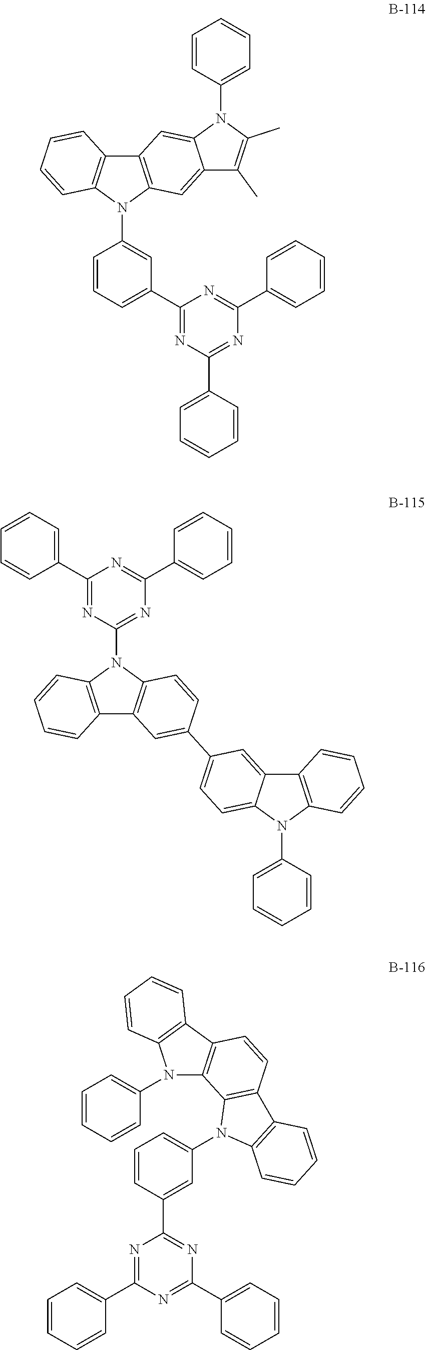 [SMILES: Cc1c(C)n(-c2ccccc2)c2cc3c4ccccc4n(-c4cccc(-c5nc(-c6ccccc6)nc(-c6ccccc6)n5)c4)c3cc12 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3cccc(-n4c5ccccc5c5ccc6c7ccccc7n(-c7ccccc7)c6c54)c3)n2)cc1] 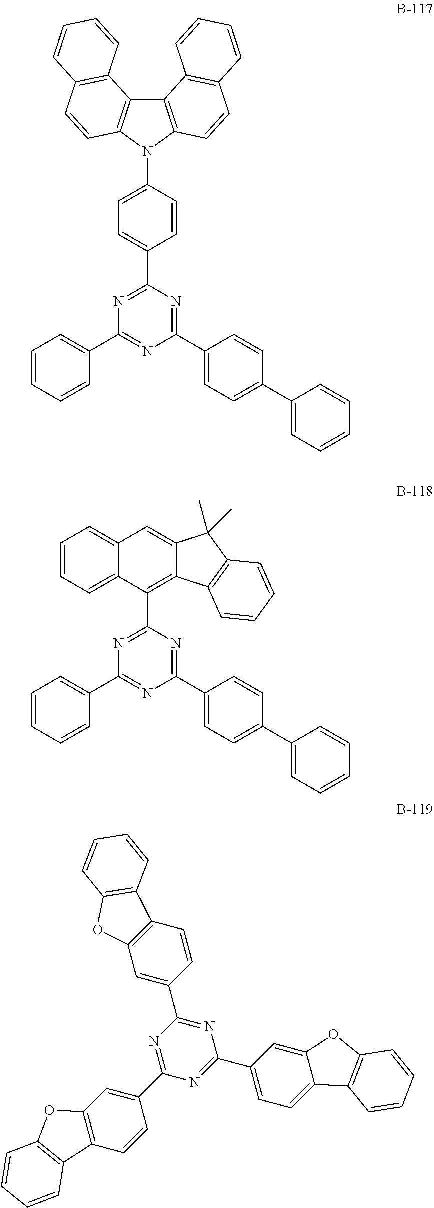 [SMILES: c1ccc(-c2ccc(-c3nc(-c4ccccc4)nc(-c4ccc(-n5c6ccc7ccccc7c6c6c7ccccc7ccc65)cc4)n3)cc2)cc1 ; CC1(C)c2ccccc2-c2c1cc1ccccc1c2-c1nc(-c2ccccc2)nc(-c2ccc(-c3ccccc3)cc2)n1 ; c1ccc2c(c1)oc1cc(-c3nc(-c4ccc5c(c4)oc4ccccc45)nc(-c4ccc5c(c4)oc4ccccc45)n3)ccc12] 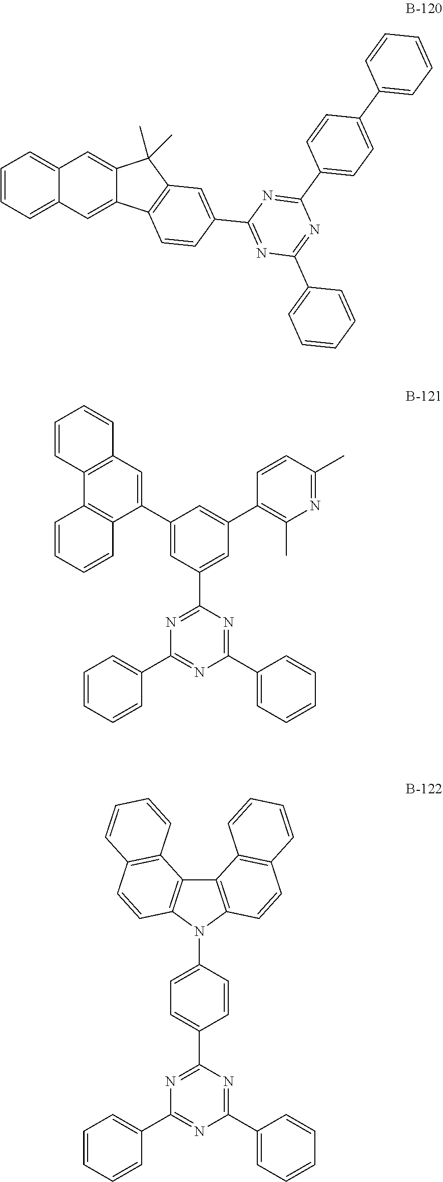 [SMILES: CC1(C)c2cc(-c3nc(-c4ccccc4)nc(-c4ccc(-c5ccccc5)cc4)n3)ccc2-c2cc3ccccc3cc21 ; Cc1ccc(-c2cc(-c3nc(-c4ccccc4)nc(-c4ccccc4)n3)cc(-c3cc4ccccc4c4ccccc34)c2)c(C)n1 ; c1ccc(-c2nc(-c3ccccc3)nc(-c3ccc(-n4c5ccc6ccccc6c5c5c6ccccc6ccc54)cc3)n2)cc1] 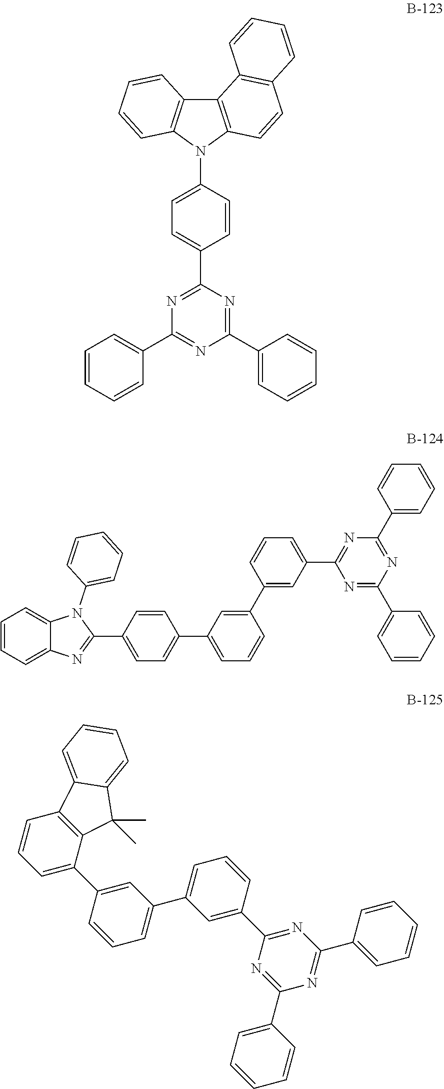 [SMILES: c1ccc(-c2nc(-c3ccccc3)nc(-c3ccc(-n4c5ccccc5c5c6ccccc6ccc54)cc3)n2)cc1 ; c1ccc(-c2nc(-c3ccccc3)nc(-c3cccc(-c4cccc(-c5ccc(-c6nc7ccccc7n6-c6ccccc6)cc5)c4)c3)n2)cc1 ; CC1(C)c2ccccc2-c2cccc(-c3cccc(-c4cccc(-c5nc(-c6ccccc6)nc(-c6ccccc6)n5)c4)c3)c21] 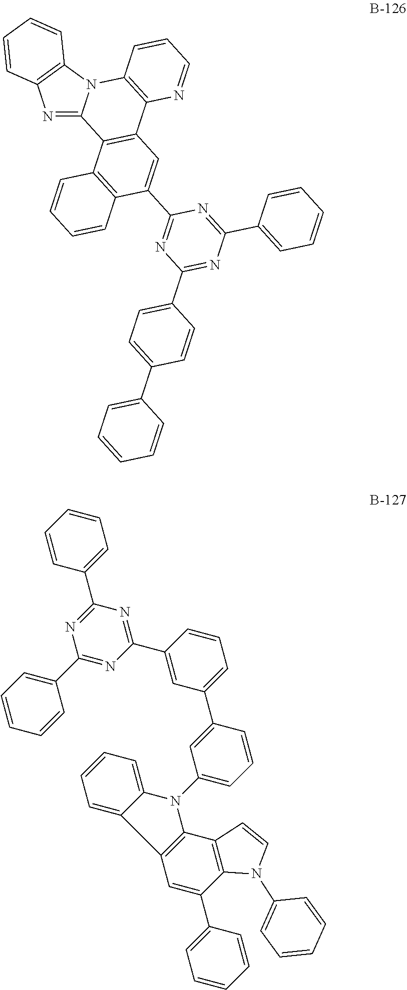 [SMILES: c1ccc(-c2ccc(-c3nc(-c4ccccc4)nc(-c4cc5c6ncccc6n6c7ccccc7nc6c5c5ccccc45)n3)cc2)cc1 ; c1ccc(-c2nc(-c3ccccc3)nc(-c3cccc(-c4cccc(-n5c6ccccc6c6cc(-c7ccccc7)c7c(ccn7-c7ccccc7)c65)c4)c3)n2)cc1] 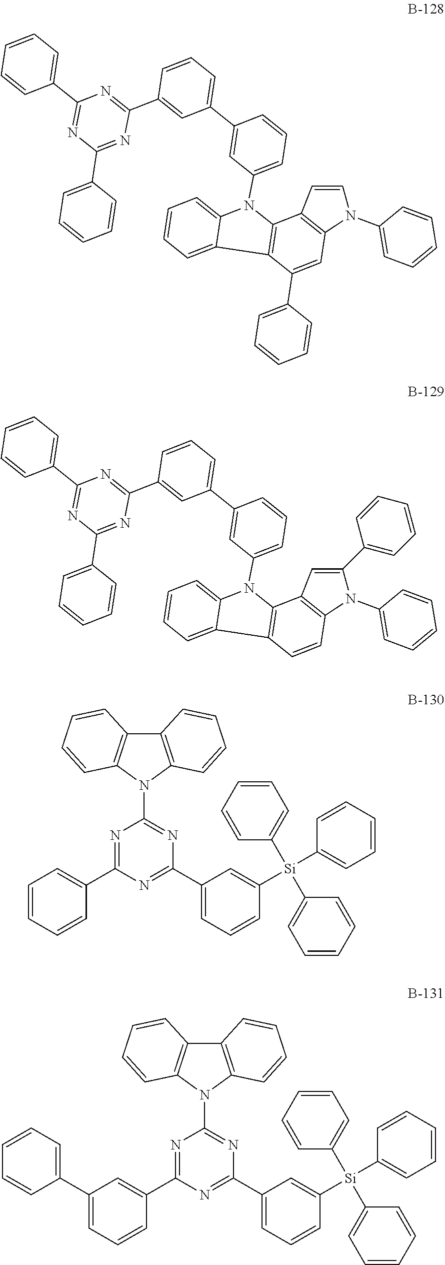 [SMILES: c1ccc(-c2nc(-c3ccccc3)nc(-c3cccc(-c4cccc(-n5c6ccccc6c6c(-c7ccccc7)cc7c(ccn7-c7ccccc7)c65)c4)c3)n2)cc1 ; c1ccc(-c2nc(-c3ccccc3)nc(-c3cccc(-c4cccc(-n5c6ccccc6c6ccc7c(cc(-c8ccccc8)n7-c7ccccc7)c65)c4)c3)n2)cc1 ; c1ccc(-c2nc(-c3cccc([Si](c4ccccc4)(c4ccccc4)c4ccccc4)c3)nc(-n3c4ccccc4c4ccccc43)n2)cc1 ; c1ccc(-c2cccc(-c3nc(-c4cccc([Si](c5ccccc5)(c5ccccc5)c5ccccc5)c4)nc(-n4c5ccccc5c5ccccc54)n3)c2)cc1] 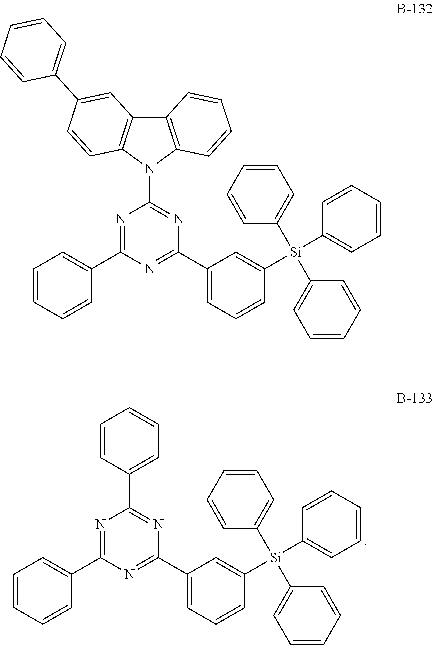 [SMILES: c1ccc(-c2ccc3c(c2)c2ccccc2n3-c2nc(-c3ccccc3)nc(-c3cccc([Si](c4ccccc4)(c4ccccc4)c4ccccc4)c3)n2)cc1 ; c1ccc(-c2nc(-c3ccccc3)nc(-c3cccc([Si](c4ccccc4)(c4ccccc4)c4ccccc4)c3)n2)cc1]
